(19) United States
(12) Patent Application Publication
(10) Pub. No.: US 2018/0186796 A1
(43) Pub. Date: Jul. 5, 2018

(54) COMPOUNDS AND USES THEREOF

(71) Applicant: LEAD DISCOVERY SIENA S.R.L., Siena (SI) (IT)

(72) Inventors: Maurizio BOTTA, Siena (SI) (IT); Adriano ANGELUCCI, Siena (SI) (IT); Elena DREASSI, Siena (SI) (IT); Silvia SCHENONE, Siena (SI) (IT); Cristina TINTORI, Siena (SI) (IT); Giulia VIGNAROLI, Siena (SI) (IT)

(73) Assignee: LEAD DISCOVERY SIENA S.R.L., Siena (SI) (IT)

(21) Appl. No.: 15/520,768

(22) PCT Filed: Oct. 29, 2015
(86) PCT No.: PCT/EP2015/075148

§ 371 Date: Apr. 20, 2017

(30) Foreign Application Priority Data
Oct. 30, 2014 (IT) RM2014A000620

(51) Int. Cl. C07D 487/04; A61K 31/5377; A61K 45/06; A61K 31/519

(52) U.S. Cl. CPC C07D 487/04; A61K 31/519; A61K 45/06; A61K 31/5377

(57) ABSTRACT

The present invention refers to 4-amino-substituted pyrazolo[3,4-d]pyrimidine and pyrrolo[2,3-d]pyrimidine derivatives of formula I and IV able to target the Src family kinases (SFKs) such as Src, Fyn and Hck tyrosine kinases as well as Abl tyrosine kinase and uses and method of preparation thereof. In particular, the compounds of the invention are for use in the treatment and/or prevention of cancer, such as neuroblastoma (NB) or glioblastoma multiforme (GBM) or for use in the treatment and/or prevention of neurodegenerative diseases such as taupathies.

### FIELD OF THE INVENTION

The present invention refers to 4-amino-substituted pyrazolo[3,4-d]pyrimidine and pyrrolo[2,3-d]pyrimidine derivatives of formula I and IV able to target the Src family kinases (SFKs) such as Src, Fyn and Hck tyrosine kinases as well as Abl tyrosine kinase and uses and method of preparation thereof. In particular, the compounds of the invention are for use in the treatment and/or prevention of cancer, such as neuroblastoma (NB) or glioblastoma multiforme (GBM) or for use in the treatment and/or prevention of neurodegenerative diseases such as taupathies.

### STATE OF THE ART

Deregulation of tyrosine kinases (TKs) has been associated with cancer development (proliferation, migration, invasion, angiogenesis, drug resistance etc), therefore small molecule TK inhibitors (TKIs), represent one of the largest drug family currently targeted by pharmaceutical companies and academia for the treatment of malignancies. Remarkably, TKIs, acting on specific molecular targets, could be related with reduced toxic side effects during antitumor treatments. Many TKIs have been tested for their in vitro antiproliferative activity and in vivo anticancer activity, and some of them have been approved in clinical trials or are currently utilized in cancer therapy.^{1,2} A subclass of non-receptor TKs as target in the treatment of human cancers is the Src-family tyrosine kinases (SFKs), which includes nine members such as Src, Fyn, Hck. On the other hand, Abl shares significant sequence homology and remarkable structural resemblance in its active state with Src family members. For this reason, several ATP-competitive inhibitors targeting the active conformation of the enzyme originally developed as Src inhibitors, showed to be also potent Abl inhibitors.^{3a}

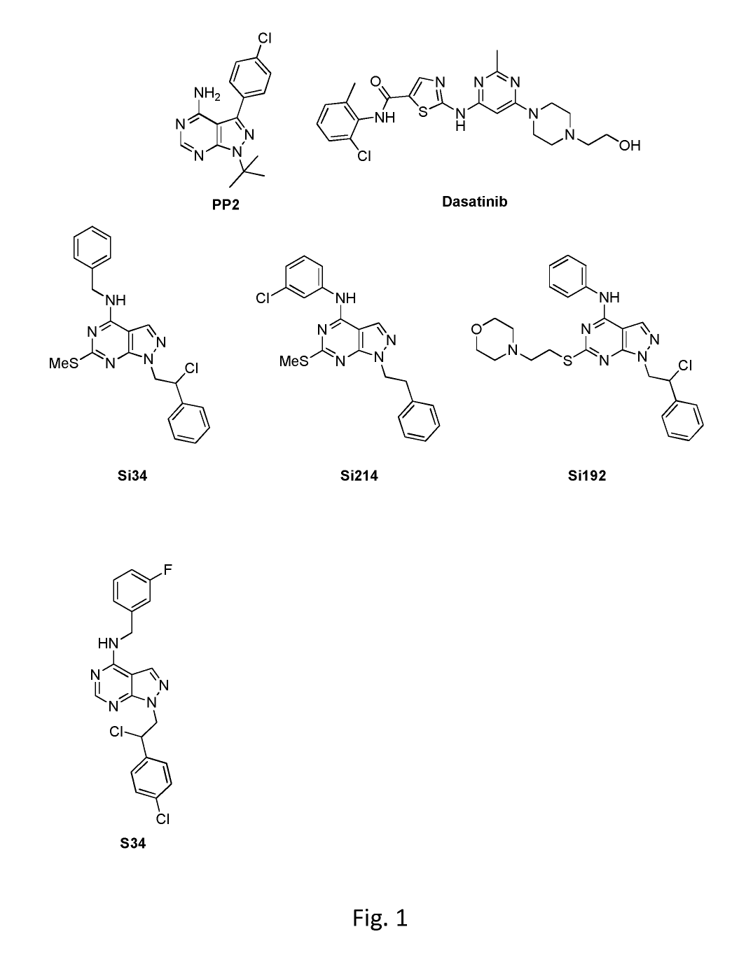

An active and promising field of study is about the role of TKs in modulating the phenotype of tumor-associated cells (TACs), including endothelial cells and fibroblasts. In fact, inhibition of TKs is potentially involved, directly or indirectly, in blocking phenotypic switch of TACs towards a phenotype that contribute to create a favorable tumor microenvironment.^{3b} The best-known symbiosis relation between cancer and stromal cells is determined by differentiation-associated fibroblast in myofibroblasts.^{3c} It was demonstrated that inhibition of signaling pathways, that include several members of TKs family, is able to effectively inhibit cancer progression through the block of cancer-associated fibroblast differentiation.^{3d}NB is a rare cancer of the sympathetic nervous system, where hyperactivation of c-Src plays a key role in the differentiation, cell-adhesion and survival of tumor cells.^{4,5} Recently, the well-known c-Src inhibitor PP2 has recently been proved to inhibit cell survival/proliferation and to reduce aggregation in NB cell lines⁶ while the dual Src/Abl inhibitor dasatinib has been proved to be effective in reducing NB growth both in vitro and in vivo (FIG. 1).⁷

NB accounts for about 9% of malignancies in patients younger than 15 years and for around 15% of all pediatric oncology deaths.⁸ It is the most common extracranial solid tumor in childhood and is a major cause of death from neoplasia in infancy.⁹ Although the substantial improvement in the treatment of certain well-defined subsets of patients, observed during the past few decades, the outcome for children with a high-risk clinical phenotype has improved only modestly, with long-term survival less than 40%.^{10,11} The therapeutic options for the clinical managing of NB consist of a multimodality approach which includes surgery, chemotherapy, radiotherapy, and biotherapy. Current chemotherapeutic treatment for high-risk NB uses dose-intensive cycles of cisplatin and etoposide alternating with vincristine, doxorubicin and cyclophosphamide. Furthermore, isotretinoin could be used during the first remission. Despite improvements in the overall cure rate of these patients, the treatment strategies are still far from satisfaction especially because of the severe side effects.^{12,13} Accordingly, novel therapeutic approaches are needed to ameliorate the prognosis of NB patients.

GBM is the most common and aggressive primary brain tumour, with an extremely poor prognosis and very few therapeutic advances in the last decade.¹⁴ Multiple challenges remain, including tumor heterogeneity, tumor location in a region where it is beyond the reach of local control, and rapid, aggressive tumor relapse. Therefore, the treatment of patients with malignant gliomas still remains palliative and encompasses surgery, radiotherapy and chemotherapy. Radiation therapy in addition to surgery or surgery combined with chemotherapy has been shown to prolong survival in patients with GBM compared to surgery alone. The addition of radiotherapy to surgery has been shown to increase survival from 3-4 months to 7-12 months,¹⁵ any period of response is short-lived because the tumor typically recurs within 1 year, resulting in further clinical deterioration.¹⁶ Different therapeutic targets have been recently identified (e.g. VEGF and EGFR) indicating that targeted therapy could represent a promising strategy. Preclinical data showing that c-Src and SRC-family kinases (SFKs) mediate intracellular signaling pathways controlling key biologic/oncogenic processes provide a strong rationale for investigating SRC/SFK inhibitors.¹⁷

Fyn is a non-receptor tyrosine kinase belonging to the Src family kinases (SFKs).³⁹ The nine members of this family are grouped into sub-classes: the SrcA subfamily which includes Src, Yes, Fyn, and Fgr, the SrcB subfamily containing Lck, Hck, Blk, and Lyn, and finally Frk in its own subfamily. Fyn is a 59-kDa protein comprising 537 amino acids, encoded by the Fyn gene, located on chromosome 6q21. Three isoforms of Fyn are known: fynB mainly expressed in the brain, fynT expressed in hematopoietic cells (T-cells) and fynDelta7 which has been identified in peripheral blood mononuclear cells.⁴⁰ In vertebrates the proteins of SFKs share a similar structure that comprises six distinct functional domains: Src homology domain 4 (SH4), a unique domain, SH3 domain, SH2 domain, a catalytic domain (SH1), and a C-terminal regulatory region. SH4 domain is a region which comprises signals for modification with fatty acids.³⁹ The unique domain is specific for each Src family protein and is suggested to be responsible for specific interactions with particular receptors and protein targets.⁴¹ SH2 and SH3 domains interact with other proteins, and these interactions regulate the tyrosine kinase activity. The kinase domain, that catalyzes the transfer of the terminal phosphate group of the ATP to a tyrosine residue of protein substrate, presents a typical bilobed structure formed by a small N-terminal lobe, involved in the binding with ATP, and larger C-terminal lobe, where an activation loop (A-loop) is present, with a conserved tyrosine residue that is auto-phosphorylated in the active form of the enzyme.⁴² The A-loop contains 28 residues, which are defined in the primary sequence as the region included between two conserved tripeptide motifs, DFG (Asp-Phe-Gly) and APE (Ala-Pro-Glu).⁴³ Besides to share the same structure, the SFKs are also characterized by the same regulatory mechanisms. In fact, the activation or inhibition of kinase activity depends on intramolecular interactions between SH2 and SH3 with kinase domain and on phosphorilation/dephosphorilation of two critical tyrosines, the first situated in the A-loop and the second in correspondence of the C-terminal region.⁴⁴ Fyn protein is able to interact with almost 300 different proteins and, through these interactions, participates in many cellular pathways, both in physiological and pathological situations. Fyn is involved in the regulation of the immune system, and in T-cell development and activation.⁴⁵ It plays a crucial role in the development of central nervous system (CNS) where is implied in myelination, morphological differentiation associated with the formation of neurite in oligodendrocytes, synapse formation and regulation, oligodendrocyte differentiation and memory formation.⁴⁶

Recent evidences suggest that Fyn hyperactivation/deregulation might contribute to Alzheimer disease (AD) pathogenesis and other tauopathies. These diseases are characterized by the alteration in the amount or the structure of the Tau protein, a microtubule-associated protein that constitutes a fundamental component of the neurofibrillary tangles of AD.⁴⁷ In normal neurons Tau is present in the cytoplasm in an unphosphorylated form. On the contrary, Tau results phosphorylated at multiple sites in AD. In particular, when associated to neurofibrillary tangles, Tau was found to be phosphorylated at its amino terminus residue Tyr18,⁴⁹ with Fyn being the solely kinase responsible for such event in AD. Mounting evidences suggest that the phopsphorylation of Tyr18 is an early event in the pathophysiology of AD that leads to conformational changes in Tau, initiating its fibrillarization.^{48a} In addition, amyloid-beta (AP) was found to activate Fyn;^{48b} moreover, overexpression of Fyn accelerate synapse loss and the onset of cognitive impairment in transgenic AD mouse model, while inhibition of Fyn expression rescued synapse loss. The AD therapeutic approaches now in clinical trials are focused on AP clearance or in the inhibition of its production or aggregation. Therefore, due to its central to AP signal transduction, Fyn represents a unique therapeutic target in AD. Fyn overexpression has been shown to drive a morphologic transformation in normal cells, leading to tumor development. In fact Fyn is overexpressed in various cancers, including glioblastoma multiformae, squamous cell carcinoma of the head and neck, melanoma,⁵⁰ breast,⁵¹ ovarian,⁵² prostate,⁵³ and pancreatic cancer.⁵⁴ Recent studies have shown its involvement also in mesothelioma.⁵⁵ Lately, Singh and colleagues⁵⁶ demonstrated that Fyn kinase activity plays a role in the progression of chronic myeloid leukemia (CML), because it contributes to BCR-ABL1 induced genomic instability, a feature of blast crisis CML.⁵⁷ The terminal, blast crisis phase of the disease remains a clinical challenge. Blast crisis CML is difficult to treat due to resistance to tyrosine kinase inhibitors, increased genomic instability and acquired secondary mutations. Knockdown of Fyn leads to decreased cell growth and proliferation in vitro and in vivo. Moreover, the group demonstrated that the complete loss of Fyn using genetic knockout models decreases the proliferation and clonogenic potential of cells transduced with BCR-ABL1 underscoring a dependency upon Fyn for BCR-ABL1 mediated growth and clonogenicity. Additionally, using a cell line model of blast crisis CML, they discovered that overexpression of constitutively active Fyn caused increased aneuploidy and genomic alterations. Because of the involvement of Fyn in such disease, the search for Fyn inhibitors represents an expanding field of studies.

### SUMMARY OF THE INVENTION

In recent years, the inventors' group conducted extensive studies on a series of novel Src, Abl, Fyn and Hck inhibitors characterized by a pyrazolo[3,4-d]pyrimidine and pyrrolo[2,3-d]pyrimidine scaffold.¹⁸ Several members of this family were found to induce apoptosis and reduce cell proliferation in different solid tumor cell lines (A431, 8701-BC, SaOS-2, and PC3). A selected member of this family, Si34 characterized by a C6 methylthio group on the pyrazolo[3,4-d]pyrimidine scaffold, displayed a promising antiproliferative activity in SH-SY5Y cell cultures of human NB (FIG. 1).¹⁹ In order to get further insight into the potentiality of such pyrazolo[3,4-d]pyrimidines for the treatment of solid tumors, such as NB and GBM, a small collection of closely related analogues characterized by the presence of a C6 methylthio group was synthesized to explore the role of different functional groups in N1 and C4 for the biological activity. Among the synthesized analogues, compound Si214, was characterized by a potent inhibitory activity against c-Src (Kᵢ=90 nM) and a considerable antiproliferative effect on SH-SY5Y NB cells (IC₅₀=80 nM) (FIG. 1). However, despite its remarkable activities, this compound suffers from a low water solubility (0.12 μg/mL) which precludes oral administration.²⁰ Accordingly, a series of more soluble pyrazolo[3,4-d]pyrimidine derivatives has been rationally designed and synthesized by the inventors' research group through the introduction of polar groups in the solvent-exposed C6 position. This study led to the identification of the C4-anilino derivative Si192 which showed a beneficial profile in term of both biological activity and ADME properties, being characterized by a high metabolic stability (95%), a good water solubility (1.7 μg/mL), an efficient membrane permeability (10×10⁶ cm/s) and a potent inhibitory activity against isolated c-Src (Kᵢ=0.21 μM).²¹ Herein, starting from Si192 data, the authors developed a second-generation inhibitors, endowed with improved affinity towards c-Src and improved ADME properties, to be tested against NB and GBM in vivo (FIG. 1). To this aim, a multidisciplinary approach combining X-ray crystallography, structure-based drug design, synthesis, in vitro ADME profiling and in vitro/in vivo biological evaluation, was applied. Starting from the crystallographic complex of Si192 and c-Src, an efficient optimization of the pyrazolo[3,4-d]pyrimidine substituents has been guided by free energy perturbation (FEP) calculations to direct the synthesis of c-Src inhibitors, herein showed, many of which are endowed with nanomolar potencies.

A subset of compounds also showed a strong antiproliferative activity against NB and GBM cells as well as optimal ADME characteristics. The compounds of the invention inhibited the proliferation of NB and GBM cell lines and demonstrated in vivo activity, displaying good ADME properties (in particular in terms of membrane permeability) and showing increased water solubility when compared with the previously reported compounds Si192 and Si181. Accordingly, further studies were conducted on compound Si306, one of the most promising derivatives, in order to test its efficacy against NB and GBM in vivo after oral administration in mice. In NB mice model, tumour growth was significantly inhibited by compound Si306 at the dose of 50 mg/kg. Subsequent observations on excised tumor masses and in vitro assays suggest that c-Src inhibitor was active on both cancer cells and tumor-associated endothelial cells inhibiting their migratory capacity and angiogenesis.

Furthermore, Si306 was administered in vivo to nude mice inoculated subcutaneously with U87 GBM cells. Mice received 50 mg/kg of Si306 every other day and the antitumoral effect of the compound was also evaluated in combination with a single radiotherapic treatment (4Gy). At the endpoint, mice that received the combination therapy showed an 80% reduction of the tumor mass.

The combination therapy of Si306 plus radiotherapy was evaluated also in vitro (U87 cells) by a low density growth assay, again the combination therapy reduced significantly the number of colonies in respect to control and to single treatments.

Si306 was tested also in combination with mitomycin C—a well known genotoxic agent—in U87 and U251 cells model; the combination treatment determined a synergic antiproliferative effect that was more pronounced in U87 cells.

Moreover, prodrugs of the compounds, described in this invention, were also synthetized in order to further enhance water solubility, in fact the improvement of this phannacokinetic property could positively influence the in plasma—as well as in vivo—distribution. Prodrugs showed a general improvement of activity towards cancer cell lines NB and GBM cancer cell lines, when compared to their respective drugs. In vivo biodistribution demonstrated the in vivo hydrolysis of proSi306 and its ability to yield the highest brain and plasma concentration.

In the present invention a structure-based drug design protocol was employed aimed at identifying novel Fyn inhibitors. Fyn is a member of the Src-family of non-receptor protein-tyrosine kinases (SFKs). Its abnormal activity has been shown to be related to various human cancers as well as to severe pathologies, such as Alzheimer's and Parkinson's diseases, thus making Fyn an attractive target for the identification of novel therapeutic agents to tauopathies and tumors.

First, a virtual screening approach was applied to screen a database of commercially available compounds by the use of docking studies within the ATP binding site of Fyn. Next, an in house library of pyrazolo[3,4-d]pyrimidine derivatives, which have previously shown to be dual Abl and c-Src inhibitors, was analysed by the same computational protocol. Slightly modifications aimed at optimizing the van der Waals contacts of the ligand within the hydrophobic region I rapidly determine an increase in the binding affinity, with the best inhibitors Si310 and Si308 having Ki of 70 nM and 95 nM, respectively. Remarkably, both compounds showed an interesting antiproliferative activity profile against the Chronic Myelogeneous Leukemia cell line K562 and were found able to inhibit the Fyn-mediated phosphorylation of the protein Tau in an Alzheimer's disease model cell line.

The present invention provides a compound of formula I

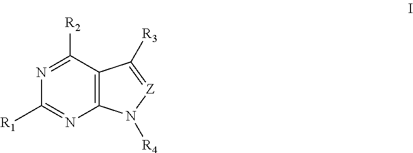

or a stereoisomer or a prodrug or a pharmaceutically acceptable salt thereof,
wherein Z represents CH or N;
R₁ represents alkyl chain with the formula:

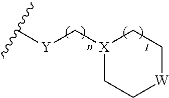

where Y is NH or O or S; X is CH or N; W is NH or NCH₃ or O; n is an integer from 0 to 4; i is an integer from 0 to 1;
or:

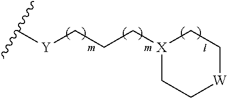

where Y is NH or O or S; V is cyclopropyl or cyclopentyl or cyclohexyl; X is CH or N; W is NH or NCH₃ or O; m is an integer from 0 to 2; i is an integer from 0 to 1;
or:

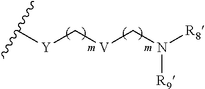

where Y is NH or O or S; V is cyclopropyl or cyclopentyl or cyclohexyl; R₈′ and R₉′ are independently H or CH₃; m is an integer from 0 to 2; i is an integer from 0 to 1;
or:

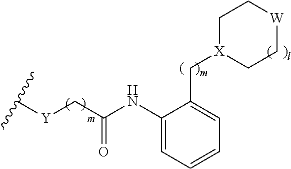

where Y is NH or O or S; X is CH or N; W is NH or NCH₃ or O; m is an integer from 0 to 2; i is an integer from 0 to 1;
R₂ represents NR₁₀′R₁₁′;
R₁₀′ and R₁₁′ are independently H, alkyl, cycloalkyl, 1-pyrrolidinyl, 4-morpholinyl, 1-hexahydroazepinyl;
or an aralkyl with the formula:

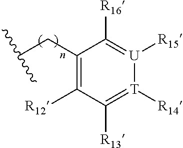

where T and U are independently C or N;
R₁₂′, R₁₃′, R₁₄′, R₁₅′, R₁₆′ are independently H, C₁₋₆ alkyl, C₂₋₆ alkenyl, C₂₋₆ alkynyl, aryl unsubstituted or substituted group, halo, haloalkyl, OCH₃, NO₂, CN, CONH₂, CONH—C₁₋₆ alkyl, CON(C₁₋₆ alkyl)₂, NH₂, NH—C₁₋₆ alkyl, N(C₁₋₆ alkyl)₂, NHC(O)alkyl, NHSO₂C₁₋₆ alkyl, SO₂NH₂, SO₂NHC₁₋₆ alkyl, SO₂N(C₁₋₆ alkyl)₂, OQ′ or SQ′ where Q′ is H, or alkyl unsubstituted or substituted group, or aryl unsubstituted or substituted group, or aralkyl unsubstituted or substituted group, n is an integer from 0 to 4;
or:

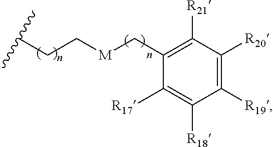

preferably

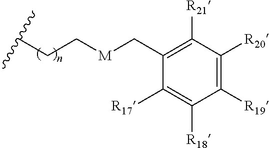

where M is NH or S or O;
R₁₇′, R₁₈′, R₁₉′, R₂₀′, R₂₁′ are independently H, C₁₋₆ alkyl, C₂₋₆ alkenyl, C₂₋₆ alkynyl, aryl unsubstituted or substituted group, halo, haloalkyl, OCH₃, NO₂, CN, CONH₂, CONH—C₁₋₆ alkyl, CON(C₁₋₆ alkyl)₂, NH₂, NH—C₁₋₆ alkyl, N(C₁₋₆ alkyl)₂, NHC(O)alkyl, NHSO₂C₁₋₆ alkyl, SO₂NH₂, SO₂NHC₁₋₆ alkyl, SO₂N(C₁₋₆ alkyl)₂, OQ′ or SQ′ where Q′ is H, or alkyl unsubstituted or substituted group, or aryl unsubstituted or substituted group, or aralkyl unsubstituted or substituted group; n is an integer from 0 to 4;
R₃ represents H
or an aralkyl with the formula:

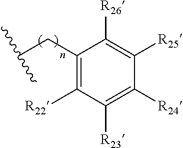

where R₂₂′, R₂₃′, R₂₄′, R₂₅′, R₂₆′ are independently H, C₁₋₆ alkyl, C₂₋₆ alkenyl, C₂₋₆ alkynyl, aryl unsubstituted or substituted group, halo, haloalkyl, OCH₃, NO₂, CN, CONH₂, CONH—C₁₋₆ alkyl, CON(C₁₋₆ alkyl)₂, NH₂, NH—C₁₋₆ alkyl, N(C₁₋₆ alkyl)₂, NHC(O)alkyl, NHCONH—C₁₋₆ alkyl, NHSO₂C₁₋₆ alkyl, SO₂NH₂, SO₂NHC₁₋₆ alkyl, SO₂N(C₁₋₆ alkyl)₂, OQ′ or SQ′ where Q′ is H, or alkyl unsubstituted or substituted group, or aryl unsubstituted or substituted group, or aralkyl unsubstituted or substituted group; n is an integer from 0 to 4;
or:

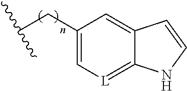

where L is CH or N; n is an integer from 0 to 4;
R₄ represents:

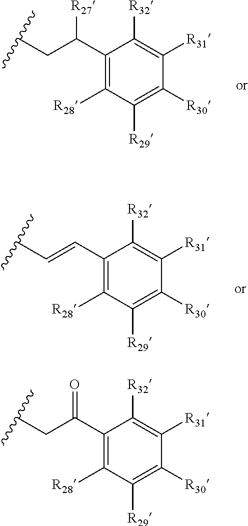

where R₂₇′ represents H, CH₃, CF₃, F, Cl, Br, OH; OMe, O-alkyl, alkyl;
where R₂₈′, R₂₉′, R₃₀′, R₃₁′, R₃₂′ are independently H, C₁₋₆ alkyl, C₂₋₆ alkenyl, C₂₋₆ alkynyl, aryl unsubstituted or substituted group, halo, haloalkyl, OCH₃, NO₂, CN, CONH₂, CONH—C₁₋₆ alkyl, CON(C₁₋₆ alkyl)₂, NH₂, NH—C₁₋₆ alkyl, N(C₁₋₆ alkyl)₂, NHC(O)alkyl, NHSO₂C₁₋₆ alkyl, OQ′ or SQ′ where Q′ is H, or alkyl unsubstituted or substituted group, or aryl unsubstituted or substituted group, or aralkyl unsubstituted or substituted group; SO₂NH₂, SO₂NHC₁₋₆ alkyl, SO₂N(C₁₋₆ alkyl, SO₂H, SO₂CH₃, PO₂, PO(CH₃)₂, POHCH₃, POH₂, SO₂J where J is:

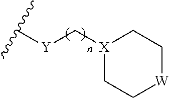

where Y is NH or O or S; X is CH or N; W is NH or NCH₃ or O; n is an integer from 0 to 4;
with the provisio that compounds:
1-(2-chloro-2-phenylethyl)-6-((2-morpholinoethyl)thio)-N-propyl-1H-pyrazolo[3,4-d]pyrimidin-4-amine (Si109);
N-benzyl-1-(2-chloro-2-phenylethyl)-6-((2-morpholinoethyl)thio)-1H-pyrazolo[3,4-d]pyrimidin-4-amine (Si110);
1-(2-chloro-2-phenylethyl)-N-(4-fluorobenzyl)-6-((2-morpholinoethyl)thio)-1H-pyrazolo[3,4-d]pyrimidin-4-amine (Si180);
1-(2-chloro-2-phenylethyl)-6-((2-morpholinoethyl)thio)-N-phenethyl-1H-pyrazolo[3,4-d]pyrimidin-4-amine (Si182);
1-(2-chloro-2-phenylethyl)-N-(3-chlorophenyl)-6-((2-morpholinoethyl)thio)-1H-pyrazolo[3,4-d]pyrimidin-4-amine (Si181);
1-(2-chloro-2-phenylethyl)-6-((2-morpholinoethyl)thio)-N-phenyl-1H-pyrazolo[3,4-d]pyrimidin-4-amine (Si192);
N-cyclohexyl-6-(2-morpholinoethoxy)-1-phenethyl-1H-pyrazolo[3,4-d]pyrimidin-4-amine (Sv2);
N⁴-(3-chlorophenyl)-N⁶-(2-morpholinoethyl)-1H-phenethyl-1H-pyrazolo[3,4-d]pyrimidine-4,6-diamine (Sv24);
2-(4-methylpiperazin-1-yl)ethyl butyl(1-(2-chloro-2-phenylethyl)-6-(ethylthio)-1H-pyrazolo[3,4-d]pyrimidin-4-yl)carbamate (proSi20);
2-(4-methylpiperazin-1-yl)ethyl (3-bromophenyl)(6-(methylthio)-1-(2-phenylpropyl)-1H-pyrazolo[3,4-d]pyrimidin-4-yl)carbamate (proSi278);
1-(2-chloro-2-phenylethyl)-N-(3-chlorobenzyl)-6-(3-morpholinopropyl)-1H-pyrazolo[3,4-d]pyrimidin-4-amine;
   and compounds of formula A

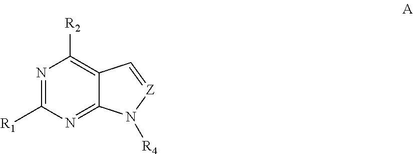

wherein when Z═N, R₁═SCH₂CH₂-4-morpholinyl and R₂ is NHCH₂CH₂C₆H₅, NHCH₂C₆H₅, NHC₆H₄mCl, 1-hexahydroazepinyl, NHC₃H₇, 4-morpholinyl or NHCH₂C₆H₄pCl are excluded.

Preferably Z is N, and/or R₁ is SCH₂CH₂4-morpholinyl and/or R₂ is NHC₆H₅ or NHC₆H₄mCl or NHC₆H₄mF or NHC₆H₄nBr or NHC₆H₄mOH and/or R₃ is H and/or R₄ is CH₂CH₂C₆H or CH₂CHClC₆H or CH₂CHMeC₆H₅ or CH₂CH₂C₆H₄ pF.

Preferably the compound is:
N-(3-Chlorophenyl)-6-[(2-morpholin-4-ylethyl)thio]-1-(2-phenylethyl)-1H-pyrazolo[3,4-d]pyrimidin-4-amine (Si303);
1-(2-chloro-2-phenylethyl)-N-(2-fluorobenzyl)-6-((2-morpholinoethyl)thio)-1H-indazol-4-amine (Si304);
6-[(2-Morpholin-4-ylethyl)thio]-N-phenyl-1-(2-phenylpropyl)-1H-pyrazolo[3,4-d]pyrimidin-4-amine (Si313);
N-(3-Fluorophenyl)-6-[(2-morpholin-4-ylethyl)thio]-1-(2-phenylpropyl)-1H-pyrazolo[3,4-d]pyrimidin-4-amine (Si314);
N-(3-Chlorophenyl)-6-[(2-morpholin-4-ylethyl)thio]-1-(2-phenylpropyl)-1H-pyrazolo[3,4-d]pyrimidin-4-amine (Si307);
N-(3-Chlorophenyl)-1-[2-(4-fluorophenyl)ethyl]-6-[(2-morpholin-4-ylethyl)thio]-1H-pyrazolo[3,4-d]pyrimidin-4-amine (Si327);
N-(3-Bromophenyl)-1-(2-chloro-2-phenylethyl)-6-[(2-morpholin-4-ylethyl)thio]-1H-pyrazolo[3,4-d]pyrimidin-4-amine (Si306);
3-{[6-[(2-Morpholin-4-ylethyl)thio]-1-(2-phenylethyl)-1H-pyrazolo[3,4-d]pyrimidin-4-yl]amino}phenol hydrochloride (Si332);
3-{[6-[(2-Morpholin-4-ylethyl)thio]-1-(2-phenylpropyl)-1H-pyrazolo[3,4-d]pyrimidin-4-yl]amino}phenol hydrochloride (Si329);
1-(2-Chloro-2-phenylethyl)-3-(4-fluorophenyl)-1H-pyrazolo[3,4-d]pyrimidin-4-amine (Si310);
3-(4-Chlorophenyl)-1-(2-chloro-2-phenylethyl)-1H-pyrazolo[3,4-d]pyrimidin-4-amine (Si308);
1-(2-Chloro-2-phenylethyl)-3-(4-methylphenyl)-1H-pyrazolo[3,4-d]pyrimidin-4-amine (Si309);
1-(2-Chloro-2-phenylethyl)-3-(4-methyoxyphenyl)-1H-pyrazolo[3,4-d]pyrimidin-4-amine (Si311);
1-(2-Chloro-2-phenylethyl)-3-phenyl-1H-pyrazolo[3,4-d]pyrimidin-4-amine hydrochloride (Si244);
3-Phenyl-1-(2-phenylpropyl)-1H-pyrazolo[3,4-d]pyrimidin-4-amine (Si312);
1-{4-[4-Amino-1-(2-phenylpropyl)-1H-pyrazolo[3,4-d]pyrimidin-3-yl]phenyl}ethanone (Si336);
3-(4-Chlorophenyl)-1-(2-phenylpropyl)-1H-pyrazolo[3,4-d]pyrimidin-4-amine (Si337);
3-(4-Methylphenyl)-1-(2-phenylpropyl)-1H-pyrazolo[3,4-d]pyrimidin-4-amine (Si338);
3-(1H-indol-5-yl)-1-(2-phenylpropyl)-1H-pyrazolo[3,4-d]pyrimidin-4-amine (Si339);
N-benzyl-6-(sec-butylthio)-1-(2-chloro-2-phenylethyl)-1H-pyrazolo[3,4-d]pyrimidin-4-amine (Si146);
6-(Sec-butylthio)-1-(2-chloro-2-phenylethyl)-N-(2-phenylethyl)-1H-pyrazolo[3,4-d]pyrimidin-4-amine (Si147);
1-(2-Chloro-2-phenylethyl)-6-(cyclopentylthio)-N-(3-fluorophenyl)-1H-pyrazolo[3,4-d]pyrimidin-4-amine (Si170);
6-(Sec-butylthio)-N-(3-chlorophenyl)-1-(2-chloro-2-phenylethyl)-1H-pyrazolo[3,4-d]pyrimidin-4-amine (Si148);
Synthesis of 2-(4-benzylamino-1-styryl-1H-pyrazolo[3,4-d]pyrimidin-6-ylamino)-ethanol (Si74);
N-[2-(3-chlorophenyl)ethyl]-6-(methylthio)-1-[2-phenylvinyl]-1H-pyrazolo[3,4-d]pyrimidin-4-amine (Si215);
N,6-dibenzyl-1-(2-chloro-2-phenylethyl)-1H-pyrazolo[3,4-d]pyrimidin-4-amine (Si164);
   or a stereoisomer or a pharmaceutically acceptable salt thereof.

Preferably the prodrug is a prodrug of formula III

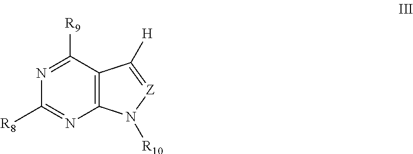

wherein Z represents CH or N;
R₈ represents H, alkylthio, alkylamino, cycloalkyl, cycloalkylthio, cycloalkylamino, alkyl, S(CH₂)ₚOH, S(CH₂)ₚNH₂, S(CH₂)ₚNHCH₃, S(CH₂)ₚN(CH₃)₂, NH(CH₂)ₚOH, NH(CH₂)₁NH₂; NH(CH₂)ₚNHCH₃, NH(CH₂)ₚNH(CH₃)₂; p is an integer from 0 to 6;
or an alkyl chain with the formula:

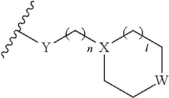

where Y is NH or O or S; X is CH or N; W is NH or NCH₃ or O; n is an integer from 0 to 4; i is an integer from 0 to 1;
or:

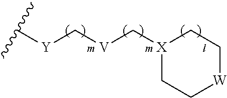

where Y is NH or O or S; V is cyclopropyl or cyclopentyl or cyclohexyl; X is CH or N; W is NH or NCH₃ or O; m is an integer from 0 to 2; i is an integer from 0 to 1;
or:

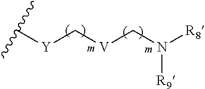

where Y is NH or O or S; V is cyclopropyl or cyclopentyl or cyclohexyl; R₈′ and R₉′ are independently H or CH₃; m is an integer from 0 to 2;
or:

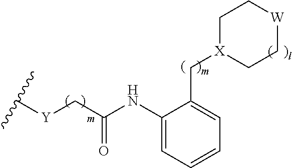

where Y is NH or O or S; X is CH or N; W is NH or NCH₃ or O; m is an integer from 0 to 2; i is an integer from 0 to 1;
R₉ represents:

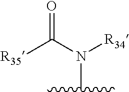

where R₃₄′ is H or alkyl or cycloalkyl or 1-pyrrolidinyl or 4-morpholinyl or 1-hexahydroazepinyl;
or an alkyl chain with the formula:

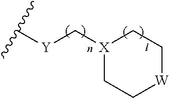

where Y is NH or O or S; X is CH or N; W is NH or NCH₃ or O; n is an integer from 0 to 4; i is an integer from 0 to 1;
or an aralkyl with the formula:

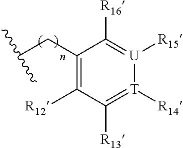

where T and U are independently C or N;
R₁₂′, R₁₃′, R₁₄′, R₁₅′, R₁₆′ are independently H, C₁₋₆ alkyl, C₂₋₆ alkenyl, C₂₋₆ alkynyl, aryl unsubstituted or substituted group, halo, haloalkyl, OCH₃, NO₂, CN, CONH₂, CONH—C₁₋₆ alkyl, CON(C₁₋₆ alkyl)₂, NH₂, NH—C₁₋₆ alkyl, N(C₁₋₆ alkyl)₂, NHC(O)alkyl, NHCONH—C₁₋₆ alkyl, NHSO₂—C₁₋₆ alkyl, SO₂NH₂, SO₂NHC₁₋₆ alkyl, SO₂N(C₁₋₆ alkyl)₂, OQ′ or SQ′ where Q′ is H, or alkyl unsubstituted or substituted group, or aryl unsubstituted or substituted group, or aralkyl unsubstituted or substituted group; n is an integer from 0 to 4;
or:

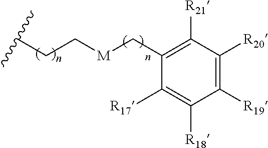

preferably

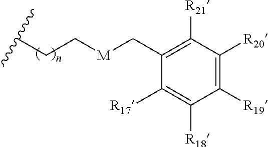

where M is NH or S or O;
R₁₇′, R₁₈′, R₁₉′, R₂₀′, R₂₁′ are independently H, C₁₋₆ alkyl, C₂₋₉ alkenyl, C₂₋₆ alkynyl, aryl unsubstituted or substituted group, halo, haloalkyl, OCH₃, NO₂, CN, CONH₂, CONH—C₁₋₆, alkyl, CON(C₁₋₆ alkyl)₂, NH₂, NH—C₁₋₆ alkyl, N(C₁₋₆ alkyl)₂, NHC(O)alkyl, NHSO₂C₁₋₆ alkyl, SO₂NH₂, SO₂NHC₁₋₆ alkyl, SO₂N(C₁₋₆ alkyl)₂, OQ′ or SQ′ where Q′ is H, or alkyl unsubstituted or substituted group, or aryl unsubstituted or substituted group, or aralkyl unsubstituted or substituted group; n is an integer from 0 to 4;
where R₃₅′ is an alkyl chain with the formula:

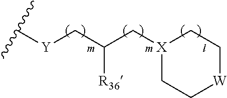

where Y is NH or O or S; R₃₆′ is H or alkyl or aryl or aralkyl; X is CH or N; W is NH or NCH₃ or O; m is an integer from 0 to 2; i is an integer from 0 to 1;
or:

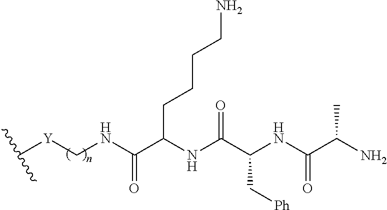

where Y is NH or O or S;
n is an integer from 0 to 4;
or:

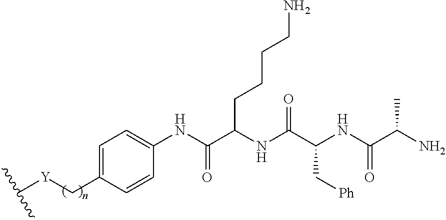

where Y is NH or O or S;
n is an integer from 0 to 4;
R₁₀ represents:

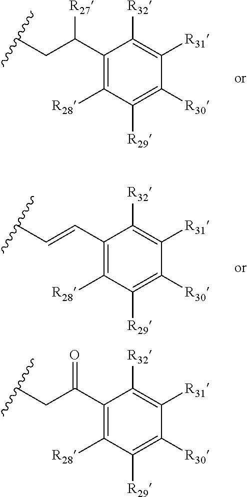

where R₂₇′ represents H, CH₃, CF₃, F, Cl, Br, OH; O-alkyl, alkyl;
where R₂₈′, R₂₉′, R₃₀′, R₃₁′, R₃₂′ are independently H, C₁₋₆ alkyl, C₂₋₆ alkenyl, C₂₋₆ alkynyl, aryl unsubstituted or substituted group, halo, haloalkyl, OCH₃, NO₂, CN, CONH₂, CONH—C₁₋₆ alkyl, CON(C₁₋₆-alkyl)₂, NH₂, NH—C₁₋₆ alkyl, N(C₁₋₆ alkyl)₂, NHC(O)alkyl, NHSO₂—C₁₋₆ alkyl, OQ′ or SQ′ where Q′ is H, or alkyl unsubstituted or substituted group, or aryl unsubstituted or substituted group, or aralkyl unsubstituted or substituted group; SO₂NH₂, SO₂NHC₁₋₆ alkyl, SO₂N(C₁₋₆ alkyl)₂, SO₂H, SO₂CH₃, PO₂, PO(CH₃)₂, POHCH₃, POH₂, SO₂J where J is:

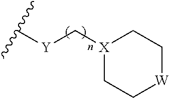

where Y is NH or O or S; X is CH or N; W is NH or NCH₃ or O; n is an integer from 0 to 4. Still preferably in the prodrug, Z is N and/or R₈ is H or SMe or SEt or SCH₂CH₂-4-mopholino; and/or

### R₉ is

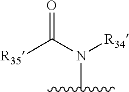

wherein R_{34′} is CH₂C₆H₅ or CH₂C₆H₄oCl or C₆H₄mCl or CH₄mBr or CH₂CH₂C₆H₅ or C₆H₅ or nBu; and wherein R₃₅′ is

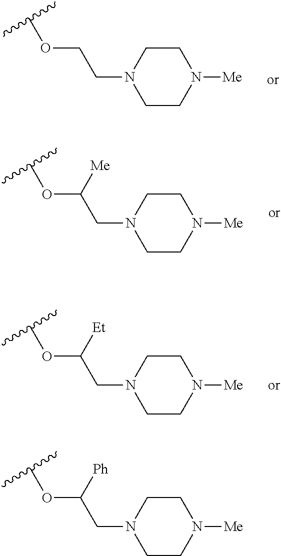 [SMILES: *OCCN1CCN(C)CC1 ; *OC(C)CN1CCN(C)CC1 ; *OC(CC)CN1CCN(C)CC1 ; *OC(CN1CCN(C)CC1)c1ccccc1]

and/or R₁₀ is

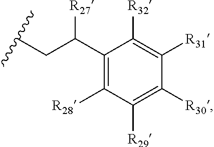

wherein R₂₇′ is H or C₁ or Me; R_{30′} is H or Br; and R₂₈′, R₂₉′, R₃₁′, R₃₂′ are H.

In a preferred embodiment the compounds of the invention are for medical use, preferably for use as SFKs inhibiting medicament, preferably in the treatment and/or prevention of cancer.

Preferably the SFK is s-Src. More preferably the cancer is a solid or liquid cancer, preferably the cancer is selected from the group consisting of neuroblastoma, glioblastoma, osteosarcoma, prostate cancer, hepatocellular carcinoma, leukemia, retinoblastoma, rhabdomyosarcoma, hepatocellular carcinoma, glioblastoma multiformae, squamous cell carcinoma of the head and neck, melanoma, breast cancer, ovarian cancer, pancreatic cancer, mesothelioma.

Preferably the compounds of the invention are for use in the treatment of a neurodegenerative disease.

The present invention provides a compound or a stereoisomer or a pharmaceutically acceptable salt thereof for use in the treatment and/or prevention of a disease selected from the group consisting of: solid tumour and neurodegenerative disease wherein said compound has the formula IV:

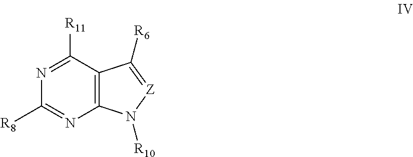

wherein:
Z represents CH or N;
R₆ represents H
or an aralkyl with the formula:

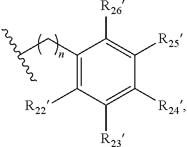

where R₂₂′, R₂₃′, R₂₄′, R₂₅′, R₂₆′ are independently H, C₁₋₆ alkyl, C₂₋₆ alkenyl, C₂₋₆ alkynyl, aryl unsubstituted or substituted group, halo, haloalkyl, OCH₃, NO₂, CN, CO(C₁₋₆ alkyl), CONH₂, CONH—C₁₋₆ alkyl, CON(C₁₋₆ alkyl)₂, NH₂, NH—C₁₋₆ alkyl, N(C₁₋₆ alkyl)₂, NHC(O)alkyl, NHCONH—C₁₋₆ alkyl, NHSO₂—C₁₋₆ alkyl, SO₂NH₂, SO₂NHC₁₋₆ alkyl, SO₂N(C₁₋₆ alkyl)₂, OQ′ or SQ′ where Q′ is H, or alkyl unsubstituted or substituted group, or aryl unsubstituted or substituted group, or aralkyl unsubstituted or substituted group; n is an integer from 0 to 4; or:

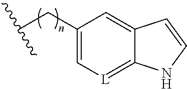

where L is CH or N; n is an integer from 0 to 4;
R₈ represents H, benzyl, alkylthio, alkylamino, cycloalkyl, cycloalkylthio, cycloalkylamino, alkyl, S(CH₂)ₚOH, S(CH₂)ₚNH₂, S(CH₂)ₚNHCH₃, S(CH₂)ₚN(CH₃)₂, NH(CH₂)ₚOH, NH(CH₂)ₚNH₂; NH(CH₂)ₚNHCH₃, NH(CH₂)ₚNH(CH₃)₂; p is an integer from 0 to 6;
or an alkyl chain with the formula:

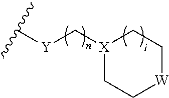

where Y is NH or O or S; X is CH or N; W is NH or NCH₃ or O;
n is an integer from 0 to 4; i is an integer from 0 to 1;
or:

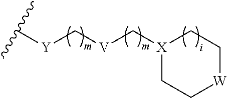

where Y is NH or O or S; V is cyclopropyl or cyclopentyl or cyclohexyl; X is CH or N; W is NH or NCH₃ or O; m is an integer from 0 to 2; i is an integer from 0 to 1;
or:

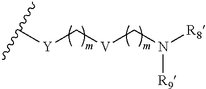

where Y is NH or O or S; V is cyclopropyl or cyclopentyl or cyclohexyl; R₈′ and R₉′ are independently H or CH₃; m is an integer from 0 to 2;
or:

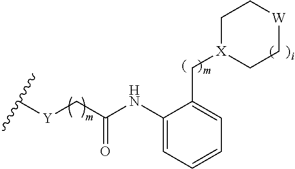

where Y is NH or O or S; X is CH or N; W is NH or NCH₃ or O; m is an integer from 0 to 2; i is an integer from 0 to 1;
R₁₀ represents

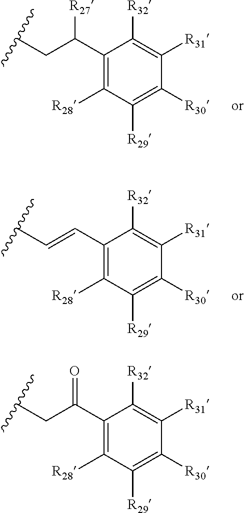

where R₂₇′ represents H, CH₃, CF₃, F, Cl, Br, OH; O-alkyl, alkyl;
where R₂₈′, R₂₉′, R₃₀′, R₃₁′, R₃₂′ are independently H, C₁₋₆ alkyl, C₂₋₆ alkenyl, C₂₋₆ alkynyl, aryl unsubstituted or substituted group, halo, haloalkyl, OCH₃, NO₂, CN, CONH₂, CONH—C₁₋₆ alkyl, CON(C₁₋₆ alkyl)₂, NH₂, NH—C₁₋₆ alkyl, N(C₁₋₆ alkyl)₂, NHC(O)alkyl, NHSO₂—C₁₋₆ alkyl, OQ′ or SQ′ where Q′ is H, or alkyl unsubstituted or substituted group, or aryl unsubstituted or substituted group, or aralkyl unsubstituted or substituted group; SO₂NH₂, SO₂NHC₁₋₆ alkyl, SO₂N(C₁₋₆ alkyl)₂, SO₂H, SO₂CH₃, PO₂, PO(CH₃)₂, POHCH₃, POH₂, SO₂J where J is:

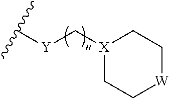

where Y is NH or O or S; X is CH or N; W is NH or NCH₃ or O; n is an integer from 0 to 4; R₃₇′ and R₃₈′ are independently H, alkyl, cycloalkyl, 1-pyrrolidinyl, 4-morpholinyl, 1-hexahydroazepinyl;
or an aralkyl with the formula:

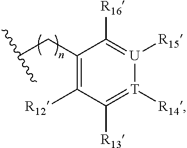

where T and U are independently C or N;
R₁₂′, R₁₃′, R₁₄′, R₁₅′, R₁₆′ are independently H, C₁₋₆ alkyl, C₂₋₆ alkenyl, C₂₋₆ alkynyl, aryl unsubstituted or substituted group, halo, haloalkyl, OCH₃, NO₂, CN, CONH₂, CONH—C₁₋₆ alkyl, CON(C₁₋₆ alkyl)₂, NH₂, NH—C₁₋₆ alkyl, N(C₁₋₆ alkyl)₂, NHC(O)alkyl, NHCONH—C₁₋₆ alkyl, NHSO₂C₁₋₆alkyl, SO₂NH₂, SO₂NHC₁₋₆alkyl, SO₂N(C₁₋₆ alkyl)₂, OQ′ or SQ′ where Q′ is H, or alkyl unsubstituted or substituted group, or aryl unsubstituted or substituted group, or aralkyl unsubstituted or substituted group, n is an integer from 0 to 4;
or:

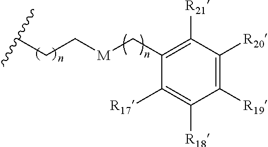

preferably R

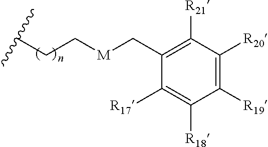

where M is NH or S or O;
R₁₇′, R₁₈′, R₁₉′, R₂₀′, R₂₁′ are independently H, C₁₋₆ alkyl, C₂₋₆ alkenyl, C₂₋₆ alkynyl, aryl unsubstituted or substituted group, halo, haloalkyl, OCH₃, NO₂, CN, CONH₂, CONH—C₁₋₆ alkyl, CON(C₁₋₆ alkyl)₂, NH₂, NH—C₁₋₆ alkyl, N(C₁₋₆ alkyl)₂, NHC(O)alkyl, NHSO₂C₁₋₆ alkyl, SO₂NH₂, SO₂NHC₁₋₆ alkyl, SO₂N(C₁₋₆ alkyl)₂, OQ′ or SQ′ where Q′ is H, or alkyl unsubstituted or substituted group, or aryl unsubstituted or substituted group, or aralkyl unsubstituted or substituted group; n is an integer from 0 to 4;
or R₁₁ represents

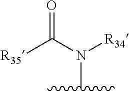

where R₃₄′ is H or alkyl or cycloalkyl or 1-pyrrolidinyl or 4-morpholinyl or 1-hexahydroazepinyl;
or an alkyl chain with the formula:

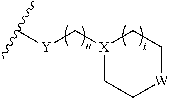

where Y is NH or O or S; X is CH or N; W is NH or NCH₃ or O; n is an integer from 0 to 4; i is an integer from 0 to 1;
or an aralkyl with the formula:

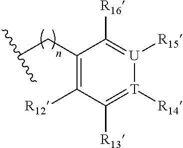

where T and U are independently C or N;
R₁₂′, R₁₃′, R₁₄′, R₁₅′, R₁₆′ are independently H, C₁₋₆ alkyl, C₂₋₆ alkenyl, C₂₋₆ alkynyl, aryl unsubstituted or substituted group, halo, haloalkyl, OCH₃, NO₂, CN, CONH₂, CONH—C₁₋₆ alkyl, CON(C₁₋₆ alkyl)₂, NH₂, NH—C₁₋₆ alkyl, N(C₁₋₆ alkyl)₂, NHC(O)alkyl, NHCONH—C₁₋₆ alkyl, NHSO₂—C₁₋₆ alkyl, SO₂NH₂, SO₂NHC₁₋₆ alkyl, SO₂N(C₁₋₆ alkyl)₂, OQ′ or SQ′ where Q′ is H, or alkyl unsubstituted or substituted group, or aryl unsubstituted or substituted group, or aralkyl unsubstituted or substituted group; n is an integer from 0 to 4;
or:

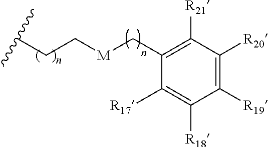

preferably

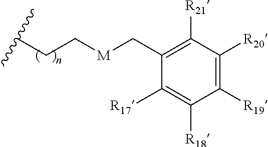

where M is NH or S or O;
R₁₇′, R₁₈′, R₁₉′, R₂₀′, R₂₁′ are independently H, C₁₋₆ alkyl, C₂₋₆ alkenyl, C₂₋₆ alkynyl, aryl unsubstituted or substituted group, halo, haloalkyl, OCH₃, NO₂, CN, CONH₂, CONH—C₁₋₆ alkyl, CON(C₁₋₆ alkyl)₂, NH₂, NH—C₁₋₆ alkyl, N(C₁₋₆ alkyl)₂, NHC(O)alkyl, NHSO₂C₁₋₆ alkyl, SO₂NH₂, SO₂NHC₁₋₆ alkyl, SO₂N(C₁₋₆ alkyl)₂, OQ′ or SQ′ where Q′ is H, or alkyl unsubstituted or substituted group, or aryl unsubstituted or substituted group, or aralkyl unsubstituted or substituted group; n is an integer from 0 to 4;
where R₃₅′ is an alkyl chain with the formula:

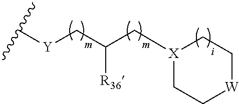

where Y is NH or O or S; R₃₆′ is H or alkyl or aryl or aralkyl; X is CH or N; W is NH or NCH₃ or O; m is an integer from 0 to 2; i is an integer from 0 to 1;
or:

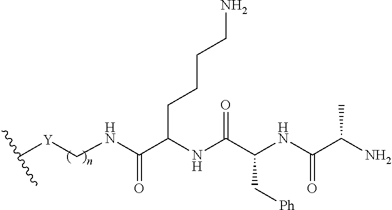

where Y is NH or O or S;
n is an integer from 0 to 4;
or:

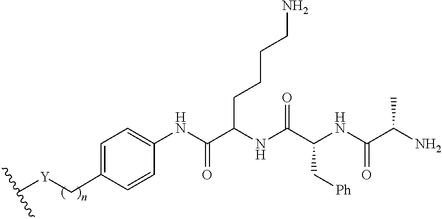

where Y is NH or O or S;
n is an integer from 0 to 4;
with the provisio that compounds:
N-(3-chlorophenyl)-6-(methylthio)-1-phenethyl-1H-pyrazolo[3,4-d]pyrimidin-4-amine (Si214);
6-(methylthio)-N-phenyl-1-(2-phenylpropyl)-1H-pyrazolo[3,4-d]pyrimidin-4-amine (Si276);
N-(3-chlorophenyl)-6-(methylthio)-1-(2-phenylpropyl)-1H-pyrazolo[3,4-d]pyrimidin-4-amine (Si277);
N-(3-bromophenyl)-6-(methylthio)-1-(2-phenylpropyl)-H-pyrazolo[3,4-d]pyrimidin-4-amine (Si278)
N-benzyl-1-(2-chloro-2-phenylethyl)-6-(methylthio)-1H-pyrazolo[3,4-d]pyrimidin-4-amine (Si34);
1-(2-chloro-2-phenylethyl)-6-(methylthio)-N-phenethyl-1H-pyrazolo[3,4-d]pyrimidin-4-amine (Si35); and
1-(2-chloro-2-phenylethyl)-N-(3-chlorophenyl)-6-(methylthio)-1H-pyrazolo[3,4-d]pyrimidin-4-amine (Si83);
   are excluded.

Preferably the compound for use is

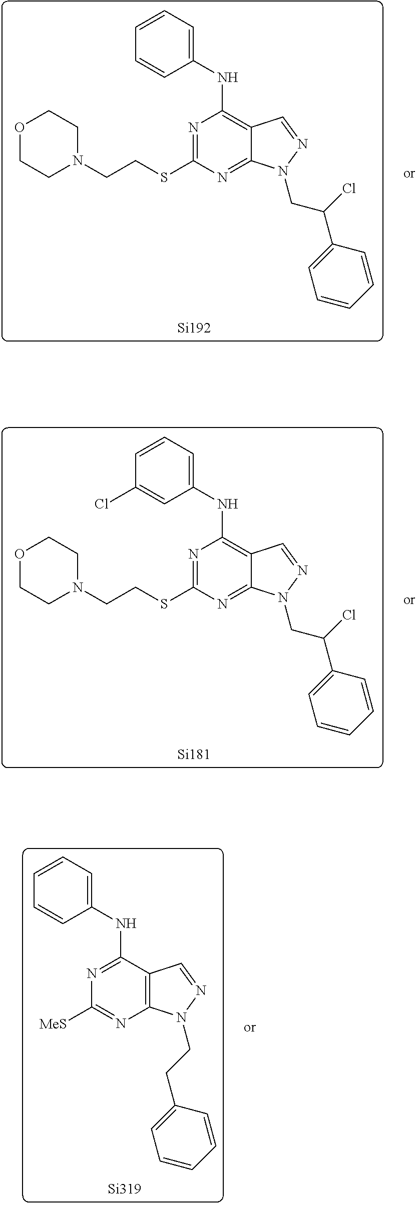 [SMILES: "Si192" ; "Si181" ; ClC(Cn1ncc2c(Nc3ccccc3)nc(SCCN3CCOCC3)nc21)c1ccccc1 ; Clc1cccc(Nc2nc(SCCN3CCOCC3)nc3c2cnn3CC(Cl)c2ccccc2)c1 ; CSc1nc(Nc2ccccc2)c2cnn(CCc3ccccc3)c2n1 ; "Si319"]

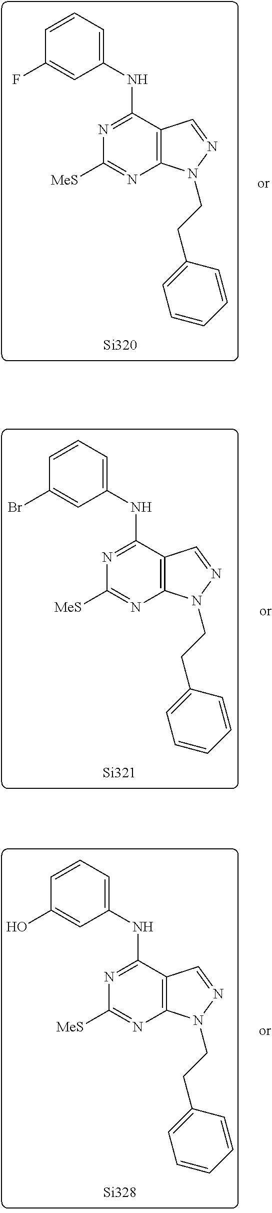 [SMILES: CSc1nc(Nc2cccc(F)c2)c2cnn(CCc3ccccc3)c2n1 ; "Si320" ; CSc1nc(Nc2cccc(Br)c2)c2cnn(CCc3ccccc3)c2n1 ; "Si321" ; CSc1nc(Nc2cccc(O)c2)c2cnn(CCc3ccccc3)c2n1 ; "Si328"]

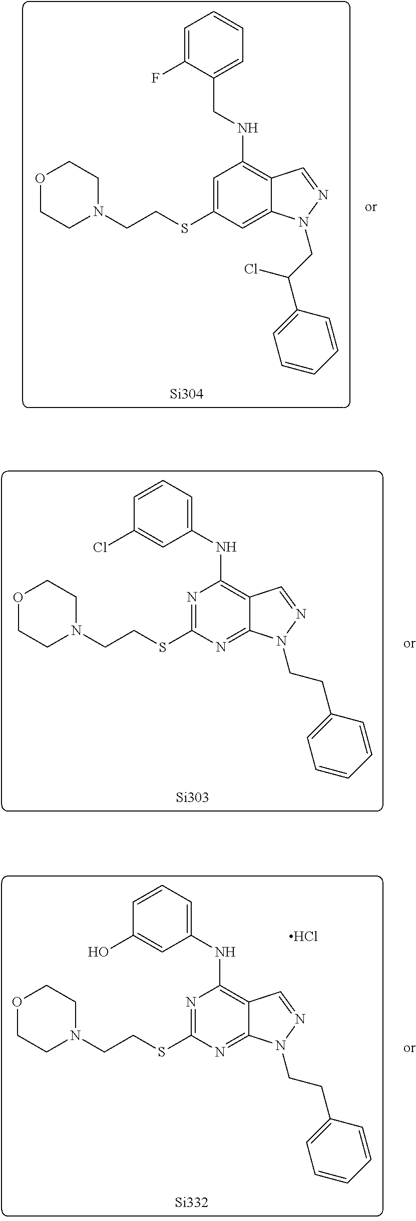

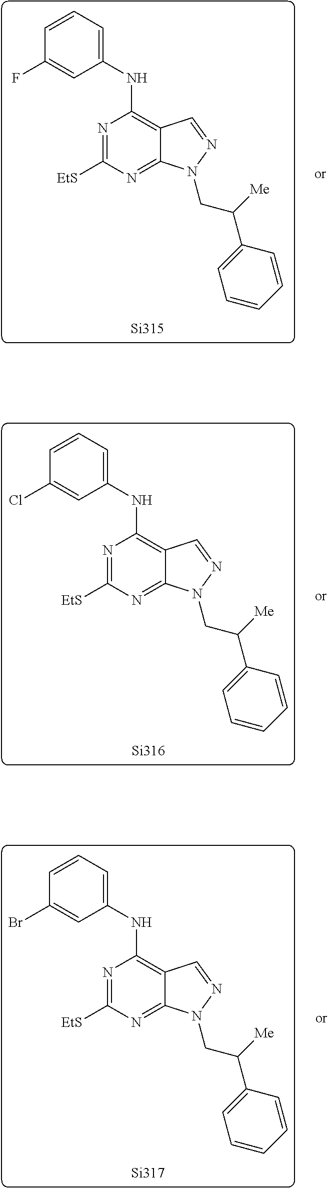

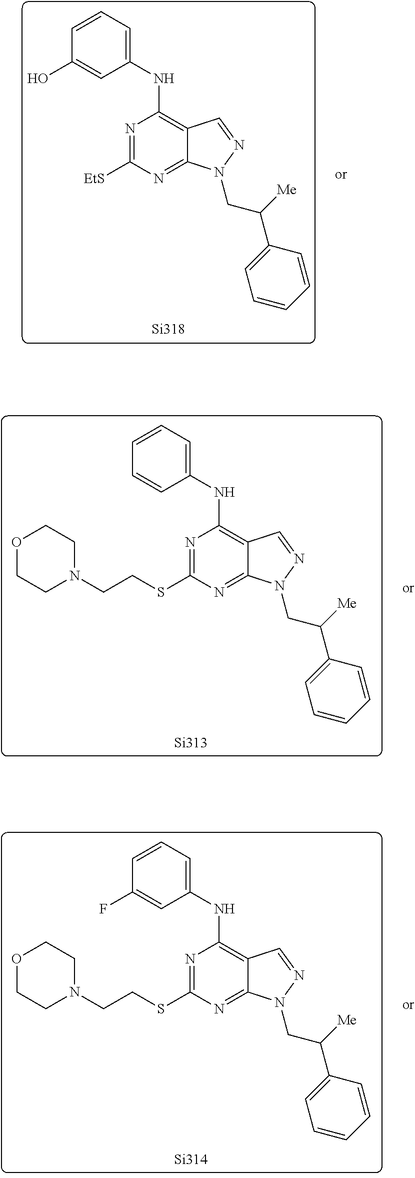

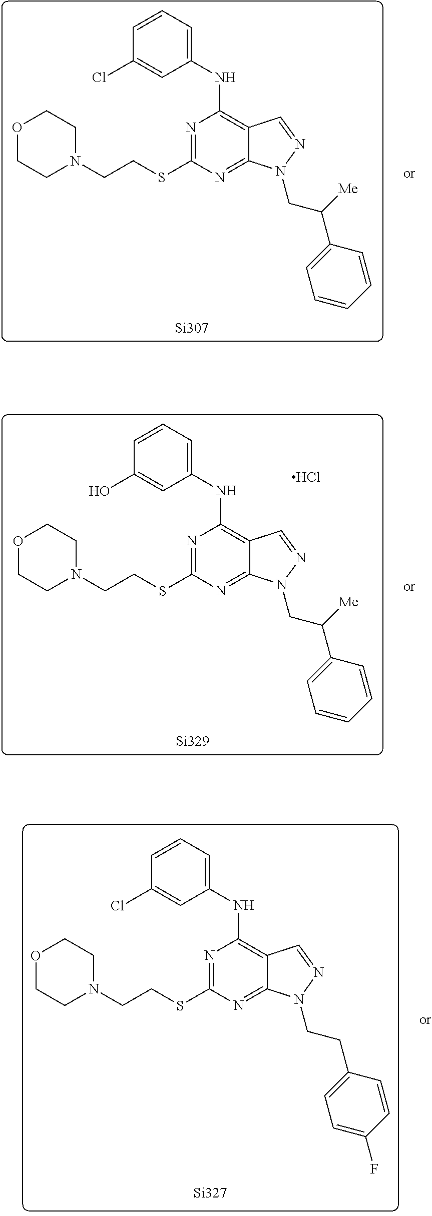

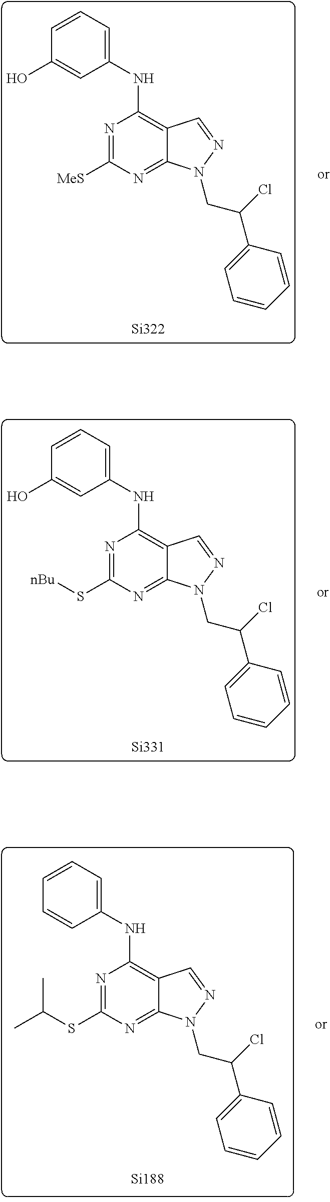

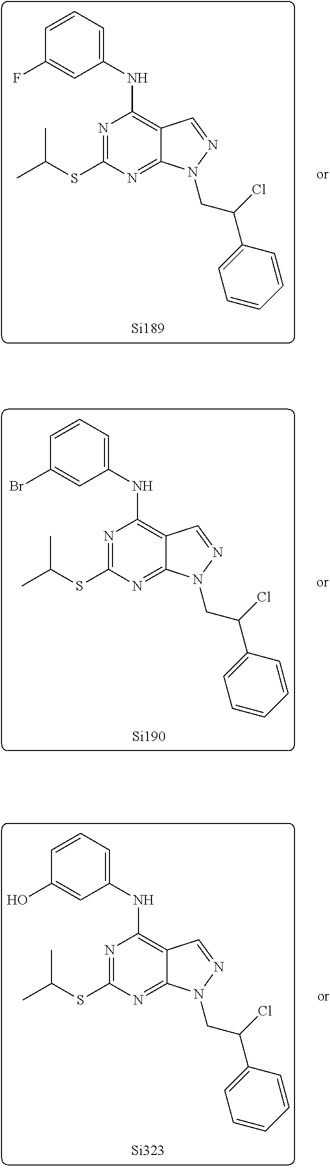 [SMILES: "Si190" ; "Si189" ; CC(C)Sc1nc(Nc2cccc(F)c2)c2cnn(CC(Cl)c3ccccc3)c2n1 ; CC(C)Sc1nc(Nc2cccc(Br)c2)c2cnn(CC(Cl)c3ccccc3)c2n1 ; CC(C)Sc1nc(Nc2cccc(O)c2)c2cnn(CC(Cl)c3ccccc3)c2n1 ; "Si323"]

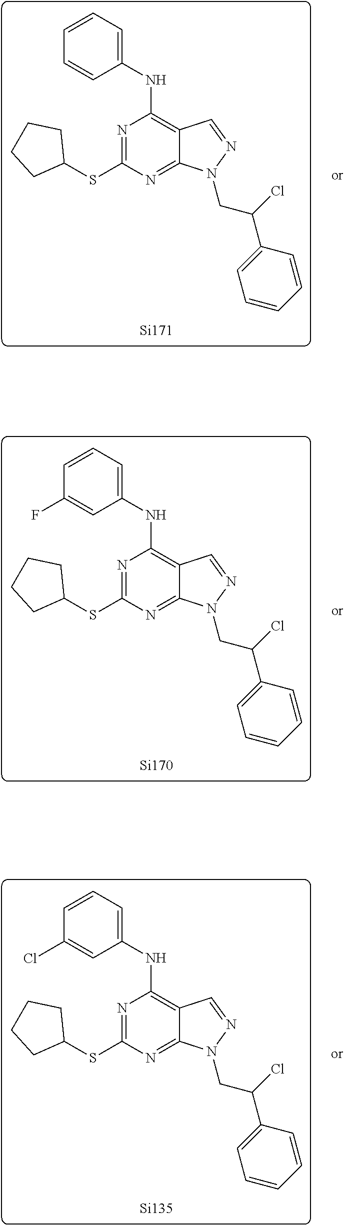 [SMILES: "Si170" ; "Si171" ; "Si135" ; ClC(Cn1ncc2c(Nc3ccccc3)nc(SC3CCCC3)nc21)c1ccccc1 ; Clc1cccc(Nc2nc(SC3CCCC3)nc3c2cnn3CC(Cl)c2ccccc2)c1 ; Fc1cccc(Nc2nc(SC3CCCC3)nc3c2cnn3CC(Cl)c2ccccc2)c1]

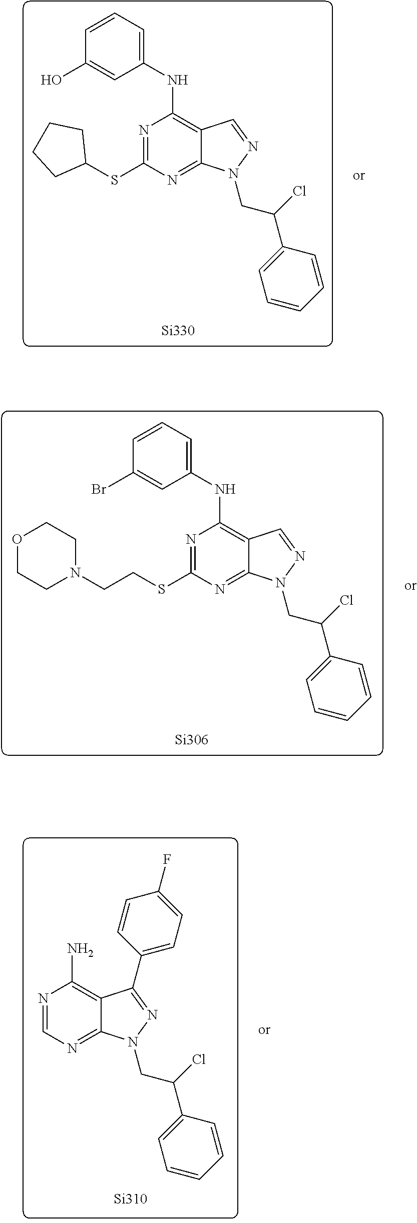 [SMILES: Oc1cccc(Nc2nc(SC3CCCC3)nc3c2cnn3CC(Cl)c2ccccc2)c1 ; "Si330" ; ClC(Cn1ncc2c(Nc3cccc(Br)c3)nc(SCCN3CCOCC3)nc21)c1ccccc1 ; "Si306" ; Nc1ncnc2c1c(-c1ccc(F)cc1)nn2CC(Cl)c1ccccc1 ; "Si310"]

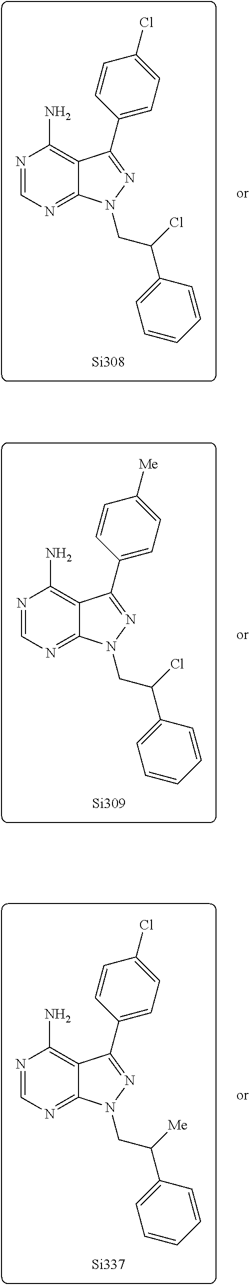 [SMILES: Nc1ncnc2c1c(-c1ccc(Cl)cc1)nn2CC(Cl)c1ccccc1 ; "Si308" ; Cc1ccc(-c2nn(CC(Cl)c3ccccc3)c3ncnc(N)c23)cc1 ; "Si309" ; CC(Cn1nc(-c2ccc(Cl)cc2)c2c(N)ncnc21)c1ccccc1 ; "Si337"]

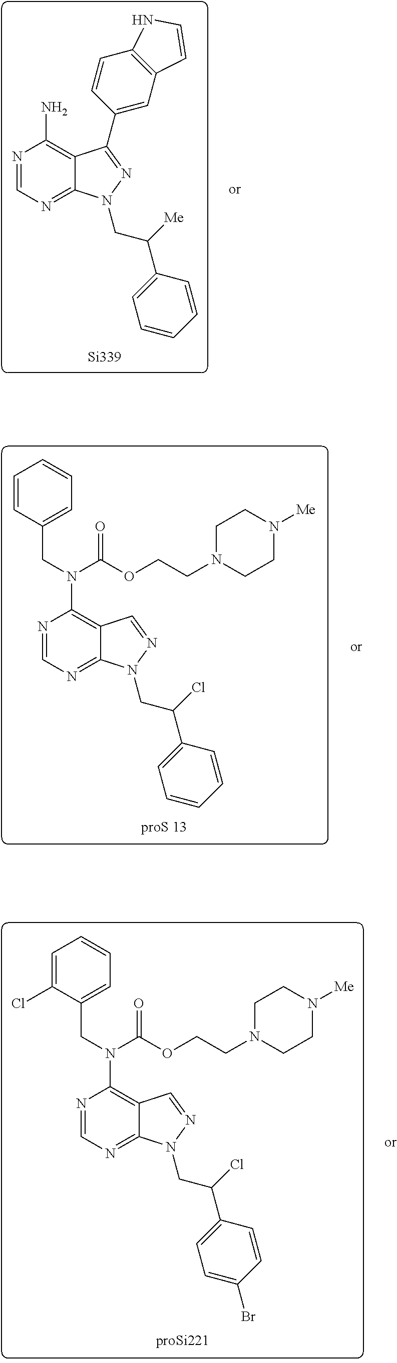

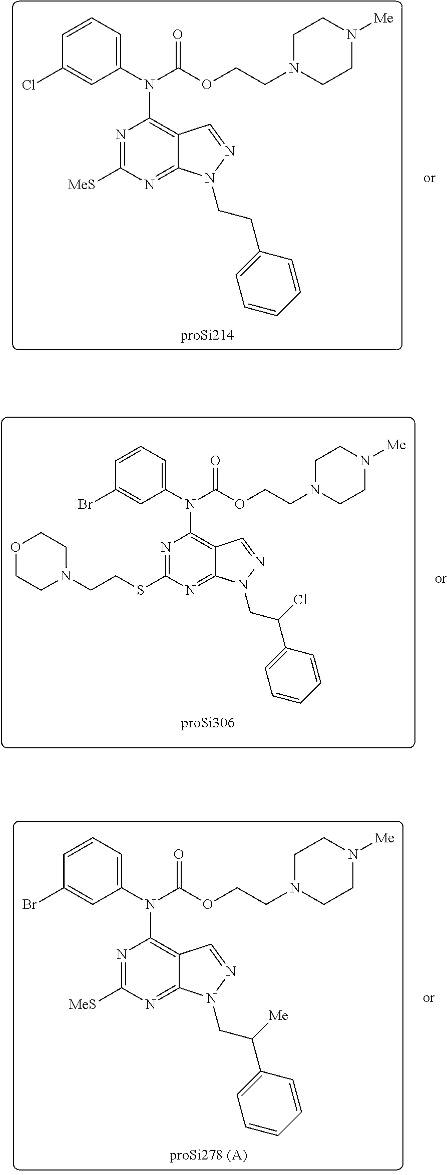 [SMILES: CSc1nc(N(C(=O)OCCN2CCN(C)CC2)c2cccc(Cl)c2)c2cnn(CCc3ccccc3)c2n1 ; "proSi214" ; CN1CCN(CCOC(=O)N(c2cccc(Br)c2)c2nc(SCCN3CCOCC3)nc3c2cnn3CC(Cl)c2ccccc2)CC1 ; "proSi306" ; CSc1nc(N(C(=O)OCCN2CCN(C)CC2)c2cccc(Br)c2)c2cnn(CC(C)c3ccccc3)c2n1 ; "proSi278 (A)"]

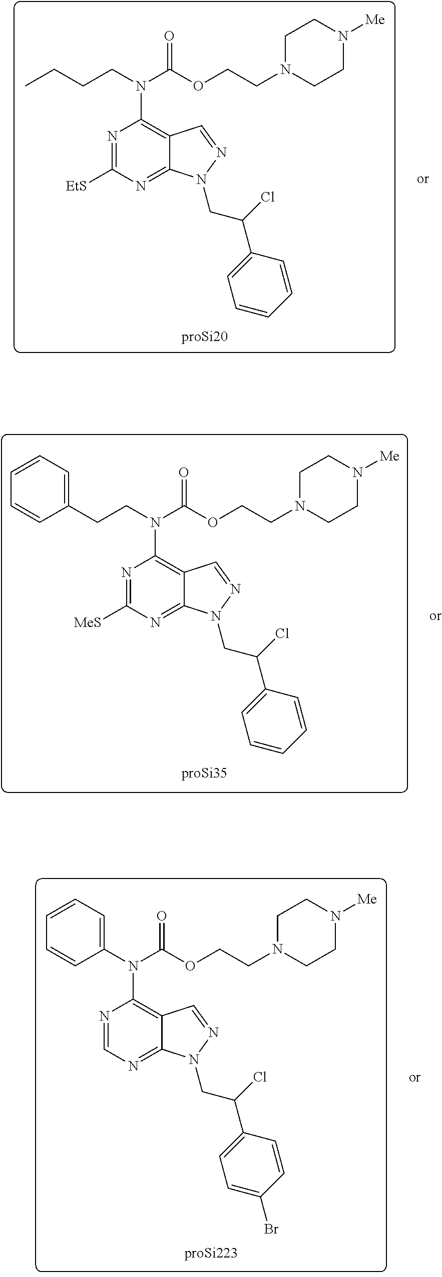

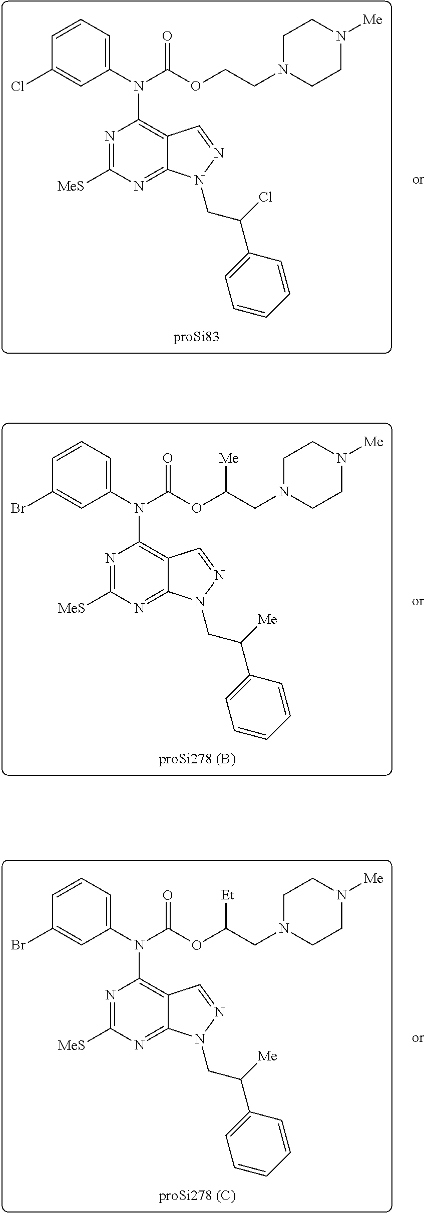 [SMILES: CSc1nc(N(C(=O)OCCN2CCN(C)CC2)c2cccc(Cl)c2)c2cnn(CC(Cl)c3ccccc3)c2n1 ; "proSi83" ; CSc1nc(N(C(=O)OC(C)CN2CCN(C)CC2)c2cccc(Br)c2)c2cnn(CC(C)c3ccccc3)c2n1 ; "proSi278 (B)" ; CCC(CN1CCN(C)CC1)OC(=O)N(c1cccc(Br)c1)c1nc(SC)nc2c1cnn2CC(C)c1ccccc1 ; "proSi278 (C)"]

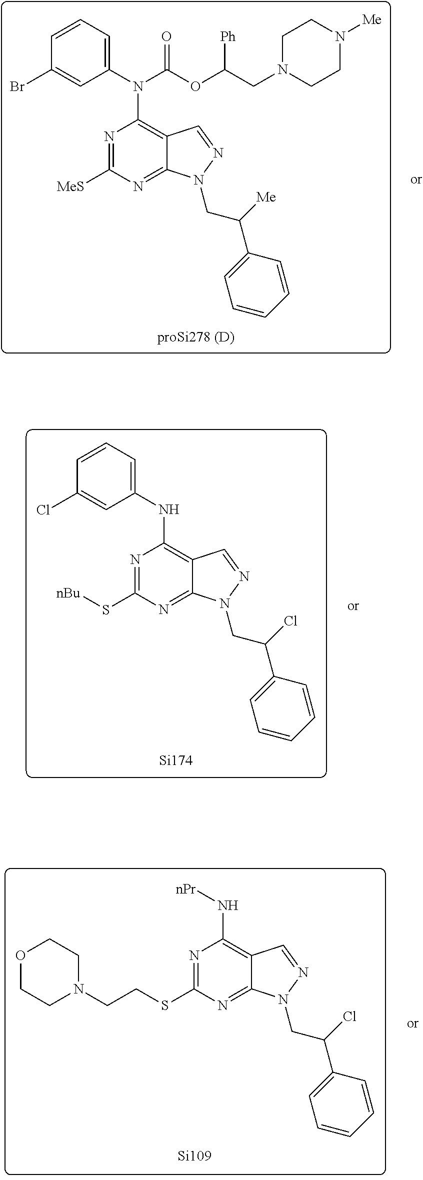

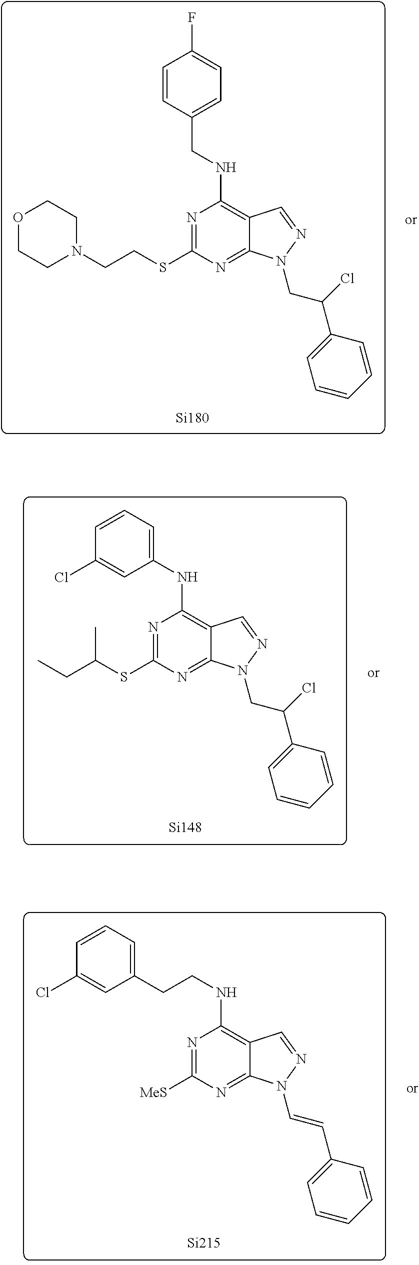 [SMILES: "Si215" ; "Si148" ; "Si180" ; Fc1ccc(CNc2nc(SCCN3CCOCC3)nc3c2cnn3CC(Cl)c2ccccc2)cc1 ; CCC(C)Sc1nc(Nc2cccc(Cl)c2)c2cnn(CC(Cl)c3ccccc3)c2n1 ; CSc1nc(NCCc2cccc(Cl)c2)c2cnn(/C=C/c3ccccc3)c2n1]

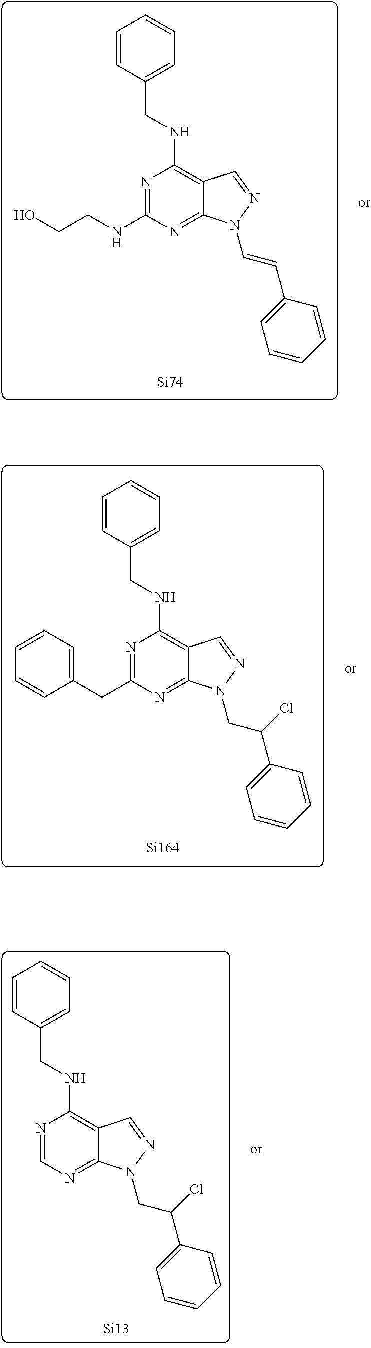 [SMILES: "Si164" ; "Si74" ; ClC(Cn1ncc2c(NCc3ccccc3)ncnc21)c1ccccc1 ; ClC(Cn1ncc2c(NCc3ccccc3)nc(Cc3ccccc3)nc21)c1ccccc1 ; OCCNc1nc(NCc2ccccc2)c2cnn(/C=C/c3ccccc3)c2n1 ; "Si13"]

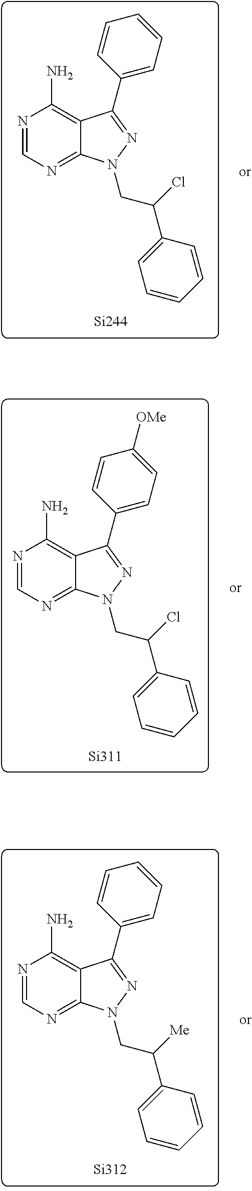 [SMILES: "Si244" ; COc1ccc(-c2nn(CC(Cl)c3ccccc3)c3ncnc(N)c23)cc1 ; Nc1ncnc2c1c(-c1ccccc1)nn2CC(Cl)c1ccccc1 ; "Si311" ; CC(Cn1nc(-c2ccccc2)c2c(N)ncnc21)c1ccccc1 ; "Si312"]

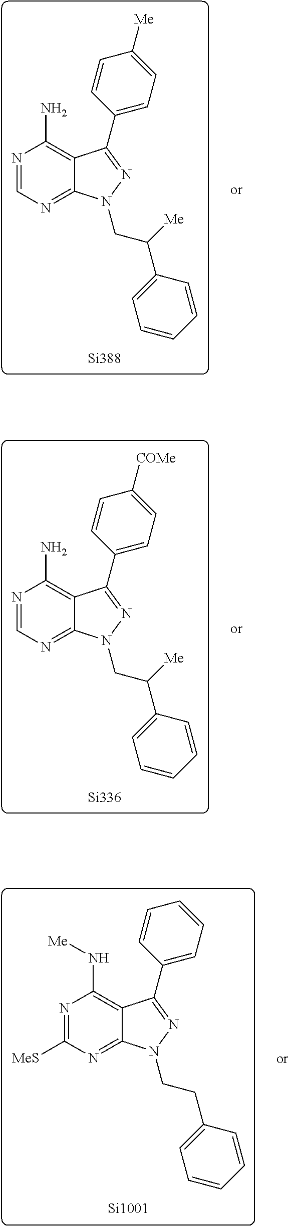

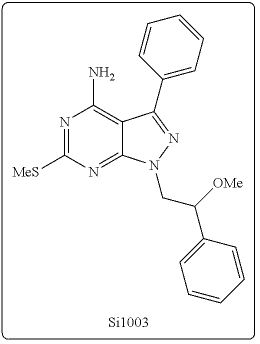 [SMILES: COC(Cn1nc(-c2ccccc2)c2c(N)nc(SC)nc21)c1ccccc1 ; "Si1003"]

or a stereoisomer or a pharmaceutically acceptable salt thereof. Preferably the tumour is selected from the group consisting of: neuroblastoma, glioblastoma, retinoblastoma, rhabdomyosarcoma, hepatocellular carcinoma, glioblastoma multiformae, squamous cell carcinoma of the head and neck, melanoma, breast cancer, ovarian cancer, pancreatic cancer and mesothelioma.

Preferably the compound is for use with a further anti-tumoral therapy. More preferably the further anti-tumoral therapy is selected from the group consisting of: radiotherapy and chemotherapy. Still preferably the chemotherapy is selected from the group consisting of: mitomycin C, cisplatin, etoposide, vincristine, doxorubicin, isotretinoin and cyclophosphamide.

The present invention provides a pharmaceutical composition comprising a compound of the formula I or a stereoisomer or a prodrug or a pharmaceutically acceptable salt thereof as defined above and pharmaceutically acceptable carrier.

Preferably the pharmaceutically acceptable carrier is selected from the group consisting of a nanoparticle such as: liposome, albumin, cyclodextrin and gold nanoparticles.

The present invention provides a process for the preparation of a prodrug of the compound of formula I as defined in claim 1, wherein said prodrug is a prodrug of formula III

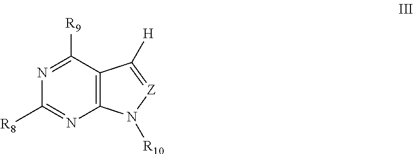

wherein

### R₁₀ is

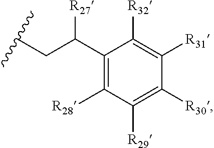

R_{28′}, R_{29′}, R_{31′} and R_{32′} are H comprising the following step:

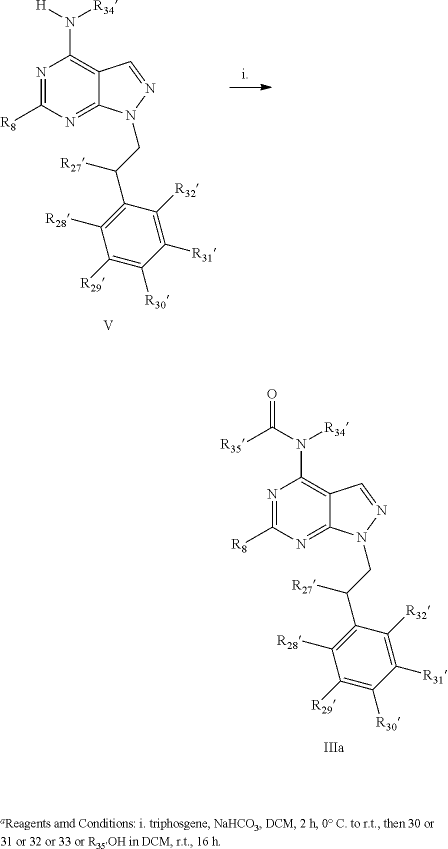

Wherein R₈, R_{27′}, R₂₈′, R₂₉′, R₃₀′, R₃₁′, R₃₂′, R₃₄′ are as defined in claim 5, and wherein R_{35′} is:
an alkyl chain with the formula:

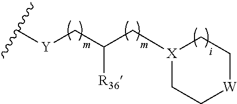

where Y is NH or O or S; R₃₆′ is H or alkyl or aryl or aralkyl; X is CH or N; W is NH or NCH₃ or O; m is an integer from 0 to 2; i is an integer from 0 to 1;
or:

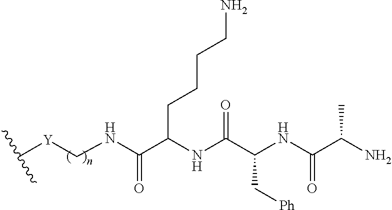

where Y is NH or O or S;
n is an integer from 0 to 4;
or:

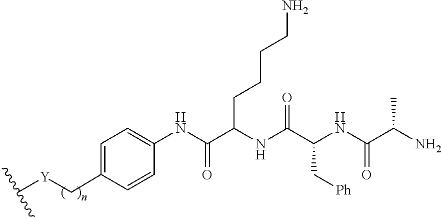

where Y is NH or O or S;
n is an integer from 0 to 4;
or a process for the preparation of a compound of formula I, said process comprising the following steps:

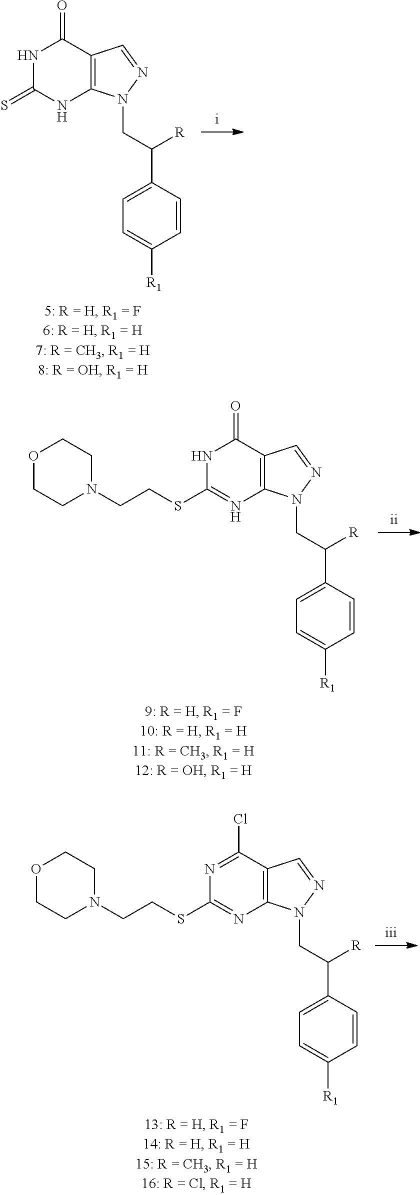

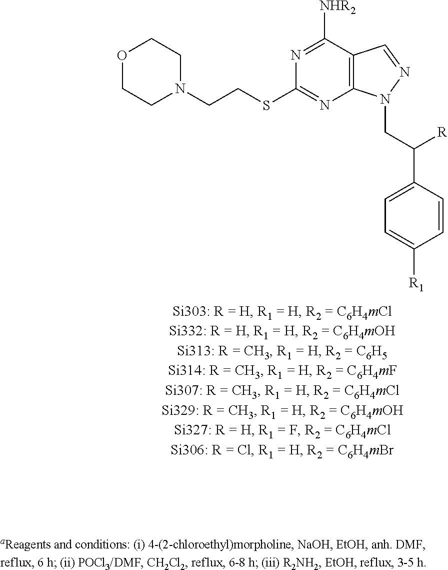

or a process for the preparation of compounds of formula IV as defined in claim 3, or salts thereof, comprising the following steps:

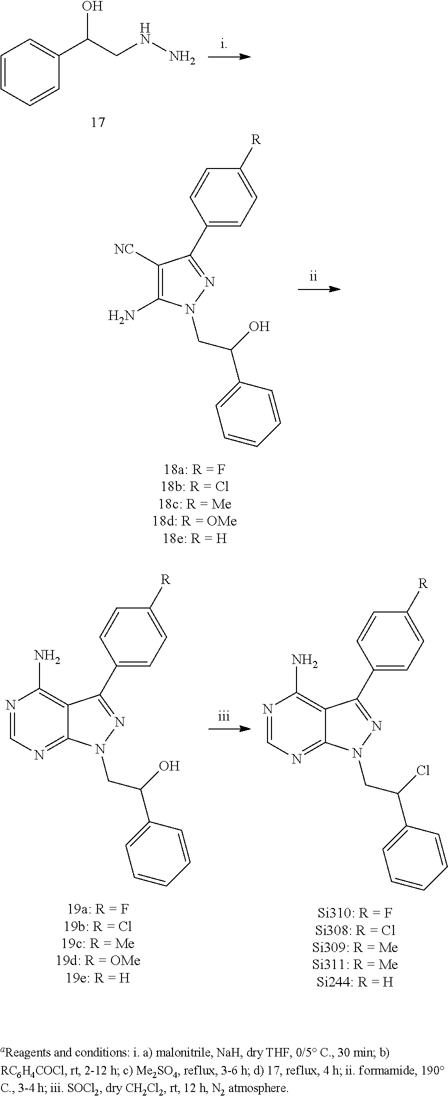

or a process for the preparation of compounds of formula IV as defined in claim 3, or salts thereof, comprising the following steps:

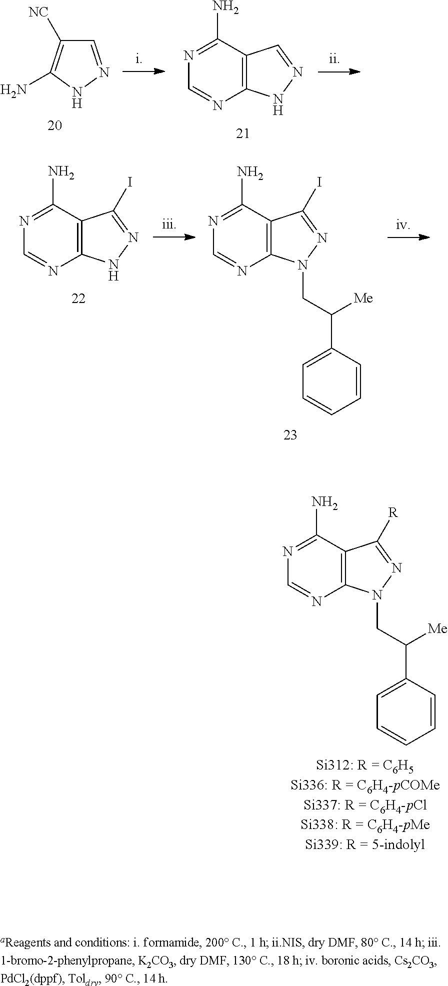

or a process for the preparation of compounds of formula IV as defined in claim 3, or salts thereof, comprising the following steps:

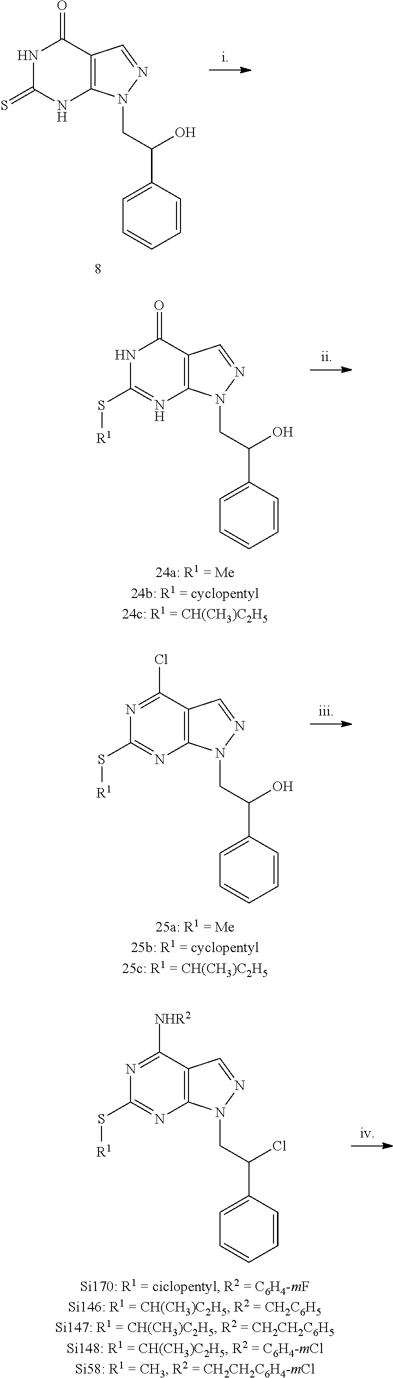

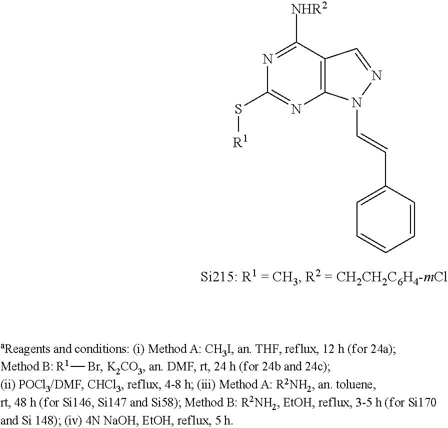

or a process for the preparation of compound Si74 of formula IV as defined in claim 3, or salts thereof, comprising the following steps:

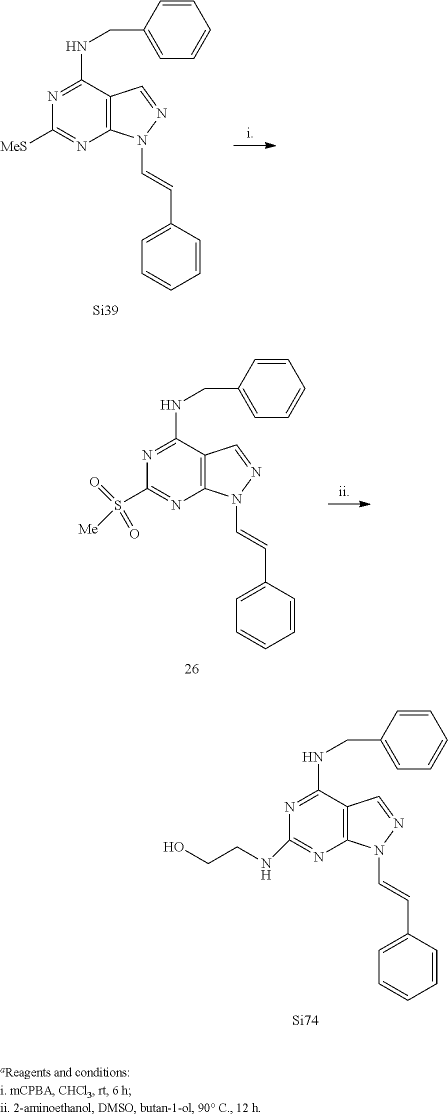 [SMILES: "Si39" ; CSc1nc(NCc2ccccc2)c2cnn(/C=C/c3ccccc3)c2n1 ; CS(=O)(=O)c1nc(NCc2ccccc2)c2cnn(/C=C/c3ccccc3)c2n1 ; "26" ; "Si74" ; OCCNc1nc(NCc2ccccc2)c2cnn(/C=C/c3ccccc3)c2n1]

or a process for the preparation of compound Si164 of formula IV as defined in claim 3, or salts thereof, comprising the following steps:

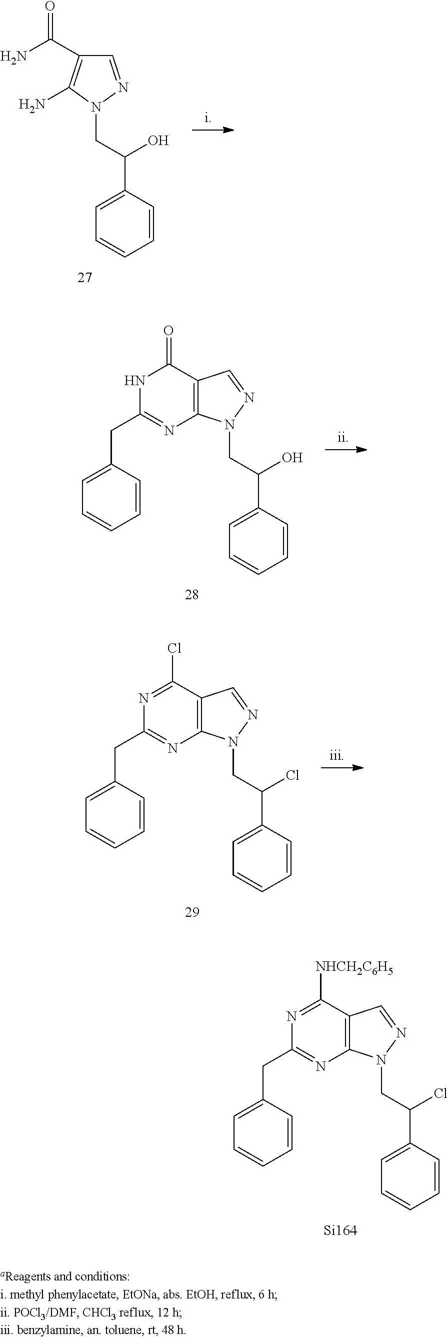

In the present invention the term “halogen” or “halo” refers to fluoro, chloro, bromo, or iodo. The term “alkyl” refers to a straight or branched hydrocarbon chain radical, consisting solely of carbon and hydrogen atoms. Suitable examples of said alkyl include but are not limited to methyl, ethyl, n-propyl, isopropyl, butyl, sec-butyl, tert-butyl, pentyl, isopentyl, tert-pentyl, hexyl, heptyl, octyl, nonyl, decanyl, hexadecanyl, eicosanyl, etc. “alkyl substituted group” means that any hydrogen atom on independently each carbon atom may be independently replaced by a substituent, suitable examples of substituent include but are not limited to F, Cl, Br, I, CF₃, CN, O—C₁₋₆ alkyl, C₁₋₆ alkyl, OH, S—C₁₋₆ alkyl, COC₁₋₆ alkyl, OCOC₁₋₆ alkyl, CO₂C₁₋₆ alkyl.

The term “C₁₋₆ alkyl” refers to a straight or branched hydrocarbon chain radical, consisting solely of carbon and hydrogen atoms, having from one to six carbon atoms. Suitable examples of C₁₋₆ alkyl include but are not limited to ethyl, n-propyl, isopropyl, butyl, sec-butyl, tert-butyl, pentyl, isopentyl, tert-pentyl, hexyl.

The term “C₂₋₆ alkyl” refers to a straight or branched hydrocarbon chain radical, consisting solely of carbon and hydrogen atoms, having from two to six carbon atoms. Suitable examples of C₂₋₆ alkyl include but are not limited to ethyl, n-propyl, isopropyl, butyl, sec-butyl, tert-butyl, pentyl, isopentyl, tert-pentyl, hexyl.

The term “C₂₋₆ alkenyl” refers to a straight or branched unsaturated hydrocarbon chain radical, containing at least one carbon-carbon double bond, consisting solely of carbon and hydrogen atoms, having from two to six carbon atoms. Suitable examples of C₂₋₆ alkenyl but are not limited to ethenyl, propenyl, allyl, isobuthenyl, pentenyl, prenyl, esenyl, etc.

The term “C₂₋₆ alkynyl” refers to a straight or branched unsaturated hydrocarbon chain radical, containing at least one carbon-carbon triple bond, consisting solely of carbon and hydrogen atoms, having from two to six carbon atoms. Suitable examples of C₂₋₆ alkynyl but are not limited to acetylenyl, ethynyl, propynyl, etc.

The term “haloalkyl” group is preferably a linear or branched C₁-C₁₀ haloalkyl group, more preferably C₁-C₈ haloalkyl group, more preferably linear or branched C₁-C₆ haloalkyl group, still more preferably linear or branched C₁-C₄ haloalkyl group, more preferably a C₁-C₂ haloalkyl group, being in particular CF₃, CHF₂, CH₂F.

The term “aryl” represents a mono or bicyclic aromatic ring system of, respectively, 6, 9 or 10 atoms, suitable examples of such an aryl are phenyl, indenyl, indanyl and naphthyl and tetrahydronaphthalenyl. “Substituted aryl” or “aryl substituted group” means that the hydrogen atom on independently each carbon atom may be independently replaced by a substituent, suitable examples of substituent include but are not limited to F, Cl, Br, I, CF₃, CN, O—C₁₋₆ alkyl, C₁₋₆ alkyl, OH, S—C₁₋₆ alkyl, COC₁₋₆ alkyl, OCOC₁₋₆ alkyl, CO₂C₁₋₆ alkyl.

The term “aralkyl” represents any univalent radical derived from an alkyl radical by replacing one or more hydrogen atoms by aryl groups, wherein the aryl is as defined herein above. “aralkyl substituted group” means that any hydrogen atom on independently each carbon atom may be independently replaced by a substituent, suitable examples of substituent include but are not limited to F, Cl, Br, I, CF₃, CN, O—C₁₋₆ alkyl, C₁₋₆ alkyl, OH, S—C₁₋₆ alkyl, COC₁₋₆ alkyl, OCOC₁₋₆ alkyl, CO₂C₁₋₆ alkyl.

The term “cycloalkyl” refers to a saturated monocyclic hydrocarbon ring system having at least three carbon atoms, preferably from three to seven carbon atoms. Suitable examples of cycloalkyl groups include cyclopropyl, cyclobutyl, cyclopentyl, and cyclohexyl etc.

The term “cycloalkylamino” refers to a cycloalkyl-NH group wherein the cycloalkyl group is as defined herein above.

The term “cycloalkylthio” refers to a cycloalkyl-S group wherein the cycloalkyl group is as defined herein above.

The term “alkylthio” refers to an alkyl-S group wherein the alkyl group is as defined herein above.

The term “alkylamino” refers to a alkyl-NH group wherein the alkyl group is as defined herein above.

Tauopathies are a class of neurodegenerative diseases associated with the pathological aggregation of tau protein in the human brain.

The best-known of these illnesses is Alzheimer's disease (AD), wherein tau protein is deposited within neurons in the form of neurofibrillary tangles (NFTs). They were first described by the eponymous Alois Alzheimer in one of his patients suffering from the disorder. Tangles are formed by hyperphosphorylation of a microtubule-associated protein known as tau, causing it to aggregate in an insoluble form. (These aggregations of hyperphosphorylated tau protein are also referred to as PHF, or “paired helical filaments”). The precise mechanism of tangle formation is not completely understood, and it is still controversial as to whether tangles are a primary causative factor in the disease or play a more peripheral role. AD is also classified as an amyloidosis because of the presence of senile plaques.

Other conditions in which neurofibrillary tangles are commonly observed include: Progressive supranuclear palsy although with straight filament rather than PHF tau, Dementia pugilistica (chronic traumatic encephalopathy), Frontotemporal dementia and parkinsonism linked to chromosome 17, however without detectable 3-amyloid plaques, Lytico-Bodig disease (Parkinson-dementia complex of Guam), Tangle-predominant dementia, with NFTs similar to AD, but without plaques, that ends to appear in the very old, Ganglioglioma and gangliocytoma, Meningioangiomatosis, Subacute sclerosing panencephalitis, as well as lead encephalopathy, tuberous sclerosis, Hallervorden-Spatz disease, and lipofuscinosis.

In Pick's disease and corticobasal degeneration tau proteins are deposited in the form of inclusion bodies within swollen or “ballooned” neurons.

Argyrophilic grain disease (AGD), another type of dementia is marked by the presence of abundant argyrophilic grains and coiled bodies on microscopic examination of brain tissue. Some consider it to be a type of Alzheimer disease. It may co-exist with other tauopathies such as progressive supranuclear palsy and corticobasal degeneration, and also Pick's disease. Some other tauopathies include: Frontotemporal dementia or Frontotemporal lobar degeneration. The non-Alzheimer's tauopathies are sometimes grouped together as “Pick's complex”.

Salts of the compounds of the present invention are also encompassed within the scope of the invention. Because of their potential use in medicine, the salts of the compounds of formula I, II, III and IV are preferably pharmaceutically acceptable. Suitable pharmaceutically acceptable salts comprise conventional non-toxic salts obtained by salification of a compound of formula I, II, III and IV with inorganic acids (e.g. hydrochloric, hydrobromic, sulphuric, or phosphoric acids), or with organic acids (e.g. acetic, propionic, succinic, benzoic, sulfanilic, 2-acetoxy-benzoic, cinnamic, mandelic, salicylic, glycolic, lactic, oxalic, malic, maleic, malonic, fumaric, tartaric, citric, p-toluenesulfonic, methanesulfonic, ethanesulfonic, or naphthalensulfonic acids). For reviews on suitable pharmaceutical salts see (37). Other salts, which are not pharmaceutically acceptable, for example the trifluoroacetate salt, may be useful in the preparation of compounds of this invention and these form a further aspect of the invention.

The invention includes within its scope all possible stoichiometric and non-stoichiometric forms of the salts of the compounds of formula I, III and IV.

In addition, the compounds of formula I, II and IV may exist in unsolvated as well as in solvated forms with pharmaceutically acceptable solvents such as water, EtOH and the like. Certain compounds of formula I, III and IV may exist in stereoisomeric forms (e.g. they may contain one or more asymmetric carbon atoms). The individual stereoisomers (enantiomers and diastereomers) and mixtures of these are included within the scope of the present invention. The present invention also covers the individual isomers of the compounds represented by formula I, III and IV as mixtures with isomers thereof in which one or more chiral centers are inverted. Likewise it is understood that compounds of formula I, III and IV may exist in tautomeric forms other than that shown in the formula and these are also included within the scope of the present invention.

The invention also includes all suitable isotopic variations of a compound of the invention. An isotopic variation of a compound of the invention is defined as one in which at least one atom is replaced by an atom having the same atomic number but an atomic mass different from the atomic mass usually found in nature. Examples of isotopes that can be incorporated into compounds of the invention include isotopes such as ²H, ³H, ¹³C, ¹⁴C, ¹³N, ¹⁷O, ¹⁸O, ³¹P, ³²P, ³⁵S, ¹⁸F and ³⁶Cl, respectively. Certain isotopic variations of the invention, for example, those in which a radioactive isotope such as ³H or ¹⁴C is incorporated, are useful in drug and/or substrate tissue distribution studies. Further, substitution with isotopes such as deuterium ²H, may afford certain therapeutic advantages resulting from greater metabolic stability. Isotopic variations of the compounds of the invention can generally be prepared by conventional procedures such as by the illustrative methods or by the preparations described in the examples hereafter using appropriate isotopic variations of suitable reagents.

The invention also provides pharmaceutical compositions comprising at least one compound of this invention or a pharmaceutical acceptable salt or solvate thereof and one or more pharmaceutically acceptable carriers, excipients and/or diluents.

The pharmaceutical compositions can be chosen based on the treatment requirements. Such compositions are prepared by blending and are suitably adapted to oral or parenteral administration, and as such can be administered in the form of tablets, capsules, oral preparations, powders, granules, pills, injectable, or infusible liquid solutions, suspensions, suppositories, preparation for inhalation.

Tablets and capsules for oral administration are normally presented in unit dose form and contain conventional excipients such as binders, fillers (including cellulose, mannitol, lactose), diluents, tableting agents, lubricants (including magnesium stearate), detergents, disintegrants (e.g. polyvinylpyrrolidone and starch derivatives such as sodium glycolate starch), coloring agents, flavoring agents, and wetting agents (for example sodium lauryl sulfate).

The oral solid compositions can be prepared by conventional methods of blending, filling or tableting. The blending operation can be repeated to distribute the active principle throughout compositions containing large quantities of fillers. Such operations are conventional.

Oral liquid preparations can be in the form of, for example, aqueous or oily suspensions, solutions, emulsions, syrups or elixirs, or can be presented as a dry product for reconstitution with water or with a suitable vehicle before use. Such liquid preparations can contain conventional additives such as suspending agents, for example sorbitol, syrup, methyl cellulose, gelatin, hydroxyethyl cellulose, carboxymethyl cellulose, aluminium stearate gel, or hydrogenated edible fats; emulsifying agents, such as lecithin, sorbitan monooleate, or acacia; non-aqueous vehicles (which can include edible oils), such as almond oil, fractionated coconut oil, oily esters such as esters of glycerine, propylene glycol, or ethyl alcohol; preservatives, such as methyl or propyl p-hydroxybenzoate or sorbic acid, and if desired, conventional flavoring or coloring agents. Oral formulations also include conventional slow-release formulations such as enterically coated tablets or granules.

Pharmaceutical preparation for administration by inhalation can be delivered from an insufflator or a nebulizer pressurized pack.

For parenteral administration fluid unit dosages can be prepared, containing the compound and a sterile vehicle. The compound can be either suspended or dissolved, depending on the vehicle and concentration. The parenteral solutions are normally prepared by dissolving the compound in a vehicle, sterilising by filtration, filling suitable vials and sealing. Advantageously, adjuvants such as local anaesthetics, preservatives and buffering agents can also be dissolved in the vehicle. To increase the stability, the composition can be frozen after having filled the vials and removed the water under vacuum. Parenteral suspensions are prepared in substantially the same manner, except that the compound can be suspended in the vehicle instead of being dissolved, and sterilized by exposure to ethylene oxide before suspending in the sterile vehicle. Advantageously, a surfactant or wetting agent can be included in the composition to facilitate uniform distribution of the compound of the invention.

For buccal or sublingual administration the compositions may be tablets, lozenges, pastilles, or gel.

The compounds can be pharmaceutically formulated as suppositories or retention enemas, e.g. containing conventional suppositories bases such as cocoa butter, polyethylene glycol, or other glycerides, for a rectal administration.

Another means of administering the compounds of the invention regards topical treatment. Topical formulations can contain for example ointments, creams, lotions, gels, solutions, pastes and/or can contain liposomes, micelles and/or microspheres. Examples of ointments include oleaginous ointments such as vegetable oils, animal fats, semisolid hydrocarbons, emulsifiable ointments such as hydroxystearin sulfate, anhydrous lanolin, hydrophilic petrolatum, cetyl alcohol, glycerol monostearate, stearic acid, water soluble ointments containing polyethylene glycols of various molecular weights. Creams, as known to formulation experts, are viscous liquids or semisolid emulsions, and contain an oil phase, an emulsifier and an aqueous phase. The oil phase generally contains petrolatum and an alcohol such as cetyl or stearic alcohol. Formulations suitable for topical administration to the eye also include eye drops, wherein the active ingredient is dissolved or suspended in a suitable carrier, especially an aqueous solvent for the active ingredient.

A further method of administering the compounds of the invention regards transdermal delivery. Typical transdermal formulations comprise conventional aqueous and non-aqueous vectors, such as creams, oils, lotions or pastes or can be in the form of membranes or medicated patches.

A reference for the formulations is the book by Remington³⁸.

The compounds of the present invention may be employed for use in the treatment and/or prevention of the above mentioned conditions alone as a sole therapy or in combination with other therapeutic agents either by separate administrations, or by including the two or more active principles in the same pharmaceutical formulation. The compounds may be administered simultaneously or sequentially.

The other therapeutic agents may be antitumor drugs or compounds currently on the market. Non-exhaustive examples of suitable additional agents include in particular drugs belonging to the group of: mitomycin C, cisplatino, etoposide, vincristine, doxorubicin, isotretinoin and cyclophosphamide.

The combination can be administered as separate compositions (simultaneous, sequential) of the individual components of the treatment or as a single dosage form containing both agents. When the compounds of this invention are in combination with others active ingredients, the active ingredients may be separately formulated into single-ingredient preparations of one of the above-described forms and then provided as combined preparations, which are given at the same time or different times, or may be formulated together into a two- or more-ingredient preparation.

Compounds of general formula I, III and IV may be administered to a patient in a total daily dose of, for example, from 0.001 to 1000 mg/kg body weight daily. Dosage unit compositions may contain such amounts of submultiples thereof to make up the daily dose. The compound may also be administered weekly or any other day. The determination of optimum dosages for a particular patient is well known to one skilled in the art. As is common practice, the compositions are normally accompanied by written or printed instructions for use in the treatment in question.

The invention will be now illustrated by means of non-limiting examples referring to the following figures:

FIG. 1. Molecular structure of PP2, Dasatinib, compounds Si34, Si214, Si192 and S34.

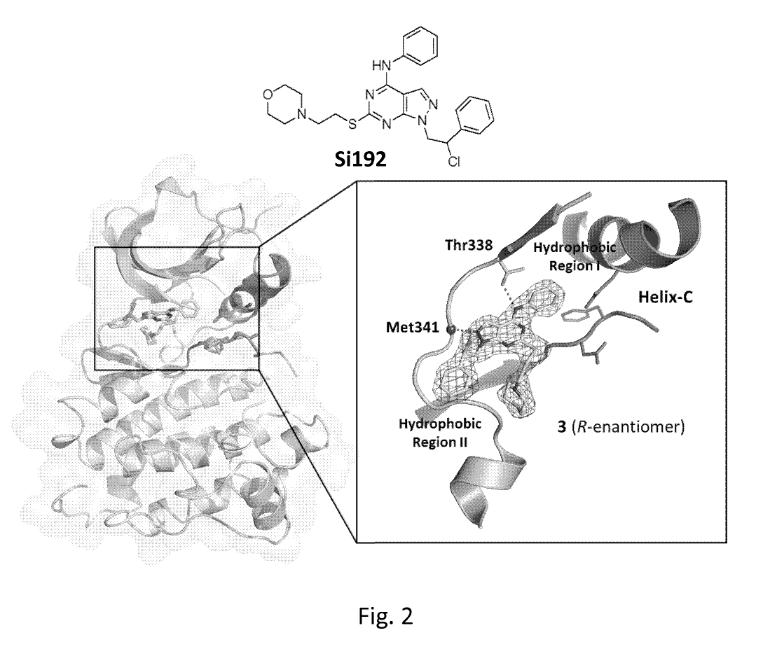

FIG. 2. Inhibitor Si192 in complex with wild-type cSrc (PDB-code: 4O2P). The experimental electron density of Si192 at 2.1 Å resolution is displayed (2Fₒ-F_{c} map contoured at 1σ). The kinase domain is in the active DFG-in conformation and hydrogen-bond interactions of the inhibitor with Thr338 (gatekeeper) and accordingly the backbone amide of Met341 are illustrated as red dotted lines. Hinge region (orange), helix C (turquoise), DFG-motif (pink) and inhibitor Si192 (yellow sticks).

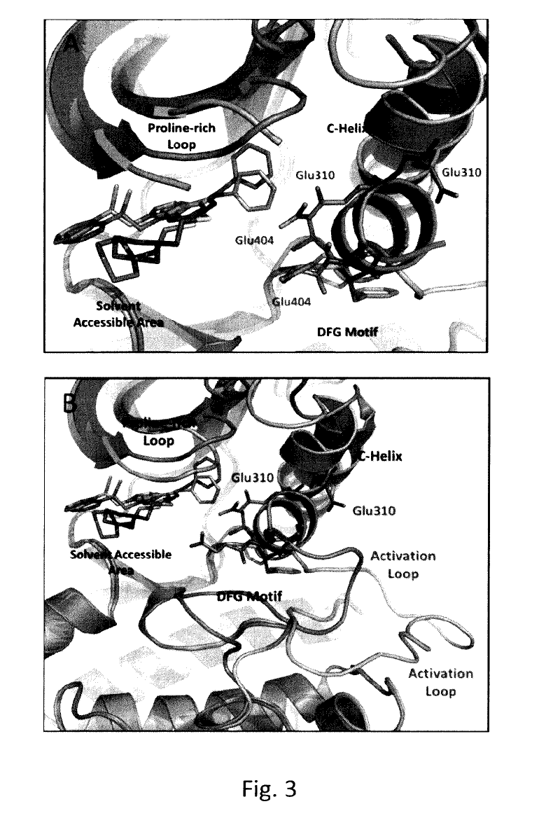

FIG. 3. A) Chain A (magenta) and B (aquamarine) of the crystal structure aligned each other. Differences between the two conformations were observed at the level of activation loop, αC-helix and P-loop. B) Chain A (magenta) and B (aquamarine) used for the MC/FEP calculations. Missing residues in crystal structure were modeled.

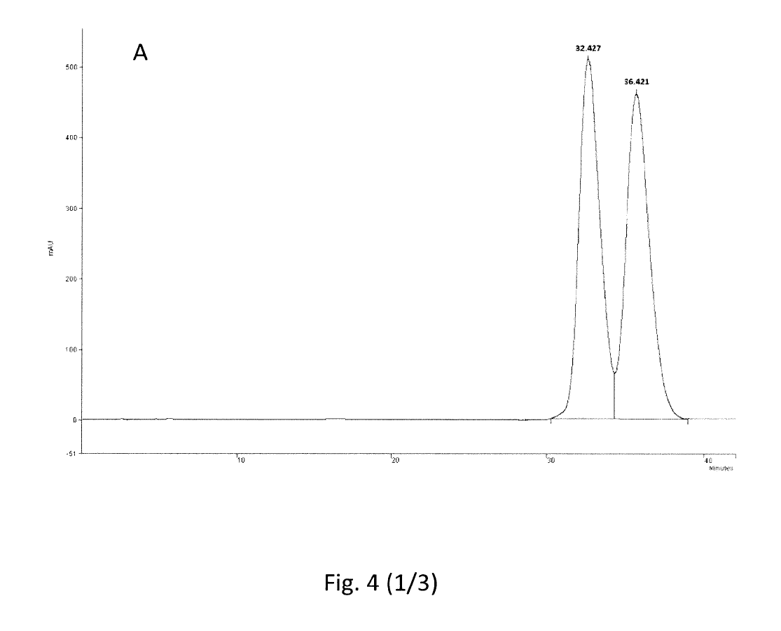

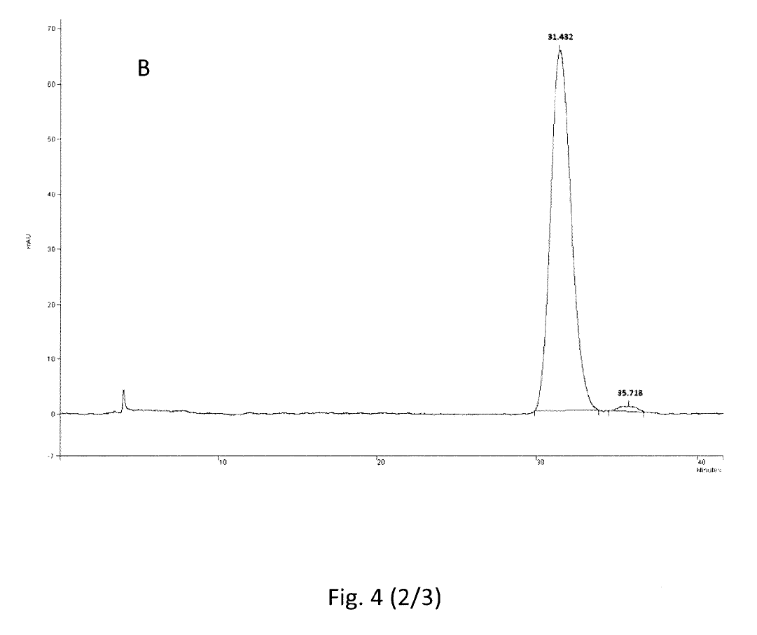

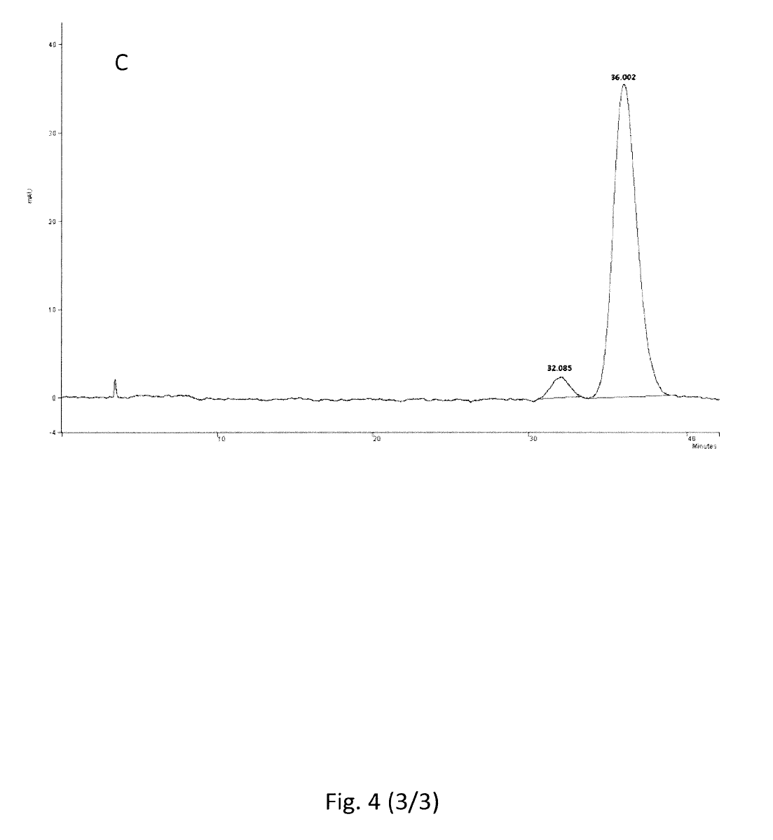

FIG. 4. Panel A: Analytical HPLC resolution of Si192 racemic compound on Chiralcel OD at flow-rate of 0.8 mL/min with a mobile phase n-hexane/2-propanol doped with 5% of acetonitrile 90:10 (v/v) (Rt1 31.33 min, Rt2 36.22 min); Panel B and C: Analytical HPLC re-runs on the single separated enantiomer on Chiralcel OD at flow-rate of 0.8 mL/min with a mobile phase n-hexane/2-propanol doped with 5% of acetonitrile 90:10 (v/v).

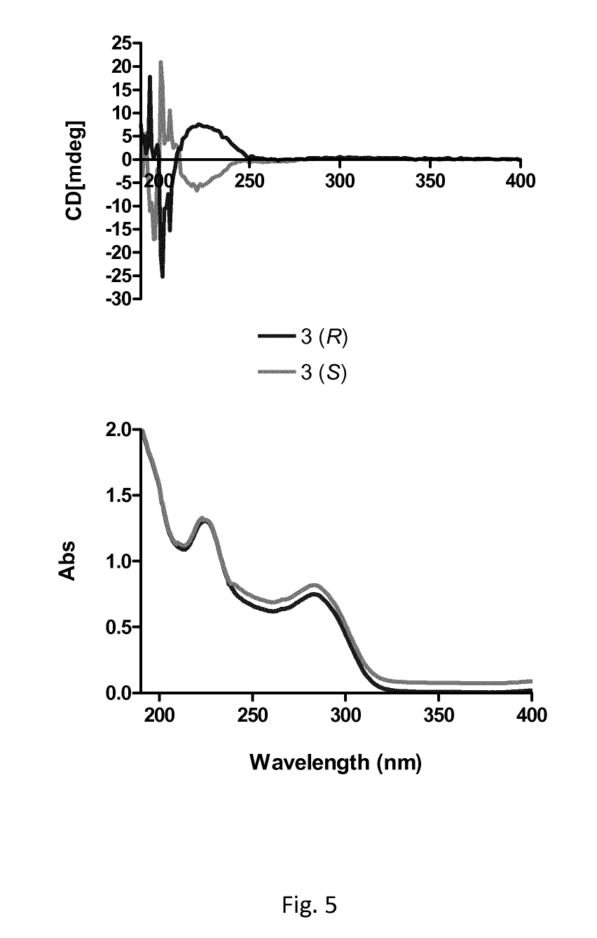

FIG. 5. CD spectra (methanol, room temperature) of the enantiomers of compounds 3 obtained from the racemic mixture separations. The first eluate is black and the second one is grey.

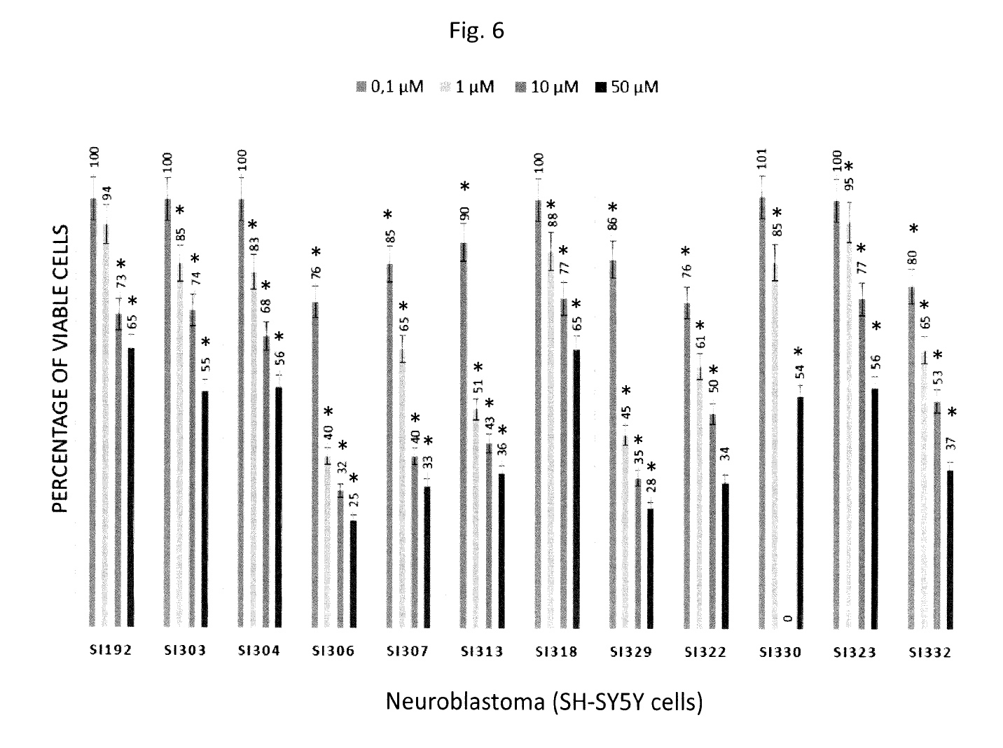

FIG. 6. Viability test on neuroblastoma SH-SY5Y cells treated with increasing concentrations (0.1, 1, 10, 50 uM) of different compounds (Si192, Si303, Si304, Si306, Si307, Si313, Si318, Si329, Si322, Si330, Si323, Si332). Percentage of viable cells respect to vehicle treated cells (control cells=100%) is shown. This experiment demonstrates the potency of each compound in inhibiting SH-SY5Y cells proliferation. Data represent the mean percentage and SD from at least three different experiments. *P<0.01 according to Student's t test respect to control cells.

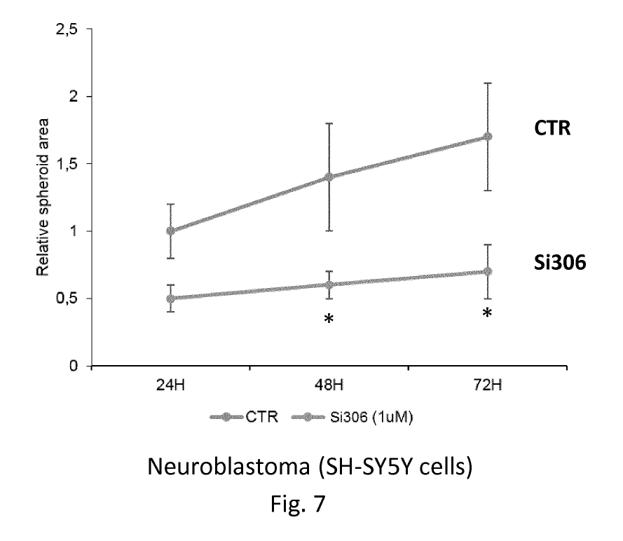

FIG. 7. Evaluation of SH-SY5Y cell growth. Cells were cultured as spheroid in presence of Si306, 1 μM. The mean area of spheroid was calculated as described in the experimental section at 24, 48 and 72 h. Representative images of cell cultures at the endpoint taken by contrast microscope are shown on the right. *p<0.01 according t-Student test. CTR=control treated with vehicle.

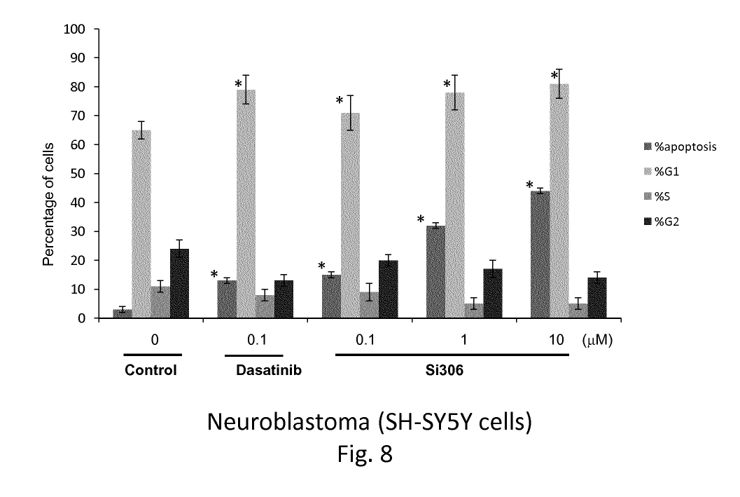

FIG. 8. Analysis of the cell cycle distribution of SH-SY5Y cells after treatment with 0.1 μM Dasatinib and increasing concentrations of Si306. SH-SY5Y cells status was investigated by cytofluorimetry after propidium iodide staining and results were expressed as percentage of cells in each phase of cell cycle respect to total viable cells. Apoptosis was evaluated by calculating the number of hypodiploid cells and was expressed as percentage of apoptotic cells respect to total cells (viable and dead cells). Results are the mean±SD of three different experiments. *p<0.01 (Student's t test) vs. value of control cells treated with vehicle (Control).

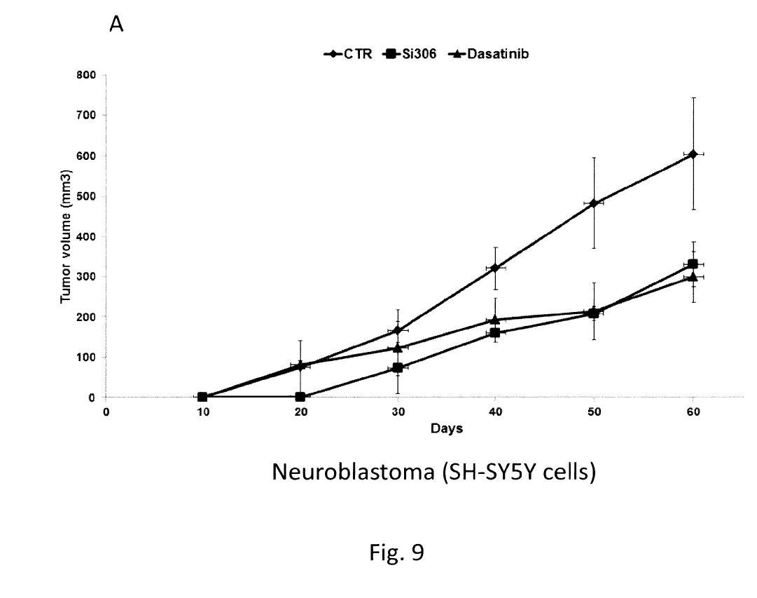

FIG. 9. Evaluation of antitumoral effect of Si306. A) Inhibition of tumor growth by Si306 (50 mg/kg) or Dasatinib (50 mg/kg) treatment in a rodent model of NB. Tumor xenografts were monitored measuring the diameters of tumor mass. B) Anti-angiogenic effect of Si306 evaluated by sprouting assay with endothelial cells. Histogram shows the mean number of sprouts per spheroid for each experimental condition. Representative images are shown on the right. *p<0.01 according t-Student test. CTR=control treated with vehicle.

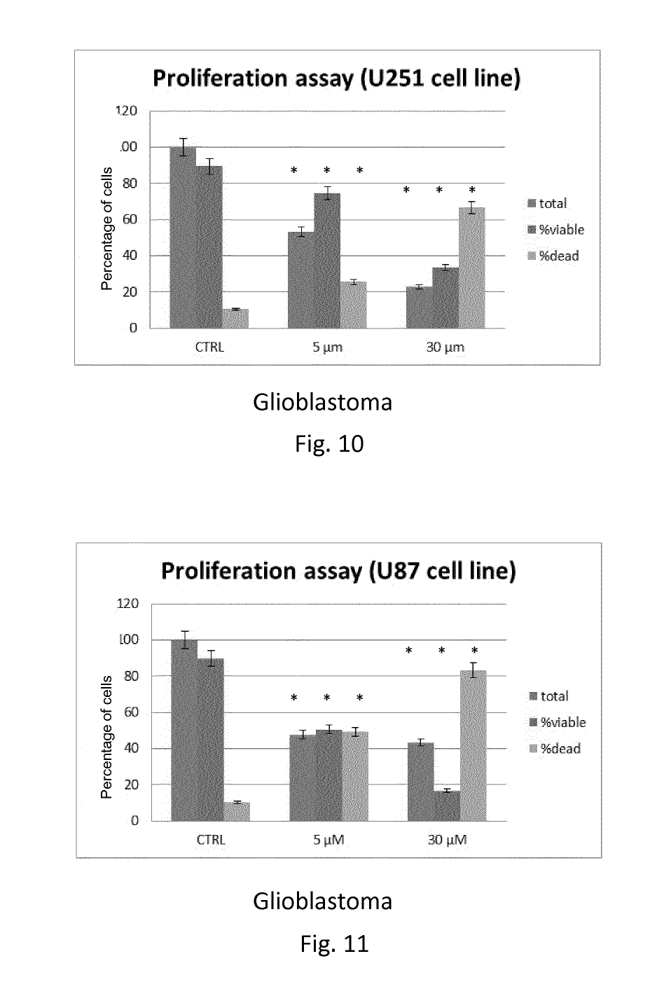

FIG. 10. Number of GBM U251 cells expressed as percentage of cells respect to CTRL (CTRL=control cells treated with vehicle). U251 cells were treated for 72 h with Si306, 5 μM and 30 μM. For each experimental point the percentage of viable and dead cells is indicated. *p<0.01 according t-Student test vs control.

FIG. 11. Number of U87 cells expressed as percentage in respect to CTRL (CTRL=control cells treated with vehicle). U87 cells were treated for 72 h with Si306, 5 μM and 30 μM. For each experimental point, the percentage of viable and dead cells is indicated. *p<0.01 according t-Student test vs control.

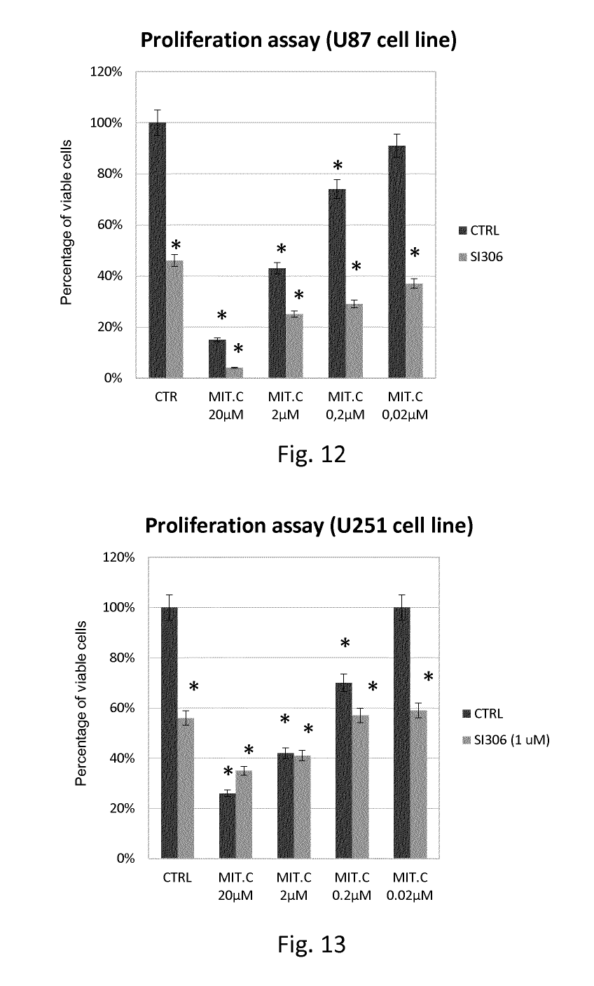

FIG. 12. Number of U87 cells expressed as percentage in respect to CTRL (CTRL=control cells treated with vehicle). U87 cells were treated for 72 h with Si306, 10 μM and increasing concentrations of mitomycin C (MIT.C, 0.02-20 μM). CTR=control cells treated with vehicle. *p<0.01 according t-Student test vs control.

FIG. 13. Number of U251 cells expressed as percentage in respect to CTRL (CTRL=control cells treated with vehicle). U251 cells were treated for 72 h with Si306, 10 μM and increasing concentrations of mitomycin C (MIT.C, 0.02-20 μM). *p<0.01 according t-Student test vs control.

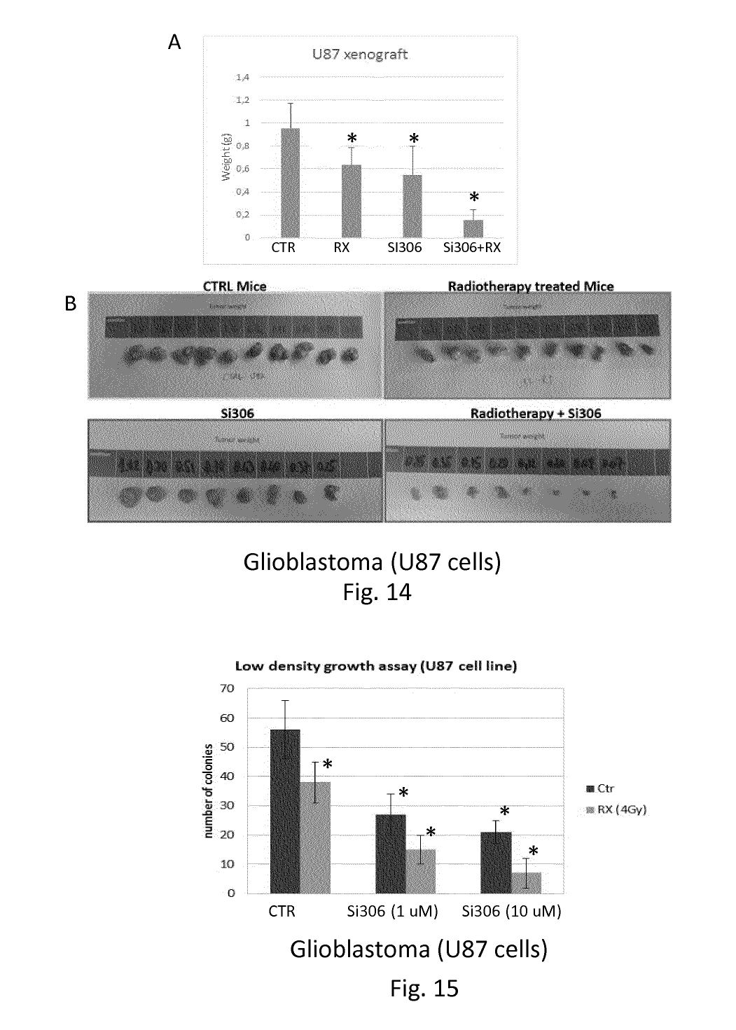

FIG. 14. In vivo model of GBM. Mice were treated with Si306 and radiotherapy (RX) (A) Histograms of tumor weight at the end of the experiment. Tumors were excided from in vivo models of GBM obtained by inoculating U87 cells subcutaneously in immunodeficient mice. Mice were treated once with radiation (RX, 4Gy) and every other day with 50 mg/kg Si306 (306) for 30 days. (B) Representative images of excided tumors are shown. CTR=control treated with vehicle. RX treatment induced a reduction of about 40% (vs CTR). Si306 induces a reduction of about 50% (vs CTR). The combined treatment (radiation+Si306) induced a reduction of about 80% (vs CTR). *p<0.01 according t-Student test vs control.

FIG. 15. Number of colonies formed by U87 cells after treatment with radiation (RX, 4Gy) and 1 μM or 10 μM Si306. CTR=control cells treated with vehicle. *p<0.01 according t-Student test vs control

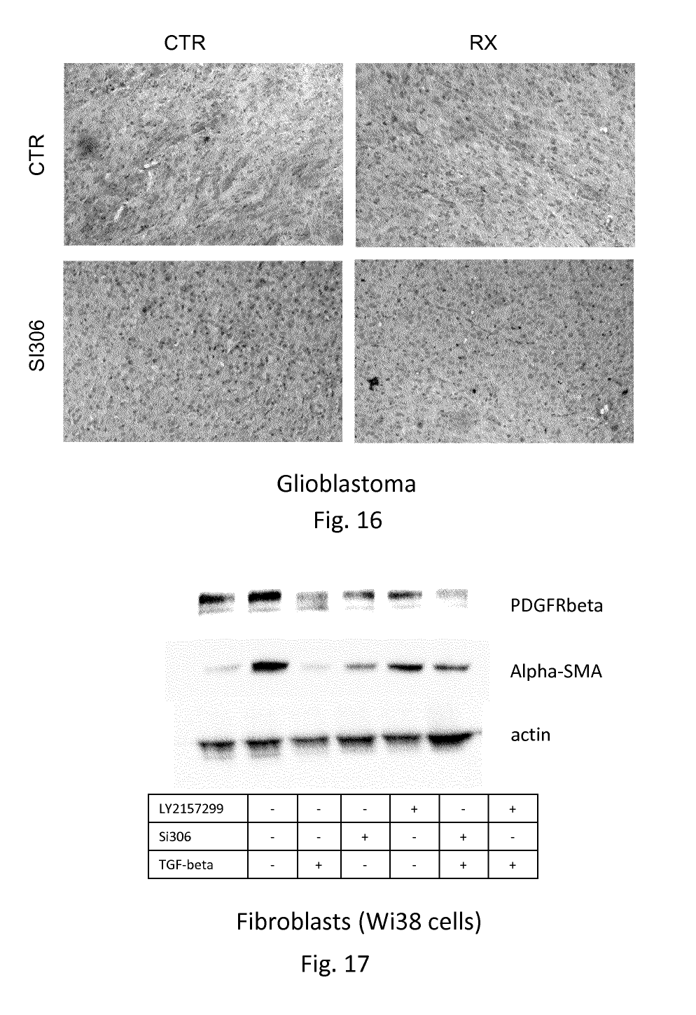

FIG. 16. Immunohistochemistry assay for alpha-SMA expression. Tumor masses from experiment as described in FIG. 14 were analyzed for the composition of stromal compartment. In particular, the expression alpha-SMA (brown staining), a marker of myofibroblasts, was evident only in tumor excised from mice that have not been treated with Si306.

FIG. 17. Western blot analysis of PDGFR-beta and alpha-SMA expression. Human fibroblasts were treated with TGF-beta, a known inducer of myofibroblast differentiation, and with LY2157299 (5 μM, inhibitor of TGF-beta receptor) or Si306 (1 μM). The TGF-beta differentiation of fibroblasts was demonstrated by the upregulation of PDGFRbeta and alpha-SMA. Si306 was able to counteract this differentiation and its effect was similar to LY2157299 (specific inhibitor of TGF-beta receptor)

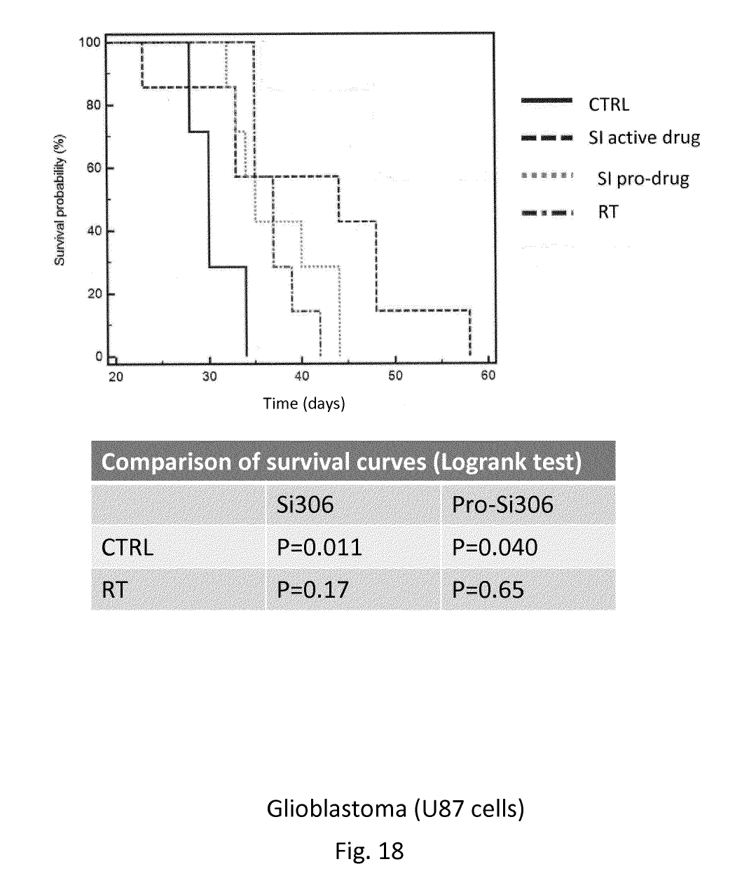

FIG. 18. Survival curves (days) of orthotopic mouse model of GBM. U87 cells were injected orthotopically in mouse brain and mice were divided in four groups (7 mice per group): control group (CTRL) receiving the vehicle; Si active drug group receiving 50 mg/kg Si306 (three times per week and for 4 weeks); Si pro-drug group receiving 50 mg/kg pro-Si306 (three times per week and for 4 weeks); RT: group treated once with radiation (RX, 4Gy). Survival time was recorded and statistical analysis was performed comparing Si306 and pro-Si306 groups with CTRL and RT groups.

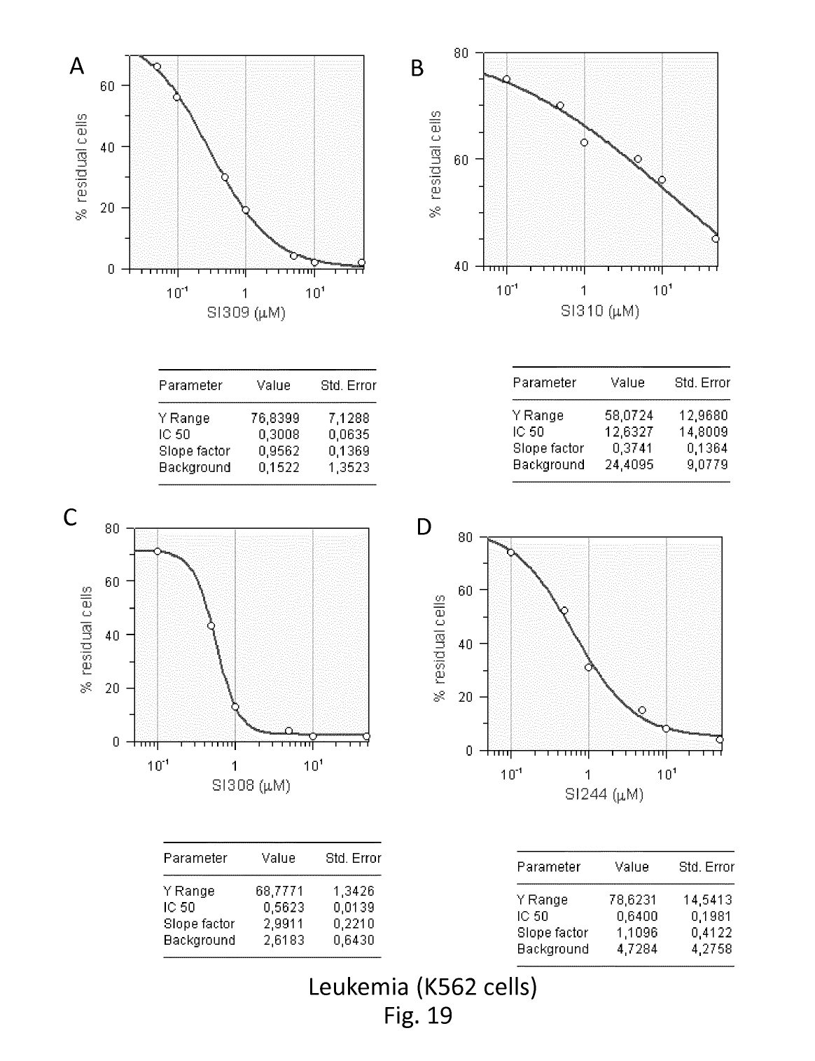

FIG. 19. Sigmoid curves generated from proliferation assays of leukemia K562 cells treated with increasing concentrations of different compounds (A, B, C, D). Mathematical characteristics of the curves, including IC₅₀ and standard deviations, are shown in the tables.

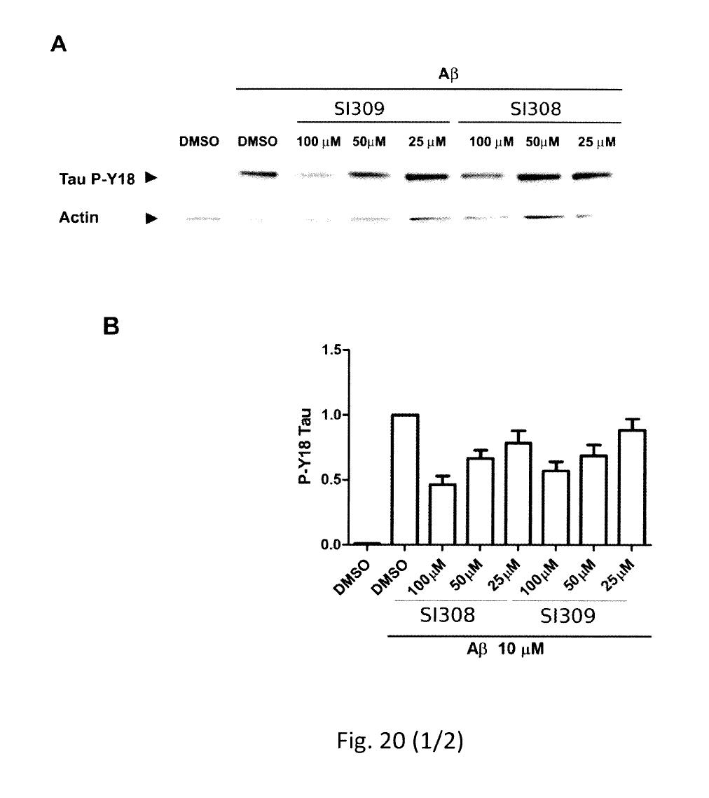

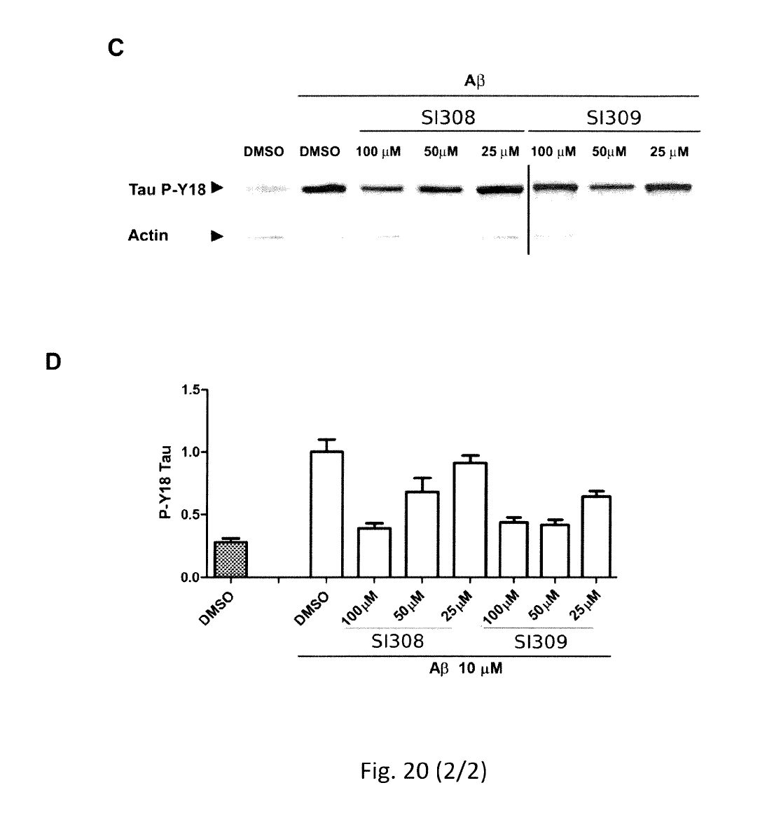

FIG. 20. Si308 and Si309 inhibits Aβ₄₂ mediated phosphorylation of Tyr17-Tau in differentiated SH-SY5Y cells. Western blot analysis of Aβ₄₂ mediated phosphorylation of Tyr17-Tau was performed after 1.5 hours (A) or 6 hours (C) from administration of different amount of compounds Si308 and Si309. (B) and (D), data were quantified by chemiluminescence. Experiments were conducted in triplicate, error bars represent ±SEM.

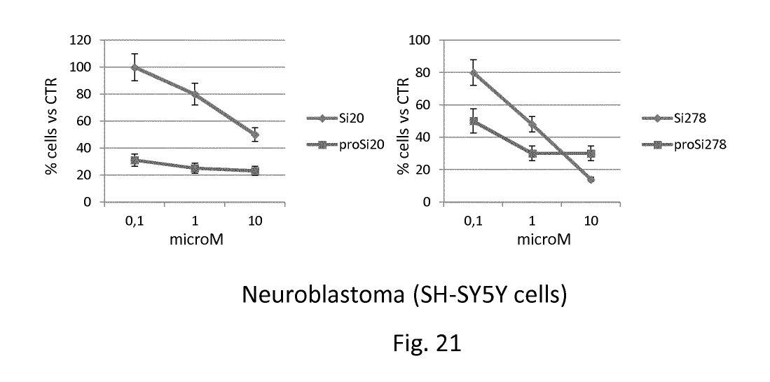

FIG. 21. Viability analysis of neuroblastoma SH-SY5Y cells treated for 72 h with Si20, proSi20, Si278 and proSi278 (0.1 μM, 1 μM and 10 μM) and expressed as percentage respect to control cells. Each graph show the comparison between the drug and the respective pro-drug. Data (mean and SD) from at least three different experiments.

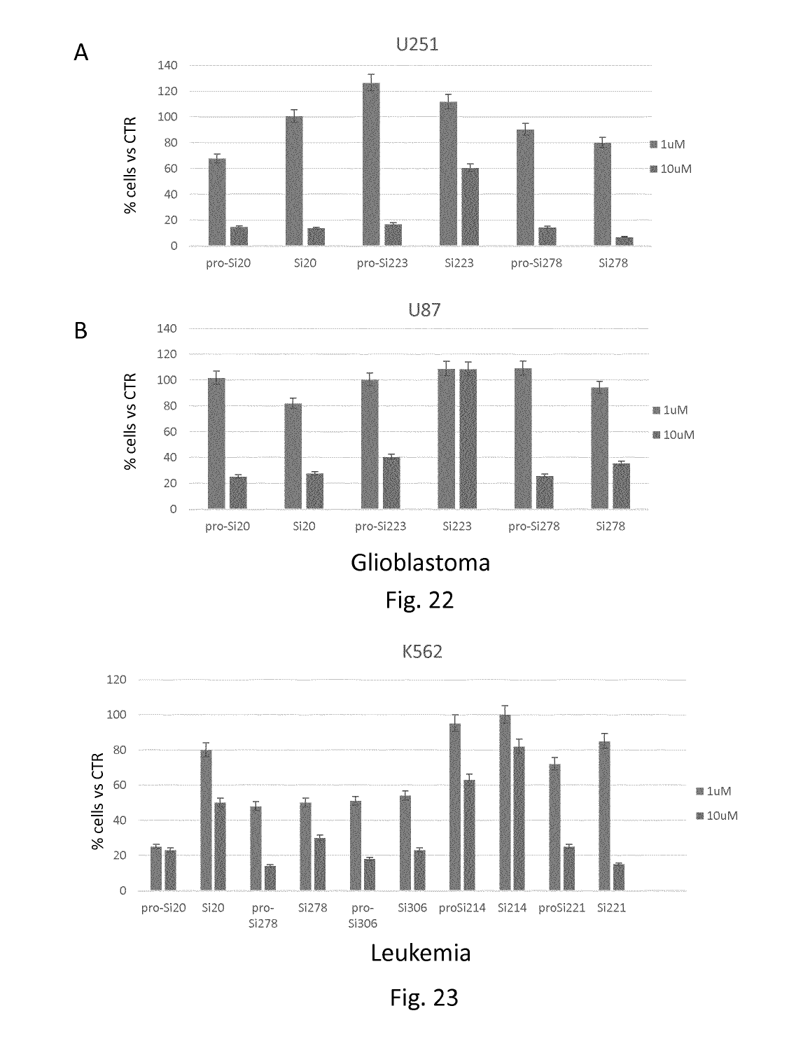

FIG. 22. Histograms show results from viability test of glioblastoma cell lines (U251 and U87) treated for 72 h with 1 and 10 μM of different drugs and respective pro-drugs. Mean and SD from three different experiments.

FIG. 23. The antitumoral activity of a panel of drugs and respective pro-drugs was tested in leukemia cells K562. Cells were treated for 72 h with 1 and 10 μM of each drug or pro-drug. Results are espressed as mean percentage and SD respect to untreated cells (three different experiments).

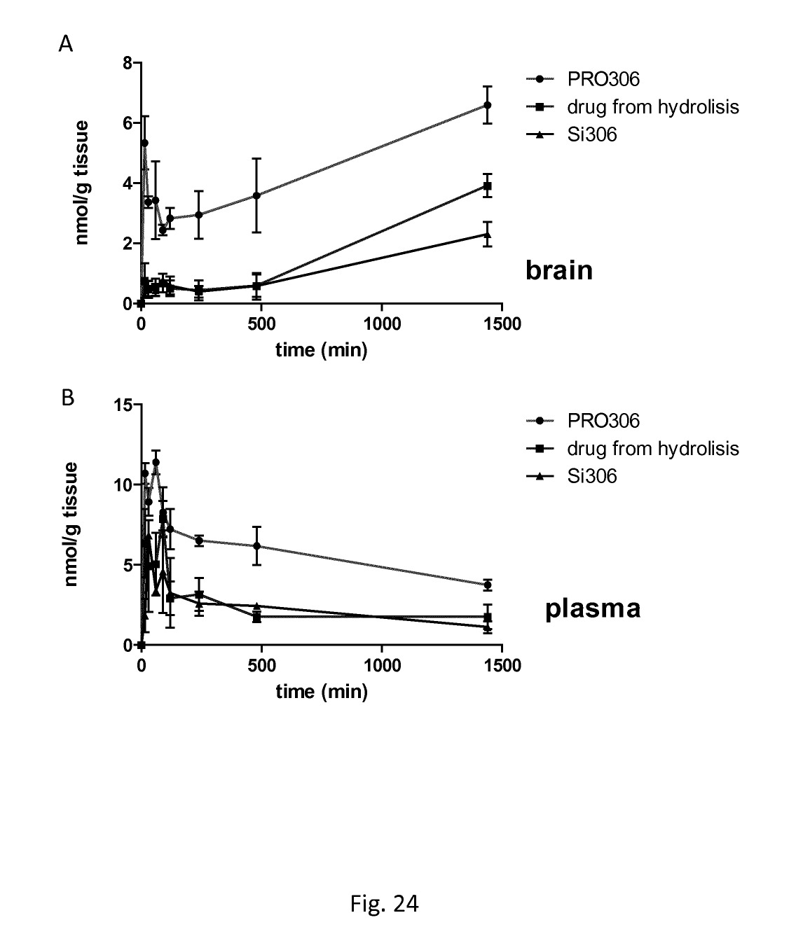

FIG. 24. Compounds (ProSi306 and its hydrolysis-derived drug Si306, and Si306) were quantified by HPLC-UV-MS analysis, in brain and plasma tissue at defined time points. Balb/c mice were treated with ProSi306 and Si306, 50 mg/Kg, by ip injection for 24 h. Experiments were performed in triplicate.

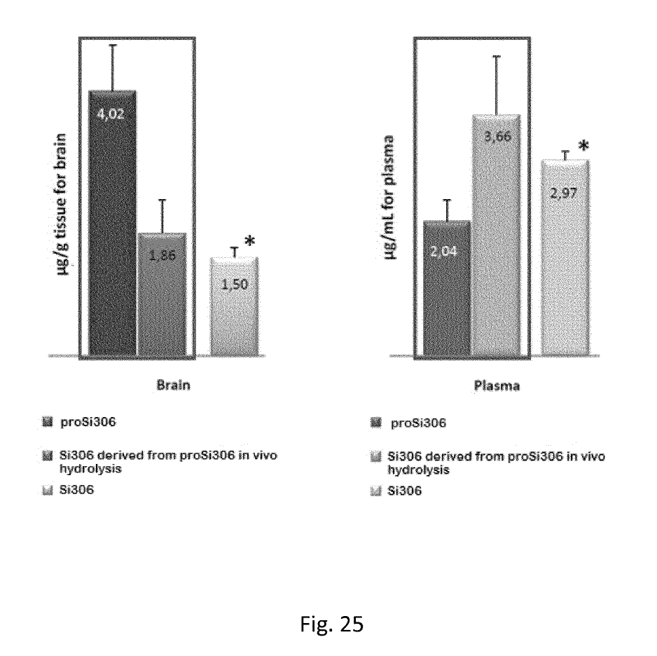

FIG. 25. Biodistribution obtained by intraperitoneal injection of compound (Si306 e proSi306) 50 mg/Kg in Balb/c mice. The framed area shows the quantity of proSi306 and Si306 (derived from hydrolysis of proSi306) found in Brain and Plasma of mice treated with Si306 only. The bars (*) represent the quantity of drug Si306 found in Brain and Plasma of mice treated with the free drug Si306. Experiments were performed in quadruplicate. Measurements were performed after 24 h treatment. Samples were analysed by HPLC-UV-MS.

### EXAMPLE 1

### Compounds Synthesis and Characterization Thereof

### 1.1—X-Ray Structure and Computational Studies

Crystallization and Structure Determination of c-Src-SI192.

Inhibitor SI192 was co-crystallized with c-Src using conditions similar to those previously reported by Michalczyk et al.⁵⁸ Briefly, final concentrations of 540 μM inhibitor (100 mM stock in DMSO) and 180 μM wild type c-Src (stored in 50 mM Tris pH 8.0, 100 mM NaCl, 1 mM DTT, 5% glycerol (v/v)) were pre-incubated for 1 h on ice to form the enzyme-inhibitor complex prior to crystallization. Crystals were grown using the hanging drop method at 20° C. after mixing 1 μL protein-inhibitor solution with 1 μL reservoir solution (0-30 mM NaCl, pH 7.0, 9-20% ethylene glycol). All crystals were frozen with further addition of 30% (v/v) glycerol. Diffraction data of the c-Src-SI192 complex crystals were collected at the PX10SA beamline of the Swiss Light Source (PSI, Villingen, Switzerland) to a resolution of 2.1 Å, using wavelengths close to 1 Å. The data set was processed with XDS⁶⁰ and scaled using XSCALE.⁵⁹⁻⁶⁰

Structure Determination and Refinement of c-Src-Si192.

The c-Src-inhibitor complex structure was solved by molecular replacement with PHASER⁶¹ using the published c-Src structure 2OIQ⁶² as template. The two c-Src molecules in the asymmetric unit were manually modified using the program COOT.⁶³ The model was first refined with CNS⁶⁴ using simulated annealing to remove model bias. The final refinement was performed with REFMAC5.⁶⁵ Inhibitor topology files were generated using the Dundee PRODRG2 server.⁶⁶ Refined structures were validated with PROCHECK.⁶⁷ Detailed data, refinement, and Ramachandran statistics are provided in Table 1.

TABLE 1
Data collection and refinement statistics
for c-Src wt in complex with Si192

| | cSrc wt with Si192 |
|---|---|
| | (4O2P) |
| Date collection | |
| Space group | P1 |
| Cell dimensions | |
| a, b, c (Å) | 42.22, 63.25, 74.81 |
| α, β, γ (°) | 101.35, 90.44, 90.07 |
| Resolution (Å) | 50.0-2.1 (2.20-2.10)^{a} |
| R_{sym} or R_{merge} (%) | 4.4 (20.1) |
| I/σI | 11.9 (4.0) |
| Completeness (%) | 96.1 (95.4) |
| Redundancy | 1.9 (1.9) |
| Refinement | |
| Resolution (Å) | 43.3-2.10 |
| No. reflections | 42544 |
| R_{work}/R_{free} | 19.7/23.9 |
| No. atoms | |
| Protein | 4257 |
| Ligand/ion | 68 |
| Water | 236 |
| B-factors | 37.4 |
| Protein | 37.5 |
| Ligand/ion | 41.3 |
| Water | 34.6 |
| R.m.s. deviations | |
| Bond lengths (Å) | 0.010 |
| Bond angles (°) | 1.142 |

| Structure | cSrc wt with 1 |
|---|---|
| (PDB-ID code) | (4O2P) |
| Wavelength (Å) | 0.978600 |
| Temperature | 90 K |
| X-ray source | SLS X10SA |
| Ramachandran Plot: | |
| Residues in | |
| most favored regions | 90.2% |
| additional allowed regions | 9.3% |
| generously allowed regions | 0.4% |
| dissallowed regions | 0.0% |

^{a}Diffraction data from a single crystal were used to determine the complex structure. Values in parenthesis are referring to the highest resolution shell.

### Computer Modeling

Loops Modeling Protocol.

The FASTA sequence of c-Src was used as query, the coordinates of the two chains of the inventors' crystal structure (c-Src in complex with SI192) were in turn employed as templates and the missing residues were built by using the program Prime.⁶⁸ For each chain, the serial loop sampling approach was applied by choosing “Extended” as level of accuracy (recommended for loop length between 6 and 11 residues) and the lowest energy conformation was saved for the next analysis. Similarly, Prime was used to fill the A-loop of the chain B by the building of the Cys277 missing residue and to construct the amino acids 300 and 301 absent in the chain A.⁶⁹ The maximum number of structures to return was set to 10. An energy cut-off of 10 kcal/mol was applied. Loop conformations were clustered and representatives of each cluster were selected. The best scoring loop structure was finally selected.

Monte Carlo/Free Energy Perturbation.

MC/FEP calculations were performed with the MCPRO program and following standard protocols.^{70,71} Z-Matrix for the c-Src-ligand complexes were obtained with the molecular growing program BOMB⁷⁰ starting from the pose of SI192 within the inventors' crystal structure (PDB-code: 4O2P). The models included the 160 amino acid residues nearest to the ligand. Short conjugate-gradient minimizations were carried out on the initial structures for all complexes to relieve any unfavorable contacts. Coordinates for the free ligands were obtained by extraction from the complexes. Next, a 1500 steps of conformational search analysis was carried out on the ligands using BOSS⁷² program with the OPLS/CM1Ax force field and GB/SA hydration. The resultant conformer with the lowest-energy was used for FEP calculation. The unbound ligands and complexes were solvated with TIP4P water spheres (“caps”) with a 25 Å radius. The water molecules in too close contact with solute atoms were removed. A few remote side chains were neutralized in order to maintain overall charged neutrality for each system. The ligand and the protein side chains within 10 Å of any ligand were sampled during the MC simulations. The only constraints were the bond lengths in side chains, and all backbone atoms were frozen after a short conjugate-gradient minimization. The energetics for the systems were evaluated with the OPLS-AAx force field for the protein and OPLS/CM1Ax for ligands.⁷³ The CM1A atomic charges were scaled by 1.14 for neutral molecules. Differences in free energies of binding were determined from the usual thermodynamic cycle that requires conversion of one ligand to another both free in water and bound to the protein. The FEP calculations utilized 11 windows of simple overlap sampling. For the unbound ligand, each window consisted of 40 M configurations of equilibration and 60 M configurations for averaging. For the bound calculations each windows covered 20 M configurations of solvent only equilibration, 40 M configurations of full equilibration and 50 M configurations of averaging. In the case of halogen bond scanning the number of configurations was increased to 60 M of equilibration and 80 M of averaging. All MC simulations were run at 298 K.

### X-Ray Structure and Computational Studies.

To gain a deeper structural understanding of C6 substituted derivatives binding mode, the inventors determined the crystal structure of a complex of the kinase domain of c-Src (aa 256-533) and the hit compound Si192. Diffraction data was collected to 2.1 Å resolution and subsequent data processing and refinement exhibited two protein molecules within the crystallographic cell unit which in this work will be referred to as chain A and chain B. Comparative analysis of the empirically determined protein-ligand structure and previous docking studies illustrated coincident binding modes of Si192 with respect to c-Src (FIG. 2).²⁰ The C4 anilino substituent and the N1 side chain are located within the hydrophobic regions I and II, respectively. Furthermore, the X-ray structure confirmed the presence of two predicted hydrogen bonds, involving the C4 amino group which interacts with Thr338 side chain and the N2 of the pyrazolopyrimidine scaffold taking contacts with the backbone of Met341. Remarkably, the same binding orientation was observed for compound Si192 within the ATP binding pocket of each chain. However, despite many residues of the activation loop were poorly defined (from 413 to 424 in chain A and from 411 to 424 in chain B) significant differences between the two chains were observed in the 3D rearrangement of such flexible loop (aa 402-423) as well as in the position of the αC-helix (aa 303-318) and in the glycine-rich loop conformation (aa 273-281) (FIG. 3A). In particular, in chain A the Glu310 side chain projects away from the ATP binding site adopting a conformation similar to the closed and inhibited one of c-Src phosphorylated on Tyr527 (PDB code: 2SRC).⁷⁵ On the contrary, in chain B, Glu310 displays its side chain turned towards the active site forming a salt bridge with Lys295 which is typical of active kinases. Moreover, in chain A the solved amino acids of activation loop (Phe405-Asp413) are arranged in a three-turn alpha helix in a similar although not identical way as in phosphorylated c-Src (PDB code: 2SRC).⁷⁵ Vice-versa, the determined activation loop of chain B recalls the one solved for the active conformation of c-Src (PDB code: 1Y57).⁷⁶ Another significant difference between chains A and B resides in the orientation of the DFG motif: in chain A Glu404 projects its side chain deeply into the ATP binding site, thereby reducing the size of the hydrophobic pocket I which harbors the C4 substituent. Structural plasticity of c-Src in the presence of small molecule inhibitors was recently described.⁷⁷ To take into account the conformational differences, both chains were used in all the subsequent computational studies. A molecular modeling protocol was firstly applied to fill the missing residues (see Experimental section above for details) and the two refined chains were aligned to each other (FIG. 3B). Starting from these completed structures, the optimization of Si192 was pursued using a computationally driven approach, primarily guided by results of Monte Carlo Free-Energy Perturbation (MC/FEP) calculations.⁷⁸ Notably, although the racemic mixture of Si192 was used for the preparation of the X-ray crystal structure, solely the R-enantiomer was found to be able to bind within the kinase active site in both the chains pushing the inventors' studies towards further investigation on the chiral center. No differences were observed in the activities of the two enantiomers against c-Src (see In vitro biological activity paragraph below, Table 6).

Next the inventors focused their attention on the C4 anilino ring with the aim of optimizing the activity of Si192 by increasing the affinity for the c-Src kinase. MC/FEP halogen (chlorine, bromine and fluorine) and hydroxyl scans were performed to identify the most promising sites and groups for substitutions of C4 anilino hydrogens. In the present calculations ortho positions 2,6 and meta positions 3,5 are not equivalent as they do not interconvert during the MC runs requiring separate simulations for each conformer. According to the ring numbering in Table 2, replacement of hydrogen by OH was predicted to be favorable (positive free energy of binding, ΔΔG_{b}) by 5.11, 4.89, 1.49 kcal/mol at C2, C3 and C4, respectively and unfavorable at C5 and C6 (ΔΔG_{b} of −6.68 and −5.08 kcal/mol, respectively) when initial complexes were built using chain B.

TABLE 2
MC/FEP results for the change in free energy of binding upon introduction of chlorine,
bromine, fluorine and hydroxyl substituents at the C4 anilino ring within Chain B.

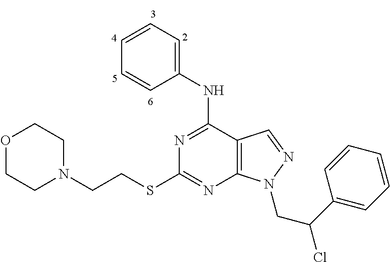 [SMILES: ClC(Cn1ncc2c(Nc3ccccc3)nc(SCCN3CCOCC3)nc21)c1ccccc1]

| | OH to H | | Cl to H | | Br to H | | F to H | |
|---|---|---|---|---|---|---|---|---|
| | ΔΔG_{b} | Σ | ΔΔG_{b} | σ | ΔΔG_{b} | σ | ΔΔG_{b} | σ |
| C2 | 5.11 | ±0.10 | 4.8 | ±0.11 | 1.28 | ±0.11 | 1.96 | ±0.05 |
| C3 | 4.89 | ±0.11 | −5.37 | ±0.09 | −6.67 | ±0.11 | 0.25 | ±0.03 |
| C4 | 1.49 | ±0.13 | −6.73 | ±0.09 | −9.33 | ±0.10 | −3 | ±0.05 |
| C5 | −6.68 | ±0.12 | −8.36 | ±0.21 | −8.71 | ±0.27 | −3.66 | ±0.08 |
| C6 | −5.08 | ±0.09 | −1.05 | ±0.07 | −5.43 | ±0.11 | 0.71 | ±0.05 |

^{a}ΔΔG_{b} is the computed change in free energy of binding (kcal/mol) for introducing the substituents;
± σ is the computed uncertainty.

Positive ΔΔG_{b} values were also found with the introduction of chlorine, bromine or fluorine at C2 (4.8, 1.28 and 1.96 kcal/mol, respectively). On the contrary, in chain A the entity of these substituents resulted to be unfavorable with negative ΔΔG_{b} (Table 3).

TABLE 3
MC/FEP results for the change in free energy of binding upon introduction of chlorine,
bromine, fluorine and hydroxyl substituents at the C4 anilino ring within Chain A.

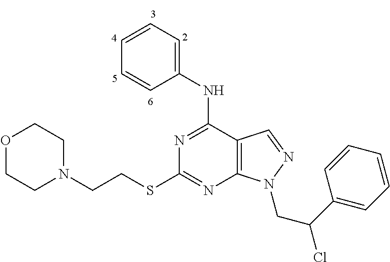 [SMILES: ClC(Cn1ncc2c(Nc3ccccc3)nc(SCCN3CCOCC3)nc21)c1ccccc1]

| | OH to H | | Cl to H | | Br to H | | F to H | |
|---|---|---|---|---|---|---|---|---|
| | ΔΔG_{b} | Σ | ΔΔG_{b} | σ | ΔΔG_{b} | σ | ΔΔG_{b} | σ |
| C2 | −1.96 | ±0.08 | −2.47 | ±0.13 | −3.47 | ±0.14 | 0.49 | ±0.05 |
| C3 | −9.41 | ±0.10 | −2.70 | ±0.16 | −4.58 | ±0.18 | −3.78 | ±0.06 |
| C4 | −11.23 | ±0.08 | −6.77 | ±0.13 | −12.79 | ±0.20 | −1.55 | ±0.06 |
| C5 | −7.51 | ±0.17 | −12.14 | ±0.19 | −10.77 | ±0.15 | −2.89 | ±0.10 |
| C6 | 0.06 | ±0.13 | −6.83 | ±0.19 | −11.03 | ±0.16 | −6.48 | ±0.07 |

^{a}ΔΔG_{b} is the computed change in free energy of binding (kcal/mol) for introducing the substituents;
± σ is the computed uncertainty.

Taking into account the MC/FEP results, a focused library of pyrazolo[3,4-d]pyrimidine derivatives bearing a m-OH substituent at the C4 anilino ring was synthesized in order to increase both water solubility and c-Src binding affinities of compounds under study. Furthermore, analogues substituted with bromine, chlorine and fluorine in meta position were also synthesized and tested in enzymatic assays to enlarge the structure-activity relationships (Table 4), despite the prediction of unfavorable outcomes. Concerning these last substitutions, the possibility of halogen bonding between the inventors' inhibitors and the ATP binding site was also investigated by halogen bond scanning on both chain A and B considering m-Br and m-Cl substituents (Table 4).

TABLE 4
Halogen bond scanning for chlorine and
bromine atoms in both Chain A and B.

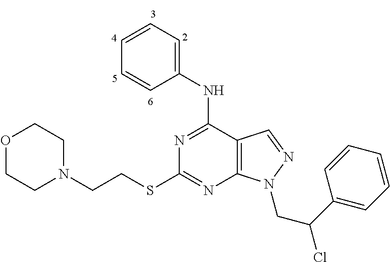 [SMILES: ClC(Cn1ncc2c(Nc3ccccc3)nc(SCCN3CCOCC3)nc21)c1ccccc1]

| | Chain A | | | | Chain B | | | |
|---|---|---|---|---|---|---|---|---|
| | Br | | Cl | | Br | | Cl | |
| | ΔΔG_{b} | σ | ΔΔG_{b} | σ | ΔΔG_{b} | Σ | ΔΔG_{b} | σ |
| C3 | −1.66 | ±0.10 | −3.42 | ±0.06 | −1.67 | ±0.03 | −0.89 | ±0.03 |
| C5 | −0.89 | ±0.06 | −0.79 | ±0.05 | 0.35 | ±0.03 | 1.14 | ±0.05 |

^{a}ΔΔG_{b} is the computed change in free energy of binding (kcal/mol) for introducing the substituents;
± σ is the computed uncertainty.

A marginal effect of halogen bond interaction was found for chlorine and bromine substituents at C5 position during chain B simulations (1.14 and 0.35 kcal/mol, respectively) while negative results were obtained in case of using chain A. The calculated positive contribution of halogen bonding was due to the interaction of Cl or Br with the carbonyl backbone of Ile336, working as Lewis base. However, this contribution has only limited effect on the total free energy of binding calculated for the introduction of bromine or chlorine at the meta position of C4 anilino group, which still remains generally negative. In summary, analysis of the MC/FEP results clearly highlighted that c-Src binding affinity may be enhanced by replacing the hydrogen by a hydroxyl group at position 3 of C4 anilino ring. This substitution allows for the stabilization of the complex between the active conformation of c-Src and the pyrazolo[3,4-d]pyrimidines studied herein. The hydrogen-bond interaction between the 3-OH of the ligand and the Glu310 side chain, usually involved in the formation of a salt bridge with Lys295, undoubtedly gives an important contribution to the binding affinity. On the other hand, the introduction of an hydroxyl group or halogens at position 2 were also predicted as favorable and will thus be subjected to the inventors' future studies.

### 1.2—Chemistry: Materials and Methods

Starting materials were purchased from Aldrich-Italia (Milan, Italy). Melting points were determined with a Büchi 530 apparatus and are uncorrected. IR spectra were measured in KBr or CHCl₃ with a Perkin-Elmer 398 spectrophotometer. ¹H NMR spectra were recorded at 400 MHz in CDCl₃ or (CH₃)₂SO on a Bruker Avance DPX400 spectrometer. Chemical shifts are reported as δ (ppm) relative to TMS as the internal standard, J in Hz. ¹H patterns are described using the following abbreviations: s=singlet, d=doublet, t=triplet, q=quartet, quint=quintet, sx=sextet, sept=septet, m=multiplet, br=broad signal, br s=broad singlet. TLC was carried out using Merck TLC plates silica gel 60 F₂₅₄. Chromatographic purifications were performed on columns packed with Merck 60 silica gel, 230-400 mesh, for flash technique.

Elemental analyses were determined with an elemental analyser EA 1110 (Fison-Instruments, Milan, Italy) and the purity of all synthesized compounds analysed was >95%. Mass spectra (MS) data were obtained using an Agilent 1100 LC/MSD VL system (G1946C) with a 0.4 mL/min flow rate using a binary solvent system of 95:5 methanol/water. UV detection was monitored at 254 nm. MS were acquired in positive ES (+) and negative ES (−) modes, scanning over the 50-1500 m/z range. The following ion source parameters were used: drying gas flow, 9 mL/min; nebulizer pressure, 40 psig; drying gas temperature, 350° C. In the present invention the following abbreviations are used:

| | |
|---|---|
| NMR | ¹H |
| (Nuclear Magnetic Resonance) | (proton) |
| MHz (Megahertz) | Hz (Hertz) |
| HPLC (High Performance Liquid | LC-MS (Liquid Chromatography |
| Chromatography) | Mass Spectrum) |
| s (seconds) | min (minutes) |
| h (hour(s)) | mg (milligrams) |
| g (grams) | μL (microlitres) |
| mL (millilitres) | mmol (millimoles) |
| nm (nanometers) | μM (micromolar) |
| M (molarity) | RT or rt (room temperature) |
| DMEM (Dulbecco's Modified Eagle's | o.n. (overnight) |
| Medium) | |
| BOC or boc (tert-butyloxycarbonyl) | DMF (dimethylformamide) |
| DCM (dichloromethane) | ACN (acetonitrile) |
| DMF (dimethylformamide) | DMSO (dimethyl sulfoxide) |
| D[6]DMSO (deuterated dimethyl | MeOH (methanol) |
| sulfoxide) | |
| Et₂O (diethyl ether) | EtOAc (ethyl acetate) |
| EtOH (ethanol) | AcOH (acetic acid) |
| iPrOH (isopropanol) | DO₂ (deuterated water) |
| TEA (triethylamine) | THF (tetrahydrofuran) |
| PE (petroleum ether) | BBB (Blood Brain Barrier) |
| t_{R} (retention time) | |

Except where indicated otherwise, all temperatures are expressed in ° C. (degrees centigrade) or K (Kelvin).

The yields were calculated assuming that products were 100% pure if not stated otherwise. Compounds (SI or Si are the same) Si192, Si181, Si319, Si320, Si321, Si328, Si315, Si316, Si1317, Si318, Si322, Si331, Si188, Si189, Si190, Si323, Si171, Si170, Si330, Si176, Si174, Si138, Si135, Si109, Si180, Si182, Si34, Si39, Si1001, Si1003 were synthesized by procedures previously reported by us, and intermediates 6, 7, 8, 12, 16, 24a-b, 24a-b, Si58 and 26 were already reported by us. ^{8,20,21,22,23}

### Enantiomers Separation (FIGS. 4 and 5)

### Chiral Separation of Racemate Si192.

### Instrumentation

The chiral separation studies were carried out on a Varian Prostar HPLC system (Varian Analytical Instruments, USA) equipped with a binary pump with a manual injection valve and model Prostar 325 UV-VIS Detector. The CD detection was achieved on a Jasco CD-815 spectropolarimeter circular dichroism detector (Jasco Corporation, Tokyo, Japan). Optical rotations were determined with a Perkin-Elmer Mod 343 polarimeter at 589 nm, using a 10⁻¹ dm microcell. Concentrations are expressed as g mL⁻¹.

### Enantioselective Columns and Chemicals

The polysaccharide-derived column was cellulose tris-3,5-dimethylphenylcarbamate (250 mm×4.6 mm, Chiralcel OD) coated on 10 μm silica gel. Chiral column was obtained from Daicel (Tokyo, Japan). All of the solvents and reagents were from Sigma Aldrich Srl (Milan, IT).

### LC (Liquid Chromatography) Enantioselective Conditions

Chromatographic separation was carried out at ambient temperature using mobile phase n-hexane/2-propanol-doped with acetonitrile 5%, 90:10 (v/v). Detection was carried out at 280 nm. The injection volume was 20 μL. Starting from 10 mg of racemate: 4 mg of Si192 (R) (t_{R}: 31′33″) and 4 mg of Si192 (S) (t_{R}: 36′20″) were obtained (FIG. 4).

### CD (Cicular Dichrolsm) Conditions

CD spectra were acquired on a Jasco J-815 dichroism spectrometer with linear data array, two accumulations and with scanning speed of 100 nm min⁻¹. A 1.0 mm path-length quartz cell was used and CD spectra were recorded at room temperature. CD spectra obtained from compounds eluted from the racemic mixture separation were acquired in the 190-400 nm range. Pure enantiomers were dissolved in methanol to obtain 0.001 mol L⁻¹ solutions. Three scans were averaged and blank-substracted to obtain the CD spectrum (FIG. 5).

All target compounds possessed a purity of ≥95% as verified by elemental analyses by comparison with the theoretical values.

### [2-(4-Flurophenyl)ethyl]hydrazine (2)

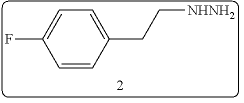 [SMILES: NNCCc1ccc(F)cc1]

A solution of 1-(2-bromoethyl)-4-fluorobenzene 1 (5 g, 24.6 mmol) in isopropanol (10 mL) was added dropwise to a solution of hydrazine monohydrate (10 mL, 206.2 mmol) in isopropanol (200 mL) and the reaction was refluxed for 10 h. After cooling to room temperature, the excess of hydrazine and the solvent were removed under reduced pressure. Then a 40% KOH solution (10 mL) was added and the aqueous phase extracted with diethyl ether (3×15 mL). The organic phases were in turn washed with H₂O (2×15 mL), dried (MgSO₄) and evaporated under reduced pressure to obtain an oil that was purified by bulb to bulb distillation, affording 2 as a pale yellow oil (3.1 g, 81%), which was used as crude in the next step.

### Ethyl 5-amino-1-[2-(4-fluorophenyl)ethyl]-1H-pyrazole-4-carboxylate (3)

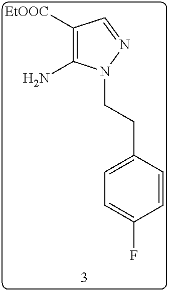 [SMILES: CCOC(=O)c1cnn(CCc2ccc(F)cc2)c1N]

A solution of [2-(4-fluorophenyl)ethyl]hydrazine 2 (1.54 g, 10 mmol) and ethyl (ethoxymethylene)cyanoacetate (1.69 g, 10 mmol) in anhydrous toluene (30 mL) was heated at 80° C. for 8 h. The solution was concentrated under reduced pressure to half of the volume and allowed to cool to room temperature. The yellow pale solid obtained was filtered and recrystallized from toluene to obtain the desired compound 3 as a white solid (2.16 g, 78%); mp: 129-131° C. ¹H NMR (CDCl₃): δ 1.27 (t, J=7.2 Hz, 3H, CH₃), 3.05 (t, J=6.8 Hz, 2H, CH₂Ar), 4.05 (t, J=6.8 Hz, 2H, CH₂N), 4.16 (q, J=7.2 Hz, 2H, CH₂O), 4.36 (br s, 2H, NH₂ disappears with D₂O), 6.94-7.35 (m, 4H Ar), 7.65 (s, 1H, H-3). IR (cm⁻¹): 3426, 3293 (NH₂), 1678 (CO). MS: m/z [M+1]⁺ 278. Anal. (C₁₄H₁₆N₃O₂F) C, H, N.

### Ethyl 5-{[(benzoylamino)carbonothioyl]amino}-1-[2-(4-fluorophenyl)ethyl]-1H-pyrazole-4-carboxylate (4)

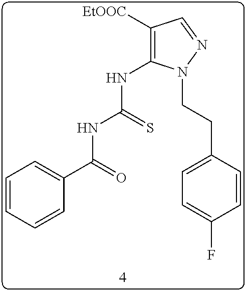 [SMILES: CCOC(=O)c1cnn(CCc2ccc(F)cc2)c1NC(=S)NC(=O)c1ccccc1]

A solution of ethyl 5-amino-1-[2-(4-fluorophenyl)ethyl]-1H-pyrazole-4-carboxylate 3 (500 mg, 1.8 mmol) and benzoylisothiocianate (0.97 mL, 7.2 mmol) in anhydrous THF (10 mL) was refluxed for 12 h. After cooling to room temperature, the solvent was removed under reduced pressure and the crude crystallized as a white solid by adding diethyl ether (20 mL) (713 mg, 90%); mp: 185-187° C. ¹H NMR (CDCl₃): δ 1.24 (t, J=7.2 Hz, 3H, CH₃), 3.20 (t, J=7.0 Hz, 2H, CH₂Ar), 4.12-4.34 (m, 4H, CH₂O+CH₂N), 7.00-7.98 (m, 9H Ar), 7.98 (s, 1H, H-3), 9.32 (s, 1H, NH disappears with D₂O), 11.80 (s, 1H, NH, disappears with D₂O). IR (cm⁻¹): 3367, 3127 (NH), 1706 (COOEt), 1662 (CONH) MS: m/z [M+1]⁺ 441. Anal. (C₂H₂₁N₄O₃FS) C, H, N, S.

### 1-[2-(4-Fluorophenyl)ethyl]6-thioxo-1,5,6,7-tetrahydr-4H-pyrazolo[3,4-d]pyrimidin-4-one (5)

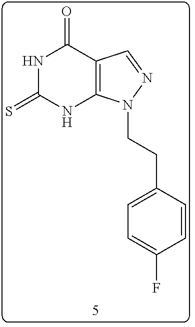 [SMILES: O=c1[nH]c(=S)[nH]c2c1cnn2CCc1ccc(F)cc1]

A solution of ethyl 5-{[(benzoylamino)carbonothioyl]amino}-1-[2-(4-fluorophenyl)ethyl]-1H-pyrazole-4-carboxylate 4 (600 mg, 1.36 mmol) in 2 N NaOH (10 mL) was refluxed for 10 min, then diluted with H₂O (10 mL) and acidified with glacial acetic acid. After 12 h at 4° C., the crystallized solid was filtered and recrystallized from absolute ethanol to give 1-[2-(4-fluorophenyl)ethyl]-6-thioxo-1,5,6,7-tetrahydro-4H-pyrazolo[3,4-d]pyrimidin-4-one 5 as a white solid (249 mg, 63%); mp: 257-259° C. ¹H NMR (CDCl₃): δ 3.22 (t, J=7.0 Hz, 2H, CH₂Ar), 4.25 (t, J=7.0 Hz, 2H, CH₂N), 7.06-7.51 (m, 4H Ar), 7.96 (s, 1H, H-3), 9.28 (s, 1H, NH disappears with D₂O). IR (cm 1): 3400-3300 (NH), 1694 (CO). MS: m/z [M+1]⁺ 291. Anal. (C₁₃H₁₁N₄OFS) C, H, N, S.

### General Procedure for the Synthesis of Compounds 9, 10, 11.

A mixture of 1-substituted 6-thioxo-1,5,6,7-tetrahydro-4H-pyrazolo[3,4-d]pyrimidin-4-one either 5 or 6 or 7 (1 mmol) with 4-(2-chloroethyl)morpholine (224 mg, 1.5 mmol), NaOH (40 mg, 1 mmol) in anhydrous DMF (1 mL) and absolute ethanol (3 mL) was stirred at reflux for 6 h. After cooling to room temperature, the solvent was evaporated under reduced pressure and the mixture was poured into cold water (20 mL). The obtained solid was filtered, washed with water and recrystallized from absolute ethanol.

### 1-[2-(4-Fluorophenyl)ethyl]-6-[(2-morpholin-1-ylethyl)thio]-1,5-dihydro-4H-pyrazolo[3,4-d]pyrimidin-4-one (9)

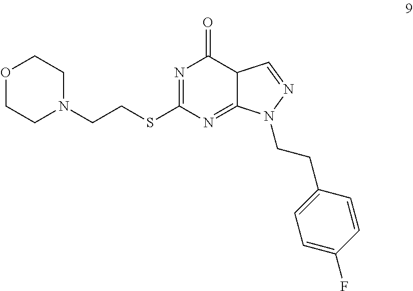 [SMILES: O=C1N=C(SCCN2CCOCC2)N=C2C1C=NN2CCc1ccc(F)cc1]

White solid (347 mg, 86%); mp: 213-215° C. ¹H NMR (CDCl₃): δ 2.42-2.68 (m, 4H, 2CH₂N morph.), 2.77-2.83 (m, 2H, CH₂N), 3.15-3.22 (m, 4H, CH₂S+CH₂Ar), 3.75-3.83 (m, 4H, 2CH₂O morph.), 4.45 (t, J=7.6 Hz, 2H, CH₂N pyraz.), 7.08-7.23 (m, 4H Ar), 8.02 (s, 1H, H-3). IR (cm₋₁): 3400-2800 (NH), 1667 (CO). MS: m/z [M+1]⁺ 404. Anal. (C₁₉H₂N₅O₂FS) C, H, N, S.

### 6-[(2-Morpholin-4-ylethyl)thio]-1-(2-phenylethyl)-1,5-dihydro-4H-pyrazolo[3,4-d]pyrimidin-4-one (10)

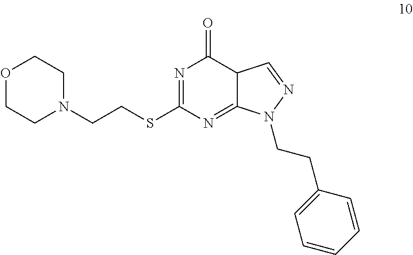 [SMILES: O=C1N=C(SCCN2CCOCC2)N=C2C1C=NN2CCc1ccccc1]

White solid (197 mg, 51%); mp: 197-198° C. ¹H NMR (CDCl₃): δ 2.55-2.70 (m, 4H, 2CH₂N morph.), 2.80-2.84 (m, 2H, CH₂N), 3.17-3.24 (m, 4H, CH₂S+CH₂Ar), 3.80-3.85 (m, 4H, 2CH₂O morph.), 4.50 (t, J=7.6 Hz, 2H, CH₂N pyraz.), 7.10-7.26 (m, 5H Ar), 8.02 (s, 1H, H-3). IR (cm⁻¹): 3500-2800 (NH), 1667 (CO). MS: m/z [M+1]⁺ 386. Anal. (C₁₉H₂₃N₅O₂S) C, H, N, S.

### 6-[(2-Morpholin-4-ylethyl)thio]-1-(2-phenylpropyl)-1,5-dihydro-4H-pyrazolo[3,4-d]pyrimidin-4-one (11)

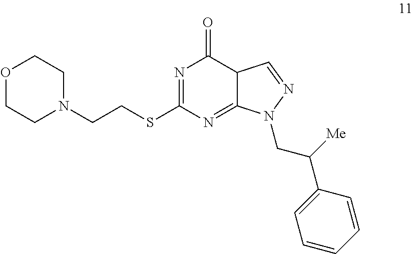 [SMILES: CC(CN1N=CC2C(=O)N=C(SCCN3CCOCC3)N=C21)c1ccccc1]

White solid (200 mg, 50%); mp: 128-130° C. ¹H NMR (CDCl₃): δ 1.24 (d, J=6.8 Hz, 3H, CH₃), 2.60-2.70 (m, 4H, 2CH₂N morph.), 2.80-2.90 and 3.15-3.25 (2m, 4H, SCH₂CH₂), 3.45-3.52 (m, 1H, CHCH₃), 3.80-4.00 (m, 4H, 2CH₂O morph.), 4.38-4.40 (m, 2H, CH₂N pyraz.), 7.18-7.28 (m, 5H Ar), 7.99 (s, 1H, H-3). IR (cm⁻¹): 3450-2900 (NH), 1678 (CO). MS: m/z [M+1]⁺ 400. Anal. (C₂₀H₂₅NSO₂S) C, H, N, S.

### General Procedure for the Synthesis of Compounds 13, 14, 15.

The Vilsmeier complex, previously prepared from POCl₃ (0.74 mL, 8 mmol) and anhydrous DMF (590 mg, 8 mmol) was added to a suspension of either 9, 10, 11 or 12 (1 mmol) in CH₂Cl₂ (10 mL). The mixture was refluxed for 6-12 h. For compounds 14 and 15, the solution was washed with a 4N NaOH solution (2×10 mL), water (2×10 mL), dried (MgSO₄), filtered, and concentrated under reduced pressure. The crude oil was purified by column chromatography (Florisil®, 100-200 mesh) using diethyl ether as eluent, to afford the pure product.

### 4-Chloro-1-[2-(4-fluorophenyl)ethyl]-6-[(2-morpholin-4-ylethyl)thio]-1H-pyrazolo-[3,4-d]pyrimidine (13)

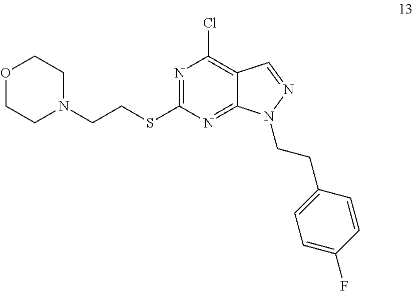 [SMILES: Fc1ccc(CCn2ncc3c(Cl)nc(SCCN4CCOCC4)nc32)cc1]

White solid (363 mg, 86%); mp: 101-102° C. ¹H NMR (CDCl₃): δ 2.50-2.85 (m, 6H, 2CH₂N morph.+CH₂N), 3.24 (t, J=7.2 Hz, 2H, CH₂Ar), 3.33-3.45 (m, 2H, SCH₂), 3.67-3.84 (m, 4H, 2CH₂O morph.), 4.61 (t, J=7.2 Hz, 2H, CH₂N pyraz.), 7.00-7.33 (m, 4H Ar), 8.04 (s, 1H, H-3). MS: m/z [M+1]⁺ 423. Anal. (C₁₉H₂₁N₅OClFS) C, H, N, S.

### 4-Chloro-6-[(2-morpholin-4-ylethyl)thio]-1-(2-phenylethyl)-1H-pyrazolo[3,4-d]pyrimidine (14)

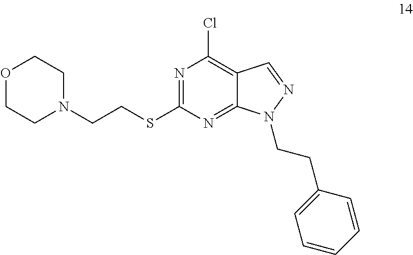 [SMILES: Clc1nc(SCCN2CCOCC2)nc2c1cnn2CCc1ccccc1]

Yellow oil (323 mg, 80%).¹H NMR (CDCl₃): δ 2.51-2.90 (m, 6H, 2CH₂N morph.+CH₂N), 3.22 (t, J=7.2 Hz, 2H, CH₂Ar), 3.30-3.40 (m, 2H, SCH₂), 3.68-3.88 (m, 4H, 2CH₂O morph.), 4.62 (t, J=7.2 Hz, 2H, CH₂N pyraz.), 7.09-7.26 (m, 5H Ar), 8.01 (s, 1H, H-3). MS: m/z [M+1]⁺ 405. Anal. (C₁₉H₂₂N₅OClS) C, H, N, S.

### 4-Chloro-6-[(2-morpholin-4-ylethyl)thio]-1-(2-phenylpropyl)-1H-pyrazolo[3,4-d]pyrimidine (15)

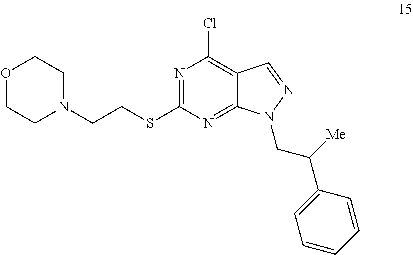 [SMILES: CC(Cn1ncc2c(Cl)nc(SCCN3CCOCC3)nc21)c1ccccc1]

Yellow oil (288 mg, 69%). ¹H NMR (CDCl₃): δ 1.22 (d, J=6.8 Hz, 3H, CH₃), 2.50-2.66, 2.73-2.82, 3.26-3.41 and 3.45-3.58 (4m, 8H, 2CH₂N morph.+SCH₂CH₂), 3.68-3.85 (m, 5H, 2CH₂O morph.+CHCH₃), 4.40-4.56 (m, 2H, CH₂N pyraz.), 7.07-7.30 (m, 5H Ar), 7.96 (s, 1H, H-3). MS: m/z [M+1]⁺ 419. Anal. (C₂₀H₂₄N₅OClS) C, H, N, S.

General procedure for the synthesis of compounds Si303, Si313, Si314, Si307, Si327, Si306.

The suitable aniline (2 mmol) was added to a solution of the 4-chloro derivative 13, 14, 15 or 16 (1 mmol) in absolute ethanol (5 mL), and the mixture was refluxed for 3-5 h. After cooling to room temperature, the obtained solid was filtered, washed with water, and recrystallized from absolute ethanol.

### N-(3-Chlorophenyl)-6-[(2-morpholin-4-ylethyl)thio]-1-(2-phenylethyl)-1H-pyrazolo[3,4-d]pyrimidin-4-amine (Si303)

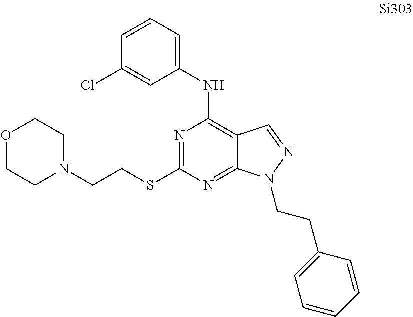 [SMILES: Clc1cccc(Nc2nc(SCCN3CCOCC3)nc3c2cnn3CCc2ccccc2)c1]

White solid (233 mg, 47%); mp: 235-237° C. ¹H NMR (CDCl₃): δ 2.88-2.95 (m, 4H, 2CH₂N morph.), 3.15 (t, J=7.0, 2H, CH₂Ar), 3.22-3.30 (m, 2H, CH₂N), 3.69-3.74 (m, 2H, SCH₂), 4.00-4.49 (m, 4H, 2CH₂O morph.), 4.64 (t, J=7.0, 2H, CH₂N pyraz.), 7.16-7.38 (m, 9H Ar), 7.51 (s, 1H, H-3). IR (cm⁻¹): 3300-3100 (NH). MS: m/z [M+1]⁺ 496. Anal. (C₂₅H₂₇N₆OClS) C, H, N, S.

### 6-[(2-Morpholin-4-ylethyl)thio]-N-phenyl-1-(2-phenylpropyl)-1H-pyrazolo[3,4-d]pyrimidin-4-amine (Si313)

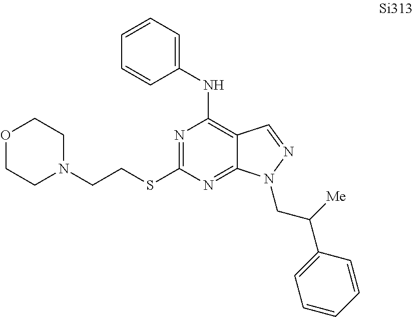 [SMILES: CC(Cn1ncc2c(Nc3ccccc3)nc(SCCN3CCOCC3)nc21)c1ccccc1]

White solid (332 mg, 70%); mp: 212-213° C. ¹H NMR (CDCl₃): δ 1.14 (d, J=6.8 Hz, 3H, CH₃), 2.80-3.00 (m, 2H, SCH₂), 3.20-3.45, 3.46-3.60, 3.72-3.85 and 4.02-4.15 (4m, 11H, 4CH₂ morph.+CH₂N+CHCH₃), 4.30-4.44 (m, 2H, CH₂N pyraz.), 6.70-6.81 and 7.07-7.35 (2m, 10H Ar), 7.49 (s, 1H, H-3). IR (cm⁻¹): 3500-2800 (NH). MS: m/z [M+1]⁺ 476. Anal. (C₂₆H₃₀N₆OS) C, H, N, S.

### N-(3-Fluorophenyl)-6-[(2-morpholin-1-ylethyl)thio]-1-(2-phenylpropyl)-1H-pyrazolo[3,4-d]pyrimidin-4-amine (Si314)

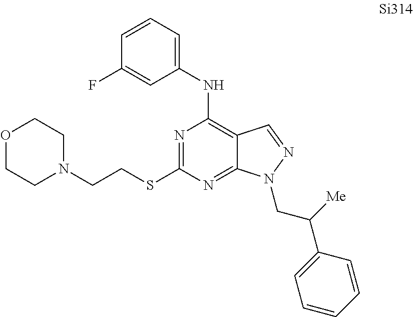 [SMILES: CC(Cn1ncc2c(Nc3cccc(F)c3)nc(SCCN3CCOCC3)nc21)c1ccccc1]

White solid (182 mg, 37%); mp: 236-237° C. ¹H NMR (CDCl₃): δ 1.24 (d, J=7.0 Hz, 3H, CH₃), 2.08-2.60, 2.78-3.17, 3.27-3.74 and 3.97-4.38 (4m, 13H, SCH₂+4CH₂ morph.+CH₂N+CHCH₃), 4.40-4.50 (m, 2H, CH₂N pyraz.), 5.90-6.40 and 7.03-7.50 (2m, 10H, 9 Ar+H-3), 9.33 (br s, 1H, NH, disappears with D₂O). IR (cm⁻¹): 3450-3100 (NH). MS: m/z [M+1]⁺ 494. Anal. (C₂₆H₂₉N₆OFS) C, H, N, S.

### N-(3-Chlorophenyl)-6-[(2-morpholin-4-ylethyl)thio]-1-(2-phenylpropyl)-1H-pyrazolo[3,4-d]pyrimidin-4-amine (Si307)

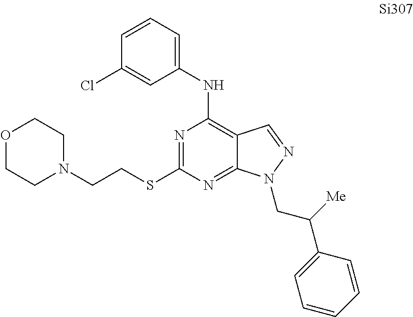 [SMILES: CC(Cn1ncc2c(Nc3cccc(Cl)c3)nc(SCCN3CCOCC3)nc21)c1ccccc1]

Pale yellow solid (265 mg, 52%); mp: 247-249° C. ¹H NMR ([D₆]DMSO): δ 1.23 (d, J=7.0 Hz, 3H, CH₃), 2.52-2.67, 2.74-2.81, 3.24-3.40 and 3.43-3.59 (4m, 8H, 2CH₂N morph.+SCH₂CH₂), 3.65-3.80 (m, 4H, 2CH₂O morph.), 3.85-3.90 (m, 1H, CHCH₃), 4.40-4.50 (m, 2H, CH₂N pyraz.), 7.20-7.40 (m, 9H Ar), 7.97 (s, 1H, H-3), 10.40 (br s, 1H, NH disappears with D₂O). IR (cm⁻¹): 3450-3100 (NH). MS: m/z [M+1]⁺ 510. Anal. (C₂₆H₂₉N₆OClS) C, H, N, S.

### N-(3-Chlorophenyl)-1-[2-(4-fluorophenyl)ethyl]-6-[(2-morpholin-1-ylethyl)thio]-1H-pyrazolo[3,4-d]pyrimidin-4-amine (Si327)

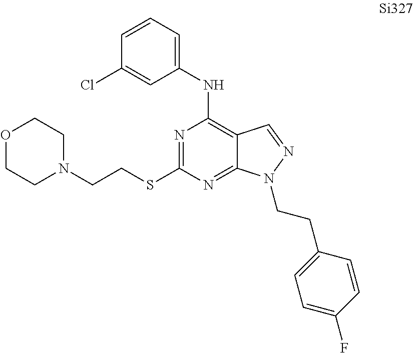 [SMILES: Fc1ccc(CCn2ncc3c(Nc4cccc(Cl)c4)nc(SCCN4CCOCC4)nc32)cc1]

White solid (159 mg, 31%); mp: 127-128° C. ¹H NMR (CDC₃): δ 2.44-2.69 and 2.80-2.91 (2m, 6H, 2CH₂N morph+CH₂N), 3.14 (t, J=6.8 Hz, 2H, SCH₂), 3.37-3.54 and 3.70-3.82 (2m, 6H, CH₂Ar+2CH₂O morph.), 4.49 (t, J=6.9 Hz, 2H, CH₂N pyraz.), 6.87-6.92, 7.05-7.08 and 7.28-7.36 (3m, 9H, 8 Ar+H-3). IR (cm⁻¹): 1558 (NH). MS: m/z [M+1]⁺ 514. Anal. (C₂₅H₂₆N₆OClFS) C, H, N, S.

### N-(3-Bromophenyl)-1-(2-chloro-2-phenylethyl)-6-[(2-morpholin-4-ylethyl)thio]-1H-pyrazolo[3,4-d]pyrimidin-4-amine (Si306)

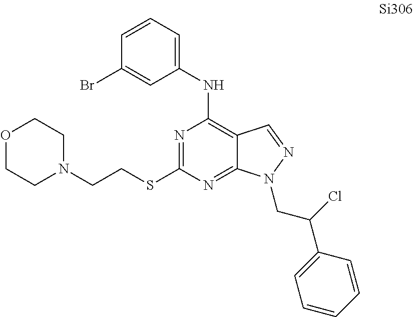 [SMILES: ClC(Cn1ncc2c(Nc3cccc(Br)c3)nc(SCCN3CCOCC3)nc21)c1ccccc1]

White solid (350 mg, 61%); mp: 232-233° C. ¹H NMR (CDCl₃): δ 2.90-3.99 (m, 12H, 4CH₂ morph.+CH₂N+CH₂S), 4.63-4.85 and 5.04-5.21 (2m, 2H, CH₂N pyraz.), 5.55-5.70 (m, 1H, CHCl), 7.03-8.52 (m, 10H, 9 Ar+H-3), 11.33 (br s, 1H, NH disappears with D₂O). IR (cm⁻¹): 3450 (NH). MS: m/z [M+1]⁺ 575. Anal. (C₂₅H₂₆N₆OBrClS) C, H, N, S.

### General Procedure for the Synthesis of Compounds Si332, Si329.

The 3-aminophenol (545 mg, 5 mmol) was added to a solution of the suitable 4-chloro derivative 14 or 15 (1 mmol) in absolute ethanol (10 mL), and the mixture was refluxed for 3-5 h. After cooling to room temperature, the solvent was evaporated under reduced pressure and the crude was solved in ethyl acetate (10 mL), washed with 0.1 N HCl solution (2×10 mL), 1 N NaOH solution (10 mL), brine (2×10 mL), dried (MgSO₄), filtered, and concentrated under reduced pressure to give a brown oil which crystallized at 4° C. by adding a 1:1 mixture of diethyl ether/petroleum ether (bp 40-60° C.). If necessary, the solid obtained was purified by Silica gel chromatography column using CH₂Cl₂ as eluent. Compounds Si332 and Si329 were obtained as hydrochloride salts.

### 3-{[6-[(2-Morpholin-4-ylethyl)thio]-1-(2-phenylethyl)-1H-pyrazolo[3,4-d]pyrimidin-4-yl]amino}phenol hydrochloride (Si332)

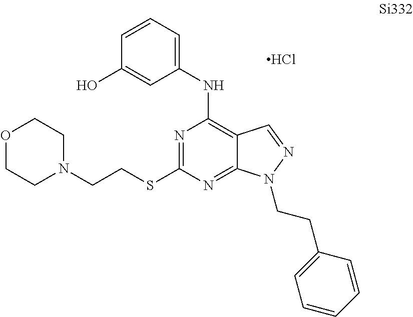 [SMILES: Oc1cccc(Nc2nc(SCCN3CCOCC3)nc3c2cnn3CCc2ccccc2)c1 ; Cl]

Pale yellow solid (261 mg, 51%); mp: 261-262° C. ¹H NMR (CDCl₃): δ 3.05-3.58 (m, 10H, CH₂Ar+2CH₂N morph.+SCH₂+CH₂N), 3.77-3.95 (m, 4H, 2CH₂O morph.), 4.57 (t, J=7.0 Hz, 2H, CH₂N pyraz.), 6.52-6.63 and 7.08-7.32 (2m, 10H, 9 Ar+H-3), 8.22 (br s, 1H, NH disappears with D₂O), 9.61 (br s, 1H disappears with D₂O), 10.18 (br s, 1H disappears with D₂O). IR (cm⁻¹): 3500-3100 (NH+OH). MS: m/z [M+1]⁺ 478. Anal. (C₂₅H₂₉N₆O₂ClS) C, H, N, S.

### 3-{[6-[(2-Morpholin-4-ylethyl)thio]-1-(2-phenylpropyl)-1H-pyrazolo[3,4-d]pyrimidin-4-yl]amino}phenol hydrochloride (Si329)

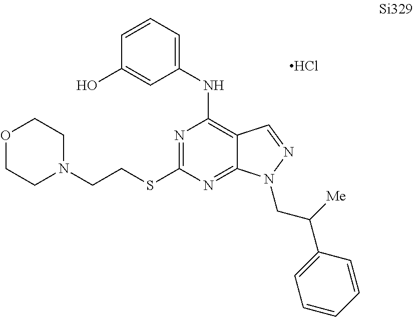 [SMILES: CC(Cn1ncc2c(Nc3cccc(O)c3)nc(SCCN3CCOCC3)nc21)c1ccccc1 ; Cl]

Pale yellow solid (248 mg, 47%); mp: 177-178° C. ¹H NMR (CDCl₃): δ 1.16-1.33 (m, 3H, CH₃), 2.60-2.80, 2.88-3.03 and 3.30-3.64 (3m, 9H, 2CH₂N morph.+CHCH₃+CH₂CH₂S), 3.78-3.97 (m, 4H, 2CH₂O morph.), 4.38-4.55 (m, 2H, CH₂N pyraz.), 6.54-6.74 and 7.09-7.33 (2m, 9H Ar), 7.58 (br s, 1H, disappears with D₂O), 7.92 (s, 1H, H-3), 8.18 (br s, 1H, disappears with D₂O). IR (cm⁻¹): 3500-3100 (NH+OH). MS: m/z [M+1]⁺ 492. Anal. (C₂₆H₃₁N₆O₂ClS) C, H, N, S.

### General Procedure for the Synthesis of 18a, 18b, 18c, 18d, 18e.

A 60% sodium hydride dispersion in mineral oil (1.21 g, 30.3 mmol) was added in small batches to a solution of malonitrile (1.00 g, 15.1 mmol) in dry THF (25 mL) precooled at 0/5° C. After 30 minutes at 0/5° C., the suitable acyl chloride (15.1 mmol) was added dropwise. The orange solution was stirred at room temperature for 2-12 h, then dimethylsulfate (1.75 mL, 18.2 mmol) was slowly added and the solution was refluxed for 3-6 h. Finally, 2-hydrazino-1-phenylethanol 17 (4.62 g, 30.2 mmol) dissolved in dry THF (2 mL) was added and the reaction was refluxed for 4 h. After cooling to room temperature, water (25 mL) and cone. NH₃ (5 mL) were added under stirring. After 15 minutes THF was removed under reduced pressure and the aqueous phase was extracted with CH₂Cl₂ (3×30 mL). Organic phases were washed with water (15 mL), brine (15 mL), dried (Na₂SO₄) and evaporated under reduced pressure. The crude was purified by flash chromatography (silica gel 0.060-0.200 mm, 40 Å) using Et₂O/PE (bp 40-60° C.) as eluent, with a gradient elution (3:1→9:1) to afford compounds 18a, 18b, 18c 18d or 18e.

### 5-Amino-3-(4-fluorophenyl)-1-2-hydroxy-2-phenylethyl)-1H-pyrazole-4-carbonitrile (18a)

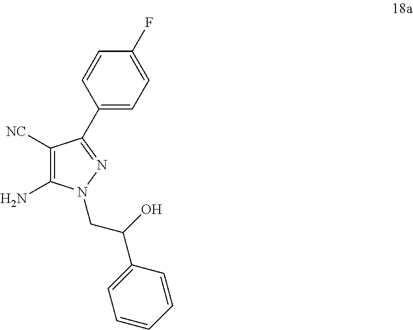 [SMILES: N#Cc1c(-c2ccc(F)cc2)nn(CC(O)c2ccccc2)c1N]

White solid (1.54 g, 32%); mp: 175-176° C. ¹H NMR: δ 4.01-4.10 and 4.15-4.19 (2m, 2H, CH₂), 5.12-5.18 (m, 1H, CH), 7.19-7.52 and 8.25-8.30 (2m, 9H Ar). IR (cm⁻¹): 3450-2900 (OH), 3388, 3323 (NH₂), 2223 (CN). MS: m/z 323 [M+1]⁺. Anal. (C₁₈H₁₅N₄FO) C, H, N.

### 5-Amino-3-(4-chlorophenyl)-1-(2-hydroxy-2-phenylethyl)-H-pyrazole-4-carbonitrile (18b)

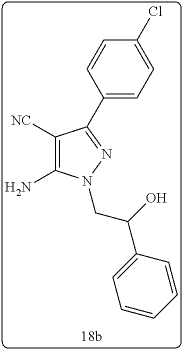 [SMILES: N#Cc1c(-c2ccc(Cl)cc2)nn(CC(O)c2ccccc2)c1N]

White solid (2.50 g, 49%); mp: 173-174° C. ¹H NMR: δ 3.99-4.15 (m, 2H, CH₂), 5.12-5.18 (m, 1H, CH), 7.50-7.54 and 7.96-7.99 (2m, 9H Ar). IR (cm⁻¹): 3450-3100 (OH), 3388, 3322 (NH₂), 2223 (CN). MS: m/z 340 [M+1]⁺. Anal. (C₁₈H₁₅N₄ClO) C, H, N.

### 5-Amino-1-(2-hydroxy-2-phenylethyl)-3-(4-methylphenyl)-1H-pyrazole-4-carbonitrile (18c)

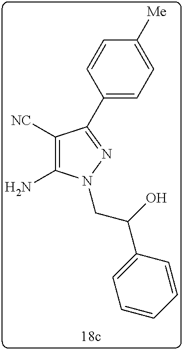 [SMILES: Cc1ccc(-c2nn(CC(O)c3ccccc3)c(N)c2C#N)cc1]

White solid (2.02 g, 42%); mp: 172-174° C. ¹H NMR: δ 2.36 (s, 3H, CH₃), 4.00-4.05 and 4.12-4.15 (2m, 2H, CH₂), 5.10-5.15 (m, 1H, CH), 7.20-7.34 and 7.57-7.91 (2m, 9H Ar). IR (cm⁻¹): 3400-3200 (OH), 3400, 3322 (NH₂), 2221 (CN). MS: m/z 319 [M+1]⁺. Anal. (C₁₉H₁₈N₄O) C, H, N.

### 5-Amino-1-(2-hydroxy-2-phenylethyl)-3-(4-methoxyphenyl)-1H-pyrazole-4-carbonitrile (18d)

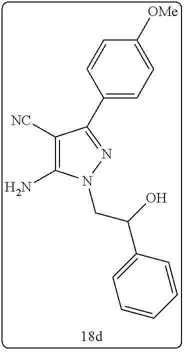 [SMILES: COc1ccc(-c2nn(CC(O)c3ccccc3)c(N)c2C#N)cc1]

White solid (2.50 g, 50%); mp: 144-145° C. ¹H NMR: δ 3.79 (s, 3H, CH₃), 4.03-4.06 and 4.10-4.15 (2m, 2H, CH₂), 5.11-5.15 (m, 1H, CH), 7.18-7.30 and 7.60-7.85 (2m, 9H Ar). IR (cm⁻¹): 3450-2900 (OH), 3409, 3351 (NH₂), 2220 (CN). MS: m/z 335 [M+1]⁺. Anal. (C₁₉H₁₈N₄O₂) C, H, N.

### 5-Amino-1-(2-hydroxy-2-phenylethyl)-3-phenyl-1H-pyrazole-4-carbonitrile (18e)

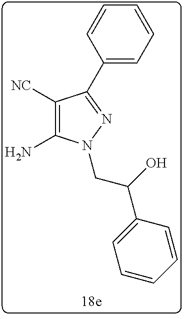 [SMILES: N#Cc1c(-c2ccccc2)nn(CC(O)c2ccccc2)c1N]

White solid (1.84 g, 40%); mp: 165-166° C. ¹H NMR: δ 3.95-4.23 (m, 2H, CH₂), 5.10-5.18 (m, 1H, CH), 7.20-7.37 and 7.79-7.81 (2m, 10H Ar). IR (cm⁻¹): 3560-3240 (OH), 3358, 3350 (NH₂), 2204 (CN). MS: m/z 305 [M+1]⁺. Anal. (C₁₈H₁₆N₄O) C, H, N.

### General Procedure for the Synthesis of 19a, 19b, 19c, 19d, 19e.

A suspension of the suitable intermediate 18a, 18b, 18c, 18d or 18e (3 mmol) in formamide (18 mL, 450 mmol) was heated at 190° C. for 3-4 h and then poured into water (40 mL). The crude solid was filtered, washed with water, suspended in ethanol and boiled with charcoal for 10 minutes. The solid dissolved at the ethanol boiling point. After charcoal filtration, compounds 19b, 19c or 19e precipitated as pure solids. Compound 19a or 19d precipitated and were further purified by flash chromatography (silica gel 0.060-0.200 mm, 40 Å) using CH₂Cl₂/CH₃OH (98:2) as eluent to afford a pure oil that slowly crystallized by adding a mixture of Et₂O/PE (bp 40-60° C.) (1:1).

### 2-[4-Amino-3-(4-fluorophenyl)-1H-pyrazolo[3,4-d]pyrimidin-1-yl]-1-phenylethanol (19a)

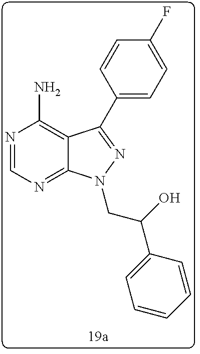 [SMILES: Nc1ncnc2c1c(-c1ccc(F)cc1)nn2CC(O)c1ccccc1]

White solid (497 mg, 48%); mp: 190-192° C. 1H NMR: δ 4.12-4.34 and 4.42-4.45 (2m, 2H, CH₂), 5.05-5.10 (m, 1H, CH), 7.19-7.30 and 7.51-7.60 (2m, 9H Ar), 8.12 (s, 1H, H-6). IR (cm⁻¹): 3500-3060 (OH), 3484, 3307 (NH₂). MS: m/z 350 [M+1]⁺. Anal. (C₁₉H₁₆N₅FO) C, H, N.

### 2-[4-Amino-3-(4-chlorophenyl)-1H-pyrazolo[3,4-d]pyrimidin-1-yl]-1-phenylethanol (19b)

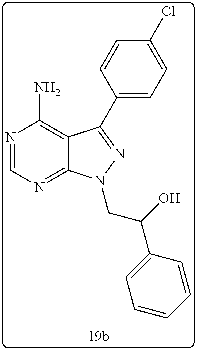 [SMILES: Nc1ncnc2c1c(-c1ccc(Cl)cc1)nn2CC(O)c1ccccc1]

White solid (509 mg, 46%); mp: 200-201° C. ¹H NMR: δ 4.21-4.28 and 4.35-4.42 (2m, 2H, CH₂), 5.15-5.18 (m, 1H, CH), 7.21-7.34 and 7.70-7.82 (2m, 9H Ar), 8.10 (s, 1H, H-6). IR (cm⁻¹): 3450-2990 (OH), 3407, 3290 (NH₂). MS: m/z 367 [M+1]⁺. Anal. (C₉H₁₆N₅ClO) C, H, N.

### 2-[4-Amino-3-(4-methyphenyl)-1H-pyrazolo[3,4-d]pyrimidin-1-yl]-1-phenylethanol (19c)

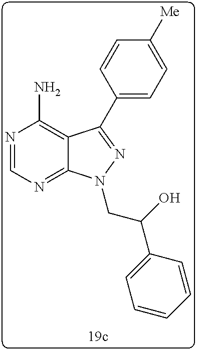 [SMILES: Cc1ccc(-c2nn(CC(O)c3ccccc3)c3ncnc(N)c23)cc1]

White solid (496 mg, 48%); mp: 93-95° C. ¹H NMR: δ 2.37 (s, 3H, CH₃), 4.20-4.25 and 4.30-4.37 (2m, 2H, CH₂), 5.10-5.15 (m, 1H, CH), 7.19-7.32 and 7.50-7.78 (2m, 9H Ar), 8.12 (s, 1H, H-6). IR (cm⁻¹): 3500-3000 (OH), 3469, 3296 (NH₂). MS: m/z 346 [M+1]⁺. Anal. (C₂₀H₁₉N₅O) C, H, N.

### 2-[4-Amino-3-(4-methoxyphenyl)-1H-pyrazolo[3,4-d]pyrimidin-1-yl]-1-phenylethanol (19d)

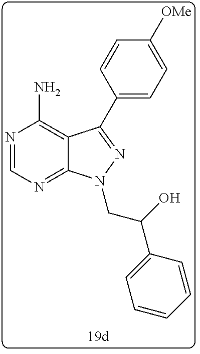 [SMILES: COc1ccc(-c2nn(CC(O)c3ccccc3)c3ncnc(N)c23)cc1]

White solid (427 mg, 39%); mp: 161-163° C. ¹H NMR: δ 3.82 (s, 3H, CH₃), 4.22-4.27 and 4.33-4.41 (2m, 2H, CH₂), 5.13-5.21 (m, 1H, CH), 7.15-7.38 and 7.45-7.90 (2m, 9H Ar), 8.10 (s, 1H, H-6). IR (cm⁻¹): 3500-2900 (OH), 3461, 3360 (NH₂). MS: m/z 362 [M+1]⁺. Anal. (C₂₀H₁₉NSO₂) C, H, N.

### 2-(4-Amino-3-phenyl-1H-pyrazolo[3,4-d]pyrimidin-1-yl)-1-phenylethanol (19e)

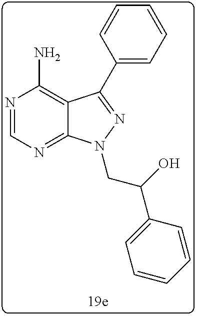 [SMILES: Nc1ncnc2c1c(-c1ccccc1)nn2CC(O)c1ccccc1]

White solid (549 mg, 55%); mp: 160-161° C. ¹H NMR: δ 4.10-4.32 and 4.40-4.38 (2m, 2H, CH₂), 5.02-5.08 (m, 1H, CH), 7.16-7.22 and 7.42-7.50 (2m, 10H Ar), 8.09 (s, 1H, H-6). IR (cm⁻¹): 3500-2900 (OH), 3474, 3315 (NH₂). MS: m/z 332 [M+1]⁺. Anal. (C₁₉H₁₇N₅O) C, H, N.

### General Procedure for the Synthesis of Si244, Si308, Si309, Si310, Si311.

SOCl₂ (80 μL, 1.1 mmol) was added dropwise to a solution of the suitable intermediate 19a, 19b, 19c, 19d or 19e (0.5 mmol) in dry CH₂Cl₂ (5 mL), and the reaction was stirred at room temperature for 12 h under nitrogen atmosphere. Water (5 mL) and IN NaOH (1 mL) were added with caution and the aqueous phase was extracted with CH₂Cl₂ (2×5 mL). Then the organic phase was washed with water (5 mL), brine (5 mL), dried (Na₂SO₄) and concentrated under reduced pressure. Final compounds Si308, Si309, Si310 or Si311 were obtained as white solids adding a mixture of Et₂O/PE (bp 40-60° C.) (1:1). Compound Si244, that resulted a yellow oil, was precipitated as hydrochloride salt, by adding a saturated solution of HCl in dry Et₂O.

### 1-(2-Chloro-2-phenylethyl)-3-(4-fluorophenyl)-1H-pyrazolo[3,4-d]pyrimidin-4-amine (Si310)

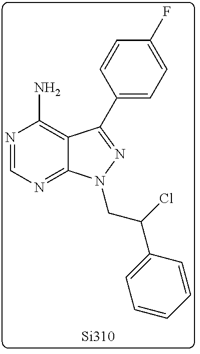 [SMILES: Nc1ncnc2c1c(-c1ccc(F)cc1)nn2CC(Cl)c1ccccc1 ; "Si310"]

White solid (125 mg, 68%); mp: 203-206° C. ¹H NMR: δ 4.77-4.82 and 4.95-5.00 (2m, 2H, CH₂N), 5.68-5.70 (m, 1H, CHCl), 7.38-7.42 and 7.51-7.64 (m, 9H Ar), 8.22 (s, 1H, H-6). IR (cm⁻¹): 3477, 3314 (NH₂). MS: m/z 369 [M+1]⁺. Anal. (C₁₉H₁₅N₅ClF) C, H, N.

### 3-(4-Chlorophenyl)-1-(2-chloro-2-phenylethyl)-1H-pyrazolo[3,4-d]pyrimidin-4-amine (Si308)

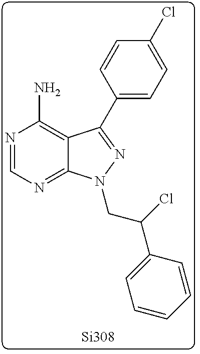 [SMILES: Nc1ncnc2c1c(-c1ccc(Cl)cc1)nn2CC(Cl)c1ccccc1 ; "Si308"]

White solid (65 mg, 34%); mp: 150-151° C. ¹H NMR: δ 4.76-4.80 and 4.97-5.02 (2m, 2H, CH₂N), 5.67-5.68 (m, 1H, CHCl), 7.34-7.36 and 7.51-7.61 (m, 9H Ar), 8.24 (s, 1H, H-6). IR (cm⁻¹): 3470, 3301 (NH₂). MS: m/z 385 [M+1]⁺. Anal. (C₁₉H₁₅N₅Cl₂) C, H, N.

### 1-(2-Chloro-2-phenylethyl)-3-(4-methylphenyl)-H-pyrazolo[3,4-d]pyrimidin-4-amine (Si309)

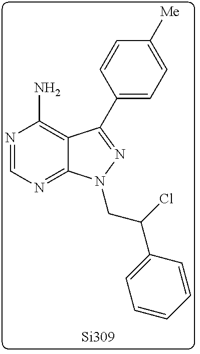 [SMILES: Cc1ccc(-c2nn(CC(Cl)c3ccccc3)c3ncnc(N)c23)cc1 ; "Si309"]

White solid (90 mg, 49%); mp: 159-160° C. ¹H NMR: δ 2.36 (s, 3H, CH₃), 4.75-4.80 and 4.95-5.01 (2m, 2H, CH₂N), 5.66-5.70 (m, 1H, CHCl), 7.24-7.39 and 7.50-7.63 (m, 9H Ar), 8.23 (s, 1H, H-6). IR (cm⁻¹): 3468, 3306 (NH₂). MS: m/z 365 [M+1]⁺. Anal. (C₂₀H₁₈N₅Cl) C, H, N.

### 1-(2-Chloro-2-phenylethyl)-3-(4-methyoxyphenyl)-1H-pyrazolo[3,4-d]pyrimidin-4-amine (Si311)

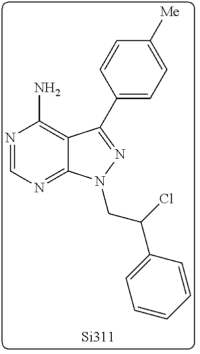 [SMILES: Cc1ccc(-c2nn(CC(Cl)c3ccccc3)c3ncnc(N)c23)cc1 ; "Si311"]

White solid (146 mg, 77%); mp: 152-153° C. ¹H NMR: δ 3.89 (s, 3H, OCH₃), 4.75-4.80 and 5.01-5.07 (2m, 2H, CH₂N), 5.58-5.62 (m, 1H, CHCl), 7.01-7.10 and 7.26-7.68 (m, 9H Ar), 8.36 (s, 1H, H-6). IR (cm⁻¹): 3470, 3305 (NH₂). MS: m/z 381 [M+1]⁺. Anal. (C₂₀H₁₈N₅ClO) C, H, N.

### 1-(2-Chloro-2-phenylethyl)-3-phenyl-1H-pyrazolo[3,4-d]pyrimidin-4-amine hydrochloride (Si244)

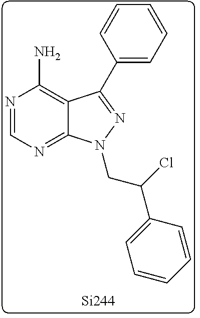 [SMILES: "Si244" ; Nc1ncnc2c1c(-c1ccccc1)nn2CC(Cl)c1ccccc1]

White solid (135 mg, 70%); mp: 129-132° C. ¹H NMR: δ 4.76-4.81 and 5.00-5.06 (2m, 2H, CH₂N), 5.50-5.54 (m, 1H, CHCl), 7.23-7.73 (m, 10H Ar), 9.49 (s, 1H, H-6). MS: m/z 387 [M+1]⁺. Anal. (C₁₉H₁₇N₅Cl₂) C, H, N.

### Synthesis of 5-amino-1H-pyrazolo-4-carbonitrile (20)

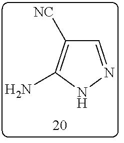

Hydrazine monohydrate (800 μL, 16.4 mmol) was added to a solution of (ethoxymethylene)malononitrile (2 g, 16.4 mmol) in absolute ethanol (10 mL) and the mixture was refluxed for 4 h. After cooling to room temperature, the solvent was evaporated under reduced pressure. Then, cold water (50 mL) was added and the crude was filtered and washed with water (3×40 mL) to give compound 20 as a red solid (1.40 g, 81%); mp: 172-174° C. (Lit. 74%; mp: 169-170° C.).

### Synthesis of 1H-pyrazolo[3,4-d]pyrimidin-4-amine (21)

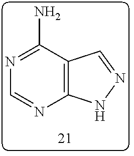

A solution of 5-amino-1H-pyrazolo-4-carbonitrile 20 (400 mg, 3.7 mmol) and formamide (5 mL, 125.8 mmol) was stirred at 200° C. for 1 h. After cooling to room temperature, water was added (20 mL) and the obtained solid was filtered. The crude product was suspended in hot water (40 mL) and conc. HCl (5 mL), then charcoal (600 mg) was added and the mixture was boiled for 15 min. After charcoal filtration, conc. NH₃ was added, the precipitated solid was filtered, giving compound 21 as a white solid (405 mg, 81%); mp 353-356° C. (Lit. 58%, m.p.>300° C.).

### Synthesis of 3-Iodo-1H-pyrazolo[3,4-d]pyrimidin-4-amine (22)

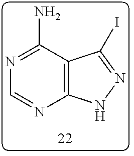 [SMILES: Nc1ncnc2[nH]nc(I)c12]

N-iodosuccinimide (2 g, 8.9 mmol) was added to a solution of 1H-pyrazolo[3,4-d]pyrimidin-4-amine 21 (800 mg, 5.9 mmol) in dry DMF (5 mL) and the mixture heated at 80° C. for 14 h under nitrogen atmosphere. After cooling to room temperature, water was added (20 mL) and the precipitated solid was filtered and washed with water (50 mL). The crude product was recrystallized from absolute ethanol, to give compound 22 as a light-yellow solid (1.31 g, 85%); mp 272-275° C. (Lit. 97%).

### Synthesis of 3-odo-1-(2-phenylpropyl)-1H-pyrazolo[3,4-d]pyrimidin-4-amine (23)

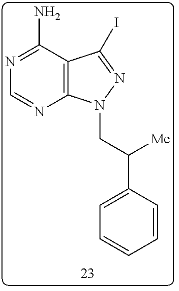 [SMILES: CC(Cn1nc(I)c2c(N)ncnc21)c1ccccc1]

K₂CO₃ (600 mg, 4.34 mmol) was added to a solution of 3-iodo-1H-pyrazolo[3,4-d]pyrimidin-4-amine 22 (400 mg, 1.53 mmol) in dry DMF (5 mL), and the mixture was heated at 50° C. for 1 h. 1-Bromo-2-phenylpropane (350 μL, 2.30 mmol) was added and the reaction was stirred at 130° C. for 18 h. After cooling to room temperature, water was added (30 mL) and the precipitated solid was filtered and purified by column chromatography (silica gel 0.060-0.200 mm, 40 Å) using a mixture of CH₂Cl₂/MeOH (95:5) as eluent to afford compound 23 as a white solid (390 mg, 67%); mp 265-268° C. ¹H NMR: δ 1.22 (m, 3H, CH₃), 3.52-3.57 (m, 1H, CH), 4.48-4.50 (m, 2H, CH₂), 5.83 (br s, 2H, NH₂ disappears with D₂O), 7.18-7.28 (m, 5H Ar), 8.28 (s, 1H, H-6). IR (cm⁻¹): 3480, 3360 (NH₂). MS: m/z 380 [M+H]⁺. Anal. (C₁₄H₁₄N₅I) C, H, N.

### General Procedure for the Synthesis of Si312, Si336, Si337, Si338, Si339.

The Suitable boronic acid (1.08 mmol) was added to a suspension of 3-iodo-1-(2-phenylpropyl)-1H-pyrazolo[3,4-d]pyrimidin-4-amine 23 (100 mg, 0.27 mmol) in dry toluene (5 mL), and the mixture was stirred at room temperature under nitrogen atmosphere for 10 minutes. Then Cs₂CO₃ (350 mg, 1.07 mmol) and PdCl₂(dppf) (20 mg, 10% mol) were added. The reaction was stirred at 90° C. for 14 h. After cooling to room temperature, water (70 mL) was added and the aqueous suspension was extracted with EtOAc (2×40 mL). The organic phase was washed with water (40 mL) and brine (40 mL), dried (Na₂SO₄) and concentrated under reduced pressure to obtain a crude, which was purified by column chromatography (silica gel 0.060-0.200 mm, 40 Å) using a mixture of CH₂Cl₂/MeOH (95:5) as eluent to afford compound Si312, Si336, Si337, Si338 or Si339.

### 3-Phenyl-1-(2-phenylpropyl)-1H-pyrazolo[3,4-d]pyrimidin-4-amine (Si312)

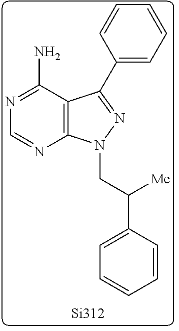

White solid (24 mg, 27%); mp: 105-109° C. ¹H NMR: δ 1.28 (d, J=6.8 Hz, 3H, CH₃), 3.60-3.65 (m, 1H, CH), 4.56-4.62 (m, 2H, CH₂N), 5.58 (br s, 2H, NH₂ disappears with D₂O), 7.17-7.27 and 7.46-7.67 (2m, 10H Ar), 8.40 (s, 1H, H-6). IR (cm⁻¹): 3476, 3298 (NH₂). MS: m/z 330 [M+1]⁺. Anal. (C₂₀H₁₉N₅) C, H, N.

### 1-{4-[4-Amino-1-(2-phenylpropyl)-1H-pyrazolo[3,4-d]pyrimidin-3-yl]phenyl}ethanone (Si336)

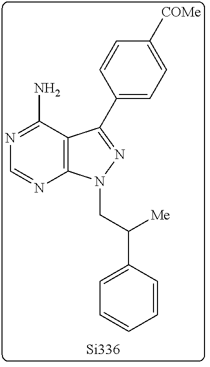

Yellow solid (50 mg, 50%); mp 232-235° C. ¹H NMR CDCl₃: δ 1.29 (d, J=6.8 Hz, 3H, CH_H₃), 2.67 (s, 3H, CH₃CO), 3.58-3.65 (m, 1H, CH), 4.58-4.60 (m, 2H, CH₂), 5.47 (br s, 2H, NH₂ disappears with D₂O), 7.19-7-27, 7.77-7.79 and 8.11-8.13 (3m, 9H Ar), 8.37 (s, 1H, H-6). IR (cm⁻¹): 3478, 3315 (NH₂). MS: m/z 372 [M+H]⁺. Anal. (C₂₂H₂₁N₅O) C, H, N.

### 3-(4-Chlorophenyl)-1-(2-phenylpropyl)-1H-pyrazlo[3,4-d]pyrimidin-4-amine (Si337)

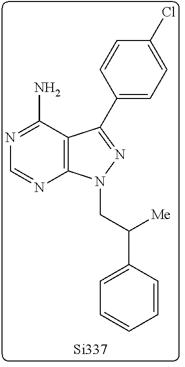

Yellow solid (42 mg, 43%); mp 153-154° C. ¹H NMR: δ 1.27 (d, J=6.8 Hz, 3H, CH₃), 3.46-3.62 (m, 1H, CH), 4.50-4.60 (m, 2H, CH₂), 5.74 (br s, 2H, NH₂ disappears with D₂O), 7.18-7.25, 7.41-7.49 and 7.51-7.61 (3m, 9H Ar), 8.33 (s, 1H, H-6). IR (cm⁻¹): 3470, 3421 (NH₂). MS: m/z 365 [M+H]⁺. Anal. (C₂₀H₁₈N₅Cl) C, H, N.

### 3-(4-Methylphenyl)-1-(2-phenylpropyl)-1H-pyrazolo[3,4-d]pyrimidin-4-amine (Si338)

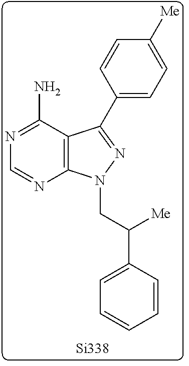

Light brown solid (30 mg, 32%); mp 152-155° C. ¹H NMR: δ 1.27 (d, J=4.4 Hz, 3H, CHCH₃), 2.43 (s, 3H, CH₃Ar), 3.47-3.66 (m, 1H, CH), 4.55-4.57 (m, 2H, CH₂), 5.31 (br s, 2H, NH₂ disappears with D₂O), 7.18-7.20, 7.32-7.44 and 7.53-7.55 (3m, 9H Ar), 8.34 (s, 1H, H-6). IR (cm⁻¹): 3480, 3325 (NH₂). MS: m/z 344 [M+H]⁺. Anal. (C₂₁H₂₁N) C, H, N.

### 3-(1H-indol-5-yl)-(2-phenylpropyl)-1H-pyrazolo[3,4-d]pyrimidin-4-amine (Si339)

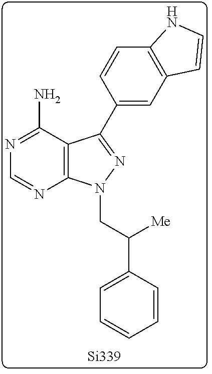

Light brown solid (30 mg, 30%); mp 240-241° C. ¹H NMR: δ 1.26 (d, J=7.2 Hz, 3H, CH₃), 3.49-3.74 (m, 1H, CH), 4.56-4.59 (m, 2H, CH₂), 5.46 (br s, 2H, NH₂ disappears with D₂O), 6.65 (m, 1H, indole H-3), 7.19-7.26, 7.27-7.32 and 7.49-7.56 (3m, 9H, 8H Ar and 1H, H-2 indole), 7.92 (s, 1H, NH), 8.35 (s, 1H, H-6). IR (cm⁻¹): 3465, 3309 (NH₂). MS: m/z 369 [M+H]⁺. Anal. (C₂₂H₂₀N₆) C, H, N.

### Synthesis of 6-(sec-butylthio)-1-(2-hydroxy-2-phenylethyl)-1,5-dihydro-4H-pyrazolo[3,4-d]pyrimidin-4-one (24c)

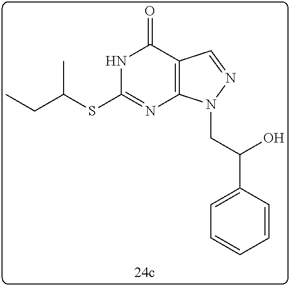 [SMILES: CCC(C)Sc1nc2c(cnn2CC(O)c2ccccc2)c(=O)[nH]1]

A mixture of 1-(2-hydroxy-2-phenylethyl)-6-thioxo-1,5,6,7-tetrahydro-4H-pyrazolo[3,4-d]pyrimidin-4-one 8 (2.88 g, 10 mmol), 2-bromobutane (1.11 mL, 10.14 mmol) and anhydrous K₂CO₃ (1.38 g, 10 mmol) in anhydrous DMF (10 mL) was stirred at room temperature for 24 h. The mixture was poured into cold water, the obtained white solid was filtered, washed with water and recrystallized with ethyl acetate. White solid (1.58 g, 46%); mp: 171-172° C. ¹H NMR: δ 1.00 (t, J=7.2 Hz, 3H, CH₂CH₃), 1.38-1.45 (m, 3H, CHCH₃), 1.62-1.78 (m, 2H, CH₂CH₃), 3.68-3.79 (m, 1H, SCH), 4.25-4.40 (m, 2H, CH₂N), 5.10-5.19 (m, 1H, CHOH), 7.18-7.41 (m, 5H Ar), 7.90 (s, 1H, H-3), 12.00 (br s, 1H, NH disappears with D₂O). IR (cm⁻¹): 3300-3030 (NH+OH), 1704 (CO). MS: m/z [M+1]⁺ 345. Anal. (C₁₇H₂₀N₄O₂S) C, H, N, S.

### Synthesis of 6-(sec-butylthio)-4-chloro-1-(2-chloro-2-phenylethyl)-1H-pyrazolo[3,4-d]pyrimidine (25c)

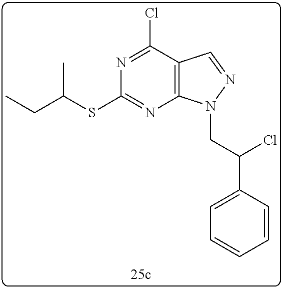 [SMILES: CCC(C)Sc1nc(Cl)c2cnn(CC(Cl)c3ccccc3)c2n1]

The Vilsmeier complex, previously prepared from POCl₃ (9.32 mL, 100 mmol) and anhydrous DMF (7.7 mL, 100 mmol) was added to a suspension of 6-(sec-butylthio)-1-(2-hydroxy-2-phenylethyl)-1,5-dihydro-4H-pyrazolo[3,4-d]pyrimidin-4-one 24c (3.44 g, 10 mmol) in CHCl₃ (50 mL). The mixture was refluxed for 8 h. The solution was washed with water (2×20 mL), dried (MgSO₄), filtered, and concentrated under reduced pressure. The crude oil was purified by column chromatography (Florisil®, 100-200 mesh) using diethyl ether as the eluent, to afford the pure product. Yellow oil (1.91 g, 50%). ¹H NMR: δ 1.03 (t, J=7.2 Hz, 3H, CH₂CH₃), 1.33-1.51 (m, 3H, CHCH₃), 1.62-1.86 (m, 2H, CH₂CH₃), 3.67-3.91 (m, 1H, SCH), 4.63-5.00 (m, 2H, CH₂N), 5.36-5.53 (m, 1H, CHCl), 7.11-7.43 (m, 5H Ar), 7.94 (s, 1H, H-3). MS: m/z [M+1]⁺ 382. Anal. (C₁₇H₁₈N₄Cl₂S) C, H, N, S.

### General Procedure for the Synthesis of Compounds Si146 and Si147.

The Suitable Amine (4 mmol) was added to a solution of 4-chloro derivative 25c (381 mg, 1 mmol) in anhydrous toluene (5 mL) and the mixture was stirred at room temperature for 48 h. The organic phase was washed with water (2×10 mL), dried (MgSO₄), filtered, and concentrated under reduced pressure. The crude oil was purified by column chromatography (Florisile, 100-200 mesh) using diethyl ether as the eluent. The compounds crystallized by adding a 1:1 mixture of Et₂O/petroleum ether (PE) (bp 40-60° C.).

### N-benzyl-6-(sec-butylthio)-1-(2-chloro-2-phenylethyl)-1H-pyrazolo[3,4-d]pyrimidin-4-amine (Si146)

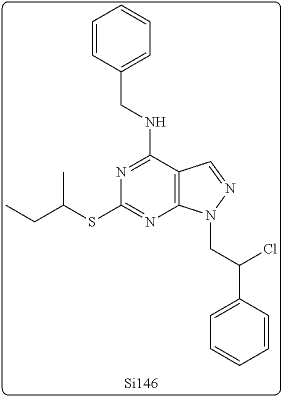 [SMILES: "Si146" ; CCC(C)Sc1nc(NCc2ccccc2)c2cnn(CC(Cl)c3ccccc3)c2n1]

White solid (271 mg, 60%); mp: 112-113° C. ¹H NMR: δ 1.05 (t, J=7.2 Hz, 3H, CH₂CH₃), 1.42-1.45 (m, 3H, CHCH₃), 1.68-1.74 and 1.80-1.85 (2m, 2H, CH₂CH₃), 3.81-3.84 (m, 1H, SCH), 4.68-4.88 (m, 4H, CH₂N+CH₂Ar), 5.49-5.53 (m, 1H, CHCl), 7.24-7.41 (m, 10H Ar), 7.69 (s, 1H, H-3). IR (cm⁻¹): 3250 (NH). MS: m/z [M+1]⁺ 453. Anal. (C₂₄H₂₆N₅ClS) C, H, N, S.

### 6-(Sec-butylthio)-1-(2-chloro-2-phenylethyl)-N-(2-phenylethyl)-1H-pyrazolo[3,4-d]pyrimidin-4-amine (Si147)

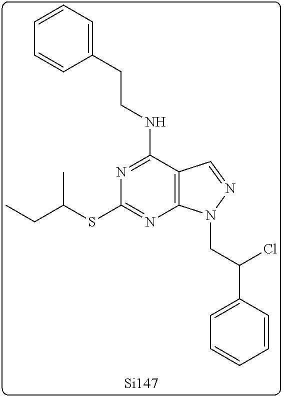 [SMILES: "Si147" ; CCC(C)Sc1nc(NCCc2ccccc2)c2cnn(CC(Cl)c3ccccc3)c2n1]

White solid (368 mg, 79%); mp: 97-98° C. ¹H NMR: δ 1.03 (t, J=7.0 Hz, 3H, CH₂CH₃), 1.34-1.50 (m, 3H, CHCH₃), 1.59-1.83 (m, 2H, CH₂CH₃), 2.91 (t, J=6.2 Hz, 2H, CH₂Ar), 3.70-3.88 (m, 3H, SCH+CH₂NH), 4.60-4.90 (m, 2H, CH₂N), 5.30 (br s, 1H, NH disappears with D₂O), 5.41-5.54 (m, 1H, CHCl), 7.04-7.41 (m, 10H Ar), 7.63 (s, 1H, H-3). IR (cm⁻¹): 3255 (NH). MS: m/z [M+1]⁺ 467. Anal. (C₂₅H₂₈N₅ClS) C, H, N, S.

### General Procedure for the Synthesis of Compounds Si170 and Si148.

The appropriate aniline (2 mmol) was added to a solution of the 4-chloro derivative 25b or 25c (1 mmol) in absolute ethanol (5 mL), and the mixture was refluxed for 3-5 h. After cooling to room temperature, the obtained solid was filtered, washed with water, and recrystallized from absolute ethanol.

### 1-(2-Chloro-2-phenylethyl)-6-(cyclopentylthio)-N-(3-fluorophenyl)-1H-pyrazolo[3,4-d]pyrimidin-4-amine (Si170)

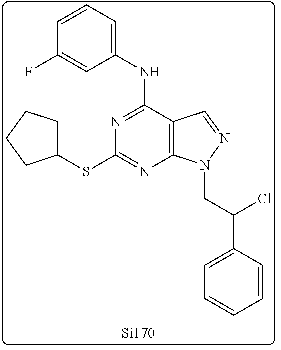 [SMILES: "Si170" ; Fc1cccc(Nc2nc(SC3CCCC3)nc3c2cnn3CC(Cl)c2ccccc2)c1]

White solid (206 mg, 44%); mp: 226-227° C. ¹H NMR: δ 1.78-1.84 and 2.12-2.43 (2m, 8H, 4CH₂ cyclopentyl), 3.98-4.17 (m, 1H, SCH), 4.54-4.68 and 4.73-4.89 (2m, 2H, CH₂N), 5.26-5.44 (m, 1H, CHCl), 5.54 (br s, 1H, NH disappears with D₂O), 6.93-7.53 (m, 10H, 9Ar+H-3). IR (cm⁻¹): 2835 (NH). MS: m/z [M+1]⁺ 469. Anal. (C₂₄H₂₃N₅ClFS) C, H, N, S.

### 6-(Sec-butylthio)-N-(3-chlorophenyl)-1-(2-chloro-2-phenylethyl)-1H-pyrazolo[3,4-d]pyrimidin-4-amine (Si148)

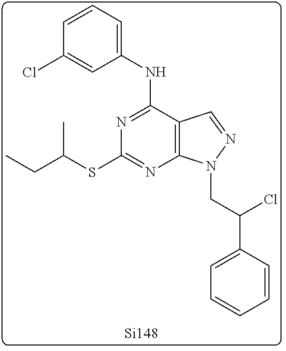 [SMILES: "Si148" ; CCC(C)Sc1nc(Nc2cccc(Cl)c2)c2cnn(CC(Cl)c3ccccc3)c2n1]

White solid (236 mg, 50%); mp: 213-214° C. ¹H NMR: δ 1.02 (t, J=7.0 Hz, 3H, CH₂CH₃), 1.40-1.44 (m, 3H, CHCH₃), 1.65-1.80 (m, 2H, CH₂CH₃), 3.80-3.85 (m, 1H, SCH), 4.75-4.80 and 4.87-4.93 (2m, 2H, CH₂N), 5.63-5.67 (m, 1H, CHCl), 7.14-7.66 (m, 9H Ar), 8.30 (s, 1H, H-3), 10.34 (br s, 1H, NH disappears with D₂O). IR (cm⁻¹): 2933 (NH). MS: m/z [M+1]⁺ 473. Anal. (C₂₃H₂₃N₅Cl₂S) C, H, N, S.

### Synthesis of N-[2-(3-chlorophenyl)ethyl]-6-(methylthio)-1-[2-phenylvinyl]-1H-pyrazolo[3,4-d]pyrimidin-4-amine (Si215)

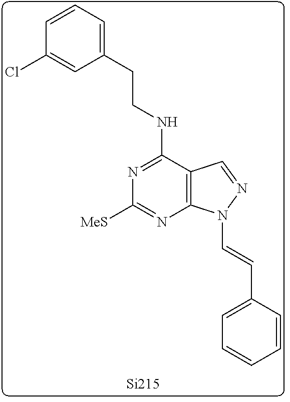 [SMILES: "Si215" ; CSc1nc(NCCc2cccc(Cl)c2)c2cnn(/C=C/c3ccccc3)c2n1]

A solution of 4N NaOH (2 mL) was added to a suspension of N-[2-(3-chlorophenyl)ethyl]-1-(2-chloro-2-phenylethyl)-6-(methylthio)-1H-pyrazolo[3,4-d]pyrimidin-4-amine Si58 (458 mg, 1 mmol) in 95% ethanol (12 mL), and the mixture was refluxed for 5 h. After cooling, the solid was filtered, washed with water, and recrystallized from absolute ethanol. White solid (273 mg, 65%); mp: 104-106° C. ¹H NMR: δ 2.64 (s, 3H, SCH₃), 2.98 (t, J=5.0 Hz, 2H, CH₂Ar), 3.87 (q, J=5.0 Hz, 2H, CH₂NH), 5.52 (br s, 1H, NH disappears with D₂O), 7.09-7.50 (m, 10H, 9Ar+CH═), 7.92 (s, 1H, H-3), 7.96 (d, Jₜᵣₐₙₛ, =16.0 Hz, 1H, CH═). IR (cm⁻¹): 3269 (NH), 1663 (C═C). MS: m/z [M+1]⁺ 423. Anal. (C₂₂H₂N₅ClS), C, H, N, S.

### Synthesis of 2-(4-benzylamino-1-styryl-1H-pyrazolo[3,4-d]pyrimidin-6-ylamino)-ethanol (Si74)

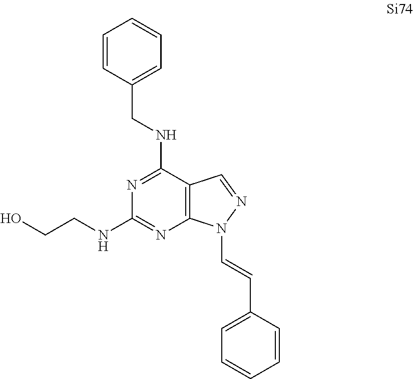 [SMILES: OCCNc1nc(NCc2ccccc2)c2cnn(/C=C/c3ccccc3)c2n1]

Ethanolamine (180 μL, 3 mmol) was added to a suspension of 26 (405 mg, 1 mmol) in butan-1-ol (16 mL) and DMSO (4 mL), and the mixture was heated at 90° C. for 12 h. After cooling to room temperature, butan-1-ol was removed under reduced pressure; then water (20 mL) was added and the solution was extracted with ethyl acetate (2×20 mL); the organic phase was washed with water (20 mL), dried (MgSO₄) and evaporated under reduced pressure. The obtained solid was filtered and recrystalized from absolute ethanol. White solid. (255 mg, 66%); mp: 148-149° C. ¹H NMR: δ 3.68 (q, J=4.8 Hz, 2H, CH₂), 3.88 (q, J=4.8 Hz, 2H, CH₂), 4.77 (d, J=4.6 Hz, 2H, CH₂Ar), 5.63 (br s, 1H, NH, disappears with D₂O), 7.22-7.54 (m, 11H, 10Ar+CH═), 7.78 (s, 1H, H-3), 7.82 (d, Jₜᵣₐₙₛ, =17.2 Hz, 1H, CH═). IR (cm⁻¹): 3281-3025 (OH+NH), 1657 (C═C). MS: m/z [M+1]⁺ 387. Anal. (C₂₂H₂₂N₆O) C, H, N.

### Synthesis of 6-benzyl-1-(2-hydroxy-2-phenylethyl)-1,5-dihydro-4H-pyrazolo[3,4-d]pyrimidin-4-one (28)

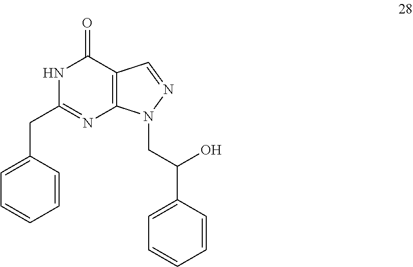 [SMILES: O=c1[nH]c(Cc2ccccc2)nc2c1cnn2CC(O)c1ccccc1]

A solution of sodium ethoxide, prepared from sodium (138 mg, 6 mmol) and absolute ethanol (5 mL), and methyl phenylacetate (900 mg, 6 mmol) were added to a solution of 5-amino-1-(2-hydroxy-2-phenylethyl)-1H-pyrazole-4-carboxamide 27 (246 mg, 1 mmol) in absolute ethanol (5 mL). The mixture was refluxed for 6 h; after cooling to room temperature, ice water (30 mL) was added and the solution was acidified with 3% acetic acid. The precipitated solid was filtered, washed with water and recrystallized from absolute ethanol to afford compound 28 White solid (200 mg, 58%); mp: 205-207° C. ¹H NMR: δ 3.98 (s, 2H, CH₂Ar), 4.06 (br s, 1H, OH disappears with D₂O), 4.44-4.51 and 4.55-4.61 (2m, 2H, CH₂N), 5.13-5.18 (m, 1H, CHOH), 7.16-7.35 (m, 10H Ar), 8.00 (s, 1H, H-3), 10.94 (br s, 1H, NH disappears with D₂O). IR (cm⁻¹): 3440-2893 (OH+NH), 1694 (CO). MS: m/z [M+1]⁺ 347. Anal. (C₂₀H₁₈N₄O₂) C, H, N.

### Synthesis of 6-benzyl-4-chloro-1-(2-chloro-2-phenylethyl)-1H-pyrazolo[3,4-d]pyrimidine (29)

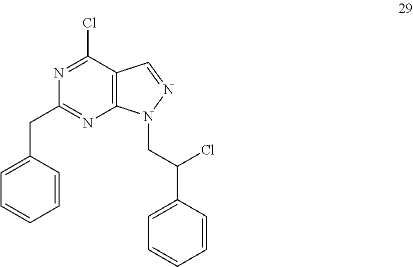 [SMILES: Clc1nc(Cc2ccccc2)nc2c1cnn2CC(Cl)c1ccccc1]

The Vilsmeier complex, previously prepared from POCl₃ (2.80 mL, 30 mmol) and anhydrous DMF (2.3 mL, 30 mmol) was added to a suspension of 6-benzyl-1-(2-hydroxy-2-phenylethyl)-1,5-dihydro-4H-pyrazolo[3,4-d]pyrimidin-4-one 28 (346 mg, 1 mmol) in CHCl₃ (10 mL). The mixture was refluxed for 12 h. The solution was washed with water (2×20 ml), dried (MgSO₄), filtered and concentrated under reduced pressure. The crude oil was purified by column chromatography (Florisile, 100-200 mesh), using diethyl ether as the eluent, to afford the compound as a yellow oil, which crystallized standing in a refrigerator by adding a 1:1 mixture of Et₂O/PE (bp 40-60° C.) (1:1). White solid (320 mg, 84%); mp: 172-173° C. ¹H NMR: δ 3.99 (s, 2H, CH₂Ar), 4.64-4.77 and 4.81-4.96 (2m, 2H, CH₂N), 5.36-5.51 (m, 1H, CHCl), 7.03-7.66 (m, 10H Ar), 8.03 (s, 1H, H-3). MS: m/z [M+1]⁺ 384. Anal. (C₂₀H₁₆N₄Cl₂) C, H, N.

### N,6-dibenzyl-1-(2-chloro-2-phenylethyl)-1H-pyrazolo[3,4-d]pyrimidin-4-amine (Si164)

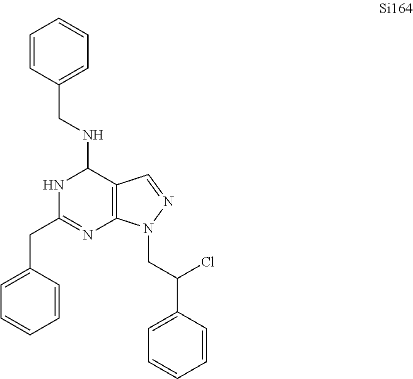 [SMILES: ClC(Cn1ncc2c1N=C(Cc1ccccc1)NC2NCc1ccccc1)c1ccccc1]

Benzylamine (440 μL, 4 mmol) was added to a solution of 4-chloro derivative 29 (383 mg, 1 mmol) in anhydrous toluene (5 mL) and the mixture was stirred at room temperature for 48 h. The organic phase was washed with water (2×10 mL), dried (MgSO₄), filtered, and concentrated under reduced pressure. The crude oil was crystallized by adding a 1:1 mixture of Et₂O/PE (bp: 40-60° C.) to give Si164. White solid (250 mg, 55%); mp: 125° C. ¹H NMR: δ 4.00 (s, 2H, CH₂Ar), 4.53-4.96 (m, 4H, CH₂N+NHCH₂ Ar), 5.40-5.54 (m, 1H, CHCl), 7.00-7.73 (in, 15H Ar), 8.04 (s, 1H, H-3). IR (cm⁻¹): 3255 (NH) MS: m/z [M+1]⁺ 455. Anal. (C₂₇H₂₄N₅C₁) C, H, N.

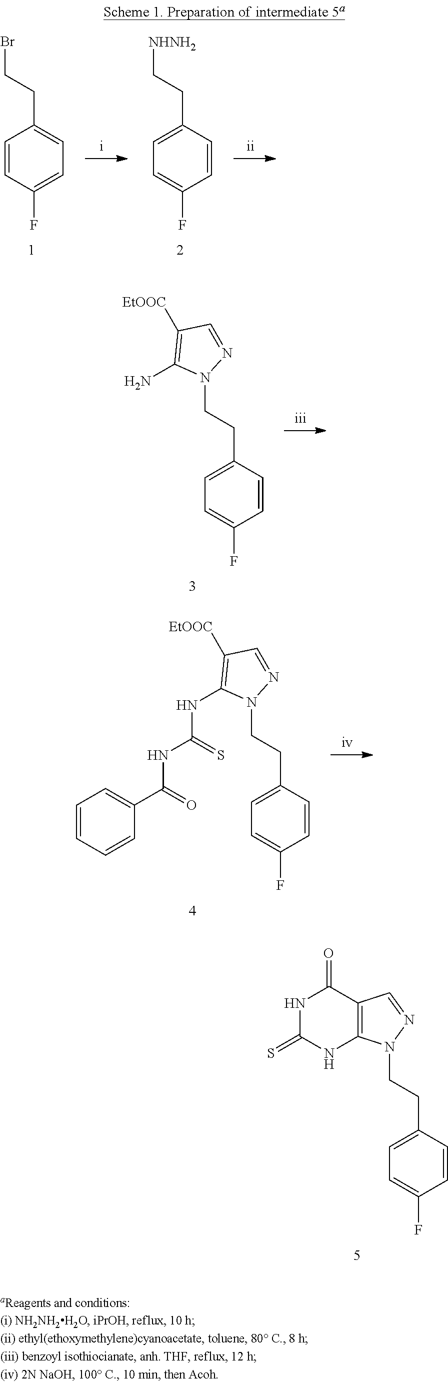 [SMILES: Fc1ccc(CCBr)cc1 ; NNCCc1ccc(F)cc1 ; CCOC(=O)c1cnn(CCc2ccc(F)cc2)c1N ; CCOC(=O)c1cnn(CCc2ccc(F)cc2)c1NC(=S)NC(=O)c1ccccc1 ; O=c1[nH]c(=S)[nH]c2c1cnn2CCc1ccc(F)cc1]

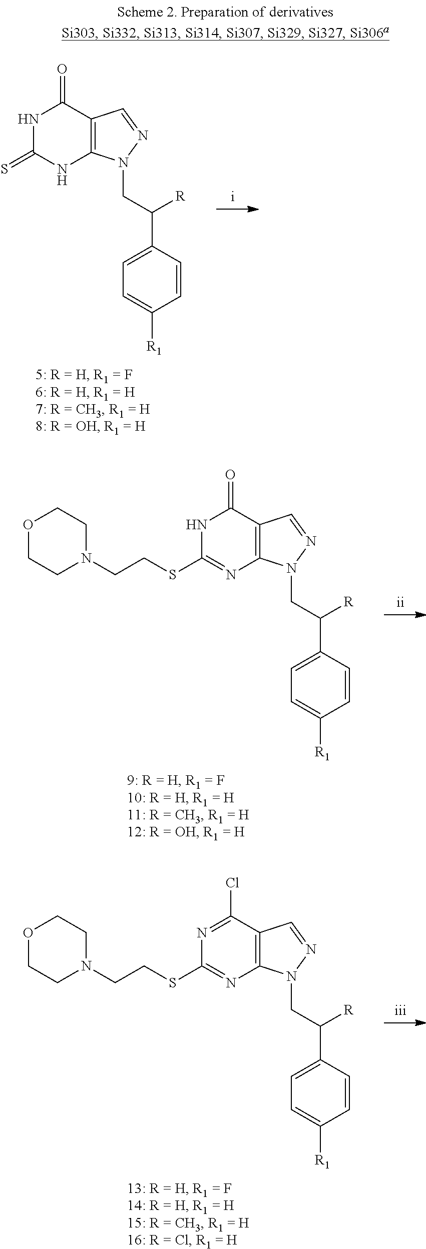

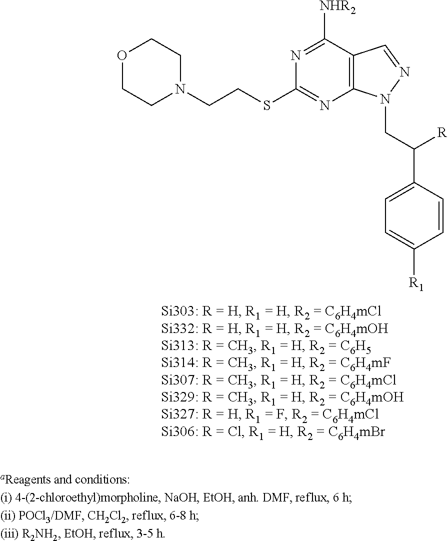

Scheme 3. Preparation of derivatives Si308, Si309, Si310, Si311, Si244^{a}

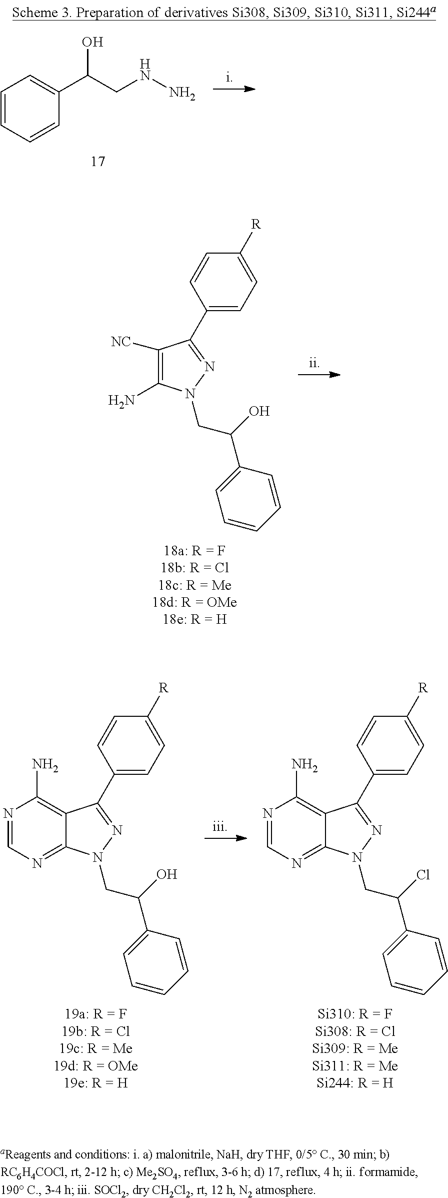

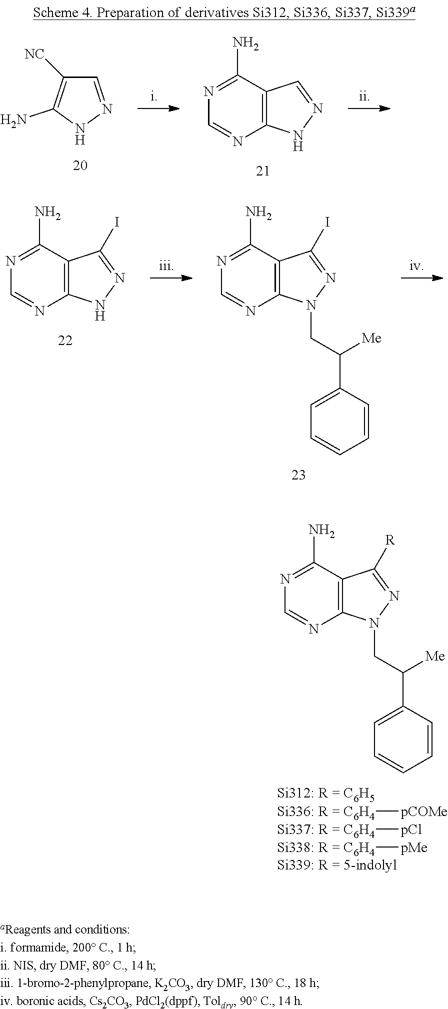

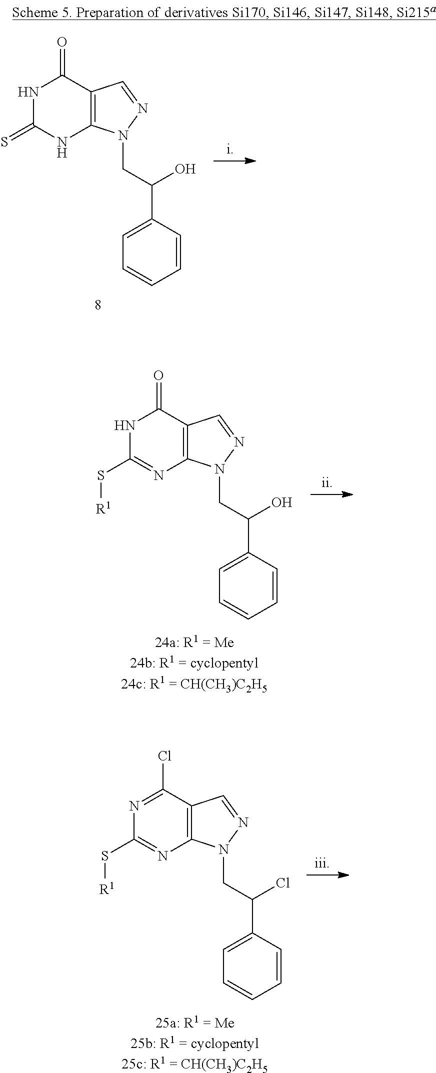

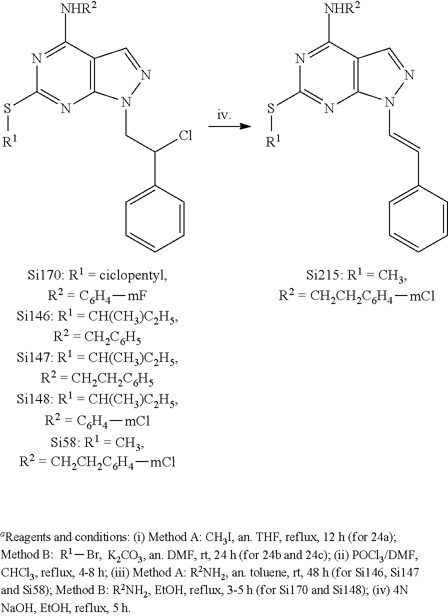

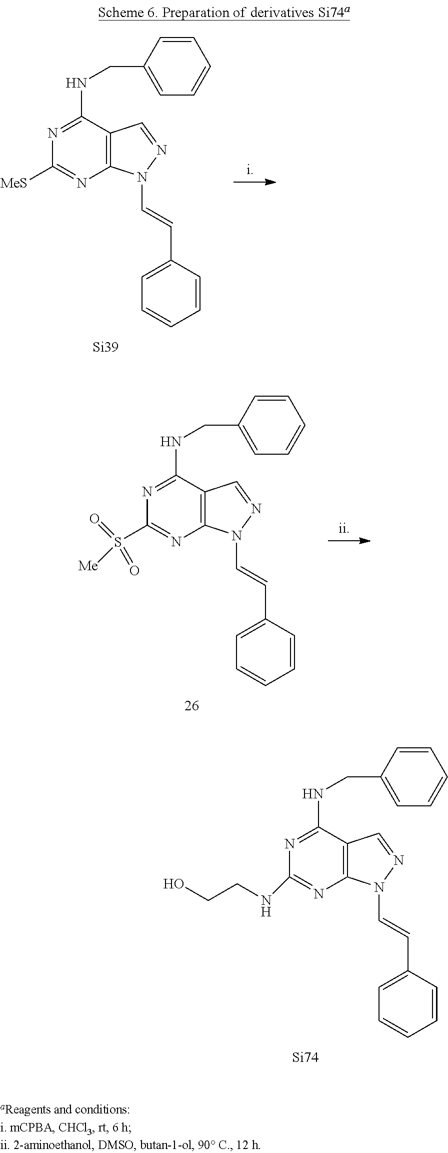 [SMILES: "Si39" ; CSc1nc(NCc2ccccc2)c2cnn(/C=C/c3ccccc3)c2n1 ; CS(=O)(=O)c1nc(NCc2ccccc2)c2cnn(/C=C/c3ccccc3)c2n1 ; "26" ; "Si74" ; OCCNc1nc(NCc2ccccc2)c2cnn(/C=C/c3ccccc3)c2n1]

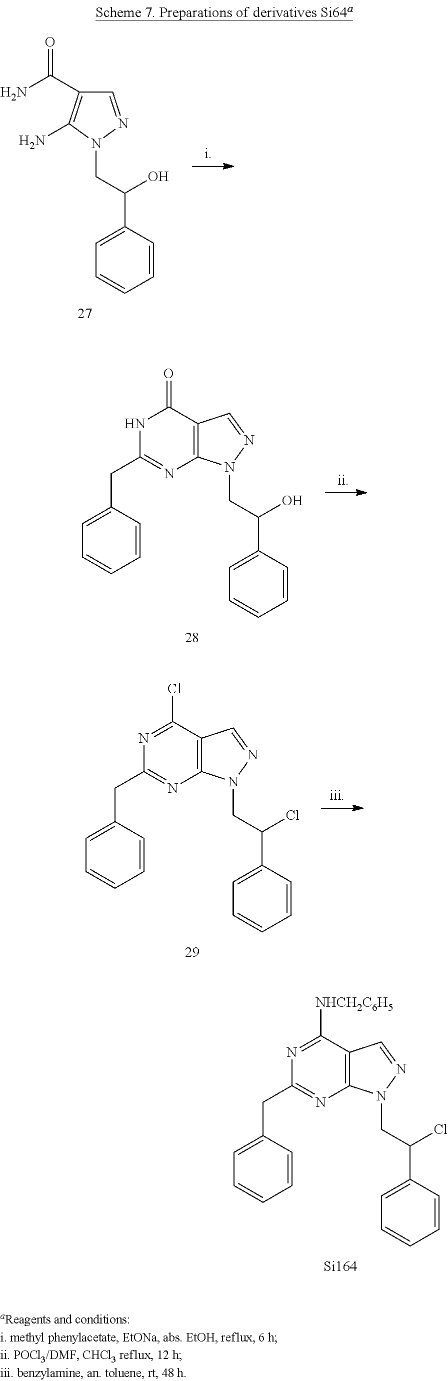

1.3—Chemistry: Discussion

Compound Si327 was synthesized starting from the [2-(4-fluorophenyl)ethyl]hydrazine 2, obtained by reaction of 1-(2-bromoethyl)-4-fluorobenzene 1 with hydrazine monohydrate in isopropanol at reflux for 10 h (Scheme 1). The hydrazine derivative 2 was reacted with ethyl(ethoxymethylene)cyanoacetate in anhydrous toluene at 80° C. for 8 h affording the ethyl 5-amino-1-[2-(4-fluorophenyl)ethyl]-1H-pyrazole-4-carboxylate 3, which was treated with benzoyl isothiocyanate in anhydrous THF at reflux for 12 h to give the intermediate 4. This compound was in turn cyclized to the pyrazolo[3,4-d]pyrimidinone 5 by treatment with 2 N NaOH at 100° C. for 10 min, followed by acidification with acetic acid. Alkylation of the thiocarbonyl group at position C₆ with 4-(2-chloroethyl)morpholine in anhydrous DMF in the presence of alcoholic NaOH solution gave compound 9, which was treated with the Vilsmeier complex (POCb/DMF, 1:1) in CH₂Cl₂ at reflux for 12 h to obtain the halogenated compound 13 (Scheme 2). Finally, the latter was reacted with an excess of 3-chloro aniline in absolute ethanol at reflux for 5 h, affording the desired compound Si327. The synthesis of the other compounds bearing a N-morpholino-ethanthio substituent in C₆ is reported in Scheme 2. Comparison compound Si181, shown herein in Table 2, has been previously reported by us.²¹ Alkylation of the thiocarbonyl group of derivatives 5-82 with 4-(2-chloroethyl)morpholine afforded the 6-alkylthio derivatives 9-12, which were in turn treated with the Vilsmeier complex (POCl₃/DMF, 1:1) in CH₂Cl₂ at reflux for 6-8 h to obtain compounds 13-16 bearing a chlorine atom in C4. Finally, reaction of 13-16 with the suitable anilines in absolute ethanol at reflux for 3-5 h gave desired Si compounds in good yields.

The synthesis of 3-substituted pyrazolo[3,4-d]pyrimidines Si244, Si308, Si309, Si310 and Si311 was performed using a three components one-pot synthesis.²⁴ Sodium hydride was added in small batches to a solution of malononitrile in dry THF precooled at 0/5° C.; after 30 minutes the suitable acyl chloride was added and the solution stirred at room temperature for 2-12 h. Then dimethylsulfate was added and the solution was refluxed for 3-6 h. Finally, 2-hydrazino-1-phenylethanol 17 dissolved in dry THF (2 mL) was added and the reaction was refluxed for 4 h to afford intermediates 18a-c, purified by flash chromatography. Compounds 18a-c were suspended in formamide and the mixture was heated at 190° C. for 3-4 h to afford the pyrazolopyrimidines 19a-c, that were in turn reacted with thionyl chloride in dry CH₂Cl₂ at room temperature for 12 h under nitrogen atmosphere to give the final compounds Si308, Si309 and Si310 (Scheme 3). Synthesis of compounds Si312, Si337, Si336, Si338 and Si339 was performed via Suzuki cross-coupling, since the one-pot reaction previously described led to very low yields. 5-Amino-1H-pyrazolo-4-carbonitrile 20,²⁰ obtained by reaction of (ethoxymethylene)malononitrile with hydrazine monohydrate, was cyclized by reaction with formamide at 200° C. for 1 h, affording 1H-pyrazolo[3,4-d]pyrimidin-4-amine 21.²⁰ Reaction of 21 with N-iodosuccinimide (NIS) in dry DMF at 80° C. for 14 h under nitrogen atmosphere gave 3-iodo-1H-pyrazolo[3,4-d]pyrimidin-4-amine 22.²⁶ This last was in turn treated with K₂CO₃ and 1-bromo-2-phenylpropane at 130° C. for 18 h to afford 3-iodo-1-(2-phenylpropyl)-1H-pyrazolo[3,4-d]pyrimidin-4-amine 23 in good yield. Finally, compound 23 was reacted with an excess of the suitable boronic acid in the presence of Cs₂CO₃ and PdCl₂(dppf) in dry toluene at 90° C. for 14 h to give compounds Si336 and Si339 (Scheme 4). The synthesis of compounds Si170, Si146, Si147, Si148 and Si74 was performed starting from 1-(2-hydroxy-2-phenylethyl)-6-thioxo-1,5,6,7-tetrahydro-4H-pyrazolo[3,4-d]pyrimidin-4-one 8, previously reported by us.²⁰ Alkylation of the C₆ thiocarbonyl group with the suitable alkyl bromide in anhydrous N,N-dimethylformamide (DMF) at room temperature afforded the 6-alkylthio derivatives 24a-c, in turn treated with the Vilsmeier complex (POCl₃/DMF, 1:1) in CHCl₃ to obtain the dichloro-derivatives 25a-c. Finally, reaction with an excess of the appropriate amines in toluene at room temperature afforded compounds Si146, Si147, Si58 and Si34 in good yields. Differently, compounds Si170 and Si148 were obtained reacting 25b,c with the suitable anilines in absolute ethanol at reflux for 3-5 h. Compound Si215 has been obtained by treatment of Si34 with a 4N NaOH solution at reflux for 5 h (Scheme 5). Oxidation of compound Si39 with meta-chloroperoxybenzoic acid (mCPBA) in CHCl₃ gave the 6-methylsulfonyl derivative 26. Finally, Si74 was obtained by nucleophilic substitution of the methylsulfonyl group with 2-aminoethanol in dimethylsulfoxide (DMSO) and butan-1-ol at 90° C. for 12 h in good yield (Scheme 6). Compound Si164 was obtained starting from 27¹⁸ this was chlorinated with the Vilsmeier complex in CHCl₃ at reflux for 12 h and gave the dichloro derivative 29, that by reaction with benzylamine, afforded Si164 (Scheme 7).

### EXAMPLE 2

### 2-ADME Assays:

### 2.1—ADME Assays: Materials and Methods

Chemicals.

All solvents, reagents, were from Sigma-Aldrich Sri (Milan, Italy), Brain Polar Lipid Extract (Porcine) were from Avanti Polar Lipids, INC. (Alabama, USA). Dodecane was purchased from Fluka (Milan, Italy). Pooled Male Donors 20 mg mL⁻¹ HLM were from BD Gentest-Biosciences (San Jose, Calif.). Milli-Q quality water (Millipore, Milford, Mass., USA) was used. Hydrophobic filter plates (MultiScreen-IP, Clear Plates, 0.45 man diameter pore size), 96-well microplates, and 96-well UV-transparent microplates were obtained from Millipore (Bedford, Mass., USA).

Parallel Artificial Membrane Permeability Assay (PAMPA).

Donor solution (0.5 mM) was prepared by diluting 1 mM dimethylsulfoxide (DMSO) compound stock solution using phosphate buffer (pH 7.4, 0.025 M). Filters were coated with 5 μL of a 1% (w/v) dodecane solution of phosphatidylcholine or 4 μL of brain polar lipid solution (20 mg mL⁻¹ 16% CHCl₃, 84% dodecane) prepared from CHCl₃ solution 10% w/v, for intestinal permeability and BBB permeability, respectively. Donor solution (150 μL) was added to each well of the filter plate. To each well of the acceptor plate were added 300 μL of solution (50% DMSO in phosphate buffer). All compounds were tested in three different plates on different days. The sandwich was incubated for 5 h at room temperature under gentle shaking. After the incubation time, the plates were separated, and samples were taken from both receiver and donor sides and analyzed using LC with UV detection at 280 nm.

LC analysis were performed with a Varian Prostar HPLC system (Varian Analytical Instruments, USA) equipped with a binary pump with a manual injection valve and model Prostar 325 UV-VIS Detector. Chromatographic separation were conducted using a Polaris C18-A column (150-4.6 mm, 5 μm particle size) at a flow rate of 0.8 mL min⁻¹ with a mobile phase composed of 60% ACN/40% H₂O-formic acid 0.1%.

Permeability (Pₐₚₚ) for PAMPA was calculated according to the following equation, obtained from Wohnsland and Faller²⁷ and Sugano et al.²⁸ equation with some modification in order to obtain permeability values in cm s⁻¹,

$${P}_{app} = \frac{{V}_{D} {V}_{A}}{( {V}_{D} + {V}_{A} ) At} - ln ( 1 - r )$$

where V_{A} is the volume in the acceptor well, V_{D} is the volume in the donor well (cm³), A is the “effective area” of the membrane (cm²), t is the incubation time (s) and r the ratio between drug concentration in the acceptor and equilibrium concentration of the drug in the total volume (V_{D}+V_{A}). Drug concentration is estimated by using the peak area integration. Membrane retentions (%) were calculated according to the following equation:

$$\% MR = \frac{[ r - ( D + A ) ] 100}{Eq}$$

where r is the ratio between drug concentration in the acceptor and equilibrium concentration, D, A, and Eq represented drug concentration in the donor, acceptor and equilibrium solution, respectively.

### Water Solubility Assay.

Each solid compound (1 mg) was added to 1 mL of water. The samples were shaked in a shaker bath at room temperature for 24-36 h. The suspensions were filtered through a 0.45 μm nylon filter (Acrodisc), and the solubilized compound determined by LC-MS-MS assay. For each compound the determination was performed in triplicate. For the quantification was used an LC-MS system consisted of a Varian apparatus (Varian Inc) including a vacuum solvent degassing unit, two pumps (212-LC), a Triple Quadrupole MSD (Mod. 320-LC) mass spectrometer with ES interface and Varian MS Workstation System Control Vers. 6.9 software. Chromatographic separation was obtained using a Pursuit C18 column (50×2.0 mm) (Varian) with 3 μm particle size and gradient elution: eluent A being ACN and eluent B consisting of an aqueous solution of formic acid (0.1%). The analysis started with 0% of eluent A, which was linearly increased up to 70% in 10 min, then slowly increased up to 98% up to 15 min. The flow rate was 0.2 mL min-⁻¹ and injection volume was 5 μL. The instrument operated in positive mode and parameters were: detector 1850 V, drying gas pressure 25.0 psi, desolvation temperature 300.0° C., nebulizing gas 40.0 psi, needle 5000 V and shield 600 V. Nitrogen was used as nebulizer gas and drying gas. Collision induced dissociation was performed using Argon as the collision gas at a pressure of 1.8 mTorr in the collision cell. The transitions as well as the capillary voltage and the collision energy used for each compound are summarized in Table 5.

TABLE 5
Chromatographic and MS parameters (monitored transition,
collision energy, capillary voltage and retention time
t_{R}) of the selected compound.

| | Transition | Collision Energy | Capillary voltage | t_{R} |
|---|---|---|---|---|
| Cpd | (m/z) | (eV) | (V) | (min) |
| Si319 | 258.0 | −27.0 | 109 | 5.38 |
| | 210.0 | −37.5 | | |
| Si320 | 275.9 | −27.0 | 113 | 5.53 |
| | 228.0 | −39.5 | | |
| Si321 | 337.9 | −28.0 | 99 | 5.85 |
| | 289.8 | −40.5 | | |
| Si328 | 264.0 | −28.5 | 114 | 4.88 |
| | 226.0 | −39.0 | | |
| Si303 | 408.1 | −25.0 | 33 | 4.04 |
| | 113.9 | −27.0 | | |
| Si332 | 390.1 | −24.5 | 73 | 5.68 |
| | 113.9 | −28.5 | | |
| Si315 | 290.0 | −24.5 | 74 | 5.87 |
| | 261.9 | −31.5 | | |
| Si316 | 277.9 | −31.5 | 79 | 5.17 |
| | 305.9 | −24.5 | | |
| Si317 | 351.9 | −27.0 | 110 | 6.17 |
| | 323.8 | −33.5 | | |
| Si318 | 260.0 | −34.5 | 93 | 6.25 |
| Si313 | 388.0 | −21.5 | 64 | 3.80 |
| | 270.0 | −36.5 | | |
| Si314 | 406.0 | −22.5 | 20 | 3.85 |
| | 288.0 | −25.0 | | |
| Si307 | 422.1 | −23.0 | 10 | 4.07 |
| | 304.1 | −38.0 | | |
| Si329 | 404.1 | −24.0 | 10 | 3.89 |
| | 286.0 | −38.5 | | |
| Si327 | 426.0 | −25.5 | 65 | 3.90 |
| | 303.9 | −41.5 | | |
| Si322 | 376.0 | −21.0 | 89 | 4.89 |
| | 303.1 | −27.5 | | |
| Si331 | 418.1 | −23.5 | 129 | 5.64 |
| | 362.0 | −30.5 | | |
| Si323 | 361.9 | −24.0 | 97 | 5.23 |
| | 404.0 | −18.0 | | |
| Si171 | 414.1 | −19.0 | 84 | 6.32 |
| | 346.1 | −26.0 | | |
| Si170 | 364.1 | −26.5 | 80 | 6.43 |
| | 432.2 | −20.0 | | |
| Si330 | 430.1 | −21.5 | 94 | 5.68 |
| | 362.0 | −28.0 | | |
| Si306 | 452.1 | −27.0 | 49 | 4.17 |
| | 539.2 | −17.0 | | |

Quantification of the single compound was made by comparison with apposite calibration curves realized with standard solutions in methanol.

Microsomal Stability Assay.

Each compound in DMSO solution was incubated at 37° C. for 60 min in 125 mM phosphate buffer (pH 7.4), 5 μL of human liver microsomal protein (0.2 mg mL⁻¹), in the presence of a NADPH-generating system at a final volume of 0.5 mL (compound final concentration, 50 μM); DMSO did not exceed 2% (final solution). The reaction was stopped by cooling on ice and adding 1.0 mL of acetonitrile. The reaction mixtures were then centrifuged, and the parent drug and metabolites were subsequently determined by LC-UV-MS.

Chromatographic analysis was performed with an Agilent 1100 LC/MSD VL system (G1946C) (Agilent Technologies, Palo Alto, Calif.) constituted by a vacuum solvent degassing unit, a binary high-pressure gradient pump, an 1100 series UV detector, and an 1100 MSD model VL benchtop mass spectrometer.

Chromatographic separation was obtained using a Varian Polaris C18-A column (150-4.6 mm, 5 μm particle size) and gradient elution: eluent A being ACN and eluent B consisting of an aqueous solution of formic acid (0.1%). The analysis started with 2% of eluent A, which was rapidly increased up to 70% in 12 min, then slowly increased up to 98% in 20 min. The flow rate was 0.8 mL min⁻¹ and injection volume was 20 μL.

The Agilent 1100 series mass spectra detection (MSD) single-quadrupole instrument was equipped with the orthogonal spray API-ES (Agilent Technologies, Palo Alto, Calif.). Nitrogen was used as nebulizing and drying gas. The pressure of the nebulizing gas, the flow of the drying gas, the capillary voltage, the fragmentor voltage, and the vaporization temperature were set at 40 psi, 9 L/min, 3000 V, 70 V, and 350° C., respectively. UV detection was monitored at 280 nm. The LC-ESI-MS determination was performed by operating the MSD in the positive ion mode. Spectra were acquired over the scan range m/z 100-1500 using a step size of 0.1 u.

The percentage of not metabolized compound was calculated by comparison with reference solutions.

### 1.2—ADME Assay: Discussion

It is well known that kinase inhibitors are generally affected by solubility issues because of their lipophilic nature. Therefore, the early evaluation of ADME properties in this field represents, more than ever, a key step to guide the drug candidate selection. Accordingly, in vitro ADME studies were conducted on the most potent c-Src inhibitors reported herein in order to early assess their absorption/stability. In particular, aqueous solubility, parallel artificial membrane permeability (PAMPA) and human liver microsomes (HLM) stability were evaluated for the most active c-Src inhibitors (Table 6). When compared to previously synthesized compounds¹⁹ characterized for their activity against neuroblastoma, the class of compounds of the present invention showed optimal ADME properties, with special regards to aqueous solubility. Indeed, previous C6-methylthio derivatives¹⁹ Si34, Si35 and Si83 showed very low water solubility values (ranging from 0.07 to 0.12 μg/mL). By contrats, the compounds of the invention have an aqueous solubility increased by about 2- to greater than 57-fold compared to that of reference C6-morpholine derivative Si192. The most soluble compound being Si332 showing a solubility value of 97 μg/mL.

### EXAMPLE 3

Enzymatic Assays and Biological Activity Against Neuroblastoma. Glioblastoma and Leukemia

### 3.1—Enzymatic Assays:

### 3.1.1—Enzymatic Assays: Materials and Methods

### Enzymatic Assay on Isolated Fyn Kinase.

Active, recombinant Fyn and the specific peptide substrates (Sre Substrate Peptide, cat 12-140) were purchased from Merk-Millipore. Kinase assays were performed in presence of 200 μM ATP and 100 μM peptide substrate. All inhibition assays were conducted with 0.01 μg active kinase, 0.33 μmol [^{γ32}P]ATP, 60 mM HEPES-NaOH pH 7.5, 3 μM Na-orthovanadate, 1.2 mM DTT, 50 μg/ml PEG_{20.0000}, 10 mM magnesium acetate, 0.004% NP40 and 10% DMSO in a final volume of 10 μL. Fyn and inhibitors were preincubated in ice for 5 min; after addition of the substrates the reaction was conducted at 30° C. for 10 min. The reaction was stopped by adding 5 μL of 3% phosphoric acid. Aliquots (10 μL) were then transferred into a P30 Filtermat (PerkinElmer), washed five times with 75 mM phosphoric acid and once with acetone for 5 minute. The filter was dried and transferred to a sealable plastic bag, and scintillation cocktail (4 mL) was added. Spotted reactions were read in a MicroBeta Liquid (Perkinelmer) scintillation counter. The ID₅₀ values were obtained according to the following equation:

v=V/(1+(I/ID₅₀)

where v is the measured reaction velocity, V is the apparent maximal velocity in the absence of inhibitor, I is the inhibitor concentration, and the ID₅₀ is the 50% inhibitory dose. Ki values toward recombinant Fyn were calculated using the equation:

Ki=(I_{D50}(1+K_{mATP}/[S_{ATP}]))

according to a competitive mechanism of inhibition toward ATP substrate, where [S_{ATP}] is the concentration of ATP. Curve fitting was performed with the program GraphPad Prism version 5.00.

### Enzymatic Assay on Isolated Src.

Recombinant human Src was purchased from Upstate (Lake Placid, N.Y.). Activity was measured in a filter-binding assay using a commercial kit (Src Assay Kit, Upstate), according to the manufacturer's protocol, using 150 μM of the specific Src peptide substrate (KVEKIGEGTYGVVYK) and in the presence of 0.125 pMol of Src and 10 μM of [γ-32P]-ATP. The apparent affinity (Km) values of the Src preparation used for its peptide and ATP substrates were determined separately and found to be 30 μM and 5 μM, respectively.

### Enzymatic Assay on Isolated Abl.

Recombinant human Abl was purchased from Upstate.

Activity was measured in a filter binding assay using an Abl specific peptide substrate (Abtide, Upstate). Reaction conditions were: 10 μM [γ-32P]-ATP, 50 μM peptide, 0.022 μM c-Abl. The apparent affinity (Km) values of the Abl preparation used for its peptide and ATP substrates were determined separately and found to be 1.5 μM and 10 μM, respectively.

### 3.1.2—Enzymatic Assays: Discussion

All synthesized compounds, including reference compound Si192, were initially tested in a cell-free assay to evaluate their affinity towards isolated c-Src (Table 6).

TABLE 6
Enzymatic activity, cellular activity and ADME properties of tested compounds

| | Enzymatic Data | | | Cellular Data | | In vitro ADME | | |
|---|---|---|---|---|---|---|---|---|
| | (Ki, μM) | | | (IC₅₀, μM) | | H₂O | | Metabolic |
| Cpd | c-Src^{a} | Abl^{a} | Fyn^{a} | SH-SY5Y^{b} | K562^{c} | Solubility^{d} | Papp^{e} | Stab.^{f} |
| 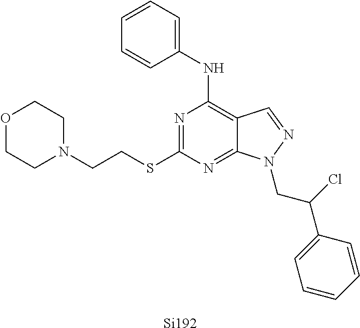 [SMILES: "Si192" ; ClC(Cn1ncc2c(Nc3ccccc3)nc(SCCN3CCOCC3)nc21)c1ccccc1] | 0.20 | 0.15 | 7.5 | | | 1.7 | 10.0 | 95 |
| 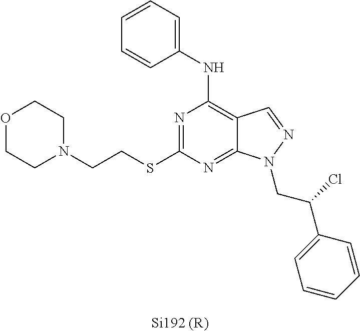 [SMILES: Cl[C@@H](Cn1ncc2c(Nc3ccccc3)nc(SCCN3CCOCC3)nc21)c1ccccc1 ; "Si192 (R)"] | 0.20 | | | | | | | |
| 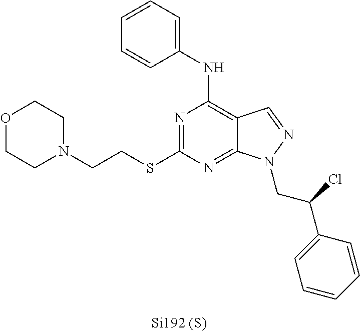 [SMILES: Cl[C@H](Cn1ncc2c(Nc3ccccc3)nc(SCCN3CCOCC3)nc21)c1ccccc1 ; "Si192 (S)"] | 0.23 | | | | | | | |
| 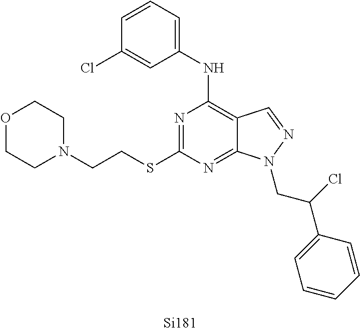 [SMILES: "Si181" ; Clc1cccc(Nc2nc(SCCN3CCOCC3)nc3c2cnn3CC(Cl)c2ccccc2)c1] | 0.19 | 0.12 | | | | 0.1 | 9.5 | 95 |
| 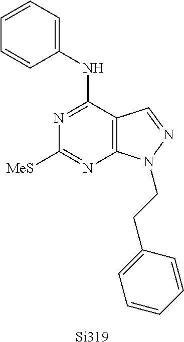 [SMILES: CSc1nc(Nc2ccccc2)c2cnn(CCc3ccccc3)c2n1 ; "Si319"] | 1.46 | | | | | | 7.60 | 90 |
| 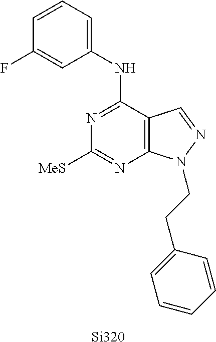 [SMILES: CSc1nc(Nc2cccc(F)c2)c2cnn(CCc3ccccc3)c2n1 ; "Si320"] | 1.06 | | | | | | 9.33 | |
| 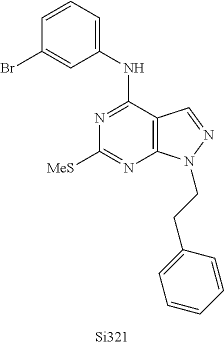 [SMILES: CSc1nc(Nc2cccc(Br)c2)c2cnn(CCc3ccccc3)c2n1 ; "Si321"] | 0.20 | | | | | | 3.26 | |
| 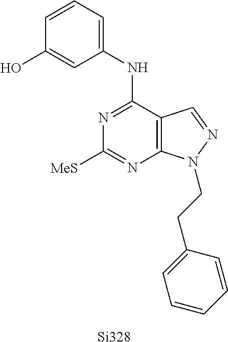 [SMILES: CSc1nc(Nc2cccc(O)c2)c2cnn(CCc3ccccc3)c2n1 ; "Si328"] | 0.62 | | | | | | 5.24 | |
| 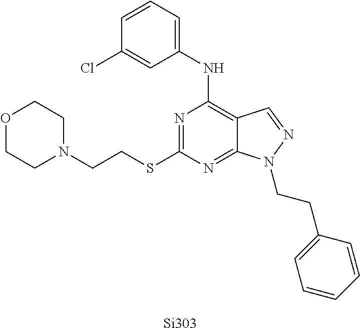 [SMILES: Clc1cccc(Nc2nc(SCCN3CCOCC3)nc3c2cnn3CCc2ccccc2)c1 ; "Si303"] | 1.20 | | | | | | 3.50 | |
| 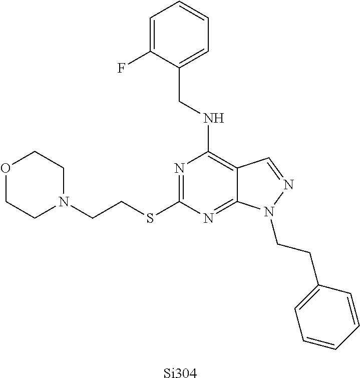 [SMILES: Fc1ccccc1CNc1nc(SCCN2CCOCC2)nc2c1cnn2CCc1ccccc1 ; "Si304"] | 0.17 | | | 63.0 | | | | |
| 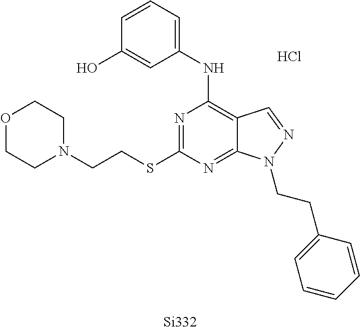 [SMILES: Oc1cccc(Nc2nc(SCCN3CCOCC3)nc3c2cnn3CCc2ccccc2)c1 ; Cl ; "Si332"] | 0.04 | 0.13 | | 0.9 | | 97 | 6.38 | 97 |
| 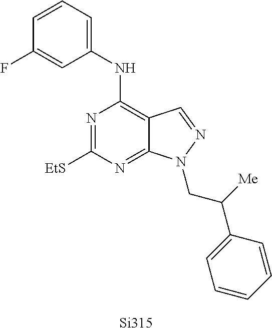 | 0.79 | | | | | | 1.72 | 94 |
| 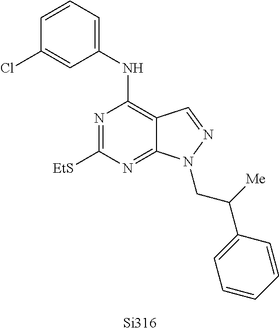 | 0.55 | | | | | | 3.81 | |
| 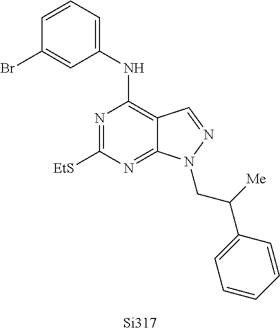 | 0.10 | | | | | | 2.03 | |
| 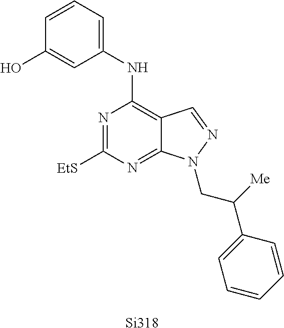 | 0.11 | | | | | | 4.91 | |
| 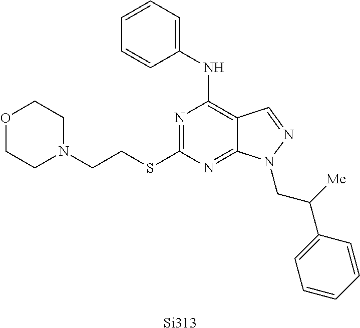 [SMILES: CC(Cn1ncc2c(Nc3ccccc3)nc(SCCN3CCOCC3)nc21)c1ccccc1 ; "Si313"] | 1.44 | | | | | | 3.66 | |
| 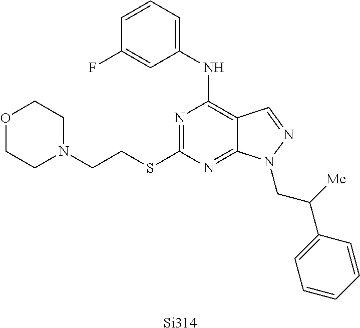 [SMILES: CC(Cn1ncc2c(Nc3cccc(F)c3)nc(SCCN3CCOCC3)nc21)c1ccccc1 ; "Si314"] | 2.34 | | | | | | 6.51 | |
| 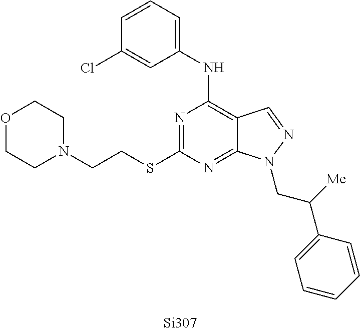 [SMILES: CC(Cn1ncc2c(Nc3cccc(Cl)c3)nc(SCCN3CCOCC3)nc21)c1ccccc1 ; "Si307"] | 0.97 | | | | | | 5.51 | |
| 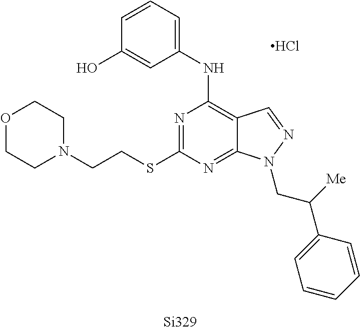 [SMILES: CC(Cn1ncc2c(Nc3cccc(O)c3)nc(SCCN3CCOCC3)nc21)c1ccccc1 ; Cl ; "Si329"] | 0.07 | 0.43 | | 0.54 | | 1 | 6.81 | 99 |
| 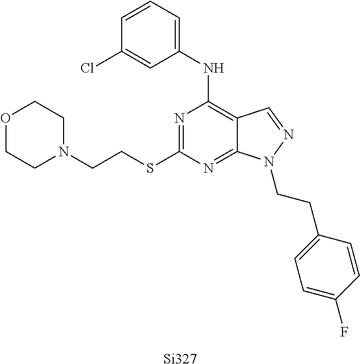 [SMILES: Fc1ccc(CCn2ncc3c(Nc4cccc(Cl)c4)nc(SCCN4CCOCC4)nc32)cc1 ; "Si327"] | 0.10 | | | | | | 2.15 | 97 |
| 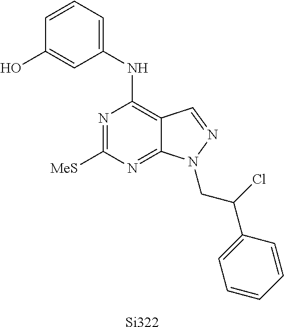 [SMILES: CSc1nc(Nc2cccc(O)c2)c2cnn(CC(Cl)c3ccccc3)c2n1 ; "Si322"] | 0.03 | 0.15 | | 0.12 | | 4.3 | 5.92 | 98 |
| 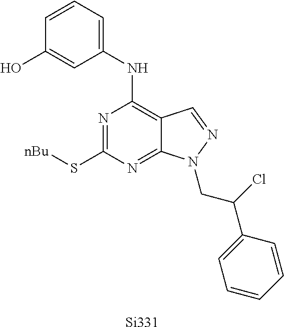 | 0.13 | | | | | | 3.43 | |
| 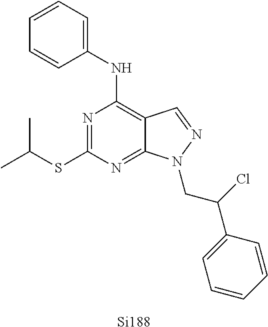 [SMILES: "Si188" ; CC(C)Sc1nc(Nc2ccccc2)c2cnn(CC(Cl)c3ccccc3)c2n1] | 0.3 | | | | | | | |
| 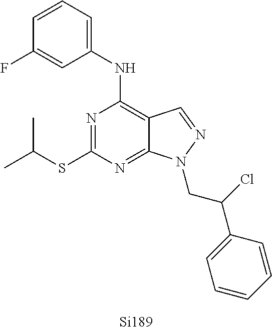 [SMILES: "Si189" ; CC(C)Sc1nc(Nc2cccc(F)c2)c2cnn(CC(Cl)c3ccccc3)c2n1] | 0.26 | | | | | | | |
| 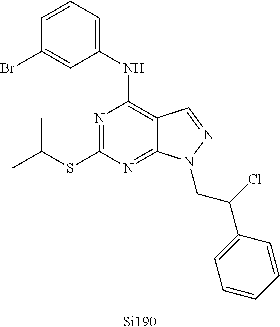 [SMILES: "Si190" ; CC(C)Sc1nc(Nc2cccc(Br)c2)c2cnn(CC(Cl)c3ccccc3)c2n1] | 0.62 | | | | | | | |
| 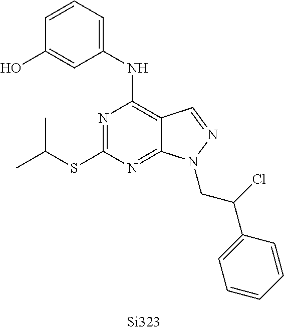 [SMILES: CC(C)Sc1nc(Nc2cccc(O)c2)c2cnn(CC(Cl)c3ccccc3)c2n1 ; "Si323"] | 0.01 | 0.12 | | 1.53 | | 0.9 | 4.53 | 95 |
| 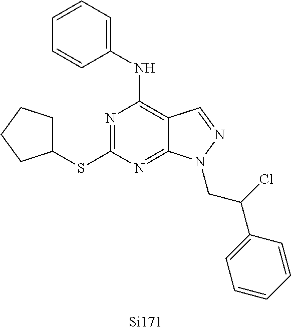 [SMILES: "Si171" ; ClC(Cn1ncc2c(Nc3ccccc3)nc(SC3CCCC3)nc21)c1ccccc1] | 0.12 | | | | | | 13 | 94 |
| 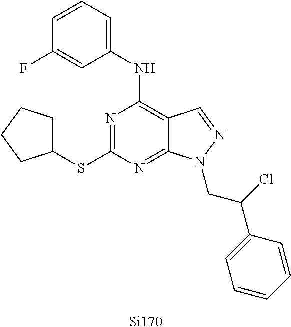 [SMILES: "Si170" ; Fc1cccc(Nc2nc(SC3CCCC3)nc3c2cnn3CC(Cl)c2ccccc2)c1] | 0.11 | NA | | | | | 14 | 95 |
| 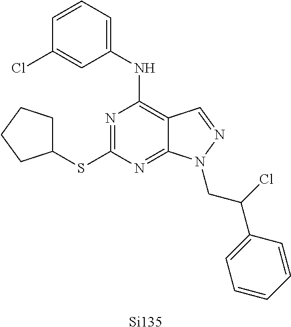 [SMILES: "Si135" ; Clc1cccc(Nc2nc(SC3CCCC3)nc3c2cnn3CC(Cl)c2ccccc2)c1] | NA | NA | NA | | | | | |
| 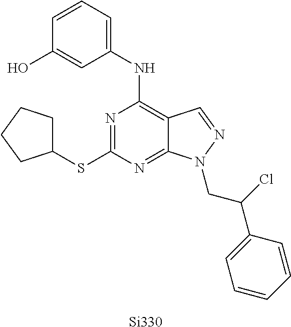 [SMILES: Oc1cccc(Nc2nc(SC3CCCC3)nc3c2cnn3CC(Cl)c2ccccc2)c1 ; "Si330"] | 0.007 | 0.15 | | 0.62 | | 0.6 | 3.91 | 91 |
| 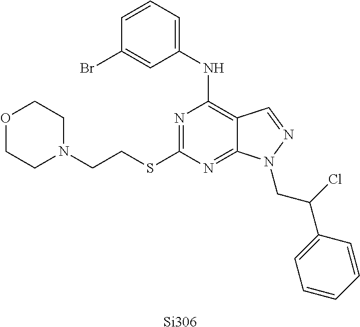 [SMILES: ClC(Cn1ncc2c(Nc3cccc(Br)c3)nc(SCCN3CCOCC3)nc21)c1ccccc1 ; "Si306"] | 0.13 | 0.12 | | 0.34 | | 3.7 | 5.27 | 96 |
| 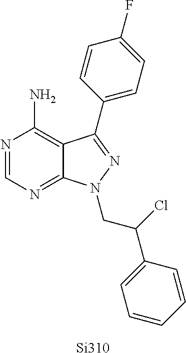 [SMILES: Nc1ncnc2c1c(-c1ccc(F)cc1)nn2CC(Cl)c1ccccc1 ; "Si310"] | | | 0.36 | | 12.63 ± 14.80 | | | |
| 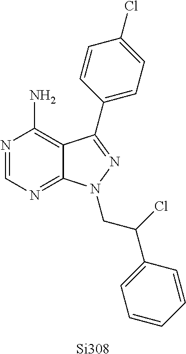 [SMILES: Nc1ncnc2c1c(-c1ccc(Cl)cc1)nn2CC(Cl)c1ccccc1 ; "Si308"] | | | 0.07 | | 0.56 ± 0.01 | | | |
| 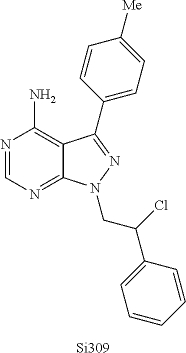 [SMILES: Cc1ccc(-c2nn(CC(Cl)c3ccccc3)c3ncnc(N)c23)cc1 ; "Si309"] | | | 0.095 | | 0.30 ± 0.06 | | | |
| 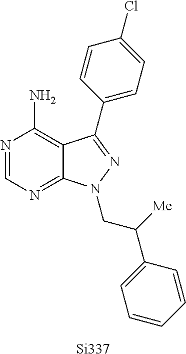 [SMILES: CC(Cn1nc(-c2ccc(Cl)cc2)c2c(N)ncnc21)c1ccccc1 ; "Si337"] | | | 0.78 | | | | | |
| 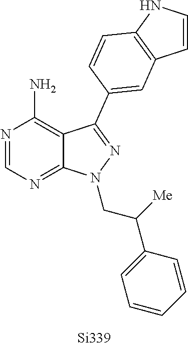 [SMILES: CC(Cn1nc(-c2ccc3[nH]ccc3c2)c2c(N)ncnc21)c1ccccc1 ; "Si339"] | | | 1.625 | | | | | |
| 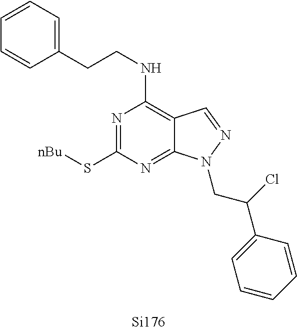 | | | NA | | | | | |
| 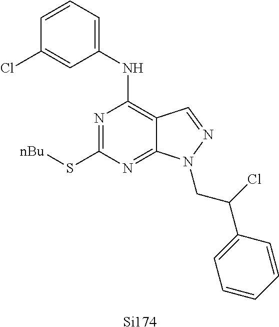 | | | 1.4 | | | | | |
| 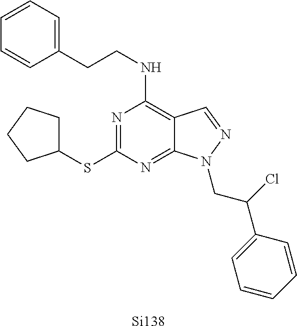 [SMILES: "Si138" ; ClC(Cn1ncc2c(NCCc3ccccc3)nc(SC3CCCC3)nc21)c1ccccc1] | | | NA | | | | | |
| 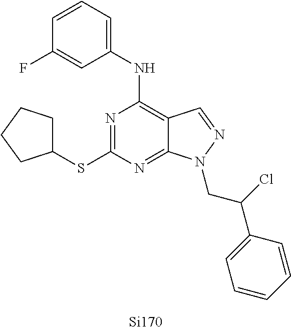 [SMILES: "Si170" ; Fc1cccc(Nc2nc(SC3CCCC3)nc3c2cnn3CC(Cl)c2ccccc2)c1] | | | NA | | | | | |
| 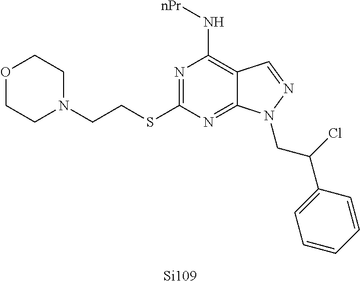 | | | 8.15 | | | | | |
| 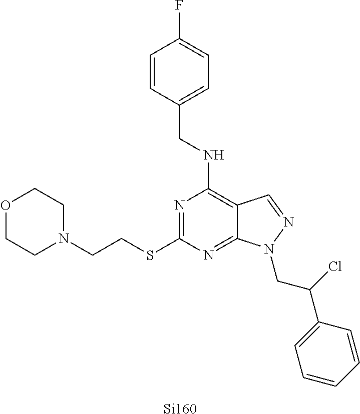 [SMILES: "Si160" ; Fc1ccc(CNc2nc(SCCN3CCOCC3)nc3c2cnn3CC(Cl)c2ccccc2)cc1] | | | 15.5 | | | | | |
| 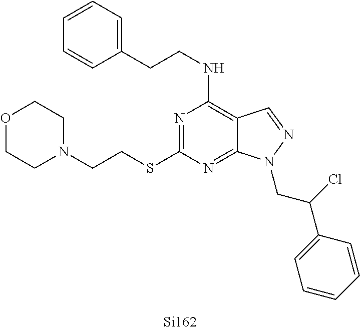 [SMILES: "Si162" ; ClC(Cn1ncc2c(NCCc3ccccc3)nc(SCCN3CCOCC3)nc21)c1ccccc1] | | | NA | | | | | |
| 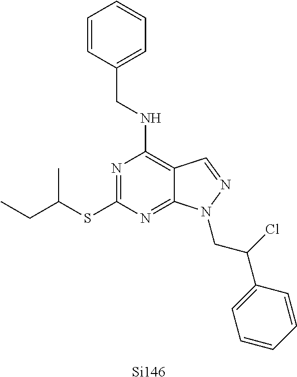 [SMILES: "Si146" ; CCC(C)Sc1nc(NCc2ccccc2)c2cnn(CC(Cl)c3ccccc3)c2n1] | | | NA | | | | | |
| 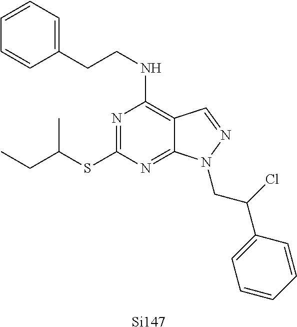 [SMILES: "Si147" ; CCC(C)Sc1nc(NCCc2ccccc2)c2cnn(CC(Cl)c3ccccc3)c2n1] | | | NA | | | | | |
| 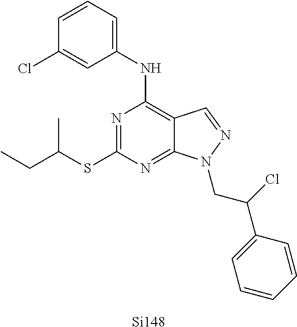 [SMILES: "Si148" ; CCC(C)Sc1nc(Nc2cccc(Cl)c2)c2cnn(CC(Cl)c3ccccc3)c2n1] | | | 12.5 | | | | | |
| 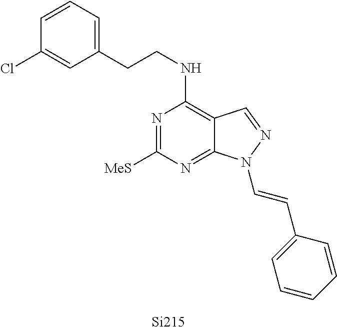 [SMILES: "Si215" ; CSc1nc(NCCc2cccc(Cl)c2)c2cnn(/C=C/c3ccccc3)c2n1] | | | 16 | | | | | |
| 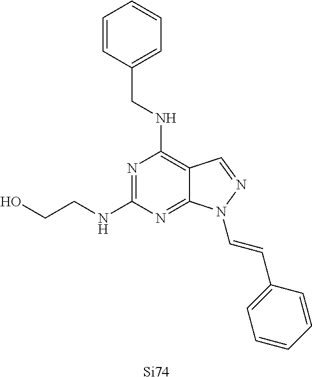 [SMILES: "Si74" ; OCCNc1nc(NCc2ccccc2)c2cnn(/C=C/c3ccccc3)c2n1] | | | 1.15 | | | | | |
| 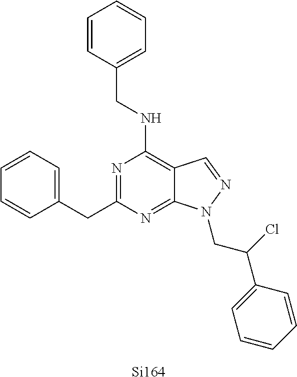 [SMILES: "Si164" ; ClC(Cn1ncc2c(NCc3ccccc3)nc(Cc3ccccc3)nc21)c1ccccc1] | | | 13 | | | | | |
| 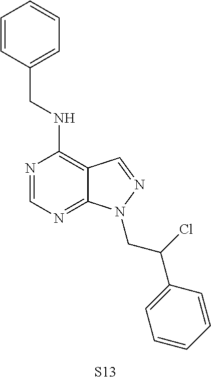 [SMILES: ClC(Cn1ncc2c(NCc3ccccc3)ncnc21)c1ccccc1 ; "S13"] | | | 3.5 | | | | | |
| 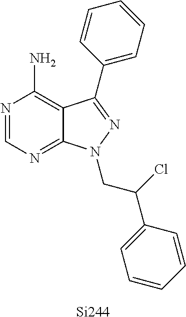 [SMILES: "Si244" ; Nc1ncnc2c1c(-c1ccccc1)nn2CC(Cl)c1ccccc1] | | | 0.9 | | | | | |
| 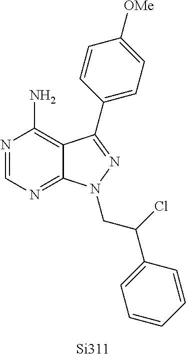 [SMILES: COc1ccc(-c2nn(CC(Cl)c3ccccc3)c3ncnc(N)c23)cc1 ; "Si311"] | | | 1.485 | | | | | |
| 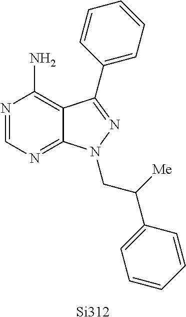 [SMILES: CC(Cn1nc(-c2ccccc2)c2c(N)ncnc21)c1ccccc1 ; "Si312"] | | | 3.35 | | | | | |
| 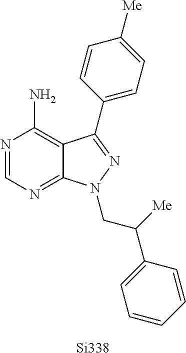 [SMILES: Cc1ccc(-c2nn(CC(C)c3ccccc3)c3ncnc(N)c23)cc1 ; "Si338"] | | | 0.995 | | | | | |
| 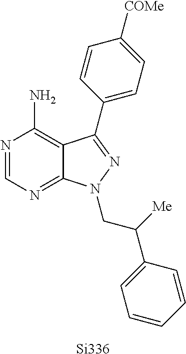 | | | >10 | | | | | |
| 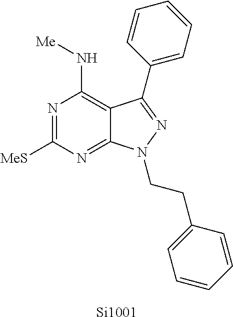 [SMILES: CNc1nc(SC)nc2c1c(-c1ccccc1)nn2CCc1ccccc1 ; "Si1001"] | | | >10 | | | | | |
| 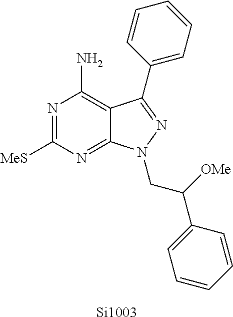 [SMILES: COC(Cn1nc(-c2ccccc2)c2c(N)nc(SC)nc21)c1ccccc1 ; "Si1003"] | | | >10 | | | | | |
| 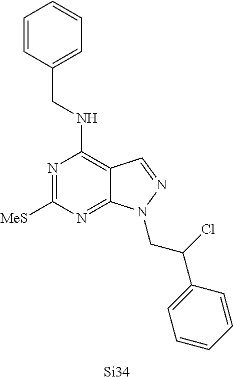 | | | | | | | | |
| 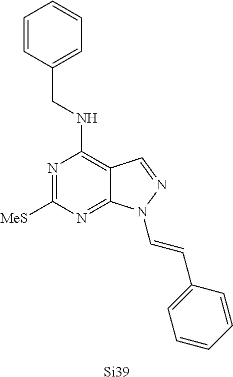 [SMILES: "Si39" ; CSc1nc(NCc2ccccc2)c2cnn(/C=C/c3ccccc3)c2n1] | | | | | | | | |

^{a}Kᵢ (μM) values are the mean of at least two experiments,
^{b}SH-SY5Y Neuroblastoma cell spheroid IC₅₀ (μM),
^{c}K562 Leukemia cell line IC₅₀ (μM) ± SD (Standard Deviation).
^{d}Expressed as μg/mL;
^{e}PAMPA, Papp expressed as 10⁻⁶ cm/sec;
^{f}Expressed as percentage (%) of unmodified parent drug.
Empty cell means not determined.
NA = Not Active (Kᵢ > 100 μM)

Compounds Si181 and Si135 already published by the inventors^{18,24} have been also inserted in Table 6 for comparison purpose. As it can be appreciated from Table 6, the rationally designed derivatives Si332, Si329, Si322, Si323 and Si330 showed potent in vitro inhibitory effect against c-Src with Kᵢ values in the low nanomolar range (40 nM, 70 nM, 30 nM, 10 nM and 7 nM, respectively). These potencies were most likely evoked due to the contribution of the hydroxyl group in meta position of the anilino ring, as hypothesized by the inventors' molecular modelling calculations and further confirmed by the observed structure activity relationships. With the simple addition of a m-OH substituent on the C4 anilino ring, potent agents were identified with 2 to 30-fold increased activities (compare Si328 with Si319, Si329 with Si313, Si323 with Si188 and Si330 with Si171 in Table 6). On the contrary, no clear trends were observed with the introduction of fluorine, chlorine or bromine at the same position. However, compounds with remarkable activities were identified also in these series such as Si317, Si327, Si170 and Si306, evoking Kᵢ values of around 100 nM. The most active c-Src inhibitors Si332, Si329, Si322, Si323, and Si330 were also tested against Abl. These compounds maintained the dual Src/Abl inhibitory profile of lead structures Si192 and Si181, but showed Kᵢ values of one order of magnitude higher than for Src, possessing a significant selectivity for c-Src over Abl. Derivative Si192 exhibited moderate activity with Kᵢ of 7.5 μM. As it can be appreciated from Table 6, derivatives Si308 and Si309 showed potent in vitro inhibitory effect against Fyn, with Kᵢ values in the nanomolar range (70 nM and 95 nM, respectively). These potencies were most likely evoked due to the contribution of a chlorine or methyl substituent in para position of the C3 phenyl ring (compare Si308 with Si244 and Si309 with Si244). Interesting activities were also found for compounds Si310, Si337 and Si338 that exhibited submicromolar affinities (0.36 μM, 0.78 μM and 0.995 μM, respectively).

Furthermore, Si174, Si74, Si3 and Si244 resulted to be endowed with Kᵢ values of 1.4 μM, 1.15 μM, 3.5 μM and 0.9 μM, respectively. Derivatives Si109, Si180, Si192, Si215, Si148, Si164 exhibited moderate activity with Kᵢ ranging from 7.5 μM to 16 μM. No activity was detected for the remaining compounds.

As a general trend, the substitution of chlorine by methyl in the N1 side chain led to a considerable reduction of the affinity with about 10-fold decreased activities (compare Si312 vs Si244, Si337 vs 51308 and Si338 vs Si309).

### 3.2—Neuroblastoma:

### 3.2.1—Neuroblastoma: Materials and Methods Antiproliferative Activity on Neuroblastoma Human Cell Line SH-SY5Y.

In vitro experiments were carried out using the human neuroblastoma cell line SH-SY5Y. Cells were purchased from American Type Culture Collection (ATCC, Manassas, Va., USA) and were cultured in DMEM medium supplemented with 100/Foetal Bovine Serum. In order to determine antiproliferative effect of drugs SH-SY5Y cells were seeded at 2×105 cells/ml density and treated with compounds at increasing concentrations from 0.01 to 50 μM. The cultures were maintained at 37° C. in 5% v/v CO2 for 72 h. Cell number and vitality were evaluated using the automatic cell counter NucleoCounter® (Chemometec, Denmark). Results from the NucleoCounter represented either total or non-viable cell concentration, depending on the sample preparation indicated by manufacturer. IC50 (drug concentration that determined the 50% of growth inhibition) was calculated by Grafit v4.0 (Erithacus Software Limited) software using the best fitting sigmoid curve.

### Spheroid Growth Assay.

The in vitro antitumoral action of inhibitors was evaluated by neuroblastoma spheroid assay. The SH-SY5Y cell line was utilized as cell model of human neuroblastoma. Cells were purchased from American Type Culture Collection (ATCC, Manassas, Va., USA) and were cultured in DMEM medium supplemented with 10% Fetal Bovine Serum. IBIDI angiogenesis micro-slides (IBIDI GmbH) coated with growth factor reduced Matrigel (BD, Bioscience) and allowed to polymerize for 30 minutes. SH-SY5Y cells were seeded in a 96-multiwell plate in the presence or not (CTR) of inhibitors. Starting from 24 h after the seeding, in basal condition, cellular aggregates with spheroidal appearance (diameter >100 μm) were visible. The size of cellular spheres, in terms of area and diameter, was determined using an Image Pro-plus v 4.5 analysis system considering 5 random fields/treatment (400× magnification). IC₅₀ (drug concentration that determined the 50% of growth inhibition) was calculated by Grafit v4.0 (Erithacus Software Limited) software using the best fitting sigmoid curve.

### Cell Cycle Analysis.

Cells (SH-SY5Y) were seeded in 60-mm petri dishes at a density of 3×10⁵. After treatment and subsequent incubation for 24 h at 37° C. and 5% CO₂ in humidified atmosphere, harvested cells were washed and fixed overnight with 70% ethanol. Then, ethanol was removed by centrifugation and the cells resuspended in PBS, stained with 50 μg/mL propidium iodide (PI) at 4° C. for 30 min in the dark. Stained cells were analyzed by Tali image based cytometer (Life Technologies, Carlsbad, Calif., USA) counting 20 fields for sample and exported fcs raw data were elaborated by Flowing software (v. 2.5.0, by Perttu Terho, University of Turku, Finland).

Animals and Experimental in vivo Model.

Male CD1 nude mice (Charles River, Milan, Italy) were maintained under the guidelines established by the inventors' Institution (University of L'Aquila, Medical School and Science and Technology School Board Regulations, complying with the Italian government regulation n. 116 Jan. 27 1992 for the use of laboratory animals). Before any invasive manipulation, mice were anesthetized with a mixture of ketamine (25 mg/mL)/xylazine (5 mg/mL). Tumor grafts were obtained by injecting s.c. 1×10⁶ SH-SY5Y cells in 100 μL of 12 mg/mL Matrigel (Becton Dickinson, Franklin Lakes, N.J., USA). Tumor growth was monitored daily by measuring the average tumor diameter. The tumor volume was expressed in mm³ according to the formula 4/3πr³. For in vivo administration Si306 was prepared as suspension in 0.5% methylcellulose solution. Each mouse received daily oral administration of methylcellulose vehicle, or of 50 mg/kg Si306.

### Sprouting Assay.

The brain microvascular endothelial cell line hBMEC was purchased from ScienCell Research Laboratory (Carlsbad, Calif., USA). HBMEC cells were suspended in culture medium containing 20% methylcellulose, seeded at a density of 1000 cells/well, into nonadhesive 96 well plate and cultured at 37° C. (5% CO₂, 100% humidity). Under these conditions, suspended endothelial cells (EC) form spontaneously within 4 h a single cell aggregate known as spheroid. Spheroids were harvested within 24 h and used for in gel sprouting angiogenesis experiments. Briefly, spheroids were seeded in micro-slides coated with Matrigel and images were observed after 24 h, captured by an inverted microscope and analysed with the NIH Image J analysis system. For statistical analysis, number of sprouts per spheroid, with a minimum of 10 spheroids for each treatment, was considered.

### 3.2.2—Neuroblastoma: Discussion

### In Vitro Biological Activity.

Selected c-Src inhibitors were evaluated for their ability to inhibit the proliferation of neuroblastoma SH-SY5Y cells (FIG. 6). Cells were treated for 72 h with increasing concentrations of the inhibitors (0.1-50 μM) and IC₅₀ values were calculated considering the mean area of spheroids respect to control. The strongest effect on SH-SY5Y was obtained by Si322 and Si306 that showed I_{C50} values of 0.12 and 0.34 μM, respectively. Antitumoral effect was also tested by spheroid formation assay. The growth rate of spheroids in presence of Si306 was significantly counteracted (FIG. 7). Biological effect of Si306 was also evaluated through analysis of cell cycle (FIG. 8). SH-SY5Y cells were treated with increasing concentration of Si306 (0.1-10 μM) and the percentage of cells in each phase of cell cycle was evaluated by fluorimetric analysis of DNA content. Si306 determined a significant and dose-dependent accumulation of cells in the G1 phase of cell cycle starting from 0.1 μM. In parallel, the inventors observed a progressive accumulation of hypodiploid cells indicating the presence of apoptotic cells. The treatment with 10 μM Si306 induced the apoptosis in about 50% of treated cells.

### In Vivo Studies.

Among the most promising c-Src inhibitors, compound Si306 was selected for the in vivo studies because it showed an appropriate balance of different ADME properties, remarkable activity in the cell-free assay, and promising submicromolar potency against SH-SY5Y neuroblastoma cells. The anticancer activity of Si306 was tested in vivo using a xenograft mouse model. Mice inoculated with SH-SY5Y neuroblastoma cells were treated daily with 50 mg/kg Si306 starting from the appearance of a visible tumor mass, and the tumor volume was evaluated at regular intervals. Si306 caused a significant reduction in tumor volume after 60 days of oral treatment with a reduction of more than 50% in mean tumor volume compared to placebo treated mice. In vivo Dasatinib treatment (50 mg/kg) determined a very similar inhibitory trend, but the appearance of palpable tumor masses was earlier in Dasatinib group respect to mice treated with Si306 (FIG. 9A). Remarkably, mice did not shown signs of distress or weight loss during the experiment. It is notable that the tumor associated angiogenesis at the endpoint was significantly compromised in mice treated with Si306 (data not shown). Thus, a three-dimensional in vitro sprouting assay was performed to analyze the effect of Si306 on angiogenic response of endothelial cells. Spheroids from endothelial HBMECs were seeded on Matrigel. 24 h after the beginning of the experiment, the inventors observed a significant reduction of angiogenesis as demonstrated by the reduction of the number of sprouts derived from spheroid treated with the compound, at 0.5 μM and 1 μM concentrations, compared with untreated control cells (FIG. 9B).

### 3.3—Glioblastoma:

### 3.3.1—Glioblastoma: Materials and Methods

### Proliferation Assay on U87 and U251 Cells.

U87 and U251 cells were purchased from European Collection of Cell Cultures (ECACC, Salisbury, UK) and were cultured in DMEM medium supplemented with 10% Foetal Bovine Serum. In order to determine antiproliferative effect of drugs tumor cells were seeded at 2×105 cells/ml density and treated with compounds at indicated concentrations. The cultures were maintained at 37° C. in 5% v/v CO₂ for 72 h. Cell number and viability were evaluated by Trypan blue exclusion test. Viable cells were expressed as percentage respect to cells treated with vehicle (=100%). Mean and SD values of at least three different experiments are shown.

### U87 Xenograft.

Male CD1 nude mice (Charles River, Milan, Italy) were maintained under the guidelines established by University of L'Aquila, Medical School and Science and Technology School Board Regulations, complying with the Italian government regulations for the use of laboratory animals. Before any invasive manipulation, mice were anesthetized with a mixture of ketamine (25 mg/ml)/xylazine (5 mg/ml). Tumor grafts were obtained by injecting s.c. 1×106 U87 cells in 100 μL of 12 mg/mL Matrigel (Becton Dickinson, Franklin Lakes, N.J., USA). Mice were divided in four groups: treated with vehicle, treated with radiotherapy, treated with Si306 and treated with Si306 in combination with radiotherapy. For in vivo administration, Si306 was prepared as suspension in 0.5% methylcellulose solution. Each mouse received daily oral administration of methylcellulose vehicle, or of 40 mg/kg Si306. At the endpoint, tumors were recovered and weighted. The tumor xenograft was irradiated once with 4Gy dose at first sign of palpable tumor mass.

Low density growth assay.

U87 cells capacity for growth at clonal density, was evaluated by plating cells at density of 10 cells/cm² in 10% fetal bovine supplemented DMEM. After 2 weeks of culture, adherent cells were fixed with cold methanol, washed with PBS/BSA and air-dried. Adherent cells were stained with 0.5% crystal violet for 15 minutes at room temperature. The stained colonies were photomicrographed and analyzed by number and size with the public domain software ImageJ (by Wayne Rasband, http://rsb.info.nih.gov/ij/). Mean and SD values of at least three different experiments are shown. Cell proliferation was tested also with Si306 in combination with mitomycin (20 μM, 2 μM, 0.2 μM and 0.02 μM) or with radiation (4 Gy, the day after the cell plating).

### Immunohistochemistry (Ihc) Analysis.

Slide-mounted tissue sections (4-μm thick) were deparaffinised in xylene, hydrated serially in 100%, 95%, and 80% ethanol, were treated whit 3% H2O2 and then were incubated with an anti-alpha Smooth Muscle Actin (alpha-SMA) antibody for 1 h at RT. Sections were washed three times in PBS and antibody binding was revealed using the Sigma fast 3,30-diaminobenzidine tablet set (Sigma, St. Louis, Mo.) according to the manufacturer's instructions. Antibodies were purchased from Cell Signaling (Cell Signaling Technology, Inc.).

Western Blot Analysis.

Total cell lysates were obtained by incubating cells in a lysis buffer containing 1% triton, 0.1% SDS, 2 mM CaCl2, 10 mg/ml orthovanadate, and 1× protease inhibitors cocktail (Sigma, St. Louis, Mich., USA). Protein content was determined using the Protein Assay Kit 2 (Bio-Rad Laboratories, Hercules, Calif., USA). Sixty micrograms of proteins were electrophoresed in 10% SDS-polyacrylamide gel. After electrophoresis gels were placed onto Trans-Blot Turbo mini nitrocellulose transfer pack and transferred using Trans-Bolt Turbo Transfer System (Bio-Rad Laboratories, Hercules, Calif., USA). The membrane was incubated with 1 μg/ml primary antibody and then with appropriate horseradish peroxidase-conjugated secondary antibodies. Primary antibodies β-actin, PDGFR-beta, alpha-SMA were purchased from Cell Signaling Technology; Protein bands were visualized using a chemiluminescent detection system (Thermo Scientific, Rockford, Ill., USA) and signals were digitally acquired by Chemidoc XRS system (Bio-Rad Laboratories).

### Orthotopic Mouse Model.

Male CD1 nude mice (Charles River, Milan, Italy) were maintained under the guidelines established by University of L'Aquila, Medical School and Science and Technology School Board Regulations, complying with the Italian government regulations for the use of laboratory animals. Before any invasive manipulation, mice were anesthetized with a mixture of ketamine (25 mg/ml)/xylazine (5 mg/ml). Tumor grafts were obtained by injecting with a Hamilton syringe mounted on a stereotactic instrument, 2*10³ U87 cells resuspended in 2 microL PBS (David Kopf Instruments, CA, USA). The cells were injected after the exposition of periosteal cranic site and the drimming of a 1 mm diameter hole localized at 4 mm from striatum and with a depth of 4 mm. The wound was treated with antibiotics and was surgically sutured.

### 3.3.2—Glioblastoma: Discussion

The antiproliferative effect of Si306 was tested in vitro in U251 and U87 cell lines (FIGS. 10 and 11). The U251 malignant glioma cell line was originally established from a 75-year-old male with GBM by Ponten and others.^{29,30} Ponten and colleagues, from a female with GBM, originally established the U87 GBM model.³⁰ These GBM cell lines are known to mimic the salient features of human GBM and as such has received significant attention over the last four decades in xenogeneic mouse models of cancer.^{29, 31} U251 and U87 cell lines differ in important molecular aspects. U87 is intrinsically more radioresistant than U251, which is partly attributable to more cycling U251 cells found in G2/M, the most radiosensitive cell stage, while more U87 cells are found in S and G1, the more radioresistant cell stages.³² U251 contains mutant p53 and U87 contains WT p53.³³

A concentration of 5 μM of Si306 induced a reduction of about 50% of the total cell number when compared to control after 72 h of treatment (FIGS. 10 and 11). In U87 cells (FIG. 11), the effect of Si306 was more pronounced than in U251 cells (FIG. 10), as more than 80% of dead cells are observed in presence of 30 μM of the compound.

Si306 was tested also in combination with mitomycin C, a well-known genotoxic agent, in U87 (FIG. 12) and U251 (FIG. 13) glioblastoma cell lines. Cells were treated with increasing mitomycin C concentration in presence of 1 μM Si306 for 72 h. The combination treatment determined a synergic antiproliferative effect that was more pronounced in U87 cells. Si306 was administered in vivo to nude mice inoculated subcutaneously with U87 cells. Mice received 50 mg/kg of Si306 every other day and the antitumoral effect of the compound was evaluated also in combination with a single radiotherapic treatment (4Gy). At the end point mice that have received the combination therapy showed the smallest tumors respect to other experimental groups (<80% respect untreated group) (FIG. 14)

The combination therapy of Si306 plus radiotherapy was evaluated also in vitro by a low density growth assay. U87 cells were seeded at low density (<100 cells/cm²) and received one irradiation (4Gy) plus 1 μM or 10 μM Si306 every other day. After 15 days, the number of colonies with more than 10 cells was counted. The combination therapy reduced significantly the number of colonies in respect to control and to single treatments (FIG. 15).

By analyzing tumor masses from in vivo experiments inventors observed a significant difference in histology pattern. Si306 treatment determined the reduction of myofibroblast content as evaluated by the expression of the differentiation marker alpha-SMA (FIG. 16).

The ability of Si306 to interfere with myofibroblast differentiation was tested in vitro on human fibroblast wi38 treated with TGF-beta. Si306 was able to block the expression of PDGFR and alpha-SMA downstream of TGF-beta signaling (FIG. 17). Then inventors evaluated the antitumoral activity of Si306 and pro-drug pro-Si306 in a orthotopic in vivo model of glioblastoma. Both Si306 and pro-Si306 demonstrated a significant ability in prolonging survival of mice respect to control group, and this ability was comparable with radiotherapic treatment (FIG. 18).

### 3.4—Leukemia:

### 3.4.1—Leukemia: Materials and Methods

### Antiproliferative Activity on Human Cell Line K562.

In vitro experiments were carried out using the human Chronic Myelogeneous Leukemia cell line K562. Cells were purchased from American Type Culture Collection (ATCC, Manassas, Va., USA) and were cultured in RPMI medium supplemented with 10% Foetal Bovine Serum. In order to determine antiproliferative effect of Fyn inhibitors K562 cells were seeded at 2×105 cells/ml density and treated with compounds at increasing concentrations from 0.01 to 50 μM. The cultures were maintained at 37° C. in 5% v/v Co₂ for 72 h. Cell number and vitality were evaluated using the automatic cell counter NucleoCounteri (Chemometec, Denmark). Results from the NucleoCounter represented either total or non-viable cell concentration, depending on the sample preparation indicated by manufacturer. I_{C50} (drug concentration that determined the 50% of growth inhibition) was calculated by Grafit v4.0 (Erithacus Software Limited) software using the best fitting sigmoid curve.

### 3.4.2—Leukemia: Discussion

Several members of the Src kinase family are upregulated and/or iperactivated in CML, and their activity regulates proliferation and differentiation of cancer cells. In K562 cells, Fyn kinase expression is under the direct control of BCR-ABL1 oncogene and its upregulation is fundamental in sustaining K562 proliferation. Tested compounds showed an effective antiproliferative activity that well correlates with the Ki values determined by in vitro inhibition assays. The most effective compounds, Si308 and Si309, showed promising IC₅₀ values for cell viability in the submicromolar range (FIG. 19). It is important to note that those compounds, when tested in human normal fibroblasts, did not showed any sign of cell toxicity.

### EXAMPLE 4

### Compounds Effect on Neurodegeneration

### 4.1—Neurodegeneration: Materials and Methods

### Cell Culture, Differentiation and Treatments on SH-SY5Y Cells.

The neuroblastoma cell line SH-SY5Y was cultured in media obtained by mixing equal volume of MEM and HAM F12 supplemented with 15% fetal calf serum (FCS, Australian origin, Lonza), 100 U/ml penicillin, 100 μg/ml streptomycin and 2 mM L-glutamine (all from Euroclone) at 37° C. in humidified air with 5% CO₂. The medium was changed every 48 hours. Cells were split at about 80% confluence and never cultured beyond passage 20. Cell differentiation was achieved by pre-treating for 5 days the SH-SY5Y cells with media containing 1% FCS and 10 μM retinoic acid (RA, Sigma Aldrich). Subsequently cells were treated for other 7 days in media with no serum and supplemented with 50 ng/ml of human recombinant brain derived neurotrophic factor (BDNF, Peprotech), 10 ng/ml human recombinant beta nerve growth factor (NGF, Peprotech), 10 ng/ml neuregulin 1 beta 2 protein (NRG, Abcam) and 9.35 μg/ml vitamin D3 (Sigma Aldrich). To confirm full neuronal differentiation, the expression of mature isoforms of Tau were checked. Differentiated cells were treated for 1.5, 3 and 6 hours with media containing 10 μM Aβ₄₂ oligomer/protofibrils and N2 supplement (Life Technologies), in the presence or in the absence of Fyn inhibitors dissolved in DMSO. As control, cells were treated with media containing equivalent amounts of DMSO.

### Aβ₄₂ Preparation.

Aβ₄₂ oligomer/protofibril samples were prepared using previously described protocols (Wong J et al., Neuroscience 210, 2012, 363-374). Briefly, Aβ₄₂ peptides (Sigma) were dissolved in 1,1,1,3,3,3-hexafluoro-2-propanol and lyophilized to completely remove the solvent. Lyophilized Aβ₄₂ peptides were reconstituted in DMSO to a working concentration of 10 mM, diluted 1:100 using HAM F12 (without phenol red and glutammine), vortexed for 15 sec and incubated 24 hours at 4° C. A042 oligomer/protofibrils were visualized by SDS-PAGE and silver staining.

### 4.2—Neurodegeneration: Discussion

In AD Fyn mediates the phosphorylation of Tau on the Tyr18 residue, an early and crucial step in the disease progression, and is therefore considered a promising therapeutic target. For this reason, the most interesting compounds identified during in vitro inhibition assays, Si308 and Si309, were evaluated for their ability to inhibit Fyn mediated phosphorylation of residue Tyr18 in Tau protein in an AD model cell line. To this aim, neuroblastoma SH-SY5Y cells were differentiated to mature neurons with the administration of retinoic acid, followed by brain derived neurotrophic factor, neuregulin 01, nerve growth factor, and vitamin D3 treatment. Once differentiated, SH-SY5Y cells were treated with amyloid beta 1-42 (A342) oligomer/protofibril in order to induce AD-like neurotoxicity. Both compounds significantly affected Aβ42 induced Tyr18-Tau phosphorylation with a similar degree and in a dose dependent manner (FIG. 20). Moreover, the inhibitory activity of Si308 and Si309 resulted constant over time, being effective up to six hours after compound administration (FIG. 20, compare panels A, B with panels C, D).

### EXAMPLE 5

### Prodrugs of Compounds of the Invention

Unless otherwise specified, materials and methods are the same as the ones previously reported for example 1, 2 and 3.

### 5.1—Chemistry: Materials and Methods

Compounds Si3, Si35, Si183, Si214, Si221, Si223, Si278 and Si306 were already reported by us.^{18,20,21,22,23}
General procedure for the synthesis of pyrazolo[3,4-d]pyrimidine prodrugs (proS13, proS13(A), proSi221, proSi214, proSi306, proSi35, proSi1223, proSi83, proSi120, proSi278(A), proSi278(B), proSi278(C), proSi278(D))

NaHCO₃ (2.25 mmol, 5.00 eq.) was added to a solution of the appropriate pyrazolo[3,4-d]pyrimidine compound (0.45 mmol, 1.00 eq.) in DCM dry (8 mL). After 5 min of stirring at r.t., the suspension was cooled with an ice-bath, then a solution of triphosgene (0.45 mmol, 1.00 eq.) in DCM dry (8 mL) was added. After 30 min the ice-bath was removed and the reaction mixture was allowed to warm to r.t. and stirred until the spot of the pyrazolo[3,4-d]pyrimidine compound disappeared from TLC (2 h, approximately). A solution of 2-(4-Methylpiperazin-1-yl)ethanol (or the appropriate alcohol) (0.90 mmol, 2.00 eq.) in DCM dry (8 mL) was added and the resulting mixture was stirred at r.t. for 16 h. The solvent was evaporated under reduced pressure and the resulting residue was purified by flash chromatography using a mixture of DCM and MeOH as eluent.

### 2-(4-Methylpiperazin-1-yl)ethyl benzyl(1-(2-chloro-2-phenylethyl)-1H-pyrazolo[3,4-d]pyrimidin-4-yl)carbamate (proSi13)

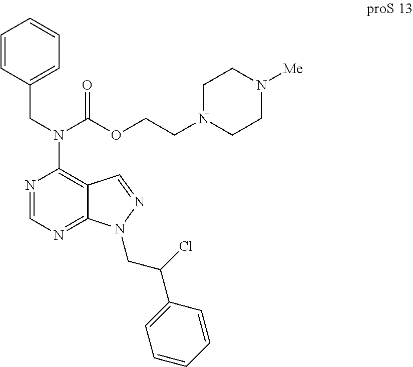 [SMILES: CN1CCN(CCOC(=O)N(Cc2ccccc2)c2ncnc3c2cnn3CC(Cl)c2ccccc2)CC1]

Fluffy white solid. Yield: 79%. ¹H-NMR (CDCl₃) δ (ppm): 8.65 (s, 1H), 8.26 (s, 1H), 7.39 (d, J=7.6 Hz, 2H), 7.24 (m, 8H), 5.52 (dd, J=6, 8.4 Hz, 1H), 5.35 (s, 2H), 5.02 (dd, J=8.8, 14.4 Hz, 1H), 4.79 (dd, J=6, 14 Hz, 1H), 4.33 (t, J=5.6 Hz, 2H), 2.55 (t, J=5.6 Hz, 2H), 2.40 (m, 8H), 2.24 (s, 3H). 3C-NMR (CDCl₃) δ (ppm): 154.7, 154.1, 154.0, 137.7, 135.7, 128.7, 128.5, 128.2, 128.1, 127.1, 127.0, 106.3, 63.9, 60.1, 56.3, 54.8, 53.7, 52.9, 49.9, 45.8. MS (ES) m/z: 535 [M+1]⁺.

### 2-(4-Methylpiperazin-1-yl)ethyl (1-(2-(4-bromophenyl)-2-chloroethyl)-1H-pyrazolo[3,4-d]pyrimidin-4-yl)(2-chlorobenzyl)carbamate (proSi221)

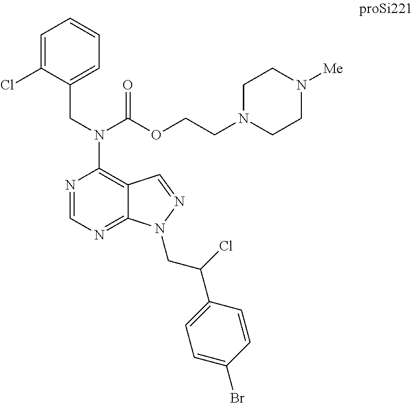 [SMILES: CN1CCN(CCOC(=O)N(Cc2ccccc2Cl)c2ncnc3c2cnn3CC(Cl)c2ccc(Br)cc2)CC1]

Fluffy white solid. Yield: 76%. ¹H-NMR (CDCl₃) δ (ppm): 8.62 (s, 1H), 8.31 (s, 1H), 7.42 (d, J=8.4 Hz, 2H), 7.18 (m, 6H), 5.50 (m, 1H), 5.44 (s, 2H), 4.99 (m, 1H), 4.82 (m, 1H), 4.34 (m, 2H), 2.54 (t, J=5.6 Hz, 2H), 2.41 (m, 8H), 2.26 (s, 3H). ¹³C-NMR (CDCl₃) δ (ppm): 154.7, 154.6, 154.2, 136.7, 135.8, 135.1, 132.3, 131.7, 129.2, 128.9, 128.0, 127.0, 126.6, 122.8, 106.1, 64.3, 59.0, 56.2, 54.7, 53.4, 52.7, 48.1, 45.6, 29.5. MS (ES) m/z: 648 [M+1]⁺, 670 [M+23]⁺.

### 2-(4-Methylpiperazin-1-yl)ethyl (3-chlorophenyl)(6-(methylthio)-1-phenethyl-1H-pyrazolo[3,4-d]pyrimidin-4-yl)carbamate (proSi214)

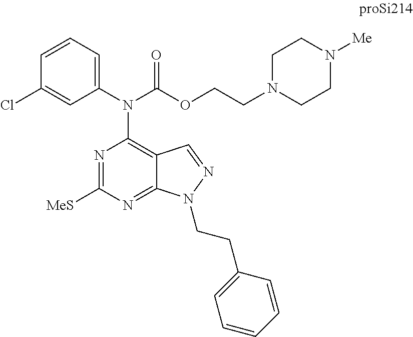 [SMILES: CSc1nc(N(C(=O)OCCN2CCN(C)CC2)c2cccc(Cl)c2)c2cnn(CCc3ccccc3)c2n1]

Transparent oil. Yield: 75%. ¹H-NMR (CDCl₃) δ (ppm): 7.93 (s, 1H), 7.35 (d, J=4.8 Hz, 2H), 7.22 (m, 7H), 4.62 (t, J=7.6 Hz, 2H), 4.39 (t, J=5.2 Hz, 2H), 3.22 (t, J=7.6 Hz, 2H), 2.60 (t, J=5.2 Hz, 2H), 2.47 (m, 8H), 2.32 (S, 3H), 2.28 (s, 3H). 3C-NMR (CDC₃) S (ppm): 168.2, 155.2, 153.7, 153.1, 140.8, 137.8, 134.3, 134.1, 129.6, 129.4, 129.1, 128.7, 128.4, 127.9, 127.0, 126.7, 126.6, 103.2, 64.5, 63.8, 56.2, 54.8, 52.7, 48.3, 48.1, 45.6, 45.5, 35.1. MS (ES) m/z: 567 [M+1]⁺.

### 2-(4-Methylpiperazin-1-yl)ethyl (3-bromophenyl) l-(2-chloro-2-phenylethyl)-6-((2-morpholinoethyl)thio)-1H-pyrazolo[3,4-d]pyrimidin-4-yl)carbamate (proSi306)

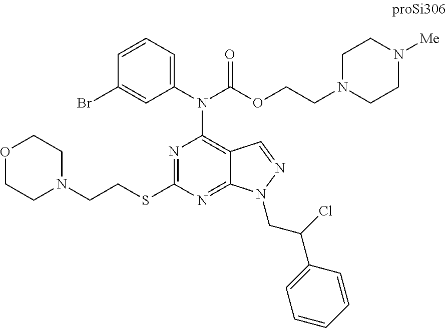 [SMILES: CN1CCN(CCOC(=O)N(c2cccc(Br)c2)c2nc(SCCN3CCOCC3)nc3c2cnn3CC(Cl)c2ccccc2)CC1]

Fluffy white solid. Yield: 51%. ¹H-NMR (CDCl₃) δ (ppm): 7.95 (s, 1H), 7.48 (d, J=8 Hz, 1H), 7.42 (m, 3H), 7.29 (m, 4H), 7.15 (d, J=8.4 Hz, 1H), 5.51 (t, J=6 Hz, 1H), 4.94 (dd, J=8.8, 14 Hz, 1H), 4.71 (dd, J=6, 14 Hz, 1H), 4.35 (t, J=5.2 Hz, 2H), 3.69 (t, J=4.4 Hz, 4H), 3.01 (m, 2H), 2.49 (m, 19H), 2.27 (s, 3H). ¹³C-NMR (CDC₃) δ (ppm): 168.1, 155.8, 153.9, 153.0, 140.8, 137.7, 135.2, 131.8, 130.9, 130.0, 128.8, 128.5, 127.4, 127.1, 121.9, 103.2, 77.1, 76.8, 76.5, 66.7, 64.5, 59.9, 57.4, 56.2, 54.8, 53.3, 53.2, 52.8, 45.6, 31.7, 30.7, 29.5, 29.1, 27.9. MS (ES) m/z: 745 [M+1]⁺, 767 [M+23]⁺.

### 2-(4-Methylpiperazin-1-yl)ethyl (3-bromophenyl)(6-(methylthio)-1-(2-phenylpropyl)-1H-pyrazolo[3,4-d]pyrimidin-4-yl)carbamate (proSi278 (A))

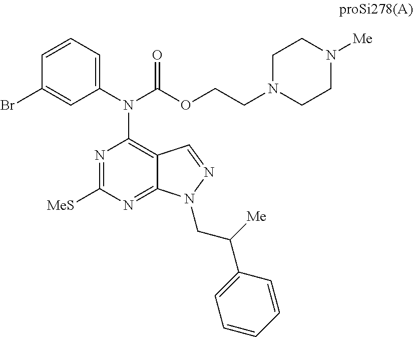 [SMILES: CSc1nc(N(C(=O)OCCN2CCN(C)CC2)c2cccc(Br)c2)c2cnn(CC(C)c3ccccc3)c2n1]

Transparent oil. Yield: 30%. 1H-NMR (CDCl₃) δ (ppm): 7.88 (s, 1H), 7.49 (d, J=8 Hz, 1H), 7.42 (s, 1H), 7.12 (m, 7H), 4.49 (t, J=7.5 Hz, 2H), 4.35 (t, J=5.2 Hz, 2H), 3.53 (m, 1H), 2.55 (m, 10H), 2.35 (s, 3H), 2.8 (s, 3H) 1.41 (s, 3H). ¹³C-NMR (CDC₃) δ (ppm): 175.5, 168.4, 155.8, 153.9, 153.3, 141.0, 134.5, 132.0, 131.1, 130.2, 128.5, 127.6, 127.2, 126.8, 122.1, 103.2, 64.6, 56.1, 54.0, 53.8, 51.8, 44.5, 39.9, 21.8, 18.8, 14.1. MS (ES) m/z: 626 [M+1]⁺, 648[M+23]⁺.

### 2-(4-Methylpiperazin-1-yl)ethyl butyl(1-(2-chloro-2-phenylethyl)-6-(ethylthio)-1H-pyrazolo[3,4-d]pyrimidin-4-yl)carbamate (proSi20)

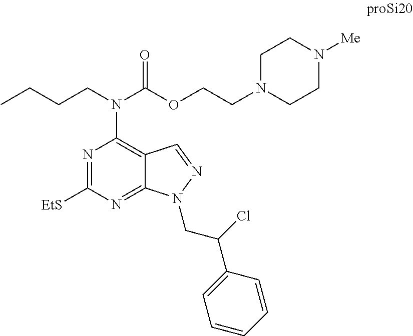

Transparent oil. Yield: 39%. ¹H-NMR (CDCl₃) δ (ppm): 8.08 (s, 1H), 7.39 (d, J=6.8 Hz, 2H), 7.27 (m, 3H), 5.50 (t, J=7.6 Hz, 1H), 4.91 (dd, J=8.4, 14.4 Hz, 1H), 4.76 (dd, J=6.4, 14 Hz, 1H), 4.37 (t, J=5.6 Hz, 2H), 4.03 (t, J=7.6 Hz, 2H), 3.17 (q, J=7.2 Hz, 2H), 2.67 (t, J=5.6 Hz, 2H), 2.54 (m, 8H), 2.31 (s, 3H), 1.66 (q, J=7.6 Hz, 2H), 1.43 (t, J=7.2 Hz, 3H), 1.31 (m, 3H), 0.93 (t, J=7.1 Hz, 3H). ¹C-NMR (CDC₃) δ (ppm): 168.2, 155.7, 154.4, 154.4, 138.1, 136.1, 128.9, 128.7, 127.4, 104.0, 63.9, 60.1, 56.6, 55.1, 53.8, 53.2, 47.3, 46.0, 30.9, 29.7, 25.4, 20.1, 14.7, 13.9. MS (ES) m/z: 561 [M+1]⁺.

### 2-(4-methylpiperazin-1-yl)ethyl (1-(2-chloro-2-phenylethyl)-6-(methylthio)-1H-pyrazolo[3,4-d]pyrimidin-4-yl)(phenethyl)carbamate (proSi35)

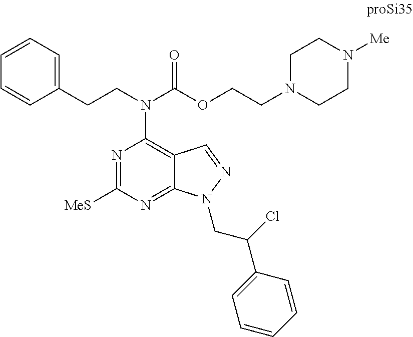 [SMILES: CSc1nc(N(CCc2ccccc2)C(=O)OCCN2CCN(C)CC2)c2cnn(CC(Cl)c3ccccc3)c2n1]

Transparent oil. Yield: 71%. ¹H-NMR (CDCl₃) δ (ppm): 8.06 (s, 1H), 7.41 (d, J=6.4 Hz, 2H), 7.24 (m, 8H), 5.51 (t, J=8 Hz, 1H), 4.93 (dd, J=8, 14 Hz, 1H), 4.78 (dd, J=6.8, 14.4 Hz, 1H), 4.30 (m, 4H), 3.02 (t, J=7.6 Hz, 2H), 2.65 (m, 11H), 2.38 (s, 3H). ¹³C-NMR (CDCl₃) δ (ppm): 168.7, 155.7, 154.1, 138.7, 138.0, 136.1, 129.0, 128.7, 128.5, 127.4, 126.5, 103.7, 63.6, 60.1, 56.4, 54.7, 53.8, 52.3, 48.8, 45.4, 35.1, 29.7, 14.3. MS (ES) m/z: 595 [M+1]⁺.

### 2-(4-Methylpiperazin-1-yl)ethyl (1-(2-(4-bromophenyl)-2-chloroethyl)-H-pyrazolo[3,4-d]pyrimidin-4-yl)(phenyl)carbamate (proSi223)

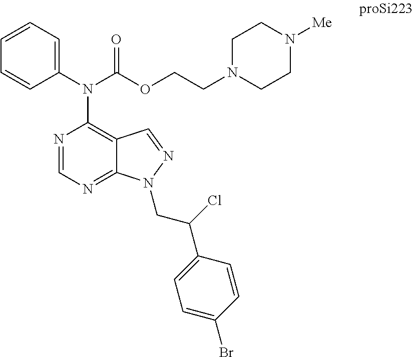 [SMILES: CN1CCN(CCOC(=O)N(c2ccccc2)c2ncnc3c2cnn3CC(Cl)c2ccc(Br)cc2)CC1]

White Solid. Yield: 25%. ¹H-NMR (CDCl₃) δ (ppm): 8.61 (s, 1H), 7.77 (s, 1H), 7.42 (m, 4H), 3.76 (m, 5H), 5.48 (t, J=8 Hz, 1H), 4.97 (dd, J=8.4, 14 Hz, 1H), 4.80 (dd, J=6.4, 14 Hz, 1H), 4.36 (t, J=5.6 Hz, 2H), 2.57 (t, J=5.6 Hz, 2H), 2.43 (m, 8H), 2.31 (S, 3H). ¹³C-NMR (CDCl₃) δ (ppm): 155.6, 155.0, 154.7, 153.6, 139.9, 136.8, 135.0, 131.9, 129.1, 128.7, 128.3, 123.1, 106.1, 65.2, 64.8, 59.2, 56.3, 56.2, 54.8, 54.7, 53.6, 53.4, 52.4, 45.5, 30.3, 29.7. MS (ES) m/z: 598 [M+1]⁺, 620 [M+23]⁺.

### 2-(4-Methylpiperazin-1-yl)ethyl (1-(2-chloro-2-phenylethyl)-6-(methylthio)-1H-pyrazolo[3,4-d]pyrimidin-4-yl)(3-chlorophenyl)carbamate (proSi83)

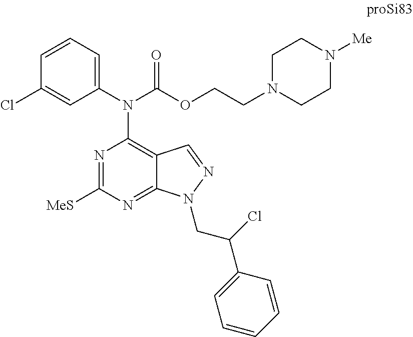 [SMILES: CSc1nc(N(C(=O)OCCN2CCN(C)CC2)c2cccc(Cl)c2)c2cnn(CC(Cl)c3ccccc3)c2n1]

Transparent oil. Yield: 85%. ¹H-NMR (CDC₃) δ (ppm): 7.95 (s, 1H), 7.40 (d, J=6.8 Hz, 2H), 7.29 (m, 6H), 7.11 (m, 1H), 5.50 (t, J=7.0 Hz, 1H), 4.93 (dd, J=8.4, 14 Hz, 1H), 4.76 (dd, J=6.4, 14 Hz, 1H), 4.35 (t, J=5.2 Hz, 2H), 2.55 (t, J=5.2 Hz, 2H), 2.41 (m, 8H), 2.27 (s, 3H), 2.26 (s, 3H).

¹³C-NMR (CDC₃) δ (ppm): 168.6, 155.8, 153.7, 153.1, 140.6, 137.7, 135.2, 134.1, 129.6, 129.0, 128.8, 128.5, 127.9, 127.2, 126.9, 103.0, 64.6, 59.8, 56.2, 54.8, 53.6, 52.8, 45.7, 29.5. MS (ES) m/z: 601 [M+1]⁺.

### 1-(4-Methylpiperazin-1-yl)propan-2-yl (3-bromophenyl)(6-(methylthio)-1-(2-phenylpropyl)-1H-pyrazolo[3,4-d]pyrimidin-4-yl)carbamate2 (proSi278 (B))

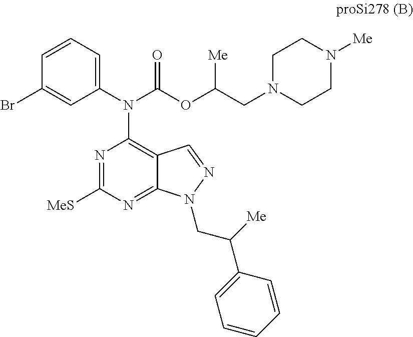 [SMILES: CSc1nc(N(C(=O)OC(C)CN2CCN(C)CC2)c2cccc(Br)c2)c2cnn(CC(C)c3ccccc3)c2n1]

Transparent oil. Yield: 30%. 1H-NMR (CDCl₃) δ (ppm): 7.88 (s, 1H), 7.46 (m, 2H), 7.24 (m, 5H), 7.16 (m, 2H); 5.20 (m, 1H), 4.50 (m, 2H), 3.52 (q, J=7.2 Hz, 1H), 2.45 (m, 10H), 2.33 (s, 3H), 2.28 (s, 3H), 1.22 (m, 6H). ¹³C-NMR (CDCl₃) δ (ppm): 168.1, 155.5, 153.9, 152.9, 143.1, 141.1, 134.29, 131.8, 130.6, 129.8, 128.2, 127.3, 127.0, 126.5, 121.7, 103.15, 71.0, 62.6, 54.8, 53.5, 52.7, 45.4, 39.7, 29.5, 18.5, 17.9. MS (ES) m/z: 639 [M+1]⁺.

### 1-(4-Methylpiperazin-1-yl)butan-2-yl (3-bromophenyl)(6-(methylthio)-1-(2-phenylpropyl)-1H-pyrazolo[3,4-d]pyrimidin-4-yl)carbamate (proSi278 (C))

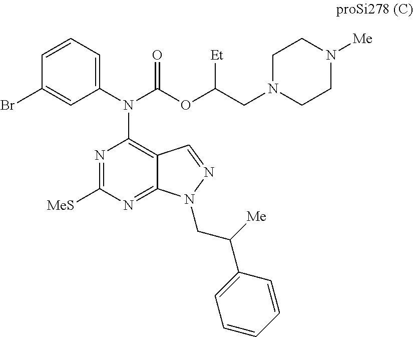 [SMILES: CCC(CN1CCN(C)CC1)OC(=O)N(c1cccc(Br)c1)c1nc(SC)nc2c1cnn2CC(C)c1ccccc1]

Transparent oil. Yield: 40%. ¹H-NMR (CDC₃) δ (ppm): 7.90 (s, 1H), 7.46 (m, 2H), 7.24 (m, 5H), 7.16 (m, 2H); 5.08 (m, 1H), 4.50 (m, 2H), 3.52 (q, J=6.8 Hz, 1H), 2.50 (m, 12H), 2.29 (s, 3H), 2.27 (s, 3H), 1.23 (s, 3H), 0.88 (m, 3H). ¹³C-NMR (CDC₃) δ (ppm): 170.2, 155.4, 154.0, 153.2, 143.1, 141.1, 134.4, 131.8, 130.5, 129.7, 128.2, 127.3, 127.0, 126.5, 121.7, 103.4, 75.4, 61.0, 54.9, 53.5, 45.6, 39.7, 29.5, 25.1, 18.5, 13.9, 9.34. MS (ES) m/z: 652 [M+1]⁺.

### 2-(4-methylpiperazin-1-yl)-1-phenylethyl (3-bromophenyl)(6-(methylthio)-1-(2-phenylpropyl)-1H-pyrazolo[3,4-d]pyrimidin-4-yl)carbamate (proSi278 (D))

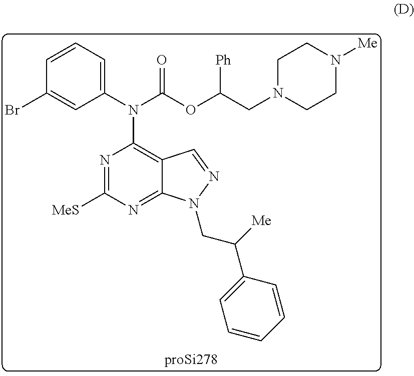 [SMILES: CSc1nc(N(C(=O)OC(CN2CCN(C)CC2)c2ccccc2)c2cccc(Br)c2)c2cnn(CC(C)c3ccccc3)c2n1]

Transparent oil. Yield: 32%. 1H-NMR (CDCl₃) δ (ppm): 7.74 (s, 1H), 7.54 (d, J=8 Hz, 1H), 7.46 (s, 1H), 7.21 (m, 12H), 6.00 (d, J=8.8 Hz, 1H); 4.48 (m, 2H), 3.53 (q, J=6.8 Hz, 1H), 2.88 (bs, 6H), 2.72 (m, 7H), 2.31 (s, 3H), 1.25 (s, 3H). 3C-NMR (CDCl₃) δ (ppm): 168.4, 155.7, 153.9, 152.8, 143.3, 141.2, 137.6, 134.4, 132.1, 131.1, 130.3, 128.7, 127.4, 127.2, 126.8, 126.3, 122.0, 103.2, 75.4, 63.2, 54.4, 53.8, 51.2, 44.4, 39.9, 29.7, 25.1, 18.7. MS (ES) m/z: 652 [M+1].

### 2-(4-Methylpiperazin-1-yl)ethanol (30)

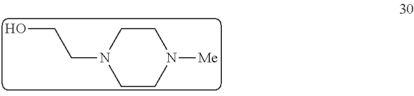

Methylpiperazine (3.54 mL, 31.9 mmol, 1.33 eq.) was dissolved in toluene (11 mL), bromoethanol (1.70 mL, 23.9 mmol, 1.00 eq.) was slowly added and the mixture was stirred o.n. at r.t. Then it was filtered and the organic phase was recovered, the solvent removed under reduced pressure to give the desired product. Yield: 80%. White solid. 1H-NMR (CDCl₃) δ (ppm): 4.51 (s, 1H); 3.14 (m, 2H); 2.01 (m, 10H); 1.78 (s, 3H). ¹³C-NMR (CDCl₃) 5 (ppm): 59.8, 58.0, 54.4, 52.6, 45.5. MS (ES) m/z: 145 [M+H]⁺.

### 1-(4-Methylpiperazin-1-yl)propan-2-ol (31)

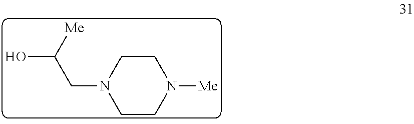 [SMILES: CC(O)CN1CCN(C)CC1]

Methylpiperazine (55.0 μL, 0.50 mmol, 2.00 eq.) was dissolved in toluene (7 mL), 1-bromo-2-propanol (23.0 j L, 0.25 mmol, 1.00 eq.) was slowly added and the mixture was stirred o.n. at r.t. Then it was filtered and the organic phase was recovered, the solvent removed under reduced pressure to give the desired product. Yield: 38%. Transparent oil. ¹H-NMR (MeOD) δ (ppm): 4.51 (s, 1H); 3.14 (m, 2H); 2.01 (m, 10H); 1.78 (s, 3H). 13C-NMR (MeOD) δ (ppm): 63.8, 63.0, 52.8, 50.6, 42.7, 19.8. MS (ES) m/z: 160 [M+H]⁺.

### 1-(4-Methylpiperazin-1-yl)butan-2-ol (32)

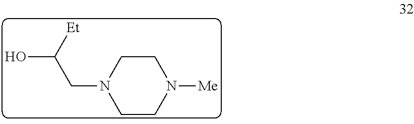 [SMILES: CCC(O)CN1CCN(C)CC1]

ZnCl₂ (12.4 mg, 0.09 mmol, 0.10 eq.) was added to a solution of methylpiperazine (110 μL, 1.00 mmol, 1.10 eq.) and 1,2-epoxybutane (79.0 μL, 0.91 mmol, 1.00 eq.) in ACN (8 mL); the mixture was stirred under reflux 16 h. Then purified by flash chromatography using PE:EtOAc:MeOH:Et₃N=10:8:1:1 as eluent. Yield: 31%. Transparent oil. 1H-NMR (MeOD) δ (ppm): 3.62 (m, 1H); 2.47 (bs, 8H); 2.32 (m, 2H); 2.26 (s, 3H); 1.43 (m, 2H); 0.92 (t, J=7.6 Hz, 3H). 3C-NMR (MeOD) δ (ppm): 71.5, 61.5, 58.2, 57.6, 46.6, 28.3, 9.5. MS (ES) m/z: 173 [M+H]⁺.

### 2-(4-Methylpiperazin-1-yl)-1-phenylethanol (33)

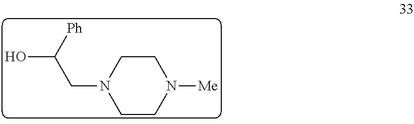 [SMILES: CN1CCN(CC(O)c2ccccc2)CC1]

Methylpiperazine (55.0 μL, 0.50 mmol, 2.00 eq.) was dissolved in toluene (7 mL) and the mixture was heated to 100° C., then stirene oxide (23.0 μL, 0.25 mmol, 1.00 eq.) was slowly added and the mixture was stirred o.n. at 130° C. H₂O was added and extraction with DCM was performed (×3); the organic phases were collected, washed with brine and dried over Na₂SO₄. The oily residue obtained after evaporation of the solvent was purified by flash chromatography using EtOAc:MeOH=8:2 as eluent. Yield: 60%. Transparent oil. 1H-NMR (CDC₃) δ (ppm): 7.29 (m, 5H); 4.71 (m, 1H); 3.93 (bs, 1H); 2.75 (bs, 2H); 2.49 (m, 8H); 2.27 (s, 3H). ¹³C-NMR (CDCl₃) δ (ppm): 142.2, 128.3, 127.5, 125.8, 68.8, 66.15, 55.2, 53.0, 46.0. MS (ES) m/z: 221 [M+H]⁺.

### Chromatographic Method

LC analysis was performed with an Agilent 1100 LC/MSD VL system (G1946C) (Agilent Technologies, Palo Alto, Calif.) constituted by a vacuum solvent degassing unit, a binary high-pressure gradient pump, an 1100 series UV detector, and an 1100 MSD model VL benchtop mass spectrometer.

Chromatographic profiles were obtained using a Varian Polaris C18-A column (150-4.6 mm, 5 μm particle size) and gradient elution: eluent A being ACN and eluent B consisting of water. The analysis started with 2% of eluent A, which was rapidly increased up to 70% in 12 min, then slowly increased up to 98% in 20 min. The flow rate was 0.8 mL min⁻¹ and injection volume was 20 μL.

The Agilent 1100 series mass spectra detection (MSD) single-quadrupole instrument was equipped with the orthogonal spray API-ES (Agilent Technologies, Palo Alto, Calif.). Nitrogen was used as nebulizing and drying gas. The pressure of the nebulizing gas, the flow of the drying gas, the capillary voltage, the fragmentor voltage, and the vaporization temperature were set at 40 psi, 9 L/min, 3000 V, 70 V, and 350° C., respectively. UV detection was monitored at 254 nm. The LC-ESI-MS determination was performed by operating the MSD in the positive ion mode. Spectra were acquired over the scan range m/z 100-1500 using a step size of 0.1 u.

### Water Solubility Assay.

Solid compound (1 mg) was added to 1 mL of water. The samples were shacked in a shaker bath at 20° C. for 24 h. The suspensions were filtered through a 0.45-μm nylon filter (Acrodisc), and the solubilised compound determined by LC-UV-MS assay. The determination was performed in triplicate.

Chromatographic analysis was performed with the method above reported and quantification of compounds was made by comparison with apposite calibration curves realized with standard solutions in methanol.

### Parallel Artificial Membrane Permeability Assay (PAMPA).

Donor solution of tested compounds (0.5 mM) was prepared by diluting 1 mM dimethylsulfoxide (DMSO) compound stock solution using phosphate buffer (pH 7.4, 25 mM). Filters were coated with 5 μL of a 1% (w/v) dodecane solution of phosphatidylcholine or 4 μL of brain polar lipid solution (20 mg/mL 16% CHCl₃, 84% dodecane) prepared from CHCl₃ solution 10% w/v, for intestinal permeability and BBB permeability, respectively. Donor solution (150 μL) was added to each well of the filter plate. To each well of the acceptor plate were added 300 μL of solution (50% DMSO in phosphate buffer). Compounds was tested in three different plates on different days. The sandwich was incubated for 5 h at room temperature under gentle shaking. After the incubation time, the plates were separated, and samples were taken from both receiver and donor sides and analyzed using LC with UV detection at 254 nm.

Chromatographic analysis were performed with the method above reported.

Permeability (Pₐₚₚ) for PAMPA was calculated according to the following equation, obtained from Wohnsland and Faller and Sugano et al.^{27, 28}.

The equation is with some modification in order to obtain permeability values in cm/s:

$${P}_{app} = \frac{{V}_{D} {V}_{A}}{( {V}_{D} + {V}_{A} ) At} - ln ( 1 - r )$$

where V_{A} is the volume in the acceptor well, V_{D} is the volume in the donor well (cm³), A is the “effective area” of the membrane (cm²), t is the incubation time (s) and r the ratio between drug concentration in the acceptor and equilibrium concentration of the drug in the total volume (V_{D}+V_{A}). Drug concentration is estimated by using the peak area integration. Membrane retention (%) was calculated according to the following equation:

$$\% MR = \frac{[ r - ( D + A ) ] 100}{Eq}$$

where r is the ratio between drug concentration in the acceptor and equilibrium concentration, D, A, and Eq represented drug concentration in the donor, acceptor and equilibrium solution, respectively.

### 5.2—Biological Activity: Materials and Methods

### Microsomal Stability Assay.

Each compound, solubilized in DMSO, were incubated at 37° C. for 60 min in 25 mM phosphate buffer (pH 7.4), 5 μL of human liver microsomal protein (0.2 mg/mL), in the presence of a NADPH-generating system at a final volume of 0.5 mL (compounds' final concentration, 50 μM); DMSO did not exceed 2% (final solution). The reaction was stopped by cooling in ice and adding 1.0 mL of acetonitrile. The reaction mixtures were then centrifuged for 15 min at 10000 rpm, and the parent drug and metabolites were subsequently determined by LC-UV-MS. Chromatographic analysis were performed with the method above reported.

The percentage of not metabolized compound was calculated by comparison with reference solutions. The determination was performed in triplicate.

### Stability Tests

Prodrug solutions (500 μM) maintained at 20° C., were prepared by dissolving the compounds in 0.0125 M pH 7.4 phosphate buffer, water and methanol, respectively. Aliquots (20 μL) withdrawn during the 48 h incubation period were analyzed by HPLC.

To determine enzymatic stability, pooled human plasma (750 μL), pH 7.4 phosphate buffer (700 μL), and 50 μL of 3.0 mM solution of prodrug in MeOH (final concentration 100 μM) were mixed in a test tube.

The tube was incubated at 37° C. and at predetermined time point, a 150 μL aliquots was removed, mixed with 600 μL of cold acetonitrile and centrifuged at 5000 rpm for 15 min. The supernatant was removed and analyzed by HPLC.

The hydrolysis of the compounds were followed by HPLC with UV-MS detection methods above reported.

The half-life of the decaying quantity (t^{1/2}) was calculated according to the following equation, obtained from Sobol et al⁷⁹.

$${t}_{1 / 2} = \frac{ln ( 2 )}{k}$$

where ln(2) is the natural logarithm of 2 (0.693) and k is the elimination rate constant. Values are expressed in minutes.

### Antiproliferative Activity on Neuroblastoma Human Cell Line SH-SY5Y.

In vitro experiments were carried out using the human neuroblastoma cell line SH-SY5Y. Cells were purchased from American Type Culture Collection (ATCC, Manassas, Va., USA) and were cultured in DMEM medium supplemented with 10% Foetal Bovine Serum. In order to determine antiproliferative effect of drugs SH-SY5Y cells were seeded at 2×105 cells/ml density and treated with compounds at increasing concentrations from 0.01 to 50 μM. The cultures were maintained at 37° C. in 5% v/v CO2 for 72 h. Cell number and vitality were evaluated using the automatic cell counter NucleoCounter® (Chemometec, Denmark). Results from the NucleoCounter represented either total or non-viable cell concentration, depending on the sample preparation indicated by manufacturer. IC50 (drug concentration that determined the 50% of growth inhibition) was calculated by Grafit v4.0 (Erithacus Software Limited) software using the best fitting sigmoid curve.

### Proliferation Assay.

U87 and U251 cells were purchased from European Collection of Cell Cultures (ECACC, Salisbury, UK) and were cultured in DMEM medium supplemented with 10% Foetal Bovine Serum. In order to determine antiproliferative effect of drugs tumor cells were seeded at 2×105 cells/ml density and treated with compounds at indicated concentrations. The cultures were maintained at 37° C. in 5% v/v CO2 for 72 h. Cell number and viability were evaluated by Trypan blue exclusion test. Viable cells were expressed as percentage respect to untreated cells (=100%/). Mean and SD values of at least three different experiments are shown.

### Antiproliferative Activity on Human Cell Line K562.

In vitro experiments were carried out using the human Chronic Myelogeneous Leukemia cell line K562. Cells were purchased from American Type Culture Collection (ATCC, Manassas, Va., USA) and were cultured in RPMI medium supplemented with 10% Foetal Bovine Serum. In order to determine antiproliferative effect of Fyn inhibitors K562 cells were seeded at 2×105 cells/ml density and treated with compounds at increasing concentrations from 0.01 to 50 μM. The cultures were maintained at 37° C. in 5% v/v CO₂ for 72 h. Cell number and vitality were evaluated using the automatic cell counter NucleoCounter® (Chemometec, Denmark). Results from the NucleoCounter represented either total or non-viable cell concentration, depending on the sample preparation indicated by manufacturer. IC₅₀ (drug concentration that determined the 50% of growth inhibition) was calculated by Grafit v4.0 (Erithacus Software Limited) software using the best fitting sigmoid curve.

### In Vivo Pharmacokinetics.

The animal protocols used were reviewed and approved by the Animal Care and Ethics Committee of the Universita` degli Studi di Siena, Italy. Male BALB/C mice (weight 20-30 g) were obtained from Charles River (Milan, Italy). The experiment was performed in triplicate and mice were divided into 3 groups; each group received 100 μL of DMSO (control), drug (Si306) or prodrug (pro-Si306) solution in DMSO (i.p., 50 mg·Kg⁻¹). Animals were treated with heparin solution and sacrificed under CO₂ at different time points (0.25 h-24 h); blood (drawn by cardiac puncture) and brain were collected for the following quantitative analysis. The blood, previously heparinized, was centrifuged at 4000 rpm for 20 minutes to separate the plasma fraction and then 500 μl were collected in a test tube. For each sample 1 ml of ACN (in the presence of compound S34 5 μM, as internal standard) was added to denature proteins and to extract drug and prodrug. Samples were centrifuged at 4000 rpm for 20 minutes, the supernatant was recovered, dried under vacuum and analyzed by LC-UV-MS. Brain was homogenized using a glass/glass Potter-Elvehjem homogenizer in presence of Tris-HCl buffer (50 mM) and compounds were recovered using 7 mL of ACN then treated as previously described for blood samples.

### 5.3—Prodrug: Discussion

The use of prodrugs—chemically modified versions of the pharmaceutically active drug which after undergoing in vivo transformations release the active drug—represents a well established strategy to improve the physicochemical, biopharmaceutical or pharmacokinetic properties of potential drug candidates.^{34,35}

The biological activity of pyrazolo[3,4-d]pyrimidines is sometimes associated with low water solubility which could influence the future development of these putative drug candidates. In order to overcome this issue, enhance pharmacokinetic properties and facilitate in vivo distribution produgs of pyrazolo[3,4-d]pyrimidine compounds have been synthesized.³⁶ These compounds are characterized by a solubilizing moiety, namely a N-methylpiperazino group, linked to the C4 position of the pyrazolo[3,4-d]pyrimidine nucleus, by an O-alkyl carbamate chain.

The development of a more rapid and versatile synthesis (with respect to the one already reported),³⁶ applicable to a wide range of previously synthesized final compounds, was an appealing goal. After the synthesis of the appropriate alcohols 30, 31, 32 and 33 (Scheme 5), a one pot-two step procedure was performed, using sodium bicarbonate as base for: chlorocarbonate formation and subsequent displacement of chlorine by alcohol (Scheme 6). All the prodrugs (proS13, proS13(A), proSi221, proSi214, proSi306, proSi35, proSi223, proSi83, proSi20, proSi1278(A), proSi1278(B), proSi1278(C), proSi278(D)) have been synthesised starting from the correspondent Si compound (Si13, Si221, Si214, Si306, Si35, Si1223, Si83, Si120, Si278) listed in Table 7.

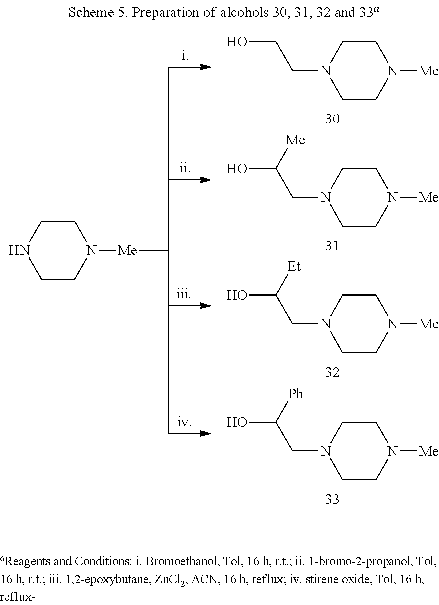

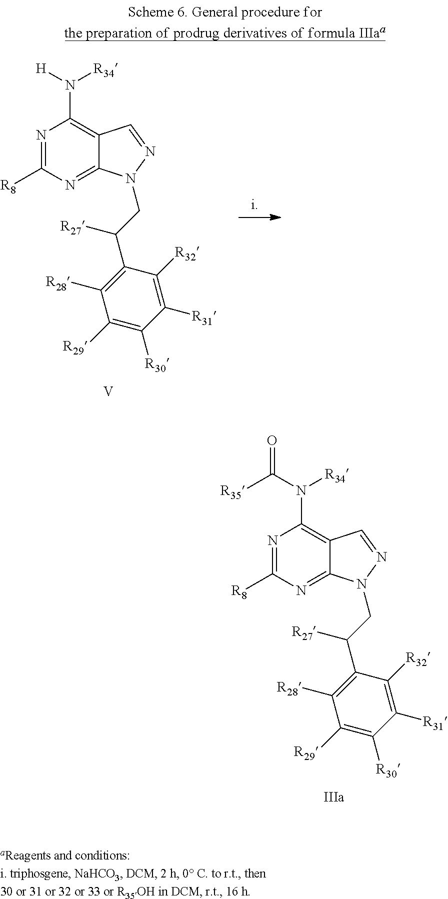

### Wherein

R₃₅′OH is an alcohol compound,
R₈, R₂₇′, R₂₈′, R₂₉′, R₃₀′, R₃₁′, R₃₂′, R₃₄′ are as defined above, and

### R₃₅′ is:

an alkyl chain with the formula:

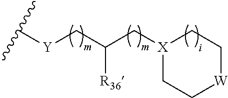

where Y is NH or O or S; R₃₆′ is H or alkyl or aryl or aralkyl; X is CH or N; W is NH or NCH₃ or O; m is an integer from 0 to 2; i is an integer from 0 to 1;

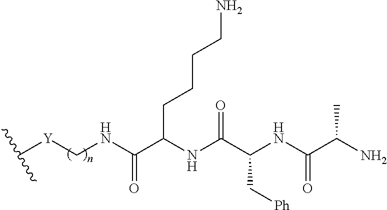

where Y is NH or O or S;
n is an integer from 0 to 4;
or:

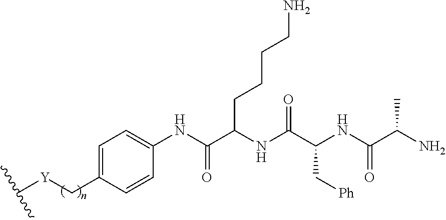

where Y is NH or O or S;
n is an integer from 0 to 4;

TABLE 7
Pyrazolo[3,4-d]pyrimidine compounds and their respective newly synthesized Prodrugs.

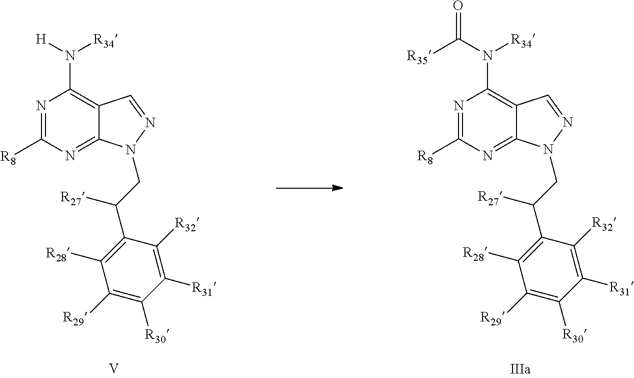

| | Compound | | | | | |
|---|---|---|---|---|---|---|
| | reference | | | | | |
| Formula | number | R_{27′} | R_{30′} | R_{34′} | R₈ | R_{35′} |
| V | S13 | Cl | H | CH₂C₆H₅ | H | — |
| IIIa | proS13 | Cl | H | CH₂C₆H₅ | H | 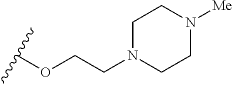 [SMILES: *OCCN1CCN(C)CC1] |
| IIIa | proS13 (A) | Cl | H | CH₂C₆H₅ | H | 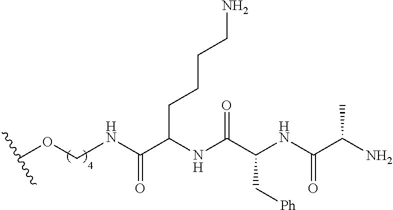 |
| V | Si221 | Cl | Br | CH₂C₆H₄oCl | H | — |
| IIIa | proSi221 | Cl | Br | CH₂C₆H₄oCl | H | 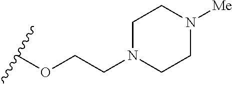 [SMILES: *OCCN1CCN(C)CC1] |
| V | Si214 | H | H | C₆H₄mCl | SMe | — |
| IIIa | proSi214 | H | H | C₆H₄mCl | SMe | 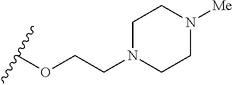 [SMILES: *OCCN1CCN(C)CC1] |
| V | Si306 | Cl | H | C₆H₄mBr | SCH₂CH₂ | — |
| | | | | | 4-morpholino | |
| IIIa | proSi306 | Cl | H | C₆H₄mBr | SCH₂CH₂ 4-morpholino | 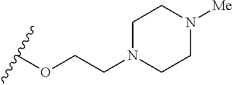 [SMILES: *OCCN1CCN(C)CC1] |
| V | Si35 | Cl | H | CH₂CH₂C₆H₅ | SMe | — |
| IIIa | proSi35 | Cl | H | CH₂CH₂C₆H₅ | SMe | 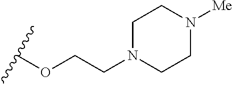 [SMILES: *OCCN1CCN(C)CC1] |
| V | Si223 | Cl | Br | C₆H₅ | H | — |
| IIIa | proSi223 | Cl | Br | C₆H₅ | H | 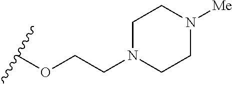 [SMILES: *OCCN1CCN(C)CC1] |
| V | Si83 | Cl | H | C₆H₄mCl | SMe | — |
| IIIa | proSi83 | Cl | H | C₆H₄mC1 | SMe | 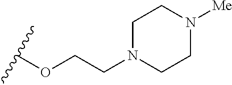 [SMILES: *OCCN1CCN(C)CC1] |
| V | Si20 | Cl | H | nBu | SEt | — |
| IIIa | proSi20 | Cl | H | nBu | SEt | 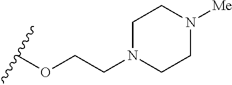 [SMILES: *OCCN1CCN(C)CC1] |
| V | Si278 | Me | H | C₆H₄mBr | SMe | — |
| IIIa | proSi278(A) | Me | H | C₆H₄mBr | SMe | 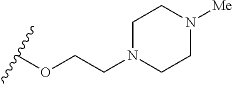 [SMILES: *OCCN1CCN(C)CC1] |
| IIIa | proSi278(B) | Me | H | C₆H₄mBr | SMe | 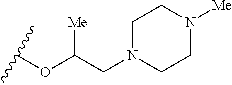 [SMILES: *OC(C)CN1CCN(C)CC1] |
| IIIa | proSi278(C) | Me | H | C₆H₄mBr | SMe | 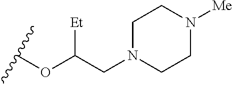 [SMILES: *OC(CC)CN1CCN(C)CC1] |
| IIIa | proSi278(D) | Me | H | C₆H₄mBr | SMe | 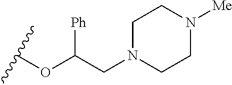 [SMILES: *OC(CN1CCN(C)CC1)c1ccccc1] |
| IIIa | proSi278(E) | Me | H | C₆H₄mBr | SMe | 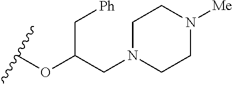 [SMILES: *OC(Cc1ccccc1)CN1CCN(C)CC1] |

Aqueous solubility, GI (gastro intestinal) and BBB (blood brain barrier) Apparent Permeability have been assessed. Stability in PBS, MeOH and plasma are also reported (Table 8).

TABLE 8
Characterization of example compounds: solubility, stability and membrane permeability

| | | | | Stability^{a} | | | |
|---|---|---|---|---|---|---|---|
| Compound | PAMPA | PAMPA | H₂O Sol.^{a} | | | Human | Metabolic |
| reference | GI^{a,b} | BBB^{a,c} | μg · mL⁻¹ | PBS pH 7.4 | MeOH | Plasma | Stab.^{a,g} |
| number | (MR %)^{h} | (MR %)^{h} | (Log S)^{d} | t_{1/2}^{e} (h) | t_{1/2}^{e}(h) | t_{1/2}^{e} (h) | % |
| S13 | 11.08 | 16.5 | 0.70 | ND^{f} | ND^{f} | ND^{f} | 78.3 |
| | (5.9) | (0) | (−5.71) | | | | |
| proS13 | 6.70 | 5.01 | 18.81 | >48 | >48 | >48 | 86.4 |
| | (40.1) | (45.6) | (−4.45) | | | | |
| Si35 | 7.4 | 6.17 | 0.07 | ND^{f} | ND^{f} | ND^{f} | 96.2 |
| | (33.0) | (24.5) | (−6.78) | | | | |
| proSi35 | 0.85 | 0.01 | 106.97 | >48 | >48 | 35.32 | ND^{f} |
| | (86.1) | (88.8) | (−3.74) | | | | |
| Si83 | 0.26 | 3.14 | 0.13 | ND^{f} | ND^{f} | ND^{f} | 91.1 |
| | (69.9) | (39.6) | (−6.53) | | | | |
| proSi83 | 4.95 | 4.14 | 4.22 | >48 | >48 | 3.28 | ND^{f} |
| | (44.7) | (47.0) | (−5.15) | | | | |
| Si214 | 0 | 1.48 | 0.12 | ND^{f} | ND^{f} | ND^{f} | 99.0 |
| | (78.0) | (55.7) | (−6.50) | | | | |
| proSi214 | 4.69 | 4.30 | 6.32 | >48 | >48 | 3.67 | ND^{f} |
| | (63.1) | (49.7) | (−4.95) | | | | |
| Si221 | 8.78 | 13.23 | <0.01 | ND^{f} | ND^{f} | ND^{f} | 95.2 |
| | (21.0) | (7.3) | (<−8.60) | | | | |
| proSi221 | 2.38 | 1.92 | 1.95 | >48 | >48 | >48 | ND^{f} |
| | (78.1) | (79.4) | (−5.51) | | | | |
| Si223 | 6.64 | 13.1 | 0.06 | ND^{f} | ND^{f} | ND^{f} | 96.4 |
| | (32.1) | (11.6) | (−6.86) | | | | |
| proSi223 | 9.91 | 6.97 | 41.56 | >48 | >48 | 4.47 | ND^{f} |
| | (16.0) | (24.9) | (−4.15) | | | | |
| Si278 | 0 | 0.50 | <0.01 | ND^{f} | ND^{f} | ND^{f} | 95.1 |
| | (80.4) | (66.3) | (<−7.60) | | | | |
| proSi278 (A) | 2.11 | 1.89 | 6.47 | >48 | >48 | 3.21 | 99.9 |
| | (67.0) | (49.8) | (−4.94) | | | | |
| proSi278 (B) | 2.15 | 2.39 | 3.40 | >48 | >48 | 10.40 | ND^{f} |
| | (73.3) | (70.6) | (−5.27) | | | | |
| proSi278 (C) | 1.45 | 0.93 | 1.96 | >48 | >48 | 11.31 | ND^{f} |
| | (88.1) | (91.4) | (−5.52) | | | | |
| Si306 | 5.27 | 7.10 | 3.70 | ND^{f} | ND^{f} | ND^{f} | 97.2 |
| | (46.1) | (41.0) | (−5.18) | | | | |
| proSi306 | 2.13 | 2.91 | 8.70 | >48 | >48 | 3.48 | ND^{f} |
| | (45.5) | (42.0) | (−4.93) | | | | |

^{a}Determined by UV/LC-MS.
^{b}Gastrointestinal Parallel Artificial Membrane Permeability Assay (10⁻⁶ cm · sec⁻¹).
^{c}Blood Brain Barrier Parallel Artificial Membrane Permeability Assay (10⁻⁶ cm · sec⁻¹⁾.
^{d}Log S = log mol · L⁻¹.
^{e}t_{1/2} = In 2 · K_{obs}⁻¹.
^{f}Not Determined.
^{b}Calculated by Discovery Studio 3.0.
^{g}Expressed as percentage of unmodified drug.
^{h}Membrane Retention (MR) expressed as percentage of compound unable to reach the acceptor compartment. Data represent mean values of at least two experiments.

Prodrugs demonstrated an enhanced water solubility with regards to the respective drugs. Furthermore increasing the bulkiness of the prodrug moiety results in an enhancement of plasma stability, thus enabling the inventors to choose the right substituent depending on the necessity (tin in human plasma: proSi278 (A) (3.21 h)<proSi278 (B) (10.4 h)<proSi278 (C) (11.31 h)).

Table 9 presents the cellular data (IC₅₀) in glioma U251 and U87 cells (FIG. 22), neuroblastoma SH-SY5Y cells (FIG. 21) and leukemia K562 cells (FIG. 23), prodrugs showed a general improvement of activity towards cancer cell lines.

TABLE 9
Biological evaluation of example compounds
IC₅₀ uM^{a}

| | proSi20 | Si20 | proSi223 | Si223 | proSi278 (A) | Si278 |
|---|---|---|---|---|---|---|
| U251 | 0.8 | 4.5 | 4.7 | 14.2 | 5.2 | 3.7 |
| U87 | 3.6 | 3.8 | 5.1 | 17.3 | 6.3 | 5.8 |
| SH-SY5Y | 0.3 | 8.5 | 12.6 | 16.8 | 0.4 | 1.2 |

U251 and U87: glioma cell lines. SH-SY5Y: neuroblastoma cell lines. Cells were treated for 72 h with different concentrations of compound (0.1 μM, 1 μM, 5 μM, 10 μM.
^{a}IC₅₀: the half maximal inhibitory concentration of the effectiveness in reducing the number of viable cells with respect to untreated cells.

FIG. 24 shows in vivo pharmacokinetics: proSi306 (and its hydrolysis-derived product, namely Si306) showed a higher brain concentration (site of glioma tumour) with respect to the drug. The same assay demonstrated the in vivo hydrolysis of proSi306, with consequent release of the drug Si306. Furthermore, plasma analysis indicated a better profile of distribution for proSi306. These results demonstrated the validity of the prodrug approach, in fact the quantity of total compound—given by the sum of proSi306 and Si306 produced by hydrolysis—able to reach respectively the brain and blood tissue results higher than the one obtained by drug Si306 administration. Table 10 describes the quantity of compounds found in bran and blood tissue at fixed time points. FIG. 25 depicts the quantity of compound found in brain and plasma ate the time point of 24 hours.

TABLE 10
Quantity of Compound Si306, pro-Si306 and Si306 hydrolysis-derived
in blood and brain tissue (nmol of compound/g of tissue)

| | Quantity of Compound (nmol of compound/g of tissue) | | | | | | | |
|---|---|---|---|---|---|---|---|---|
| Time points | 15′ | 30′ | 1 h | 1.5 h | 2 h | 4 h | 8 h | 24 h |
| BRAIN | | | | | | | | |
| Prodrug (Pro-Si306) | 5.3 ± 1.5 | 3.4 ± 0.33 | 3.4 ± 2.24 | 2.4 ± 0.24 | 2.8 ± 0.60 | 2.9 ± 1.38 | 3.6 ± 2.12 | 6.6 ± 1.08 |
| Drug from hydrolysis | 0.8 ± 0.98 | 0.5 ± 0.45 | 0.5 ± 0.49 | 0.7 ± 0.43 | 0.5 ± 0.46 | 0.4 ± 0.57 | 0.6 ± 0.65 | 3.9 ± 0.67 |
| (Si306 form Pro-Si306) | | | | | | | | |
| Drug (Si306) | 0.3 ± 0.18 | 0.5 ± 0.49 | 0.4 ± 0.36 | 0.8 ± 0.03 | 0.6 ± 0.52 | 0.4 ± 0.33 | 0.6 ± 0.63 | 2.3 ± 0.70 |
| BLOOD | | | | | | | | |
| Prodrug (Pro-Si306) | 10.7 ± 1.11 | 8.9 ± 1.22 | 11.4 ± 1.29 | 8.3 ± 2.21 | 7.2 ± 2.17 | 6.5 ± 0.58 | 6.2 ± 2.07 | 3.7 ± 0.60 |
| Drug from hydrolysis | 6.3 ± 3.71 | 4.9 ± 4.04 | 5.1 ± 3.38 | 7.9 ± 1.56 | 2.9 ± 1.48 | 3.2 ± 1.78 | 1.8 ± 0.57 | 1.8 ± 1.33 |
| (Si306 form Pro-Si306) | | | | | | | | |
| Drug (Si306) | 1.8 ± 1.80 | 10.2 ± 4.82 | 7.5 ± 5.73 | 6.4 ± 3.82 | 3.2 ± 3.74 | 2.6 ± 1.31 | 2.4 ± 0.29 | 1.1 ± 0.65 |

### REFERENCES

(1) Arora, A.; Scholar, E. M. J. Pharmacol. Exp. Ther. 2005, 315, 971-979.
(2) Pytel, D.; Anticancer Agents Med. Chem. 2009, 9, 66-76.
(3) (a) Schenone S, et al. Expert Opin Investig Drugs. 2010 19, 931-45. (b) Hanahan, D.; Coussens, L. M. Cancer Cell 2012 21, 309-22. (c) Kalluri, R.; Zeisberg, M. Nature reviews Cancer 2006 9, 392-401. (d) Togo, S. et al., Cancers 2013 5, 149-169.
(4) (a) Bolen, J. B.; Rosen, N.; Israel, M. A. Proc. Natl. Acad Sci. U.S.A. 1985, 82, 7275-7279. (b) O'Shaughnessy, J et al., Oncogene Res. 1987, 2, 1-18. (c) Bjelfman, C.; Hedborg, F.; Johansson, L; Nordenskjold, M.; Pahlman. S. Cancer Res. 1990, 50, 6908-6914. (d) Matsunaga, T.; Takahashi, H.; Ohnuma, N.; Tanabe, M.; Yoshida, H.; Iwai, J.; Shirasawa, H.; Simizu, B. Cancer Res. 1991, 51, 3148-3152. (e) Finlay, D.; Vuori, K. Cancer Res. 2007, 67, 11704-11711.
(5) a) Matsunaga, T.; Shirasawa, H.; Tanabe, M.; Ohnuma, N.; Takahashi, H.; Simizu, B. Cancer Res. 1993, 53, 3179-3185. b) Matsunaga, T.; Shirasawa, H.; Tanabe, M.; Ohnuma, N.; Kawamura, K.; Etoh, T.; Takahashi, H.; Simizu B. Int. J. Cancer, 1994, 58, 793-798.
(6) Hishiki, T.; Saito, T.; Sato, Y.; Mitsunaga, T.; Terui, E.; Matsuura, G.; Saito, E.; Shibata, R.; Mise, N.; Yokoyama, Y.; Yoshida, H. Pediatr. Surg. Int. 2011, 27, 225-230.
(7) Vitali, R.; Mancini, C.; Cesi, V.; Tanno, B.; Piscitelli, M.; Mancuso, M.; Sesti, F.; Pasquali, E.; Calabretta, B.; Dominici, C.; Raschellà G. Int. J. Cancer 2009, 125, 2547-2555.
(8) National cancer institute. Surveillance, Epidemiology and End Results Database. http://seer.cancer.gov (accessed November, 2005).
(9) Brodeur, G. M.; Maris, J. M. Neuroblastoma. In: Pizzo, P. A., Poplack, D. G. eds. Principles and practice of pediatric oncology, 5th edn. Philadelphia: J B Lippincott Company, 2006, 933-970.
(10) De Bernardi, B.; Nicolas, B.; Boni, L. Indolfi, P.; Carli, M. et al. J. Clin. Oncol. 2003, 21, 1592-601.
(11) Matthay, K. K.; Villablanca, J. G.; Seeger, R. C. et al. N. Engl. J. Med. 1999, 341, 1165-1173.
(12) Brodeur, G. M. Nat. Rev. Cancer 2003, 3, 203-216.
(13) Maris, J. M.; Hogarty, M. D.; Bagatell, R.; Cohn, S. L. Lancet 2007, 369, 2106-2120.
(14) Wen P Y, Kesari S. N Engl J Med. 2008 359, 492-507.
(15) Stupp R, Mason W P, van den Bent M J, Weller M, Fisher B, Taphoorn M J. N Engl J Med. 2005 352, 987-96.
(16) Hochberg F H, Pruitt A. Neurology 1980 30, 907-11.
(17) Ahluwalia M S l, de Groot J, Liu W M, Gladson C L Cancer Lett. 2010 8, 139-49.
(18) (a) Carraro, F.; Pucci, A.; Naldini, A.; Schenone, S.; Bruno, O.; Ranise, A.; Bondavalli, F.; Brullo, C.; Fossa, P.; Menozzi, G.; Mosti, L.; Manetti, F.; Botta, M. J. Med. Chem. 2004, 47, 1595-1598. (b) Carraro, F.; Naldini, A.; Pucci, A.; Locatelli, G. A.; Maga, G.; Schenone, S.; Bruno, O.; Ranise, A.; Bondavalli, F.; Brullo, C.; Fossa, P.; Menozzi, G.; Mosti, L.; Modugno, M.; Tintori, C.; Manetti, F.; Botta, M. J. Med Chem. 2006, 49, 1549-1561. (c) Manetti, F.; Santucci, A.; Locatelli, G. A.; Maga, G.; Spreafico, A.; Serchi, T.; Orlandini, M.; Bernardini, G.; Caradonna, N. P.; Spallarossa, A.; Brullo, C.; Schenone, S.; Bruno, O.; Ranise, A.; Bondavalli, F.; Hoffmann, O.; Bologna, M.; Angelucci, A.; Botta, M. J. Med. Chem. 2007, 50, 5579-5588. (d) Angelucci, A.; Schenone, S.; Gravina, G. L.; Muzi, P.; Festuccia, C.; Vicentini, C.; Botta, M.; Bologna, M. Eur. J. Cancer 2006, 42, 2838-2845. (e) O. Bruno, C. Brullo, F. Bondavalli, A. Ranise, S. Schenone, M. S. Falzarano, K. Varani, S. Spisani Bioorg. Med. Chem. Lett. 2007, 17, 3696-3701. (f) Vignaroli, G.; Mencarelli, M.; Sementa, D.; Crespan, E.; Kissova, M.; Maga, G.; Schenone, S.; Radi, M.; Botta, M. ACS Comb. Sci. 2014, 16, 168-175.
(19) Navarra, M.; Celano, M.; Maiuolo, J.; Schenone, S.; Botta, M.; Angelucci, A.; Bramanti, P.; Russo, D. BMC Cancer 2010, 10, 602.
(20) (a) Radi, M.; Brullo, C.; Crespan, E.; Tintori, C.; Musumeci, F.; Biava, M.; Schenone, S.; Dreassi, E.; Zamperini, C.; Maga, G.; Pagano, D.; Angelucci, A.; Bologna, M.; Botta, M. Bio Med. Chem. Lett. 2011, 21, 5928-5933. (b) Silvia Schenone, S.; Bruno, O.; Bondavalli, F.; Ranise. A.; Mosti, L.; Menozzi, G.; Fossa, P.; Manetti, F.; Morbidelli, L.; Trincavelli, L.; Martini, C.; Lucacchini, A. European Journal of Medicinal Chemistry 2004, 39, 153-160
(21) Radi, M.; Dreassi, E.; Brullo, C.; Crespan, E.; Tintori, C.; Bernardo, V.; Valoti, M.; Zamperini, C.; Daigl, H.; Musumeci, F.; Carraro, F.; Naldini, A.; Filippi, L; Maga, G.; Schenone, S.; Botta, M. J. Med. Chem. 2011, 54, 2610-2626.
(22) a) Falchi, F.; Manetti, F.; Carraro, F.; Naldini, A.; Maga, G.; Crespan, E.; Schenone, S.; Bruno, O.; Brullo, C.; Botta, M. Chem Med Chem. 2009, 4, 976-987. b) Alcaro, S.; Artese, A.; Botta, M.; Zizzarri, A.; Orallo, F.; Ortuso, F.; Schenone, S.; Brullo, C.; Yifiez, M. Chem Med Chem 2010, 5, 1242-1246.
(23) Kruewel, T.; Schenone, S.; Radi, M.; Maga, G.; Rohrbeck, A.; Botta, M.; Borlak, J. PloS One 2010, 5, e14143.
(24) Hanefeld, U.; Rees, C. W.; White, A. J. P.; Williams, D. J. J. Chem. Soc., Perkin Trans 1, 1996, 1545-1552
(25) a) Robins, Roland K. Potential purine antagonists. I. JACS 1956 78, 784-90.
(26) Hirst, Gavin C.; Calderwood, David; Wishart, Neil; Rafferty, Paul; Ritter, Kurt; Arnold, Lee D.; Friedman, Michael M. Preparation of pyrazolopyrimidines as protein kinase inhibitors. PCT Int. Appl. (2001), WO2001019829, A220010322.
(27) Wohnsland, F.; Faller, B. J. Med. Chem. 2001 44, 923-930.
(28) Sugano, K.; Hamada, H.; Machida, M.; Ushio, H. J. Biomol. Screen. 2001, 6, 189-196.
(29) Houchens D P, Ovejera A A, Riblet S M, Slagel D E. Eur J Cancer Clin Oncol. 1983 19, 799-805.
(30) Ponten J. In: Fogh J, editor. In Human Tumor Cells in Vitro. New York: Plenum Press 1975, 175-185
(31) Radaelli E, Ceruti R, Patton V, Russo M, Degrassi A, Croci V, Caprera F, Stortini G, Scanziani E, Pesenti E, Alzani R. Histol Histopalhol. 2009 24, 879-891.
(32) Naidu M D I, Mason J M, Pica R V, Fung H, Peña L A. J Radiat Res. 2010; 51, 393-404.
(33) Bourbeau D I, Lavoie G, Nalbantoglu J, Massie B. J Gene Med. 2004 6, 1320-32.
(34) [Rautio, J.; Kumpulainen, H.; Heimbach, T.; Oliyai, R.; Oh, D.; Järvinen, T.; Savolainen, J. Nat. Rev. 2008 7, 255-270.
(35) Huttunen, K. M.; Raunio, H.; Rautio, J. Pharmacol. Rev. 2011 63, 750-771.
(36) Vignaroli, G.; Zamperini, C.; Dreassi, E.; Radi, M.; Angelucci, A.; Sanità, P.; Crespan, E.; Kissova, M.; Maga, G.; Schenone, S.; Musumeci, F.; Botta, M. ACS Med. Chem. Lett. 2013 4, 622-626.
(37) Berge S. M. et al., J. Pharm. Sci. 1977, 66, 1-19; Gould P. L. Int. J. Pharm 1986, 33, 201-217; Bighley et al. Encyclopedia of Pharmaceutical Technology, Marcel Dekker Inc, New York 1996, Volume 13, page 453-497.
(38) Remington “The Science and Practice of Pharmacy”, Lippincott Williams & Wilkins, 2000.
(39) Brown M T and Cooper J A. (1996). Biochim. Biophys. Acta, 1287, 121-149 Regulation, substrates and functions of src.
(40) Goldsmith, J. F., Hall, C. G Atkinson, T. P (2002) Biochem Biophys Res Commun 298:501-504.
(41) Thomas, S. M; Brugge J. S. Annu. Rev. Cell. Dev. Bio. 1997, 13, 513-609.
(42) Hanks, S. K., Quinn, A. M Hunter, T 1988 Science 241:42-52.
(43) Rybin, V. O., Guo, J, Gertsberg, Z, Feinmark, S. J Steinbergs, S. F (2008) J Biol Chem 283:17777-17788.
(44) Salmond, R J., Filby, A, Qureshi, I, Caserta, S Zamoyska, R. Immunol. Rev. 2009 228, 9-22.
(45) Salmond, R. J.; Filby, A.; Qureshi, I.; Caserta, S.; Zamoyska, R. Immunol. Rev. 2009, 228, 9-22.
(46) S. Schenone, C. Brullo, F. Musumeci, M. Biava, F. Falchi, M. Botta Curr Med Chem 2001 18, 2921-2942.
(47) Lee, G. Biochim. Biophys. Acta, Mol. Basis Dis. 2005, 1739, 323-330.
(48) (a) Lee, G.; Thangavel, R.; Sharma, V. M.; Litersky, J. M.; Bhaskar, K.; Fang, S. M.; Do,
L. H.; Andreadis, A.; Van Hoesen, G.; Ksiezak-Reding, H. J. Neurosci. 2004, 24, 2304-2312. (b) Nygaard, H. B.; van Dyck, C. H.; Strittmatter, S. M. Alzheimer's Research & Therapy 2014, 6, 8.
(49) Kai Yang Jillian Belrose Catherine H. Trepanier, Gang Lei, Michael F. Jackson and John F. MacDonald Journal of Alzheimer's Disease 27 (2011) 243-252.
(50) Yoshihito D. Saito M D, ana R. Jensen B S, Ravi Salgia M D, Edwin M Posadas M D. Cancer 2010 7:1629-1637.
(51) Kostic, A.; Lynch, C. D.; Sheetz, M. P. PLoS One 2009, 4, e6361.
(52) Huang, R. Y. J.; Wang, S. M.; Hsieh, C. Y.; Wu, J. C. Int. J. Cancer 2008, 123, 801-809.
(53) Posadas, E. M.; Al-Ahmadie, H.; Robinson, V. L.; Jagadeeswaran, R.; Otto, K.; Kasza, K. E.; Tretiakov, M.; Siddiqui, J.; Pienta, K. J.; Stadler, W. M.; Rinker-Schaeffer, C.; Salgia, R. BJU Int. 2009, 103, 171-177].
(54) Chen, Z. Y.; Cai, L.; Zhu, J.; Chen, M.; Chen, J.; Li, Z. H.; Liu, X. D.; Wang, S. G.; Bie, P.; Jiang, P.; Dong, J. H.; Li, X. W. Carcinogenesis 2011, 32, 1419-1426.
(55) Eguchi, R.; Kubo, S.; Takeda, H.; Ohta, T.; Tabata, C.; Ogawa, H.; Nakano, T.; Fujimori, Y. Carcinogenesis 2012, 33, 969-975.
(56) Singh M M, Howard A, Irwin M E, Gao Y, Lu X, Multani A, Chandra J PLoS One 2012, 7:e51611.
(57) ban k, gao Y, Amin H M, Howard A, Miller C, Lin Q, Leng X, Munsell M, Bar-Eli M, Arlinghaus R B, Chandra J, 2008 Blood 111:2904-2908.
(58) Michalczyk, A.; Kliiter, S.; Rode, H. B.; Simard, J. R.; Griitter, C.; Rabiller, M.; Rauh, D. Bioorg. Med. Chem. 2008, 16, 3482-3488.
(59) Kabsch, W. J. Appl. Cryst. 1993, 26, 795-800.
(60) Zeng, X.; Gipson, B.; Zheng, Z. Y.; Renault, L.; Stahlberg, H. J. Struct. Biol. 2007, 160, 353-361.
(61) Read, R. J. Acta Crystallogr. D Biol. Crystallogr. 2001, 57, 1373-1382.
(62) Seeliger, M. A.; Nagar, B.; Frank, F.; Cao, X.; Henderson, M. N.; Kuriyan, J. Structure 2007, 15, 299-311.
(63) Emsley, P.; Cowtan, K. Acta Crystallogr. D Biol. Crystallogr. 2004, 60, 2126-2132.
(64) Brünger, A. T.; Adams, P. D.; Clore, G. M.; DeLano, W. L.; Gros, P.; Grosse-Kunstleve, R. W.; Jiang, J. S.; Kuszewski, J.; Nilges, M.; Parmu, N. S.; Read, R. J.; Rice, L. M.; Simonson, T.; Warren, G. L. Acta Crystallogr. D Biol. Crystallogr. 1998, 54, 905-921.
(65) Murshudov, G. N.; Vagin, A. A.; Dodson, E. J. D Biol. Crystallogr. 1997, 53, 240-255.
(66) Schüttelkopf, A. W.; van Aalten, D. M. Acta Crystallogr. D Biol. Crystallogr. 2004, 60, 1355-1363.
(67) Laskowski, R. A.; MacArthur, M. W.; Moss, D. S.; Thornton, J. M. J. Appl. Crystallogr. 1993, 26, 283-291.
(68) Prime, v3.1, Schrodinger, LLC, New York, N.Y., 2012.
(69) Jacobson, M. P.; Pincus, D. L.; Rapp, C. S.; Day, T. J. F.; Honig, B.; Shaw, D. E.; Friesner, R. A. A. Proteins: Structure, Function and Bioinformatics 2004, 55, 351-367.
(70) Zeevaart, J. G.; Wang, L.; Thakur, V. V.; Leung, C. S.; Tirado-Rives, J.; Bailey, C. M.; Domaoal, R. A.; Anderson, K. S.; Jorgensen, W. L. J. Am. Chem. Soc. 2008, 130, 9492-9499.
(71) Leung, C. S.; Zeevaart, J. G.; Domaoal, R. A.; Bollini, M.; Thakur, V. V.; Spasov, K.; Anderson, K. S.; Jorgensen, W. L. Bioorg. Med. Chem. Lett. 2010, 20, 2485-2488.
(72) Jorgensen, W. L.; Tirado-Rives, J. Comput. Chem. 2005, 26, 1689-1700.
(73) Jorgensen, W. L.; Schyman, P. J. Chem. Theory Comput. 2012, 8, 3895-3801.
(74) Radi, M.; Dreassi, E.; Brullo, C.; Crespan, E.; Tintori, C.; Bernardo, V.; Valoti, M.; Zamperini, C.; Daigl, H.; Musumeci, F.; Carraro, F.; Naldini, A.; Filippi, I.; Maga, G.; Schenone, S.; Botta, M. J. Med. Chem. 2011, 54, 2610-2626.
(75) Xu, W.; Doshi, A.; Lei, M.; Eck, M. J.; Harrison, S. C. Cell 1999, 3, 629-638.
(76) Cowan-Jacob, S. W.; Fendrich, G.; Manley, P. W.; Jahnke, W.; Fabbro, D.; Liebetanz, J.; Meyer T. Structure 2005, 13, 861-871.
(787) (a) Simard, J. R.; Kliiter, S.; Griitter, C.; Getlik, M.; Rabiller, M.; Rode, H. B.; Rauh, D. Nat. Chem. Biol. 2009, 5, 394-396. (b) Getlik, M.; Gritter, C.; Simard, J. R.; Kliter, S.; Rabiller, M.; Rode, H. B.; Robubi, A.; Rauh, D. J. Med. Chem. 2009, 52, 3915-3926.
(78) Jorgensen, W. L.; Thomas, L. T. J. Chem. Theory Comput. 2008, 4, 869-876.
(79) Sobol, E.; Bialer, M. Biopharm Drug Dispos. 2004.

1. A compound of formula I 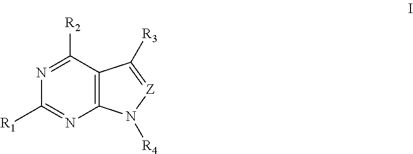
or a stereoisomer or a prodrug or a pharmaceutically acceptable salt thereof,
wherein Z represents CH or N;
R₁ represents alkyl chain with the formula: 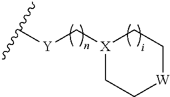
where Y is NH or O or S; X is CH or N; W is NH or NCH₃ or O; n is an integer from 0 to 4; i is an integer from 0 to 1;
or: 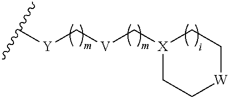
where Y is NH or O or S; V is cyclopropyl or cyclopentyl or cyclohexyl; X is CH or N; W is NH or NCH₃ or O; m is an integer from 0 to 2; i is an integer from 0 to 1;
or: 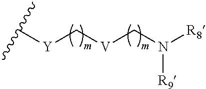
where Y is NH or O or S; V is cyclopropyl or cyclopentyl or cyclohexyl; R₈′ and R₉′ are independently H or CH₃; m is an integer from 0 to 2; i is an integer from 0 to 1;
or: 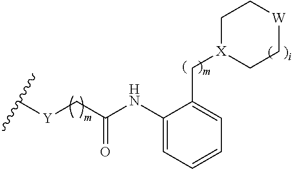
where Y is NH or O or S; X is CH or N; W is NH or NCH₃ or O; m is an integer from 0 to 2; i is an integer from 0 to 1;
R₂ represents NR₁₀′R₁₁′;
R₁₀′ and R₁₁′ are independently H, alkyl, cycloalkyl, 1-pyrrolidinyl, 4-morpholinyl, 1-hexahydroazepinyl;
or an aralkyl with the formula: 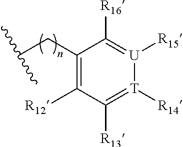
where T and U are independently C or N;
R₁₂′, R₁₃′, R₁₄′, R₁₅′, R₁₆′ are independently H, C₁₋₆ alkyl, C₂₋₆ alkenyl, C₂₋₆ alkynyl, aryl unsubstituted or substituted group, halo, haloalkyl, OCH₃, NO₂, CN, CONH₂, CONH—C₁₋₆ alkyl, CON(C₁₋₆ alkyl)₂, NH₂, NH—C₁₋₆ alkyl, N(C₁₋₆ alkyl)₂, NHC(O)alkyl, NHSO₂C₁₋₆ alkyl, SO₂NH₂, SO₂NHC₁₋₆ alkyl, SO₂N(C₁₋₆ alkyl)₂, OQ′ or SQ′ where Q′ is H, or alkyl unsubstituted or substituted group, or aryl unsubstituted or substituted group, or aralkyl unsubstituted or substituted group, n is an integer from 0 to 4;
or: 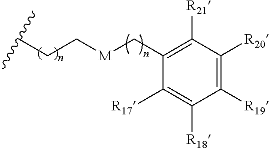
where M is NH or S or O;
R₁₇′, R₁₈′, R₁₉′, R₂₀′, R₂₁′ are independently H, C₁₋₆ alkyl, C₂₋₆ alkenyl, C₂₋₆ alkynyl, aryl unsubstituted or substituted group, halo, haloalkyl, OCH₃, NO₂, CN, CONH₂, CONH—C₁₋₆ alkyl, CON(C₁₋₆ alkyl)₂, NH₂, NH—C₁₋₆ alkyl, N(C₁₋₆ alkyl)₂, NHC(O)alkyl, NHSO₂C₁₋₆ alkyl, SO₂NH₂, SO₂NHC₁₋₆alkyl, SO₂N(C₁₋₆ alkyl)₂, OQ′ or SQ′ where Q′ is H, or alkyl unsubstituted or substituted group, or aryl unsubstituted or substituted group, or aralkyl unsubstituted or substituted group; n is an integer from 0 to 4;
R₃ represents H
or an aralkyl with the formula: 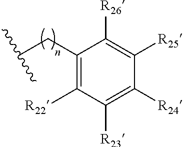
where R₂₂′, R₂₃′, R₂₄′, R₂₅′, R₂₆′ are independently H, C₁₋₆ alkyl, C₂₋₆ alkenyl, C₂₋₆ alkynyl, aryl unsubstituted or substituted group, halo, haloalkyl, OCH₃, NO₂, CN, CONH₂, CONH—C₁₋₆ alkyl, CON(C₁₋₆ alkyl)₂, NH₂, NH—C₁₋₆ alkyl, N(C₁₋₆ alkyl)₂, NHC(O)alkyl, NHCONH—C₁₋₆ alkyl, NHSO₂C₁₋₆alkyl, SO₂NH₂, SO₂NHC₁₋₆ alkyl, SO₂N(C₁₋₆ alkyl)₂, OQ′ or SQ′ where Q′ is H, or alkyl unsubstituted or substituted group, or aryl unsubstituted or substituted group, or aralkyl unsubstituted or substituted group; n is an integer from 0 to 4;
or: 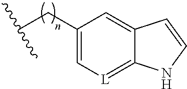
where L is CH or N; n is an integer from 0 to 4;
R represents: 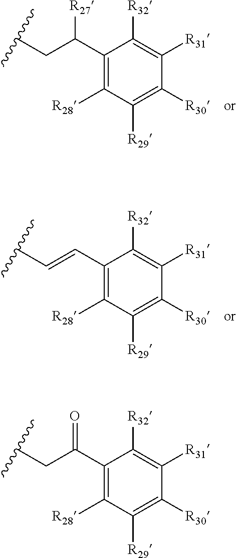
where R₂₇′ represents H, CH₃, CF₃, F, Cl, Br, OH; OMe, O-alkyl, alkyl;
where R₂₈′, R₂₉′, R₃₀′, R₃₁′, R₃₂′ are independently H, C₁₋₆ alkyl, C₂₋₆ alkenyl, C₂₋₆ alkynyl, aryl unsubstituted or substituted group, halo, haloalkyl, OCH₃, NO₂, CN, CONH₂, CONH—C₁₋₆ alkyl, CON(C₁₋₆ alkyl)₂, NH₂, NH—C₁₋₆ alkyl, N(C₁₋₆ alkyl)₂, NHC(O)alkyl, NHSO₂C₁₋₆ alkyl, OQ′ or SQ′ where Q′ is H, or alkyl unsubstituted or substituted group, or aryl unsubstituted or substituted group, or aralkyl unsubstituted or substituted group; SO₂NH₂, SO₂NHC₁₋₆ alkyl, SO₂N(C₁₋₆ alkyl)₂, SO₂H, SO₂CH₃, PO₂, PO(CH₃)₂, POHCH₃, POH₂, SO₂J where J is: 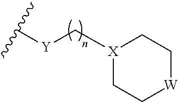
where Y is NH or O or S; X is CH or N; W is NH or NCH₃ or O; n is an integer from 0 to 4;
with the provisio that compounds:
1-(2-chloro-2-phenylethyl)-6-((2-morpholinoethyl)thio)-N-propyl-1H-pyrazolo[3,4-d]pyrimidin-4-amine (Si109);
N-benzyl-1-(2-chloro-2-phenylethyl)-6-((2-morpholinoethyl)thio)-1H-pyrazolo[3,4-d]pyrimidin-4-amine (Si110);
1-(2-chloro-2-phenylethyl)-N-(4-fluorobenzyl)-6-((2-morpholinoethyl)thio)-1H-pyrazolo[3,4-d]pyrimidin-4-amine (Si180);
1-(2-chloro-2-phenylethyl)-6-((2-morpholinoethyl)thio)-N-phenethyl-1H-pyrazolo[3,4-d]pyrimidin-4-amine (Si182);
1-(2-chloro-2-phenylethyl)-N-(3-chlorophenyl)-6-((2-morpholinoethyl)thio)-1H-pyrazolo[3,4-d]pyrimidin-4-amine (Si181);
1-(2-chloro-2-phenylethyl)-6-((2-morpholinoethyl)thio)-N-phenyl-1H-pyrazolo[3,4-d]pyrimidin-4-amine (Si192);
N-cyclohexyl-6-(2-morpholinoethoxy)-1-phenethyl-1H-pyrazolo[3,4-d]pyrimidin-4-amine (Sv12);
N⁴-(3-chlorophenyl)-N⁶-(2-morpholinoethyl)-1-phenethyl-1H-pyrazolo[3,4-d]pyrimidine-4,6-diamine (Sv24);
2-(4-methylpiperazin-1-yl)ethyl butyl(1-(2-chloro-2-phenylethyl)-6-(ethylthio)-1H-pyrazolo[3,4-d]pyrimidin-4-yl)carbamate (proSi20);
2-(4-methylpiperazin-1-yl)ethyl (3-bromophenyl)(6-(methylthio)-1-(2-phenylpropyl)-1H-pyrazolo[3,4-d]pyrimidin-4-yl)carbamate (proSi278);
1-(2-chloro-2-phenylethyl)-N-(3-chlorobenzyl)-6-(3-morpholinopropyl)-1H-pyrazolo[3,4-d]pyrimidin-4-amine;
and compounds of formula A 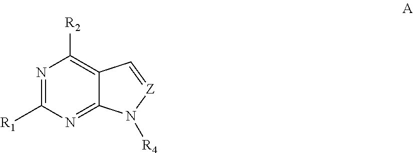
wherein when Z═N, R₁═SCH₂CH₂-4-morpholinyl and R₂ is NHCH₂CH₂C₆H₅, NHCH₂C₆H₅, NHC₆H₄mCl, 1-hexahydroazepinyl, NHC₃H₇, 4-morpholinyl or NHCH₂C₆H₄pCl are excluded.

2. The compound according to claim 1 wherein Z is N, and/or R₁ is SCH₂CH₂4-morpholinyl and/or R₂ is NHC₆H₅ or NHC₆H₄mCl or NHC₆H₄mF or NHC₆H₄mBr or NHC₆H₄mOH and/or R₃ is H and/or R₄ is CH₂CH₂C₆H₅ or CH₂CHClC₆H₅ or CH₂CHMeC₆H₅ or CH₂CH₂C₆H₄pF.

3. The compound according to claim 1 being:
N-(3-Chlorophenyl)-6-[(2-morpholin-4-ylethyl)thio]-1-(2-phenylethyl)-1H-pyrazolo[3,4-d]pyrimidin-4-amine (Si303);
1-(2-chloro-2-phenylethyl)-N-(2-fluorobenzyl)-6-((2-morpholinoethyl)thio)-1H-indazol-4-amine (Si304);
6-[(2-Morpholin-4-ylethyl)thio]-N-phenyl-1-(2-phenylpropyl)-1H-pyrazolo[3,4-d]pyrimidin-4-amine (Si313);
N-(3-Fluorophenyl)-6-[(2-morpholin-4-ylethyl)thio]-1-(2-phenylpropyl)-1H-pyrazolo[3,4-d]pyrimidin-4-amine (Si314);
N-(3-Chlorophenyl)-6-[(2-morpholin-4-ylethyl)thio]-1-(2-phenylpropyl)-1H-pyrazolo[3,4-d]pyrimidin-4-amine (Si307);
N-(3-Chlorophenyl)-1-[2-(4-fluorophenyl)ethyl]-6-[(2-morpholin-4-ylethyl)thio]-1H-pyrazolo[3,4-d]pyrimidin-4-amine (Si327);
N-(3-Bromophenyl)-1-(2-chloro-2-phenylethyl)-6-[(2-morpholin-4-ylethyl)thio]-1H-pyrazolo[3,4-d]pyrimidin-4-amine (Si306);
3-{[6-[(2-Morpholin-4-ylethyl)thio]-1-(2-phenylethyl)-1H-pyrazolo[3,4-d]pyrimidin-4-yl]amino}phenol hydrochloride (Si332);
3-{[6-[(2-Morpholin-4-ylethyl)thio]-1-(2-phenylpropyl)-1H-pyrazolo[3,4-d]pyrimidin-4-yl]amino}phenol hydrochloride (Si329);
1-(2-Chloro-2-phenylethyl)-3-(4-fluorophenyl)-1H-pyrazolo[3,4-d]pyrimidin-4-amine (Si310);
3-(4-Chlorophenyl)-1-(2-chloro-2-phenylethyl)-1H-pyrazolo[3,4-d]pyrimidin-4-amine (Si308);
1-(2-Chloro-2-phenylethyl)-3-(4-methylphenyl)-1H-pyrazolo[3,4-d]pyrimidin-4-amine (Si309);
1-(2-Chloro-2-phenylethyl)-3-(4-methyoxyphenyl)-1H-pyrazolo[3,4-d]pyrimidin-4-amine (Si311);
1-(2-Chloro-2-phenylethyl)-3-phenyl-1H-pyrazolo[3,4-d]pyrimidin-4-amine hydrochloride (Si244);
3-Phenyl-1-(2-phenylpropyl)-1H-pyrazolo[3,4-d]pyrimidin-4-amine (Si312);
1-{4-[4-Amino-1-(2-phenylpropyl)-1H-pyrazolo[3,4-d]pyrimidin-3-yl]phenyl}ethanone (Si336);
3-(4-Chlorophenyl)-1-(2-phenylpropyl)-1H-pyrazolo[3,4-d]pyrimidin-4-amine (Si337);
3-(4-Methylphenyl)-1-(2-phenylpropyl)-1H-pyrazolo[3,4-d]pyrimidin-4-amine (Si338);
3-(1H-indol-5-yl)-1-(2-phenylpropyl)-1H-pyrazolo[3,4-d]pyrimidin-4-amine (Si339);
N-benzyl-6-(sec-butylthio)-1-(2-chloro-2-phenylethyl)-1H-pyrazolo[3,4-d]pyrimidin-4-amine (Si146);
6-(Sec-butylthio)-1-(2-chloro-2-phenylethyl)-N-(2-phenylethyl)-1H-pyrazolo[3,4-d]pyrimidin-4-anine (Si147);
1-(2-Chloro-2-phenylethyl)-6-(cyclopentylthio)-N-(3-fluorophenyl)-1H-pyrazolo[3,4-d]pyrimidin-4-amine (Si170);
6-(Sec-butylthio)-N-(3-chlorophenyl)-1-(2-chloro-2-phenylethyl)-1H-pyrazolo[3,4-d]pyrimidin-4-amine (Si148);
Synthesis of 2-(4-benzylamino-1-styryl-1H-pyrazolo[3,4-d]pyrimidin-6-ylamino)-ethanol (Si74);
N-[2-(3-chlorophenyl)ethyl]-6-(methylthio)-1-[2-phenylvinyl]-1H-pyrazolo[3,4-d]pyrimidin-4-amine (Si215);
N,6-dibenzyl-1-(2-chloro-2-phenylethyl)-1H-pyrazolo[3,4-d]pyrimidin-4-amine (Si164);
or a stereoisomer or a pharmaceutically acceptable salt thereof.

4. The prodrug of the compound of formula I according to claim 1 to 3, wherein said prodrug is a prodrug of formula II 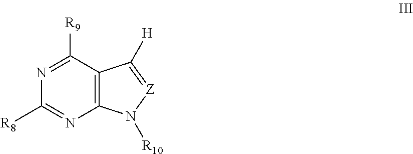
wherein Z represents CH or N;
R₈ represents H, alkylthio, alkylamino, cycloalkyl, cycloalkylthio, cycloalkylamino, alkyl, S(CH₂)ₚOH, S(CH₂)ₚNH₂, S(CH₂)ₚNHCH₃, S(CH₂), N(CH₃)₂, NH(CH₂)ₚOH, NH(CH₂)ₚNH₂; NH(CH₂)ₚNHCH₃, NH(CH₂)ₚNH(CH₃)₂; p is an integer from 0 to 6;
or an alkyl chain with the formula: 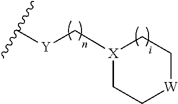
where Y is NH or O or S; X is CH or N; W is NH or NCH₃ or O; n is an integer from 0 to 4; i is an integer from 0 to 1;
or: 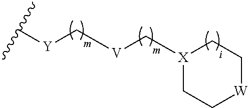
where Y is NH or O or S; V is cyclopropyl or cyclopentyl or cyclohexyl; X is CH or N; W is NH or NCH₃ or O; m is an integer from 0 to 2; i is an integer from 0 to 1;
or: 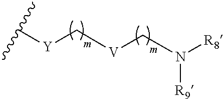
where Y is NH or O or S; V is cyclopropyl or cyclopentyl or cyclohexyl; R₈′ and R₉′ are independently H or CH₃; m is an integer from 0 to 2;
or: 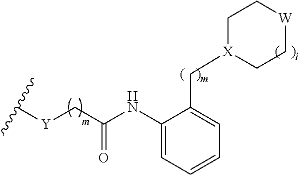
where Y is NH or O or S; X is CH or N; W is NH or NCH₃ or O; m is an integer from 0 to 2; i is an integer from 0 to 1;
R₉ represents: 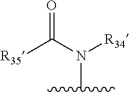
where R₃₄′ is H or alkyl or cycloalkyl or 1-pyrrolidinyl or 4-morpholinyl or 1-hexahydroazepinyl;
or an alkyl chain with the formula: 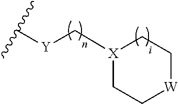
where Y is NH or O or S; X is CH or N; W is NH or NCH₃ or O; n is an integer from 0 to 4; i is an integer from 0 to 1;
or an aralkyl with the formula: 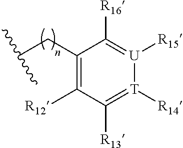
where T and U are independently C or N;
R₁₂′, R₁₃′, R₁₄′, R₁₅′, R₁₆′ are independently H, C₁₋₆ alkyl, C₂₋₆ alkenyl, C₂₋₆ alkynyl, aryl unsubstituted or substituted group, halo, haloalkyl, OCH₃, NO₂, CN, CONH₂, CONH—C₁₋₆ alkyl, CON(C₁₋₆ alkyl)₂, NH₂, NH—C₁₋₆ alkyl, N(C₁₋₆ alkyl)₂, NHC(O)alkyl, NHCONH—C₁₋₆ alkyl, NHSO₂—C₁₋₆ alkyl, SO₂NH₂, SO₂NHC₁₋₆ alkyl, SO₂N(C₁₋₆ alkyl)₂, OQ′ or SQ′ where Q′ is H, or alkyl unsubstituted or substituted group, or aryl unsubstituted or substituted group, or aralkyl unsubstituted or substituted group; n is an integer from 0 to 4;
or: 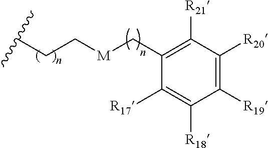
where M is NH or S or O;
R₁₇′, R₁₈′, R₁₉′, R₂₀′, R₂₁′ are independently H, C₁₋₆ alkyl, C₂₋₆ alkenyl, C₂₋₆ alkynyl, aryl unsubstituted or substituted group, halo, haloalkyl, OCH₃, NO₂, CN, CONH₂, CONH—C₁₋₆ alkyl, CON(C₁₋₆ alkyl)₂, NH₂, NH—C₁₋₆ alkyl, N(C₁₋₆ alkyl)₂, NHC(O)alkyl, NHSO₂C₁₋₆ alkyl, SO₂NH₂, SO₂NHC₁₋₆-alkyl, SO₂N(C₁₋₆ alkyl)₂, OQ′ or SQ′ where Q′ is H, or alkyl unsubstituted or substituted group, or aryl unsubstituted or substituted group, or aralkyl unsubstituted or substituted group; n is an integer from 0 to 4;
where R₃₅′ is an alkyl chain with the formula: 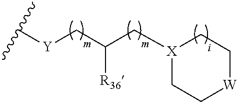
where Y is NH or O or S; R₃₆′ is H or alkyl or aryl or aralkyl; X is CH or N; W is NH or NCH₃ or O; m is an integer from 0 to 2; i is an integer from 0 to 1;
or: 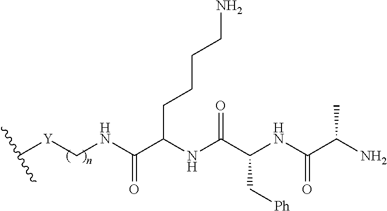
where Y is NH or O or S;
n is an integer from 0 to 4;
or: 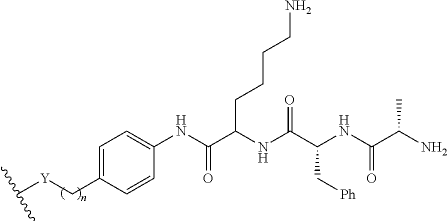
where Y is NH or O or S;
n is an integer from 0 to 4;
R₁₀ represents: 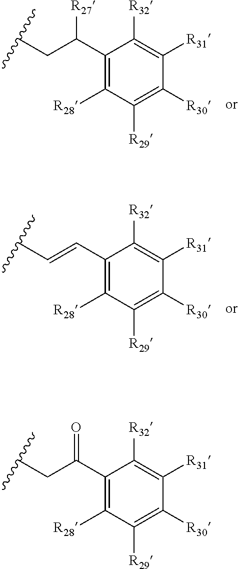
where R₂₇′ represents H, CH₃, CF₃, F, Cl, Br, OH; O-alkyl, alkyl;
where R₂₈′, R₂₉′, R₃₀′, R₃₁′, R₃₂′ are independently H, C₁₋₆ alkyl, C₂₋₆ alkenyl, C₂₋₆ alkynyl, aryl unsubstituted or substituted group, halo, haloalkyl, OCH₃, NO₂, CN, CONH₂, CONH—C₁₋₆ alkyl, CON(C₁₋₆ alkyl)₂, NH₂, NH—C₁₋₆ alkyl, N(C₁₋₆ alkyl)₂, NHC(O)alkyl, NHSO₂—C₁₋₆ alkyl, OQ′ or SQ′ where Q′ is H, or alkyl unsubstituted or substituted group, or aryl unsubstituted or substituted group, or aralkyl unsubstituted or substituted group; SO₂NH₂, SO₂NHC₁₋₆ alkyl, SO₂N(C₁₋₆ alkyl)₂, SO₂H, SO₂CH₃, PO₂, PO(CH₃)₂, POHCH₃, POH₂, SO₂J where J is: 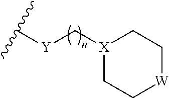
where Y is NH or O or S; X is CH or N; W is NH or NCH₃ or O; n is an integer from 0 to 4.

5. The prodrug according to claim 4 wherein Z is N and/or R₈ is H or SMe or SEt or SCH₂CH₂-4-mopholino; and/or
R₉ is 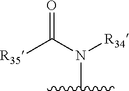
wherein R₃₄′ is CH₂C₆H₅ or CH₂C₆H₄oCl or C₆H₄mCl or C₆H₄mBr or CH₂CH₂C₆H₅ or C₆H₅ or nBu; and wherein R₃₅′ is 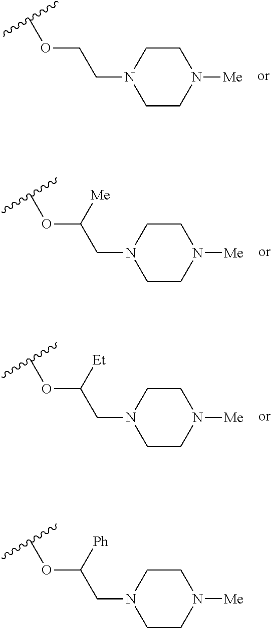
and/or R₁₀ is 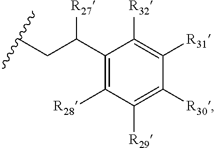
wherein R_{27′} is H or Cl or Me; R₃₀′ is H or Br; and R₂₈′, R₂₉′, R₃₁′, R₃₂′ are H.

6. The compound according to any of claims 1 to 5 for medical use.

7. The compound for use according to claim 6 for use as SFKs inhibiting medicament in the treatment and/or prevention of cancer.

8. The compound for use according to claim 7 wherein the SFK is s-Src.

9. The compound for use according to claims 7 or 8 wherein the cancer is a solid or liquid cancer, preferably the cancer is selected from the group consisting of neuroblastoma, glioblastoma, osteosarcoma, prostate cancer, hepatocellular carcinoma, leukemia, retinoblastoma, rhabdomyosarcoma, hepatocellular carcinoma, glioblastoma multiformae, squamous cell carcinoma of the head and neck, melanoma, breast cancer, ovarian cancer, pancreatic cancer, mesothelioma.

10. The compound according to any of claims 1 to 6 for use in the treatment of a neurodegenerative disease.

11. A compound or a stereoisomer or a pharmaceutically acceptable salt thereof for use in the treatment and/or prevention of a disease selected from the group consisting of: solid tumour and neurodegenerative disease wherein said compound has the formula IV: 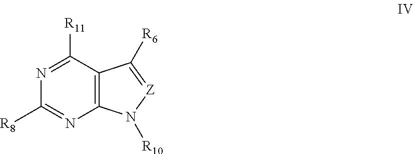
wherein:
Z represents CH or N;
R₆ represents H
or an aralkyl with the formula: 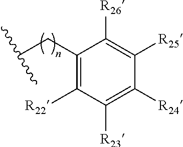
where R₂₂′, R₂₃′, R₂₄′, R₂₅′, R₂₆′ are independently H, C₁₋₆ alkyl, C₂₋₆ alkenyl, C₂₋₆ alkynyl, aryl unsubstituted or substituted group, halo, haloalkyl, OCH₃, NO₂, CN, CO(C₁₋₆ alkyl), CONH₂, CONH—C₁₋₆ alkyl, CON(C₁₋₆ alkyl)₂, NH₂, NH—C₁₋₆ alkyl, N(C₁₋₆ alkyl)₂, NHC(O)alkyl, NHCONH—C₁₋₆ alkyl, NH SO₂—C₁₋₆ alkyl, SO₂NH₂, SO₂NHC₁₋₆ alkyl, SO₂N(C₁₋₆ alkyl)₂, OQ′ or SQ′ where Q′ is H, or alkyl unsubstituted or substituted group, or aryl unsubstituted or substituted group, or aralkyl unsubstituted or substituted group; n is an integer from 0 to 4;
or: 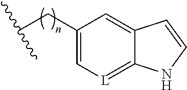
where L is CH or N; n is an integer from 0 to 4;
R₈ represents H, benzyl, alkylthio, alkylamino, cycloalkyl, cycloalkylthio, cycloalkylamino, alkyl, S(CH₂)ₚOH, S(CH₂)ₚNH₂, S(CH₂)ₚNHCH₃, S(CH₂)ₚN(CH₃)₂, NH(CH₂)ₚOH, NH(CH₂)ₚNH₂; NH(CH₂)ₚNHCH₃, NH(CH₂)ₚNH(CH₃)₂; p is an integer from 0 to 6;
or an alkyl chain with the formula: 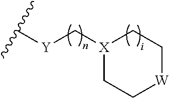
where Y is NH or O or S; X is CH or N; W is NH or NCH₃ or O;
n is an integer from 0 to 4; i is an integer from 0 to 1;
or: 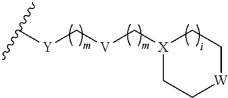
where Y is NH or O or S; V is cyclopropyl or cyclopentyl or cyclohexyl; X is CH or N; W is NH or NCH₃ or O; m is an integer from 0 to 2; i is an integer from 0 to 1;
or: 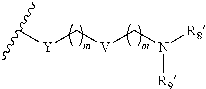
where Y is NH or O or S; V is cyclopropyl or cyclopentyl or cyclohexyl; R₈′ and R₉′ are independently H or CH₃; m is an integer from 0 to 2;
or: 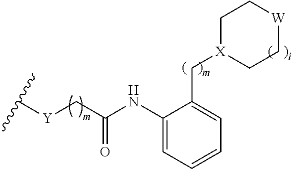
where Y is NH or O or S; X is CH or N; W is NH or NCH₃ or O; m is an integer from 0 to 2; i is an integer from 0 to 1;
R₁₀ represents 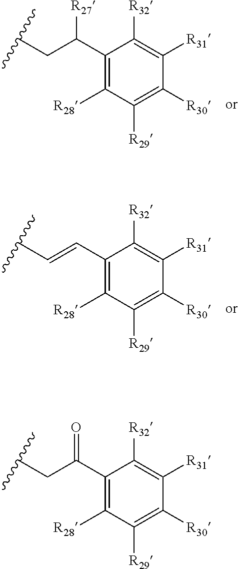
where R₂₇′ represents H, CH₃, CF₃, F, Cl, Br, OH; O-alkyl, alkyl;
where R₂₈′, R₂₉′, R₃₀′, R₃₁′, R₃₂′ are independently H, C₁₋₆ alkyl, C₂₋₆ alkenyl, C₂0.6 alkynyl, aryl unsubstituted or substituted group, halo, haloalkyl, OCH₃, NO₂, CN, CONH₂, CONH—C₁₋₆ alkyl, CON(C₁₋₆ alkyl)₂, NH₂, NH—C₁₋₆ alkyl, N(C₁₋₆ alkyl)₂, NHC(O)alkyl, NHSO₂—C₁₋₆ alkyl, OQ′ or SQ′ where Q′ is H, or alkyl unsubstituted or substituted group, or aryl unsubstituted or substituted group, or aralkyl unsubstituted or substituted group; SO₂NH₂, SO₂NHC₁₋₆ alkyl, SO₂N(C₁₋₆ alkyl)₂, SO₂H, SO₂CH₃, PO₂, PO(CH₃)₂, POHCH₃, POH₂, SO₂J where J is: 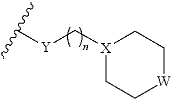
where Y is NH or O or S; X is CH or N; W is NH or NCH₃ or O; n is an integer from 0 to 4;
R₃₇′ and R₃₈′ are independently H, alkyl, cycloalkyl, 1-pyrrolidinyl, 4-morpholinyl, 1-hexahydroazepinyl;
or an aralkyl with the formula: 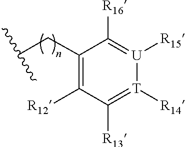
where T and U are independently C or N;
R₁₂′, R₁₃′, R₁₄′, R₁₅′, R₁₆′ are independently H, C₁₋₆ alkyl, C₂₋₆ alkenyl, C₂₋₆ alkynyl, aryl unsubstituted or substituted group, halo, haloalkyl, OCH₃, NO₂, CN, CONH₂, CONH—C₁₋₆ alkyl, CON(C₁₋₆ alkyl)₂, NH₂, NH—C₁₋₆ alkyl, N(C₁₋₆ alkyl)₂, NHC(O)alkyl, NHCONH—C₁₋₆ alkyl, NHSO₂C₁₋₆ alkyl, SO₂NH₂, SO₂NHC₁₋₆alkyl, SO₂N(C₁₋₆ alkyl)₂, OQ′ or SQ′ where Q′ is H, or alkyl unsubstituted or substituted group, or aryl unsubstituted or substituted group, or aralkyl unsubstituted or substituted group, n is an integer from 0 to 4;
or: 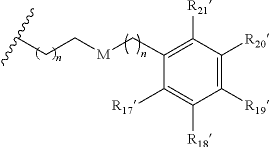
where M is NH or S or O;
R₁₇′, R₁₈′, R₁₉′, R₂₀′, R₂₁′ are independently H, C₁₋₆ alkyl, C₂₋₆ alkenyl, C₂₋₆ alkynyl, aryl unsubstituted or substituted group, halo, haloalkyl, OCH₃, NO₂, CN, CONH₂, CONH—C₁₋₆ alkyl, CON(C₁₋₆ alkyl)₂, NH₂, NH—C₁₋₆ alkyl, N(C₁₋₆ alkyl)₂, NHC(O)alkyl, NHSO₂C₁₋₆ alkyl, SO₂NH₂, SO₂NHC₁₋₆ alkyl, SO₂N(C₁₋₆alkyl)₂, OQ′ or SQ′ where Q′ is H, or alkyl unsubstituted or substituted group, or aryl unsubstituted or substituted group, or aralkyl unsubstituted or substituted group; n is an integer from 0 to 4;
or R₁₁ represents 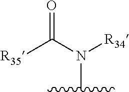
where R₃₄′ is H or alkyl or cycloalkyl or 1-pyrrolidinyl or 4-morpholinyl or 1-hexahydroazepinyl;
or an alkyl chain with the formula: 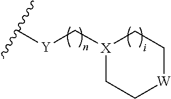
where Y is NH or O or S; X is CH or N; W is NH or NCH₃ or O; n is an integer from 0 to 4; i is an integer from 0 to 1;
or an aralkyl with the formula: 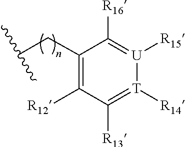
where T and U are independently C or N;
R₁₂′, R₁₃′, R₁₄′, R₁₅′, R₁₆′ are independently H, C₁₋₆ alkyl, C₂₋₆ alkenyl, C₂₋₆ alkynyl, aryl unsubstituted or substituted group, halo, haloalkyl, OCH₃, NO₂, CN, CONH₂, CONH—C₁₋₆ alkyl, CON(C₁₋₆ alkyl)₂, NH₂, NH—C₁₋₆ alkyl, N(C₁₋₆ alkyl)₂, NHC(O)alkyl, NHCONH—C₁₋₆ alkyl, NHSO₂—C₁₋₆ alkyl, SO₂NH₂, SO₂NHC₁₋₆ alkyl, SO₂N(C₁₋₆ alkyl)₂, OQ′ or SQ′ where Q′ is H, or alkyl unsubstituted or substituted group, or aryl unsubstituted or substituted group, or aralkyl unsubstituted or substituted group; n is an integer from 0 to 4;
or: 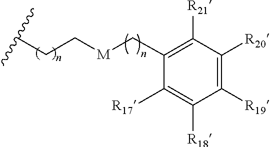
where M is NH or S or O;
R₁₇′, R₁₈′, R₁₉′, R₂₀′, R₂₁′ are independently H, C₁₋₆ alkyl, C₂₋₆ alkenyl, C₂₋₆ alkynyl, aryl unsubstituted or substituted group, halo, haloalkyl, OCH₃, NO₂, CN, CONH₂, CONH—C₁₋₆ alkyl, CON(C₁₋₆ alkyl)₂, NH₂, NH—C₁₋₆ alkyl, N(C₁₋₆ alkyl)₂, NHC(O)alkyl, NHSO₂C₁₋₆ alkyl, SO₂NH₂, SO₂NHC₁₋₆ alkyl, SO₂N(C₁₋₆ alkyl)₂, OQ′ or SQ′ where Q′ is H, or alkyl unsubstituted or substituted group, or aryl unsubstituted or substituted group, or aralkyl unsubstituted or substituted group; n is an integer from 0 to 4;
where R₃₅′ is an alkyl chain with the formula: 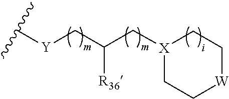
where Y is NH or O or S; R₃₆′ is H or alkyl or aryl or aralkyl; X is CH or N; W is NH or NCH₃ or O; m is an integer from 0 to 2; i is an integer from 0 to 1;
or: 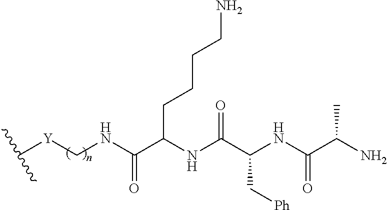
where Y is NH or O or S;
n is an integer from 0 to 4;
or: 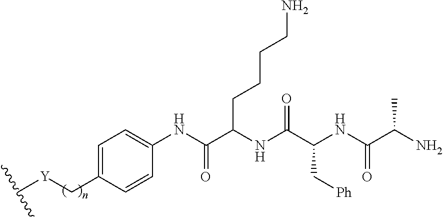
where Y is NH or O or S;
n is an integer from 0 to 4;
with the provisio that compounds:
N-(3-chlorophenyl)-6-(methylthio)-1-phenethyl-1H-pyrazolo[3,4-d]pyrimidin-4-amine (Si214);
6-(methylthio)-N-phenyl-1-(2-phenylpropyl)-1H-pyrazolo[3,4-d]pyrimidin-4-amine (Si276);
N-(3-chlorophenyl)-6-(methylthio)-1-(2-phenylpropyl)-1H-pyrazolo[3,4-d]pyrimidin-4-amine (Si277);
N-(3-bromophenyl)-6-(methylthio)-1-(2-phenylpropyl)-1H-pyrazolo[3,4-d]pyrimidin-4-amine (Si278)
N-benzyl-1-(2-chloro-2-phenylethyl)-6-(methylthio)-1H-pyrazolo[3,4-d]pyrimidin-4-amine (Si34);
1-(2-chloro-2-phenylethyl)-6-(methylthio)-N-phenethyl-1H-pyrazolo[3,4-d]pyrimidin-4-amine (Si35); and
1-(2-chloro-2-phenylethyl)-N-(3-chlorophenyl)-6-(methylthio)-1H-pyrazolo[3,4-d]pyrimidin-4-amine (Si83);
are excluded.

12. The compound for use according to claim 11 being: 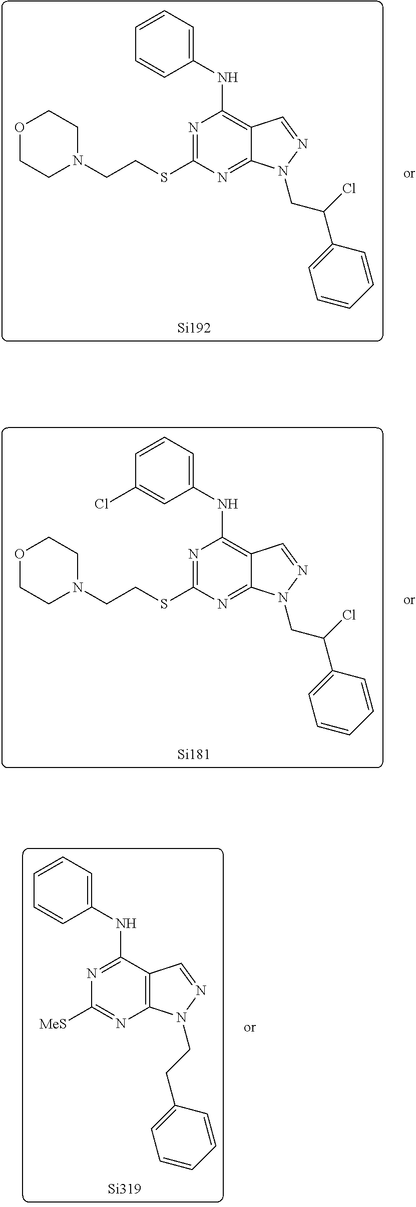 [SMILES: "Si192" ; "Si181" ; ClC(Cn1ncc2c(Nc3ccccc3)nc(SCCN3CCOCC3)nc21)c1ccccc1 ; Clc1cccc(Nc2nc(SCCN3CCOCC3)nc3c2cnn3CC(Cl)c2ccccc2)c1 ; CSc1nc(Nc2ccccc2)c2cnn(CCc3ccccc3)c2n1 ; "Si319"] 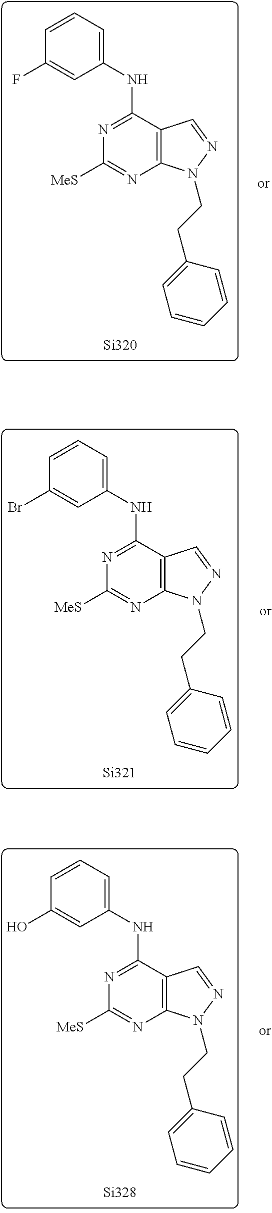 [SMILES: CSc1nc(Nc2cccc(F)c2)c2cnn(CCc3ccccc3)c2n1 ; "Si320" ; CSc1nc(Nc2cccc(Br)c2)c2cnn(CCc3ccccc3)c2n1 ; "Si321" ; CSc1nc(Nc2cccc(O)c2)c2cnn(CCc3ccccc3)c2n1 ; "Si328"] 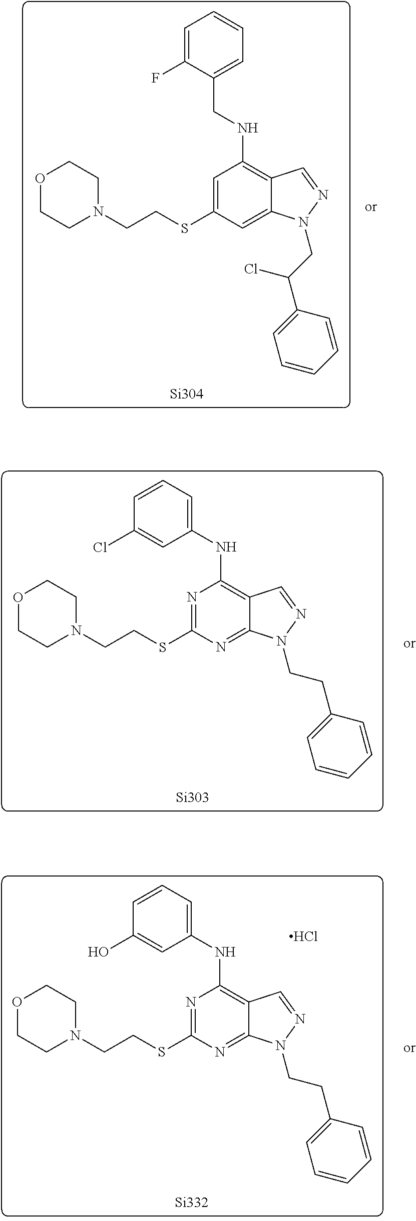 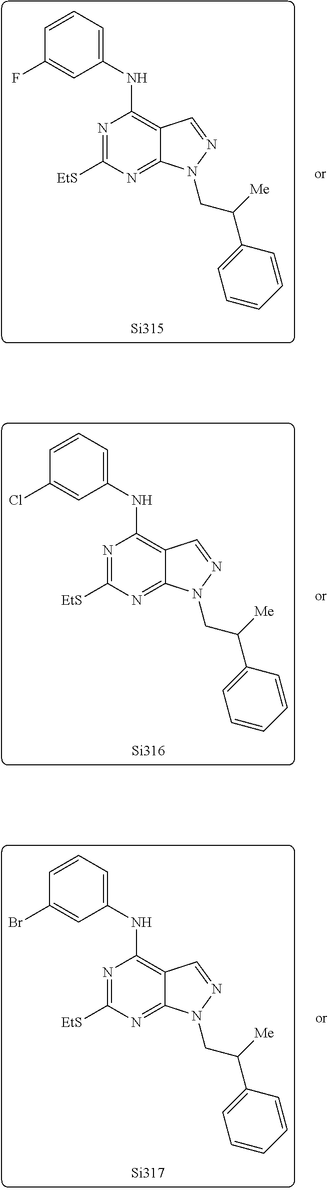 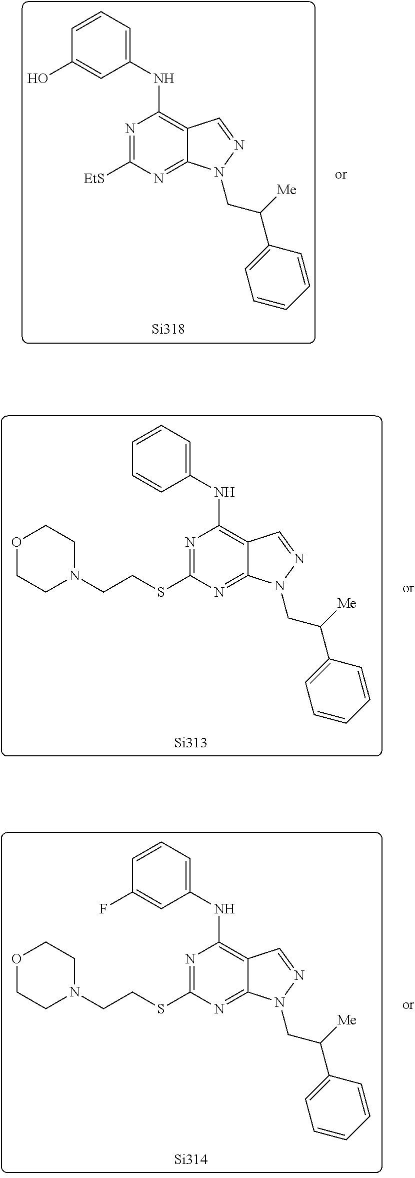 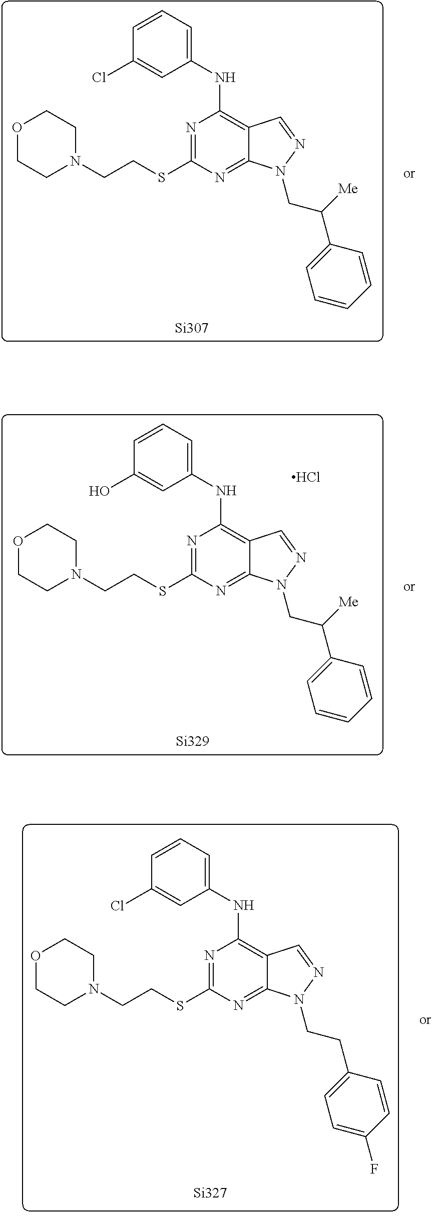 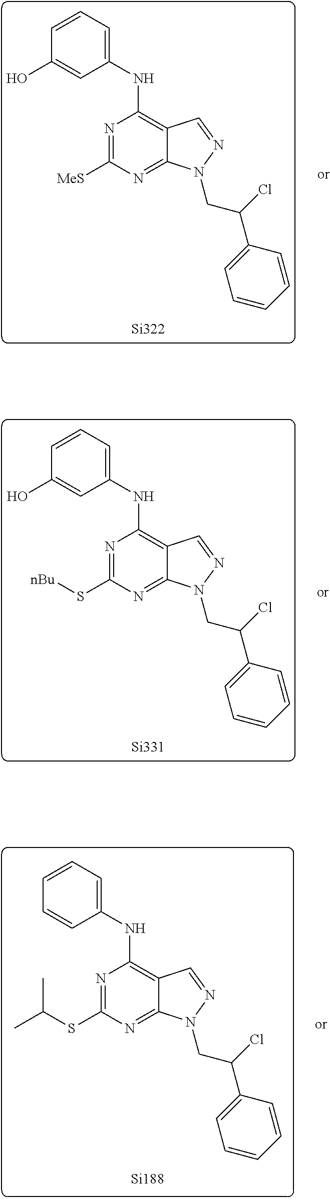 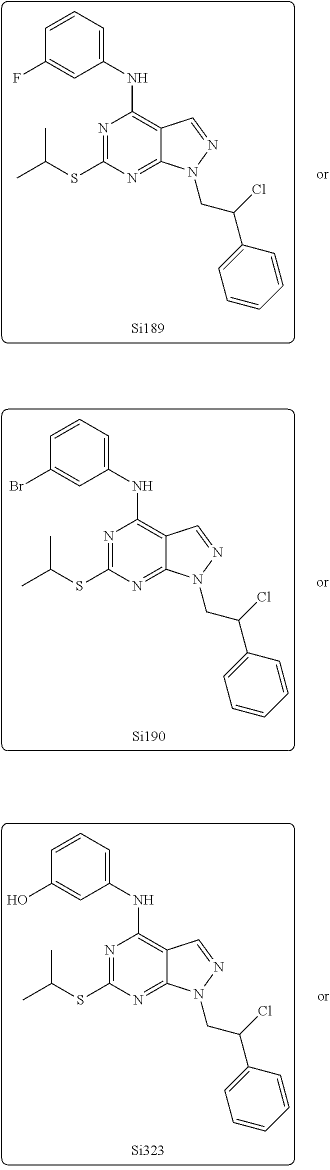 [SMILES: "Si190" ; "Si189" ; CC(C)Sc1nc(Nc2cccc(F)c2)c2cnn(CC(Cl)c3ccccc3)c2n1 ; CC(C)Sc1nc(Nc2cccc(Br)c2)c2cnn(CC(Cl)c3ccccc3)c2n1 ; CC(C)Sc1nc(Nc2cccc(O)c2)c2cnn(CC(Cl)c3ccccc3)c2n1 ; "Si323"] 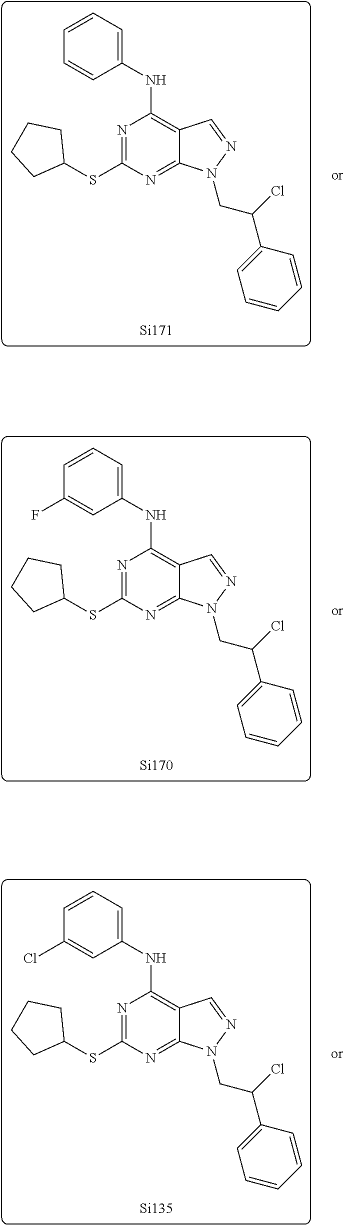 [SMILES: "Si170" ; "Si171" ; "Si135" ; ClC(Cn1ncc2c(Nc3ccccc3)nc(SC3CCCC3)nc21)c1ccccc1 ; Clc1cccc(Nc2nc(SC3CCCC3)nc3c2cnn3CC(Cl)c2ccccc2)c1 ; Fc1cccc(Nc2nc(SC3CCCC3)nc3c2cnn3CC(Cl)c2ccccc2)c1] 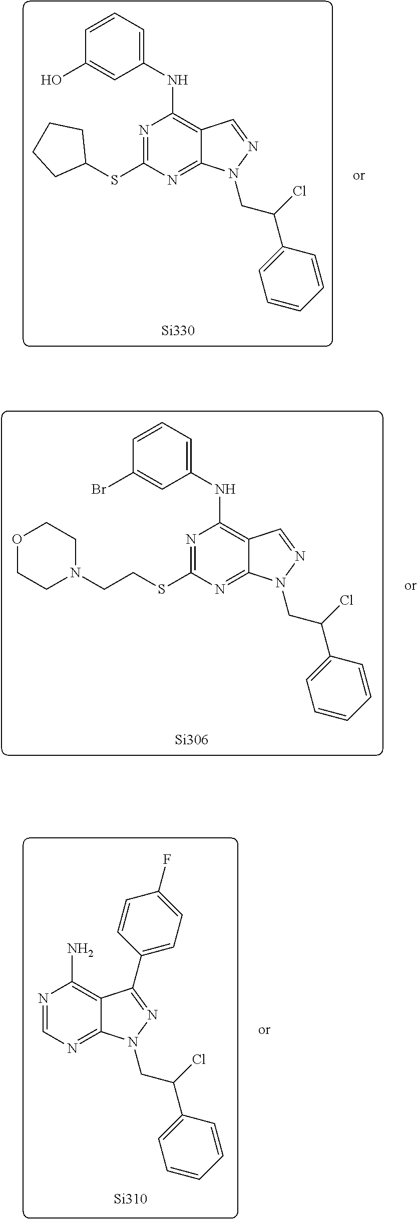 [SMILES: Oc1cccc(Nc2nc(SC3CCCC3)nc3c2cnn3CC(Cl)c2ccccc2)c1 ; "Si330" ; ClC(Cn1ncc2c(Nc3cccc(Br)c3)nc(SCCN3CCOCC3)nc21)c1ccccc1 ; "Si306" ; Nc1ncnc2c1c(-c1ccc(F)cc1)nn2CC(Cl)c1ccccc1 ; "Si310"] 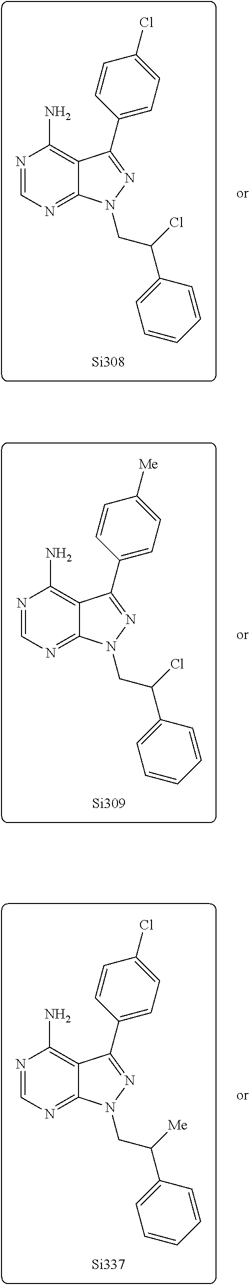 [SMILES: Nc1ncnc2c1c(-c1ccc(Cl)cc1)nn2CC(Cl)c1ccccc1 ; "Si308" ; Cc1ccc(-c2nn(CC(Cl)c3ccccc3)c3ncnc(N)c23)cc1 ; "Si309" ; CC(Cn1nc(-c2ccc(Cl)cc2)c2c(N)ncnc21)c1ccccc1 ; "Si337"] 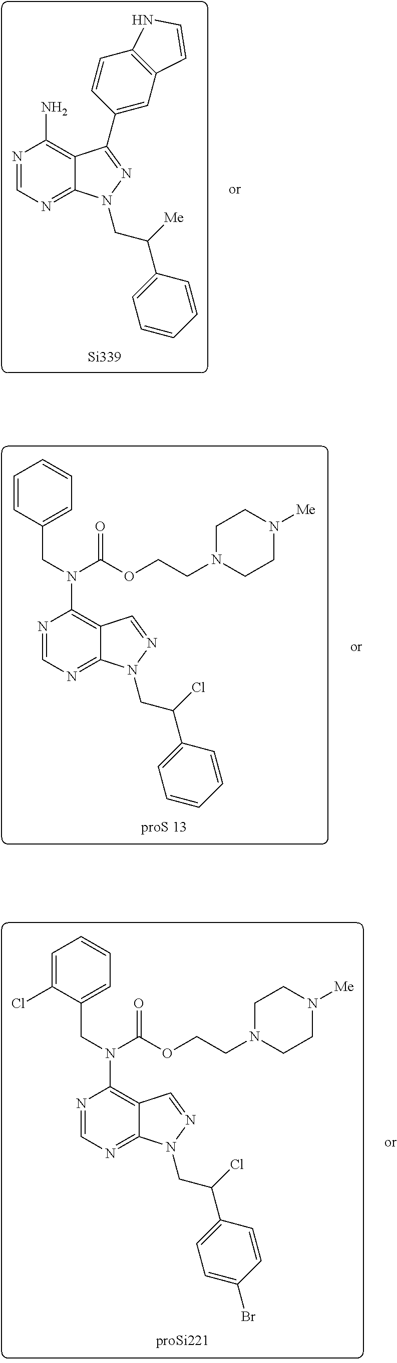 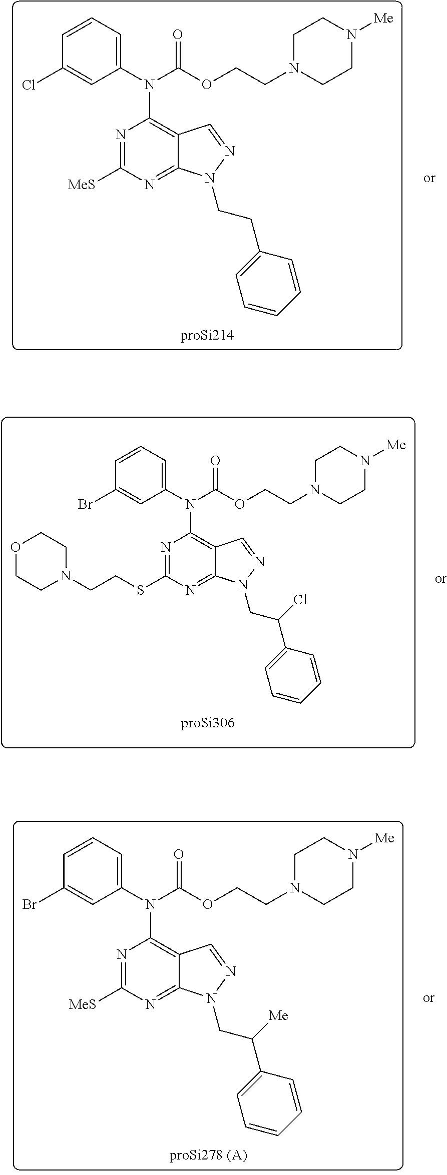 [SMILES: CSc1nc(N(C(=O)OCCN2CCN(C)CC2)c2cccc(Cl)c2)c2cnn(CCc3ccccc3)c2n1 ; "proSi214" ; CN1CCN(CCOC(=O)N(c2cccc(Br)c2)c2nc(SCCN3CCOCC3)nc3c2cnn3CC(Cl)c2ccccc2)CC1 ; "proSi306" ; CSc1nc(N(C(=O)OCCN2CCN(C)CC2)c2cccc(Br)c2)c2cnn(CC(C)c3ccccc3)c2n1 ; "proSi278 (A)"] 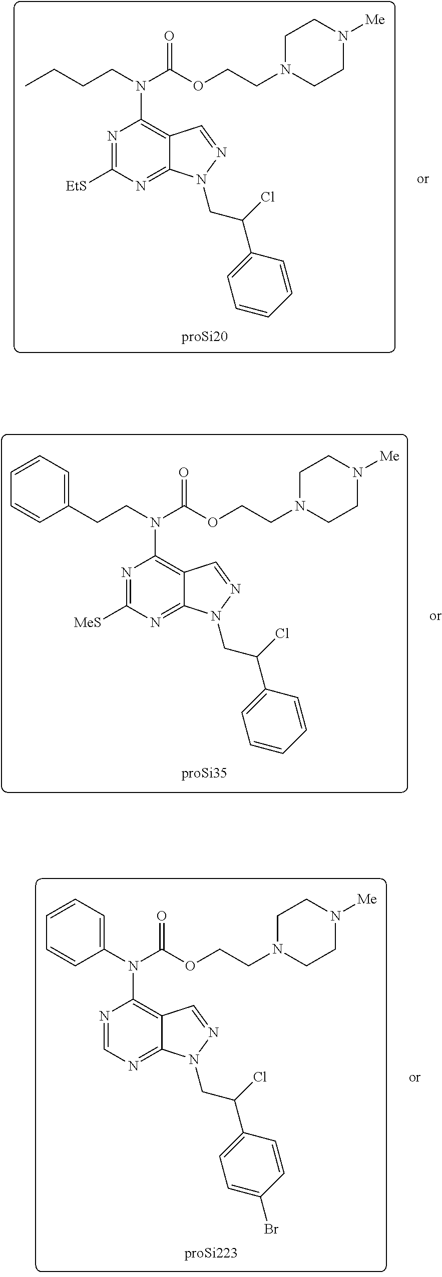 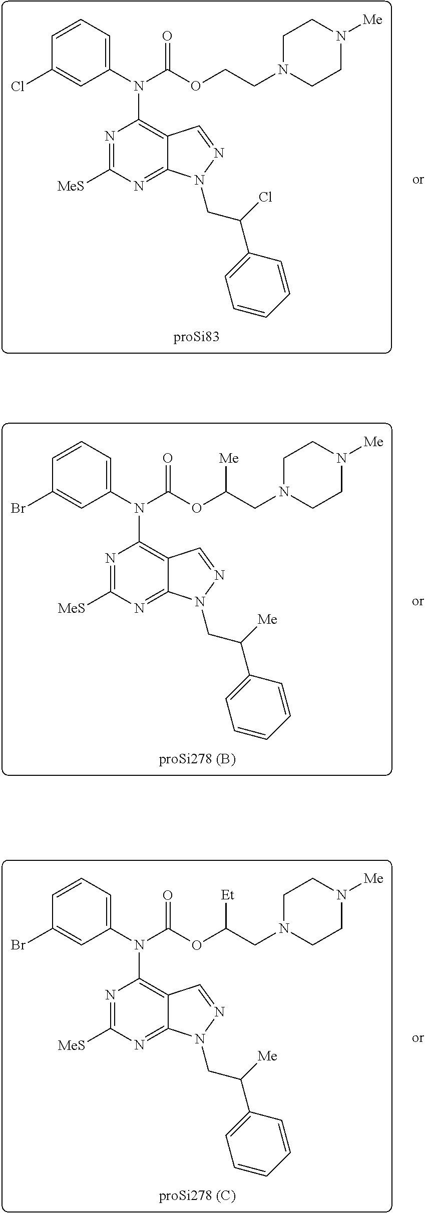 [SMILES: CSc1nc(N(C(=O)OCCN2CCN(C)CC2)c2cccc(Cl)c2)c2cnn(CC(Cl)c3ccccc3)c2n1 ; "proSi83" ; CSc1nc(N(C(=O)OC(C)CN2CCN(C)CC2)c2cccc(Br)c2)c2cnn(CC(C)c3ccccc3)c2n1 ; "proSi278 (B)" ; CCC(CN1CCN(C)CC1)OC(=O)N(c1cccc(Br)c1)c1nc(SC)nc2c1cnn2CC(C)c1ccccc1 ; "proSi278 (C)"] 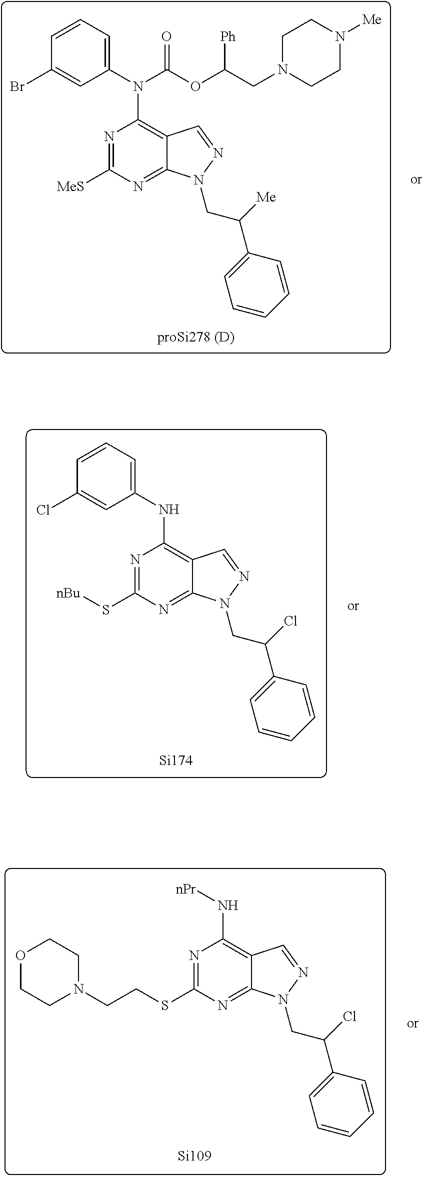 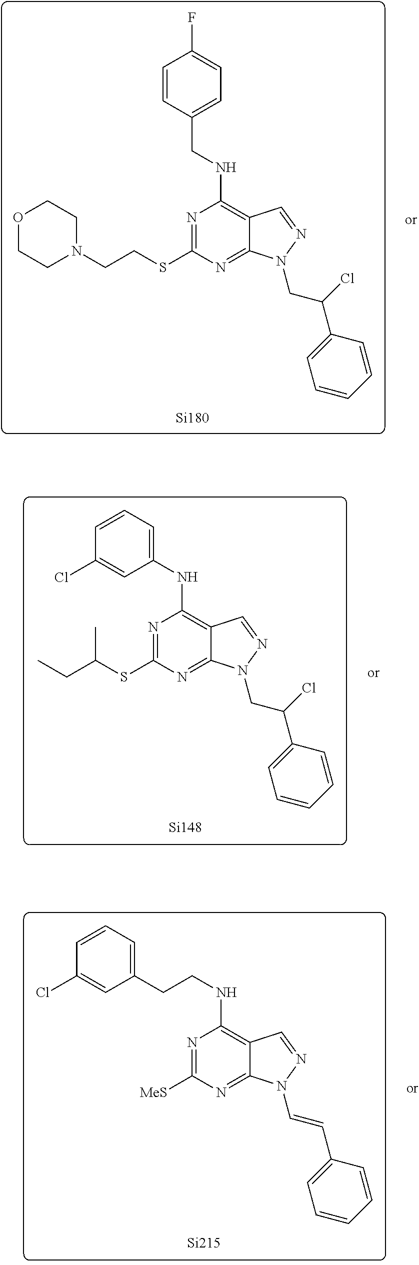 [SMILES: "Si215" ; "Si148" ; "Si180" ; Fc1ccc(CNc2nc(SCCN3CCOCC3)nc3c2cnn3CC(Cl)c2ccccc2)cc1 ; CCC(C)Sc1nc(Nc2cccc(Cl)c2)c2cnn(CC(Cl)c3ccccc3)c2n1 ; CSc1nc(NCCc2cccc(Cl)c2)c2cnn(/C=C/c3ccccc3)c2n1] 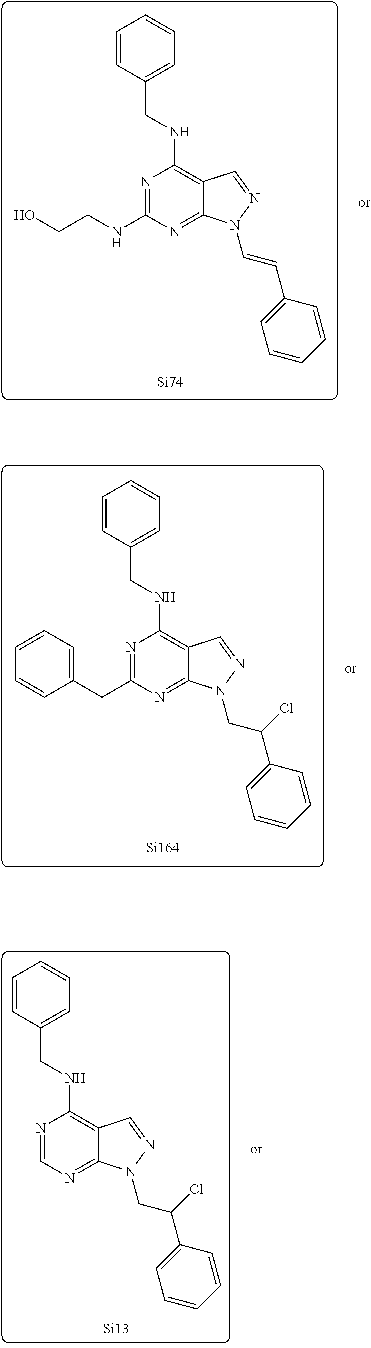 [SMILES: "Si164" ; "Si74" ; ClC(Cn1ncc2c(NCc3ccccc3)ncnc21)c1ccccc1 ; ClC(Cn1ncc2c(NCc3ccccc3)nc(Cc3ccccc3)nc21)c1ccccc1 ; OCCNc1nc(NCc2ccccc2)c2cnn(/C=C/c3ccccc3)c2n1 ; "Si13"] 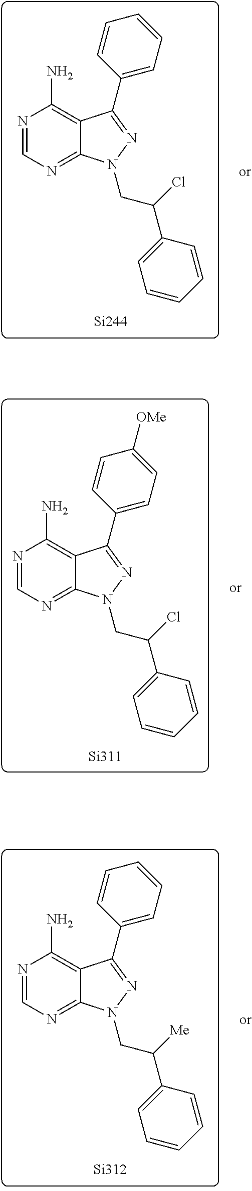 [SMILES: "Si244" ; COc1ccc(-c2nn(CC(Cl)c3ccccc3)c3ncnc(N)c23)cc1 ; Nc1ncnc2c1c(-c1ccccc1)nn2CC(Cl)c1ccccc1 ; "Si311" ; CC(Cn1nc(-c2ccccc2)c2c(N)ncnc21)c1ccccc1 ; "Si312"] 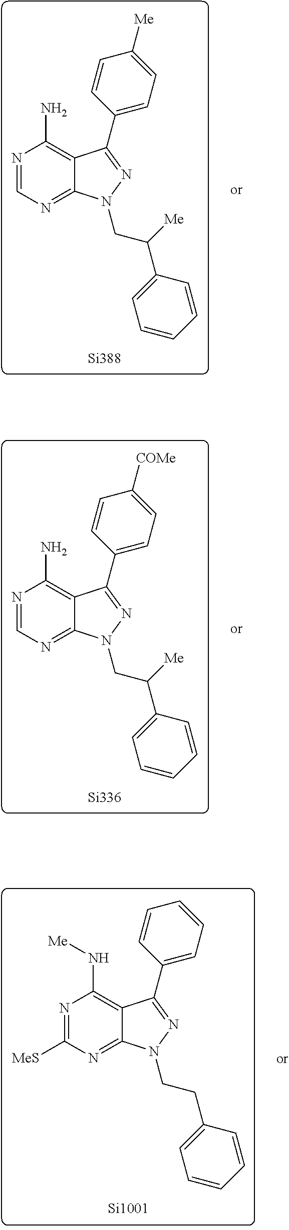 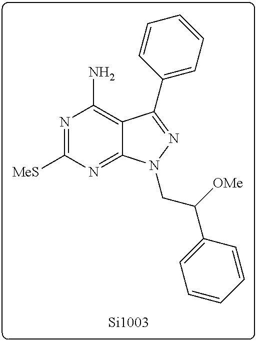 [SMILES: COC(Cn1nc(-c2ccccc2)c2c(N)nc(SC)nc21)c1ccccc1 ; "Si1003"]
or a stereoisomer or a pharmaceutically acceptable salt thereof.

13. The compound for use according to claim 11 or 12 wherein the tumour is selected from the group consisting of: neuroblastoma, glioblastoma, retinoblastoma, rhabdomyosarcoma, hepatocellular carcinoma, glioblastoma multiformae, squamous cell carcinoma of the head and neck, melanoma, breast cancer, ovarian cancer, pancreatic cancer and mesothelioma.

14. The compound according to claim 1 to 13 for use with a further anti-tumoral therapy.

15. The compound according to claim 14 wherein the further anti-tumoral therapy is selected from the group consisting of: radiotherapy and chemotherapy.

16. The compound according to claim 15 wherein the chemotherapy is selected from the group consisting of: mitomycin C, cisplatin, etoposide, vincristine, doxorubicin, isotretinoin and cyclophosphamide.

17. A pharmaceutical composition comprising a compound of the formula I or a stereoisomer or a prodrug or a pharmaceutically acceptable salt thereof as defined in claim 1 to 5 and pharmaceutically acceptable carrier.

18. The pharmaceutical composition according to claim 17 wherein the pharmaceutically acceptable carrier is selected from the group consisting of a nanoparticle such as: liposome, albumin, cyclodextrin and gold nanoparticles.

19. A process for the preparation of a prodrug of the compound of formula I as defined in claim 1, wherein said prodrug is a prodrug of formula II 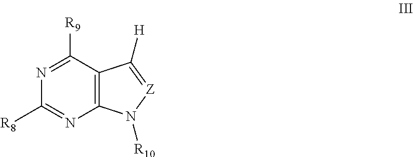
wherein
R₁₀ is 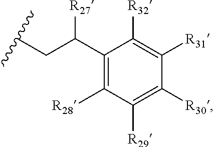
R_{28′}, R_{29′}, R_{31′} and R_{32′} are H comprising the following step: 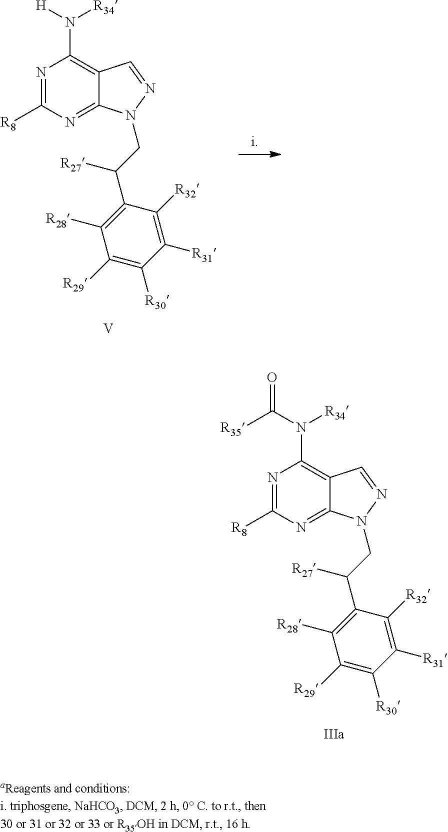
Wherein R₈, R_{27′}, R₂₈′, R₂₉′, R₃₀′, R₃₁′, R₃₂′, R_{34′} are as defined in claim 4, and
wherein R_{35′} is:
an alkyl chain with the formula: 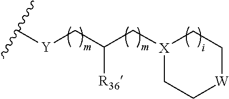
where Y is NH or O or S; R₃₆′ is H or alkyl or aryl or aralkyl; X is CH or N; W is NH or NCH₃ or O; m is an integer from 0 to 2; i is an integer from 0 to 1;
or: 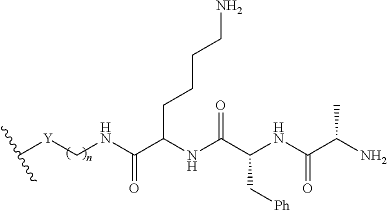
where Y is NH or O or S;
n is an integer from 0 to 4;
or: 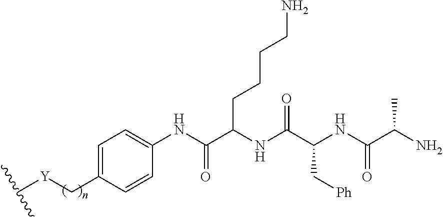
where Y is NH or O or S;
n is an integer from 0 to 4;
or a process for the preparation of a compound of formula I, said process comprising the following steps: 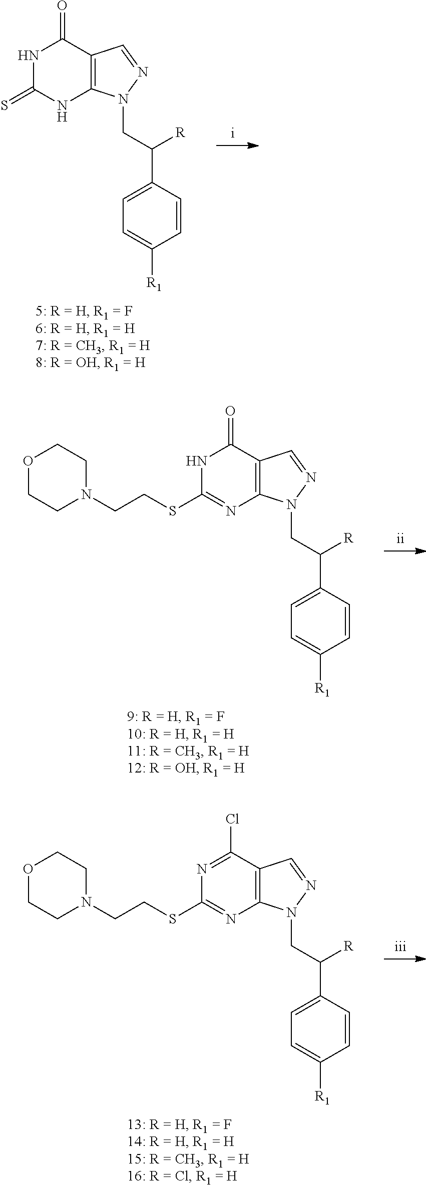 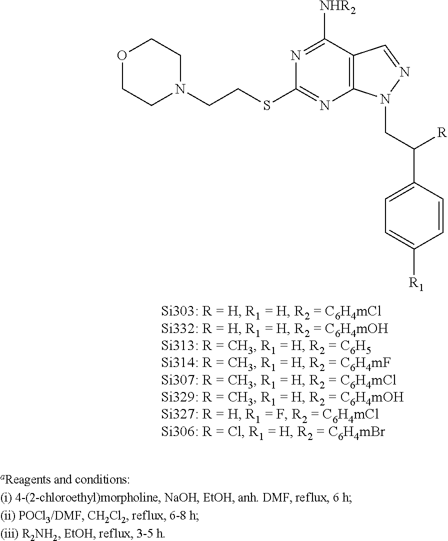
or a process for the preparation of compounds of formula IV as defined in claim 11, or salts thereof, comprising the following steps: 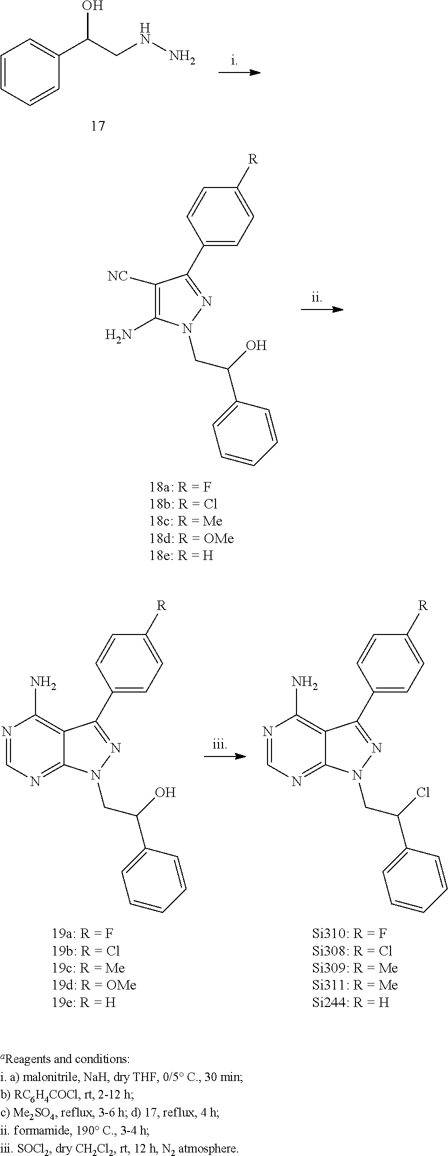
or a process for the preparation of compounds of formula IV as defined in claim 11, or salts thereof, comprising the following steps: 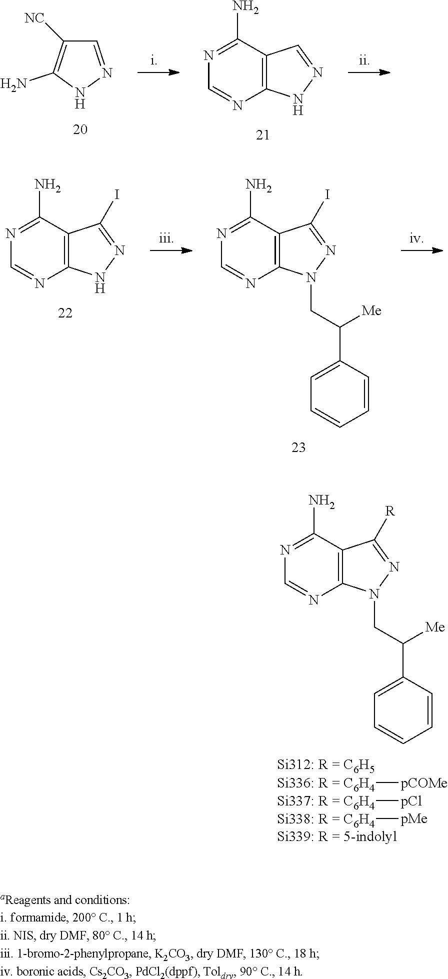
or a process for the preparation of compounds of formula IV as defined in claim 11, or salts thereof, comprising the following steps: 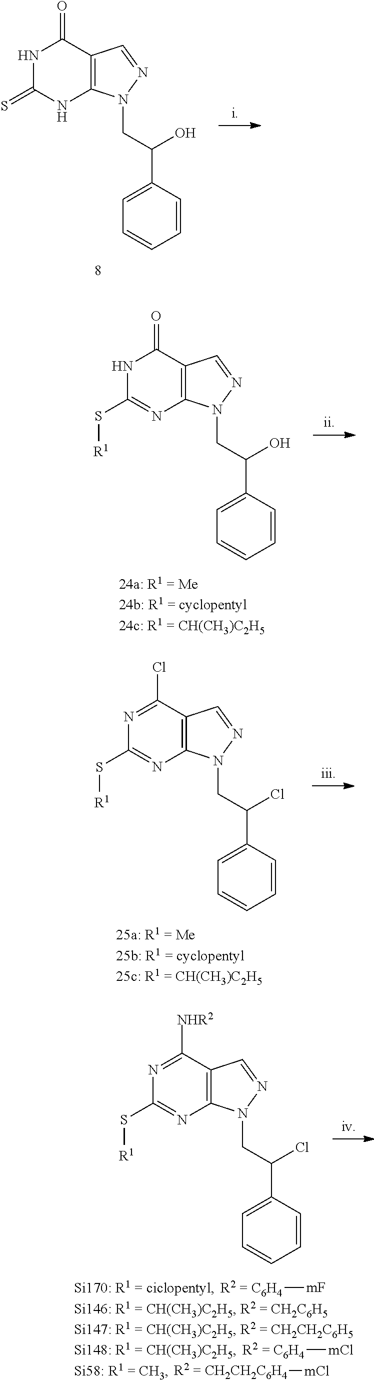 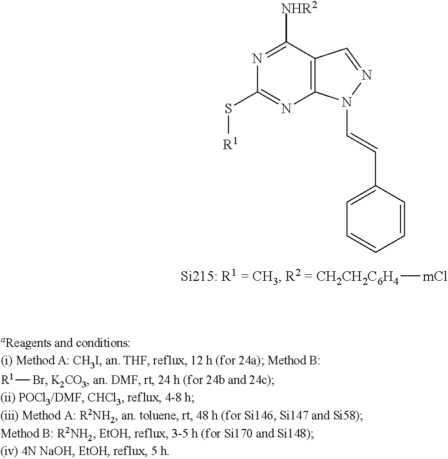
or a process for the preparation of compound Si74 of formula IV as defined in claim 11, or salts thereof, comprising the following steps: 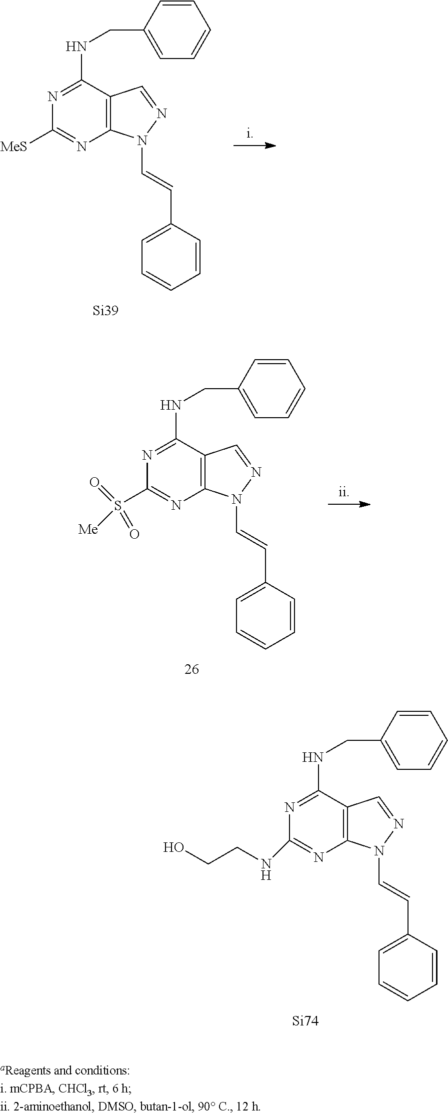 [SMILES: "Si39" ; CSc1nc(NCc2ccccc2)c2cnn(/C=C/c3ccccc3)c2n1 ; CS(=O)(=O)c1nc(NCc2ccccc2)c2cnn(/C=C/c3ccccc3)c2n1 ; "26" ; "Si74" ; OCCNc1nc(NCc2ccccc2)c2cnn(/C=C/c3ccccc3)c2n1]
or a process for the preparation of compound Si164 of formula IV as defined in claim 11, or salts thereof, comprising the following steps: [chemical structure]
